(19) United States
(12) Patent Application Publication
(10) Pub. No.: US 2025/0297242 A1
(43) Pub. Date: Sep. 25, 2025

(54) COMPOSITION AND METHOD FOR GENOME EDITING

(71) Applicant: QUIDDITAS SA, Marche-en-Famenne (BE)

(72) Inventor: François CHERBONNEAU, MONTLHÉRY (FR)

(73) Assignee: QUIDDITAS SA, Marche-en-Famenne (BE)

(21) Appl. No.: 18/573,692

(22) PCT Filed: Jun. 24, 2022
(86) PCT No.: PCT/EP2022/067351

§ 371 Date: Dec. 22, 2023

(30) Foreign Application Priority Data
Jun. 25, 2021 (FR) FR2106868

(51) Int. Cl. C12N 15/10; C12N 9/12; C12N 15/113; C12N 15/90

(52) U.S. Cl. CPC C12N 15/102; C12N 9/1241; C12N 15/113; C12N 15/907

(57) ABSTRACT

A molecular complex that contains: a first single-stranded nucleic acid molecule including at least two binding half-sites of a transposase, and a second single-stranded nucleic acid molecule including at least one binding half-site of a transposase. The complex is such that the first and second single-stranded nucleic acids are paired according to the base complementarity defined by Watson and Crick so as to define two double-stranded binding sites of said transposase. Also the use of the complex, in particular for DNA editing.

### REFERENCE TO A SEQUENCE LISTING

In accordance with 37 CFR § 1.821, the present specification makes reference to a Sequence Listing submitted electronically via Patent Center. The name of the file is “Corrected Sequence Listing.txt”. The .txt file was generated on Feb. 12, 2025 is 207,130 bytes in size. The entire contents of the Sequence Listing are hereby incorporated by reference.

### FIELD

The invention relates to a composition and a method for genome editing.

### BACKGROUND

The desire to understand the effects of modifying the genetic information of living cells dates back to the first steps of genetics.

First of all, conventional genetics attempts to understand genetic modifications, and the phenotype resulting therefrom, by selecting specific genetic sites.

Subsequently, biochemists have used radiation and chemical mutagenic agents to increase the probability of genetic mutations in experimental organisms. Although very useful, these methods are expensive, and do not allow easy control of the modifications introduced into the genetic material.

Molecular biology and knowledge of molecular mechanisms for cell repair or defense against host organisms have made it possible to develop numerous technologies, allowing the development of so-called reverse genetics, the objective of which is the opposite of so-called conventional genetic screening.

Reverse genetics aims to introduce mutations into genetic material with the aim of measuring and analyzing the resulting phenotypic effects.

Genome editing strategies have evolved over the last three decades, and the most recent innovation and revolution in the context of the targeted gene modification is the CRISPR/CRISPR associated with protein 9 (Cas9) system (CRISPR/Cas-9). In this respect, two patents—EP 3 144 390 B1 and U.S. Pat. No. 8,697,359 B1—may be mentioned, both of which describe this technology.

The CRISPR/Cas-9 system has therefore imposed itself as the reference tool for genetic modifications. However, this CRISPR revolution is similar to molecular scissors, which is not sufficient for allowing total recombination of an entire exon.

More recently, the transposase encoded by the CRISPR (Transposon-encoded CRISPR-Cas systems) system is a half-response to the deficiencies of CRISPR technology. Indeed, this technology uses both an integration targeted via transposon Tn7 followed by the addition of a sequence into the genome by CRISPR technology. But in no case is it a recombination.

Prime Editing technology is another possibility for gene replacement associating the CRISPR tool with a reverse transcriptase. This latest evolution in genome editing allows a replacement, depending on the size, of one of the two DNA strands, which remains a strong limitation that is inherent to and contingent on the cellular integration and repair system.

### SUMMARY

Also, the invention aims in particular to overcome these disadvantages of the prior art.

One of the aims of the invention is to provide a recombination tool that allows genome editing.

Another aim of the invention is for this new tool not to be dependent on the size of the manipulated sequence, and for this system to be controlled and controllable by the user.

Yet another aim of the invention is to provide a tool that enables, at the will of the user, a single-stranded or double-stranded replacement of a molecule of interest, using the DNA repair system in a controlled and limited manner.

The invention relates to a first single-stranded nucleic acid molecule comprising or consisting essentially of an A sequence allowing the insertion of a complementary sequence of a nucleic acid of interest,
said A sequence binding at 5′ to a first T-rich sequence of 40 to 60 nucleotides in length and at 3′ to a second T-rich sequence of 40 to 60 nucleotides in length, said first and second T-rich sequences respectively comprising a first and a second domain of 6 to 12 G/C-rich nucleotides, the sequence of the first domain being complementary to the sequence of the second domain, said first and second domains being positioned 15 to 52 nucleotides from said A sequence, said first molecule comprising at its 5′ end a first sequence oriented 5′-to-3′ for recognizing a transposase and at its 3′ end at least one second sequence for recognizing said transposase.

Also, the invention relates to a molecular complex comprising:
a first single-stranded nucleic acid molecule comprising or consisting essentially of an A sequence allowing the insertion of a complementary sequence of a nucleic acid of interest,
said A sequence binding at 5′ to a first A/T-rich, especially T-rich, sequence of 40 to 60 nucleotides in length and at 3′ to a second A/T-rich, especially T-rich, sequence of 40 to 60 nucleotides in length, said first and second A/T-rich, especially T-rich, sequences respectively comprising a first and a second domain of 6 to 12 G/C-rich nucleotides, the sequence of the first domain being complementary to the sequence of the second domain, said first and second domains being positioned 15 to 52 nucleotides from said A sequence, said first molecule comprising at its 5′ end a first sequence oriented 5′-to-3′ for recognizing a transposase and at its 3′ end at least one second sequence for recognizing said transposase; and
a second single-stranded nucleic acid molecule comprising or consisting essentially at its 5′ end of at least one complementary sequence of said second sequence for recognizing said transposase,
said complex being such that the first and second single-stranded nucleic acid molecules are paired according to the base complementarity defined by Watson and Crick so as to define two double-stranded binding sites of said transposase.

This means that the invention relates to a molecular complex comprising a first single-stranded nucleic acid molecule and a second single-stranded nucleic acid molecule, said second single-stranded nucleic acid molecule comprising or consisting essentially at its 5′ end of at least one complementary sequence of said second sequence for recognizing said transposase,
said complex being such that the first and second single-stranded nucleic acid molecules are paired according to the base complementarity defined by Watson and Crick so as to define two double-stranded binding sites of said transposase.

### BRIEF DESCRIPTION OF THE FIGURES

The invention will be better understood from reading the following examples and figures:

FIG. 1 is a schematic depiction of the first nucleic acid molecule in linear form. The rectangles with arrow heads depict sites for binding to a transposase, and the rectangles with oblique bars depict the G/C-rich regions.

FIG. 2 is a schematic depiction of the first nucleic acid molecule in structured form. The captions are the same as for [FIG. 1].

FIG. 3 is a schematic depiction of the complex according to the invention. The captions are the same as for [FIG. 1].

FIG. 4 is a schematic depiction of two versions of the complex according to the invention. The captions are the same as for [FIG. 1]. The different options are depicted in dotted lines.

FIG. 5 is a schematic depiction of a first embodiment of the complex according to the invention. The captions are the same as for [FIG. 1].

FIG. 6 is a schematic depiction of a second (6A) and a third (6B) embodiment of the complex according to the invention, or the 3′ region of the first sequence comprises two transposase half-sites. The captions are the same as for [FIG. 1].

FIG. 7 is a schematic depiction of a third embodiment of the complex according to the invention. The captions are the same as for [FIG. 1].

FIG. 8 is a schematic depiction of an ensemble according to the invention. The captions are the same as for [FIG. 1].

FIG. 9 is a schematic depiction of the sequence of certain steps of replacing a target sequence of a single-stranded molecule with a sequence of interest.
A: represents the unpaired target molecule and ensemble.
B: represents the paired target molecule and ensemble. The transposases are depicted in dotted lines, and the cleavages are depicted by scissors. It should be noted that the cleavage on the third molecule of the ensemble is carried out between the two transposase binding sites, on either side of the molecule (two cleavages).
C: represents the molecule resulting from tagmentation. The deletion of nine bases at 5′ of the replaced region is shown in dotted lines.

FIG. 10 is a schematic depiction of the sequence of certain steps of replacing a target sequence of a double-stranded molecule with a sequence of interest, itself double-stranded.
A: represents the unpaired target molecule and ensemble.
B: represents the paired target molecule and ensemble. The transposases are depicted in dotted lines, and the cleavages are depicted by scissors. It should be noted that the cleavage on the third molecule of the ensemble is carried out between the two transposase binding sites, on either side of the molecule (two cleavages).
C: represents the molecule resulting from tagmentation. The deletion of nine bases at 5′ of the replaced region is shown in dotted lines.

FIG. 11 depicts agarose gels showing the tagmentation according to the invention. a. Agarose gel with three groups of samples; test of the different transposase complexes (negative control, Tn5 WT, Tn5 Me and Tn5 DREAMT, i.e. according to the invention) with mCherry-CD9 plasmid, PCR amplification of HEK 293T total mRNAs, and PCR amplification of HEK 293T total mRNAs transfected with the mCherry-CD9 plasmid (addition of PCR+/−control). b. Agarose gel of different transposase mixes (see a.) ten times more concentrated on mCherry-CD9 plasmids.

FIG. 12 depicts the result of Sanger sequencing of the positive amplified band (Gel [FIG. 10] a) for the Tn5 DREAMT mix. The sequence (SEQ ID NO: 429) obtained and the corresponding chromatogram are presented. ** depicts the GFP insertion zone. The GFP sequence is flanked, and the mCherry sequence is underlined.

FIG. 13 depicts an agarose gel with three groups of samples; test of the different transposase complexes (negative control, Tn5 WT, Tn5 Me and Tn5 DREAMT) with mCherry-CD9 plasmid, PCR amplification of HEK 293T total cDNA, and PCR amplification of HEK 293T total cDNA transfected with mCherry-CD9 plasmid (addition of PCR+/−control).

FIG. 14 shows the result of Sanger sequencing of the positive amplified band (Gel [FIG. 13]) for the Tn5 DREAMT mix. The sequence obtained (SEQ ID NO: 430) and the corresponding chromatogram are presented. ** depicts the GFP insertion zone. The CD9 sequence is flanked.

FIG. 15 depicts an agarose gel with three groups of samples; test of the different transposase complexes (negative control, Tn5 WT, Tn5 Me and Tn5 DREAMT) with mCherry-CD9 plasmid, PCR amplification of HEK 293T total cDNA, and PCR amplification of HEK 293T total cDNA transfected with mCherry-CD9 plasmid (addition of PCR+/−control).

FIG. 16 shows the result of Sanger sequencing of the positive amplified band (Gel [FIG. 15]) for the Tn5 DREAMT mix. The sequence obtained (SEQ ID NO: 431) and the corresponding chromatogram are presented. ** depicts the GFP insertion zone. The CD9 sequence is flanked.

FIG. 17 depicts the test of the “new design” UVRD-mSA/Tn5/DREAMT complex on the mCherry-CD9 plasmid. Injection (transfection) of the mCherry-CD9 plasmid to the HEK 293T cells and then DREAMT technology, search for a visible color change (red->green as mentioned in the figure)

FIG. 18 depicts the transfection of the technology according to the invention in the HEK 293T cell line stably expressing mCherry-CD9, then the search for a visible color change (red->green as mentioned in the figure).

FIG. 19 depicts the Sanger sequencing results of the amplified GFP fragment from the cDNA library originating from the mCherry-CD9+ cell line of HEK 293T cells transfected with the “new design” DREAMT technology (cells used on D18)—SEQ ID NO: 432—the GFP replacement is shown flanked. The sequence SEQ ID NO: 433 depicts the theoretical GFP sequence (flanked part).

### DETAILED DESCRIPTION

The invention is based on the unexpected observation made by the inventor that the use of specific single-stranded guides capable of targeting a region of a nucleic acid of interest makes it possible to mobilize transposases in a controlled and “site-specific” manner, and thus to use the recombination properties of said transposases to replace sequences in molecules of interest.

The aforementioned molecular complex is in fact the basic unit of the technology defined in the invention. This basic unit is useful for guiding the recombinases to a specific site where the recombination, and therefore the sequence replacement, must take place. Unlike the CRISPR/Cas9 system, which requires the presence of PAM-type (NGG) sequences, the molecular tool defined herein may be used on any target sequence, regardless of its sequence.

The aforementioned molecular complex is therefore the basic unit to be completed by:
a homology region of the target sequence, and
a replacement region of the target sequence.

This is therefore an intermediate product of the tool as described hereinafter.

The molecular complex consists of two single-stranded nucleic acid molecules, which may be DNA molecules, RNA molecules or mixed RNA and DNA molecules.

These two molecules are partially complementary with one another, according to the base complementarity of nucleic acids defined by Watson and Crick, that is to say that adenine pairs with thymidine or uracil, and cytosine pairs with guanine, and vice versa.

More particularly, the two molecules forming the aforementioned complex each comprise the sequence of one of the strands of a double-stranded molecule corresponding to the binding sequence of a transposase. Also, each single-stranded molecule therefore comprises a transposase binding “half sequence” and therefore cannot allow an interaction with said corresponding transposase. On the other hand, when the two molecules of the complex interact together, by base pairing as defined hereinbefore, a double-stranded molecule is thus formed, reconstituting a double-stranded binding site of said transposase, the latter thus being able to interact with the molecule formed.

### The First Molecule.

The first molecule of the complex is the molecule that comprises, once modified, a nucleic acid sequence that makes it possible to specifically target a region of interest of a nucleic acid molecule of interest. This sequence of interest is selected by the user of the system according to the selected target. This sequence of interest is inserted into the first molecule of said complex at the A region. This A region corresponds at least to two nucleic acids between which the sequence that makes it possible to target the target molecule is inserted. In view of the oriented structure of the nucleic acids (5′-to-3′ direction), it is important for the sequence that makes it possible to target the region of interest to be positioned in the correct direction, in order to allow pairing with the target sequence.

Also, advantageously, the A region comprises one or more sites recognizing restriction enzymes in order to promote an oriented insertion. One or more of the following sites may be present in the A region:

TABLE 1

| | | | |
|---|---|---|---|
| SEQ ID NO: | 29 | AA/CGTT | AcII |
| SEQ ID NO: | 30 | A/AGCTT | HindIII |
| SEQ ID NO: | 31 | AAT/ATT | SspI |
| SEQ ID NO: | 32 | /AATT | MluCI |
| SEQ ID NO: | 33 | A/CATGT | PciI |
| SEQ ID NO: | 34 | JA/CCGGT | AgeI |
| SEQ ID NO: | 35 | ACCTGC(4/8) | BfuAI BspMI |
| SEQ ID NO: | 36 | A/CCWGGT | SexAI |
| SEQ ID NO: | 37 | A/CGCGT | MluI |
| SEQ ID NO: | 38 | ACGGC(12/14) | BceAI |
| SEQ ID NO: | 39 | A/CGT | HpyCH4IV |
| SEQ ID NO: | 40 | ACN/GT | HpyCH4III |
| SEQ ID NO: | 41 | (10/15)ACNNNNGTAYC(12/7) | BaeI |
| SEQ ID NO: | 42 | (9/12)ACNNNNNCTCC(10/7) | BsaXI |
| SEQ ID NO: | 43 | A/CRYGT | AflIII |
| SEQ ID NO: | 44 | A/CTAGT | SpeI |
| SEQ ID NO: | 45 | ACTGG(1/−1) | BsrI |
| SEQ ID NO: | 46 | ACTGGG(5/4) | BmrI |
| SEQ ID NO: | 47 | A/GATCT | BglII |
| SEQ ID NO: | 48 | JAGC/GCT | AfeI |
| SEQ ID NO: | 49 | AG/CT | AluI |
| SEQ ID NO: | 50 | AGG/CCT | StuI |
| SEQ ID NO: | 51 | AGT/ACT | ScaI− |
| SEQ ID NO: | 52 | AT/CGAT | ClaI BspDI |
| SEQ ID NO: | 53 | ATCTATGTCGGGTGCGGAGAAAGAGG | PI−SceI |
| | | TAAT(−15/−19) | |
| SEQ ID NO: | 54 | ATGCA/T | NsiI |
| SEQ ID NO: | 55 | AT/TAAT | AseI |
| SEQ ID NO: | 56 | ATTT/AAAT | SwaI |
| SEQ ID NO: | 57 | (11/13)CAANNNNNGTGG(12/10) | CspCI |
| SEQ ID NO: | 58 | C/AATTG | MfeI |
| SEQ ID NO: | 59 | CACCTGC(4/8) | PaqCI |
| SEQ ID NO: | 60 | CACGAG | Nb.BssSI |
| SEQ ID NO: | 61 | CACGAG(−5/−1) | BssSI−v2 |
| SEQ ID NO: | 62 | CACGTC(−3/−3) | BmgBI |
| SEQ ID NO: | 63 | CAC/GTG | PmII |
| SEQ ID NO: | 64 | CACNNN/GTG | DralII |
| SEQ ID NO: | 65 | CACNN/NNGTG | AleI−v2 |
| SEQ ID NO: | 66 | CAGCAG(25/27) | EcoP151 |
| SEQ ID NO: | 67 | CAG/CTG | PvuII |
| SEQ ID NO: | 68 | CAGNNN/CTG | AlwNI |
| SEQ ID NO: | 69 | CAGTG(2/0) | BtsIMutI |
| SEQ ID NO: | 70 | CA/TATG | NdeI |
| SEQ ID NO: | 71 | CATG/ | NlaIII |
| SEQ ID NO: | 72 | /CATG | FatI |
| SEQ ID NO: | 73 | C/ATG | CviAII |
| SEQ ID NO: | 74 | CAYNN/NNRTG | MsII |
| | | CC(12/16) | FspEI |
| SEQ ID NO: | 75 | CCANNNNN/NNNNTGG | XcmI |
| SEQ ID NO: | 76 | CCANNNNN/NTGG | BstXI |
| SEQ ID NO: | 77 | CCANNNN/NTGG | PflMI |
| SEQ ID NO: | 78 | CCATC(4/5) | BccI |
| SEQ ID NO: | 79 | C/CATGG | NcoI |
| SEQ ID NO: | 80 | CCCAGC(−5/−1) | BseYI |
| SEQ ID NO: | 81 | CCCGC(4/6) | FauI |
| SEQ ID NO: | 82 | CCC/GGG | SmaI |
| SEQ ID NO: | 83 | C/CCGGG(0/−1)CCD | TspMI XmaI |
| | | | Nt.CviPII |
| SEQ ID NO: | 84 | CCDG(10/14) | LpnPI |
| SEQ ID NO: | 85 | CCGC(−3/−1) | AciI |
| SEQ ID NO: | 86 | CCGC/GG | SacII |
| SEQ ID NO: | 87 | CCGCTC(−3/−3) | BsrBI |
| SEQ ID NO: | 88 | C/CGG | MspI HpaII |
| SEQ ID NO: | 89 | CC/NGG | ScrFI |
| SEQ ID NO: | 90 | /CCNGG | StyD4I |
| SEQ ID NO: | 91 | C/CNNGG | BsaJI |
| SEQ ID NO: | 92 | CCNNNNN/NNGG | BsII |
| SEQ ID NO: | 93 | C/CRYGG | BtgIl |
| SEQ ID NO: | 94 | CC/SGG | NciI |
| SEQ ID NO: | 95 | C/CTAGG | AvriI |
| SEQ ID NO: | 96 | CCTC(7/6) | MnII |
| SEQ ID NO: | 97 | CCTCAGC | Nb.BbvCI |
| SEQ ID NO: | 98 | CCTCAGC(−5/−7) | Nt.BbvCI |
| SEQ ID NO: | 99 | CCTCAGC(−5/−2) | BbvCI |
| SEQ ID NO: | 100 | CCTGCA/GG | SbfI |
| SEQ ID NO: | 101 | CCTNAGC(−5/−2) | Bpu10I |
| SEQ ID NO: | 102 | CC/TNAGG | Bsu36I |
| SEQ ID NO: | 103 | CCTNN/NNNAGG | EcoNI |
| SEQ ID NO: | 104 | CCTTC(6/5) | HpyAV |
| SEQ ID NO: | 105 | /CCWGG | PspGI |
| SEQ ID NO: | 106 | CC/WGG | BstNI |
| SEQ ID NO: | 107 | C/CWWGG | StyI |
| SEQ ID NO: | 108 | (10/12)CGANNNNNNTGC(12/10) | BcgI |
| SEQ ID NO: | 109 | CGAT/CG | PvuI |
| SEQ ID NO: | 110 | CG/CG | BstUI |
| SEQ ID NO: | 111 | C/GGCCG | EagI |
| SEQ ID NO: | 112 | CG/GWCCG | RsrII |
| SEQ ID NO: | 113 | CGRY/CG | BsiEI |
| SEQ ID NO: | 114 | C/GTACG | BsiWI |
| SEQ ID NO: | 115 | CGTCTC | BsmBI−v2 |
| SEQ ID NO: | 116 | CGTCTC(1/5) | Esp3I |
| SEQ ID NO: | 117 | CGWCG/ | Hpy99I |
| SEQ ID NO: | 118 | CMG/CKG | MspA1I |
| SEQ ID NO: | 119 | CNNNNNNNNNNN/NNNNNNNNNG | AbaSI |
| SEQ ID NO: | 120 | CNNR(9/13) | MspJI |
| SEQ ID NO: | 121 | CR/CCGGYG | SgrAI |
| SEQ ID NO: | 122 | C/TAG | BfaI |
| SEQ ID NO: | 123 | CTCAG(9/7) | BspCNI |
| SEQ ID NO: | 124 | C/TCGAG | XhoI PaeR7I |
| SEQ ID NO: | 125 | CTCTTC(1/4) | EarI |
| SEQ ID NO: | 126 | CTGAAG(16/14) | AcuI |
| SEQ ID NO: | 127 | CTGCA/G | PstI |
| SEQ ID NO: | 128 | CTGGAG(16/14) | BpmI |
| SEQ ID NO: | 129 | C/TNAG | DdeI |
| SEQ ID NO: | 130 | C/TRYAG | SfcI |
| SEQ ID NO: | 131 | C/TTAAG | AfIII |
| SEQ ID NO: | 132 | CTTGAG(16/14) | BpuEI |
| SEQ ID NO: | 133 | C/TYRAG | SmII |
| SEQ ID NO: | 134 | C/YCGRG | BsoBI AvaI |
| SEQ ID NO: | 135 | GAAGA(8/7) | MboII |
| SEQ ID NO: | 136 | GAAGAC(2/6) | BbsI |
| SEQ ID NO: | 137 | GAANN/NNTTC | XmnI |
| SEQ ID NO: | 138 | GAATGC(1/−1) | BsmI |
| SEQ ID NO: | 139 | GAATGC | Nb.BsmI |
| SEQ ID NO: | 140 | G/AATTC | EcoRI |
| SEQ ID NO: | 141 | GACGC(5/10) | HgaI |
| SEQ ID NO: | 142 | GACGT/C | AatIIII |
| SEQ ID NO: | 143 | GAC/GTC | ZraI |
| SEQ ID NO: | 144 | GACN/NNGTC | PfIFI Tth111I |
| SEQ ID NO: | 145 | GACNN/NNGTC | PshAI |
| SEQ ID NO: | 146 | GACNNN/NNGTC | AhdI |
| SEQ ID NO: | 147 | GACNNNN/NNGTC | DrdI |
| SEQ ID NO: | 148 | GAG/CTC | Eco53kI |
| SEQ ID NO: | 149 | GAGCT/C | SacI |
| SEQ ID NO: | 150 | GAGGAG(10/8) | BseRI |
| SEQ ID NO: | 151 | GAGTC(4/−5) | Nt.BstNBI |
| SEQ ID NO: | 152 | GAGTC(4/5) | PleI |
| SEQ ID NO: | 153 | GAGTC(5/5) | MlyI |
| SEQ ID NO: | 154 | G/ANTC | HinfI |
| SEQ ID NO: | 155 | GAT/ATC | EcoRV |
| SEQ ID NO: | 156 | GA/TC | DpnI |
| SEQ ID NO: | 157 | /GATC | Sau3AI DpnII |
| | | | MboI |
| SEQ ID NO: | 158 | GATNN/NNATC | BsaBI |
| SEQ ID NO: | 159 | G/AWTC | TfiI |
| SEQ ID NO: | 160 | GCAATG | Nb.BsrDI |
| SEQ ID NO: | 161 | GCAATG(2/0) | BsrDI |
| SEQ ID NO: | 162 | GCAGC(8/12) | BbvI |
| SEQ ID NO: | 163 | GCAGTG(2/0) | BtsI−v2 |
| SEQ ID NO: | 164 | GCAGTG | Nb.BtsI |
| SEQ ID NO: | 165 | GCANNNN/NTGC | BstAPI |
| SEQ ID NO: | 166 | GCATC(5/9) | SfaNI |
| SEQ ID NO: | 167 | GCATG/C | SphI |
| SEQ ID NO: | 168 | GCCC/GGGC | SrfI |
| SEQ ID NO: | 169 | GCCGAG(21/19) | NmeAIII |
| SEQ ID NO: | 170 | G/CCGGC | NgoMIV |
| SEQ ID NO: | 171 | GCC/GGC | NaeI |
| SEQ ID NO: | 172 | GCCNNNN/NGGC | BglI |
| SEQ ID NO: | 173 | GCGAT/CGC | AsiSI |
| SEQ ID NO: | 174 | GCGATG(10/14) | BtgZI |
| SEQ ID NO: | 175 | GCG/C | HhaI |
| SEQ ID NO: | 176 | G/CGC | HinP1I |
| SEQ ID NO: | 177 | G/CGCGC | BssHII |
| SEQ ID NO: | 178 | GC/GGCCGC | NotI |
| SEQ ID NO: | 179 | GC/NGC | Fnu4HI |
| SEQ ID NO: | 180 | GCN/NGC | Cac8I |
| SEQ ID NO: | 181 | GCNNNNN/NNGC | MwoI |
| SEQ ID NO: | 182 | G/CTAGC | NheI |
| SEQ ID NO: | 183 | GCTAG/C | BmtI |
| SEQ ID NO: | 184 | GCTCTTC(1/−7) | Nt.BspQI |
| SEQ ID NO: | 185 | GCTCTTC(1/4) | SapI BspQI |
| SEQ ID NO: | 186 | GC/TNAGC | BlpI |
| SEQ ID NO: | 187 | G/CWGC | ApeKI TseI |
| SEQ ID NO: | 188 | GDGCH/C | Bsp1286I |
| SEQ ID NO: | 189 | GGATC(4/5) | AlwI |
| SEQ ID NO: | 190 | GGATC(4/−5) | Nt.AlwI |
| SEQ ID NO: | 191 | G/GATCC | BamHI |
| SEQ ID NO: | 192 | GGATG(9/13) | FokI |
| SEQ ID NO: | 193 | GGATG(2/0) | BtsCI |
| SEQ ID NO: | 194 | GG/CC | HaeIII |
| SEQ ID NO: | 195 | GGCCGG/CC | FseI |
| SEQ ID NO: | 196 | GGCCNNNN/NGGCC | SfiI |
| SEQ ID NO: | 197 | G/GCGCC | KasI |
| SEQ ID NO: | 198 | 8GG/CGCC | NarI |
| SEQ ID NO: | 199 | GGCGC/C | PluTI |
| SEQ ID NO: | 200 | GGC/GCC | SfoI |
| SEQ ID NO: | 201 | GG/CGCGCC | AscI |
| SEQ ID NO: | 202 | GGCGGA(11/9) | EciI |
| SEQ ID NO: | 203 | GGGAC(10/14) | BsmFI |
| SEQ ID NO: | 204 | GGGCC/C | ApaI |
| SEQ ID NO: | 205 | G/GGCCC | PspOMI |
| SEQ ID NO: | 206 | G/GNCC | Sau96I |
| SEQ ID NO: | 207 | GGN/NCC | NlaIV |
| SEQ ID NO: | 208 | G/GTACC | Acc65I |
| SEQ ID NO: | 209 | GGTAC/C | KpnI |
| SEQ ID NO: | 210 | GGTCTC(1/5) | BsaI v2 |
| SEQ ID NO: | 211 | GGTGA(8/7) | HphI |
| SEQ ID NO: | 212 | G/GTNACC | BstEII |
| SEQ ID NO: | 213 | G/GWCC | AvaII |
| SEQ ID NO: | 214 | G/GYRCC | BanI |
| SEQ ID NO: | 215 | GKGCM/C | BaeGI |
| SEQ ID NO: | 216 | GR/CGYC | BsaHI |
| SEQ ID NO: | 217 | GRGCY/C | BanII |
| SEQ ID NO: | 218 | GT/AC | RsaI |
| SEQ ID NO: | 219 | G/TAC | CviQI |
| SEQ ID NO: | 220 | GTATAC | BstZ17I |
| SEQ ID NO: | 221 | GTATCC(6/5) | BciVI |
| SEQ ID NO: | 222 | G/TCGAC | SaII |
| SEQ ID NO: | 223 | GTCTC(1/5) | BsmAI BcoDI |
| SEQ ID NO: | 224 | GTCTC(1/−5) | Nt.BsmAl |
| SEQ ID NO: | 225 | G/TGCAC | ApaLI |
| SEQ ID NO: | 226 | GTGCAG(16/14) | BsgI |
| SEQ ID NO: | 227 | GT/MKAC | AccI |
| SEQ ID NO: | 228 | GTN/NAC | Hpy166II |
| SEQ ID NO: | 229 | /GTSAC | Tsp45I |
| SEQ ID NO: | 230 | GTT/AAC | HpaI |
| SEQ ID NO: | 231 | GTTT/AAAC | PmeI |
| SEQ ID NO: | 232 | GTY/RAC | HincII |
| SEQ ID NO: | 233 | GWGCW/C | BsiHKAI |
| SEQ ID NO: | 234 | NNCASTGNN/ | TspRI |
| SEQ ID NO: | 235 | R/AATTY | ApoI |
| SEQ ID NO: | 236 | RCATG/Y | NspI |
| SEQ ID NO: | 237 | R/CCGGY | BsrFI−v2 |
| SEQ ID NO: | 238 | R/GATCY | BstYI |
| SEQ ID NO: | 239 | RGCGC/Y | HaeII |
| SEQ ID NO: | 240 | RG/CY | CviKI−1 |
| SEQ ID NO: | 241 | RG/GNCCY | EcoO109I |
| SEQ ID NO: | 242 | RG/GWCCY | PpuMI |
| SEQ ID NO: | 243 | TAACTATAACGGTCCTAAGGTAGCGAA | I−CeuI |
| | | (−9/−13) | |
| SEQ ID NO: | 244 | TAC/GTA | SnaBI |
| SEQ ID NO: | 245 | TAGGGATAACAGGGTAAT(−9/−13) | I−SceI |
| SEQ ID NO: | 246 | T/CATGA | BspHI |
| SEQ ID NO: | 247 | T/CCGGA | BspEI |
| SEQ ID NO: | 248 | TCCRAC(20/18) | MmeI |
| SEQ ID NO: | 249 | T/CGA | TagI−v2 |
| SEQ ID NO: | 250 | TCG/CGA | NruI |
| SEQ ID NO: | 251 | TCN/GA | Hpy188I |
| SEQ ID NO: | 252 | TC/NNGA | Hpy188III |
| SEQ ID NO: | 253 | T/CTAGA | XbaI |
| SEQ ID NO: | 254 | T/GATCA | BclI |
| SEQ ID NO: | 255 | TG/CA | HpyCH4V |
| SEQ ID NO: | 256 | TGC/GCA | FspI |
| SEQ ID NO: | 257 | TGGCAAACAGCTATTATGGGTATTATG | PI−PspI |
| | | GGT(−13/−17) | |
| SEQ ID NO: | 258 | TGG/CCA | MscI |
| SEQ ID NO: | 259 | T/GTACA | BsrGI |
| SEQ ID NO: | 260 | T/TAA | MseI |
| SEQ ID NO: | 261 | TTAAT/TAA | PacI |
| SEQ ID NO: | 262 | TTA/TAA | PsiI−v2 |
| SEQ ID NO: | 263 | TT/CGAA | BstBI |
| SEQ ID NO: | 264 | TTT/AAA | DraI |
| SEQ ID NO: | 265 | VC/TCGAGB | PspXI |
| SEQ ID NO: | 266 | W/CCGGW | BsaWI |
| SEQ ID NO: | 267 | YAC/GTR | BsaAI |
| SEQ ID NO: | 268 | Y/GGCCR | EaeI |

Obviously, in the context of a chemical synthesis of the first molecule, it is not necessary to have cloning (or insertion) sites of the sequence making it possible to target the target region, but rather to take good care to provide a correctly oriented sequence. This is of course however possible.

The first molecule further consists, on either side of the A region, of A/T-rich sequences, or in the case of RNA, of A/U-rich sequences, in order to allow a certain flexibility of the structure. A/T-rich or A/U rich are understood in the invention to mean a sequence that comprises more than 50% of A or T, or U, preferably more than 50% of T or U, with respect to the total number of nucleotides that constitute the sequence. These sequences on either side of the A region have a size in nucleotides ranging from 10 nucleotides to 60 nucleotides.

The flexibility of these sequences flanking the A region, due to the presence of numerous A, T or U bases, may have the effect of allowing a recombination via the recombinases that is not sufficiently controlled, maybe even when the complex still has not recognized the target molecule.

Also, in order to overcome this problem, GC-rich sequences are introduced into each of the A/T-rich, especially T-rich, or A/U-rich, sequences bordering the A region. These G/C-rich regions consist of 6 to 12 nucleotides, in which the amount of C or G bases is greater than 50% of the nucleotides contained in said G/C-rich sequence.

In order to stabilize the structure of the first molecule and, as described hereinbefore, prevent inadvertent recombination, the G/C-rich regions are positioned 15 to 52 nucleotides from the end of the A region.

For greater clarity, if the A region consists of three nucleotides, the central nucleotide corresponding to position 0, the A/T-rich or A/U-rich region begins on the left at position-2, and on the right at position +2. Therefore, on the left, the G/C-rich region is positioned from position-17 to position-54 and, on the right, from position +17 to position +54.

Another important element: the G/C-rich sequence to the right (or 5′) of the A region is necessarily complementary (according to the Watson and Crick pairing rule) to the G/C-rich region to the right (or 3′) of the A region. Also, the first single-stranded molecule pairs with itself at the G/C-rich regions, which prevents any recombination by the transposases, as long as there is no interaction with the complementary target sequence of the region that is inserted into the A region of the first molecule.

Finally, the first molecule comprises at its 5′ end a sequence corresponding to a first site for binding to a transposase, and at its 3′ end a second site for binding to said transposase.

The first binding site and the second binding site are advantageously the same, and above all both correspond to the same strand of the double-stranded binding site of said transposase. This means that the first transposase binding site present in the 5′ region of the first molecule can only be paired integrally, and therefore stably, with the transposase binding site present in the 3′ region.

The first binding site and the second binding site are advantageously the same, but each correspond to a different strand of the double-stranded transposase binding site. Also, for example, if the first transposase binding site corresponds to the sense strand, the second transposase binding site corresponds to the sequence of the complementary strand. It is then possible to have two configurations: either i) the second binding site which corresponds to the complementary strand is oriented in the 3′-to-5′ direction, in which case it is able to pair with the first transposase binding site and form the double-stranded site, or ii) the second binding site which corresponds to the complementary strand is oriented in the 5′-to-3′ direction, in which case it is not able to pair with the first transposase binding sequence, due to their orientation not being complementary. In the aforementioned case i), if the first single-stranded molecule pairs with itself at the first and second binding sites, it is not possible to form the aforementioned complex, since there are no more single-stranded complementary regions available to pair with the second molecule so as to form two double-stranded transposase binding sites.

Also, the first molecule, when it lacks a complementary sequence of the target region in the A part, or when it contains such a target sequence but the latter does not interact (does not pair) with said target sequence, forms a three-dimensional structure wherein the entire molecule is single-stranded with the exception of the region corresponding to the G/C-rich regions that pair with one another.

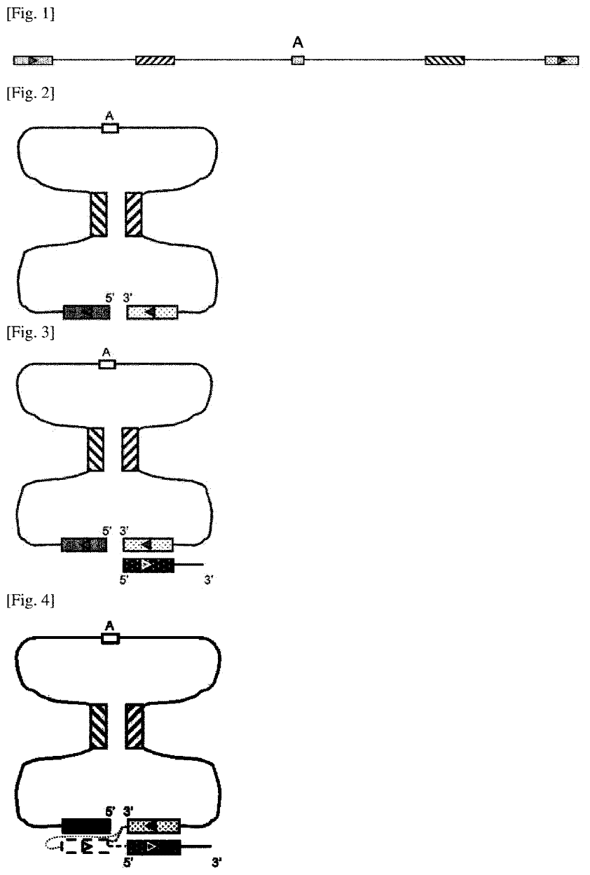

A linear schematic depiction of the first molecule is depicted in [FIG. 1], and a schematic depiction of its paired form is depicted in [FIG. 2].

### The Second Molecule.

The second molecule of the aforementioned complex is simpler than the first. It comprises, in its 5′ part, a transposase binding site which is complementary to the site for binding to said transposase present in the 3′ part of the first molecule. Therefore, when the complex is formed, the (single-stranded) transposase binding half-site located at 3′ of the first molecule may pair with the (single-stranded) transposase binding half-site located at 5′ of the second molecule so as to form a double-stranded transposase binding site, a double-stranded site on which the transposase can bind.

In the 3′ part of the second molecule is a region similar to the A region of the first molecule, this region making it possible to receive a specific sequence, which corresponds to the sequence to be inserted instead of the target molecule of interest. The following is a more detailed description of how to prepare a second molecule allowing this substitution.

### The Complex

The complex formed of the first molecule and the second molecule is depicted schematically in [FIG. 3].

The complex is such that when the first molecule and the second molecule are paired, via the transposase binding half-sites, the complex is capable of binding a transposase dimer, a functional dimer that allows the recombination.

Also, either one of the first or second molecules further comprises a complementary sequence of the transposase binding half-site located at 5′ of the first molecule. This complementary region of the transposase binding site located at 5′ of the first molecule can be located at 5′ or 3′ of the first molecule, or even at 5′ of the second molecule, preferably at 5′ of the complementary sequence of the transposase binding site located at 3′ of the first molecule.

In the invention “said complex being such that the first and second single-stranded nucleic acid molecules are paired according to the base complementarity defined by Watson and Crick so as to define two double-stranded binding sites of said transposase”. As the first molecule comprises at least one transposase binding half-site in its 5′ region and at least one transposase binding half-site in its 3′ region, and the second molecule also comprises at least one transposase binding half-site, this means that, during the pairing between the first and the second molecule, two complete sites are formed because
either
the first molecule comprises in its 5′ region a first sequence of a first transposase binding site and the complementary sequence of the first sequence of the first transposase binding site, and in its 3′ part a second sequence of a second transposase binding site, and
the second molecule comprises the complementary sequence of the second sequence of the second transposase binding site,
either
the first molecule comprises in its 5′ region a first sequence of a first transposase binding site, and in its 3′ part a second sequence of a second transposase binding site and the complementary sequence of the first sequence of the first transposase binding site, and
the second molecule comprises the complementary sequence of the second sequence of the second transposase binding site,
either
the first molecule comprises in its 5′ region a first sequence of a first transposase binding site, and in its 3′ part a second sequence of a second transposase binding site, and
the second molecule comprises the complementary sequence of the first sequence of the first transposase binding site, and the complementary sequence of the second sequence of the second transposase binding site.

The terminology used “at least”, and the fact that the molecule “comprises” sequences forming transposase recognition sites allow a person skilled in the art to select the position of the half-sequences forming a binding site, so that ultimately, when the complex is formed two whole sites are reconstituted.

Three options, two of which are detailed below, are depicted schematically in [FIG. 4].

Advantageously, the invention relates to the aforementioned complex, wherein said A sequence comprises a complementary sequence of a nucleic acid of interest.

As mentioned above, the A region may contain a complementary sequence of a nucleic acid of interest. More particularly, the sequence contained in the A region of the first molecule of the aforementioned complex is complementary to a sequence at 5′ or at 3′ of a sequence of a molecule of interest, so that the complex allows the specific recognition of this region of the nucleic acid molecule, and allows the complex to replace a region adjacent to the region complementary to the region complementary to the sequence contained in the A region.

In other words, the invention advantageously relates to the aforementioned complex, said complex comprising:
a first single-stranded nucleic acid molecule comprising or consisting essentially of an A sequence allowing the insertion of a complementary sequence of a nucleic acid of interest, said complementary A sequence binding at 5′ to a first A/T-rich, especially T-rich, sequence of 40 to 60 nucleotides in length and at 3′ to a second A/T-rich, especially T-rich, sequence of 40 to 60 nucleotides in length, said first and second A/T-rich, especially T-rich, sequences respectively comprising a first and a second domain of 6 to 12 G/C-rich nucleotides, the sequence of the first domain being complementary to the sequence of the second domain, said first and second domains being positioned 15 to 52 nucleotides from said A sequence, said first molecule comprising at its 5′ end a first sequence oriented 5′-to-3′ for recognizing a transposase and at its 3′ end at least one second sequence for recognizing said transposase; and
a second single-stranded nucleic acid molecule comprising or consisting essentially at its 5′ end of at least one complementary sequence of said second sequence for recognizing said transposase,
the first and second single-stranded nucleic acid molecules being paired according to the base complementarity defined by Watson and Crick so as to define two double-stranded binding sites of said transposase.

In one advantageous embodiment, the invention relates to an aforementioned complex, wherein said first molecule comprises at its 5′ end a first sequence oriented 5′-to-3′ for recognizing a transposase and at its 3′ end a second sequence oriented 5′-to-3′ for recognizing said transposase and
wherein the second molecule comprises its 5′ end a first complementary sequence of said first sequence for recognizing said transposase followed by a second complementary sequence of said second sequence for recognizing said transposase.

In this advantageous embodiment of the complex of the invention, the first molecule comprises a first transposase recognition sequence at position 5′ and a second transposase recognition sequence at position 3′. The second molecule in turn comprises at position 5′ a first complementary sequence of the first transposase recognition sequence of the first molecule, followed by a second complementary sequence of the second transposase recognition sequence of the first molecule. Also, each molecule of the complex comprises two transposase binding half-sites, so that, when the complex is formed, that is to say, when the first molecule pairs with the second molecule, two adjacent double-stranded transposase binding site are formed, and a transposase dimer can then bind thereto.

FIG. 5 schematically depicts this embodiment.

Advantageously, the invention relates to the aforementioned complex, wherein said first molecule comprises at its 5′ end a first sequence oriented 5′-to-3′ for recognizing a transposase and at its 3′ end a second sequence for recognizing said transposase, followed by a first complementary sequence of said first sequence for recognizing said transposase and

wherein the second molecule comprises at its 5′ end a complementary sequence of said second sequence for recognizing said transposase.

In this advantageous embodiment of the complex of the invention, the first molecule comprises a first transposase recognition sequence at position 5′ and a second transposase recognition sequence at position 3′, the latter being immediately followed by a first complementary sequence of the transposase recognition sequence located at 5′ of the first molecule. The second molecule in turn comprises at position 5′ a second complementary sequence of the second transposase recognition sequence of the first molecule.

Also, in this embodiment, the first molecule can reform a transposase recognition double-stranded binding site, by pairing the first recognition sequence at 5′ of the first molecule and the first recognition sequence at 3′ of the molecule. The second molecule in turn must be paired with the first molecule in order to reconstitute the second double-stranded transposase binding site using the second recognition sequence at 3′ of the first molecule and the second complementary transposase recognition sequence located at 5′ of the second molecule.

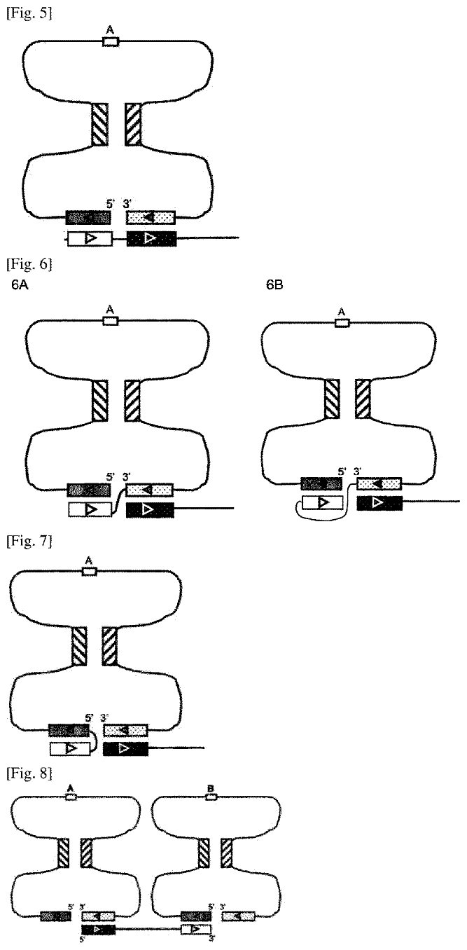

FIG. 6B schematically depicts this embodiment.

It is also possible to envisage another advantageous embodiment of the complex according to the invention wherein the first molecule comprises at 5′ a first complementary sequence of a first transposase recognition site, followed by first transposase recognition site. Furthermore, at 3′, the first molecule comprises a second transposase recognition site. The second molecule in turn remains unchanged with respect to the previously described embodiment.

Herein, the 5′ part of the first molecule folds onto itself so as to reconstitute, by pairing, a double-strand transposase recognition site, by means of the first recognition site and the immediately adjacent complementary sequence. This embodiment is presented in [FIG. 7].

An additional similar embodiment exists wherein the first molecule comprises at 5′ the first complementary sequence of the first transposase site immediately followed by the first site for recognizing the first transposase.

Advantageously, the aforementioned transposase is a bacterial-type transposase selected from the transposase of transposon Tn5, the transposase of transposon Tn9, the transposase of transposon Tn10, Tn903, Tn602, or even the transposase of the transposon Tc1, or more generally of the mariner transposon superfamily.

Other examples of transposases that can be used in the context of the invention are: the Vibrio harveyi transposase (transposase characterized by Agilent and used in the product SureSelect QXT), the MutA transposase and a Mu transposase recognition site comprising the terminal sequences R1 and R2, the transposase of Staphylococcus aureus transposon Tn552, the transposase of transposon Tn7, the Tn/O and IS10 transposase, the transposase of transposon Tn3.

The Tn5 transposase is the best known. It is coded by the Tnp gene of transposon Tn5. The transposase initiates the transposition by forming a transposase dimer which binds to its target sequences. In the context of this complex, the transposase then catalyzes four phosphoryl transfer reactions (DNA cleavage, DNA hairpin formation, hairpin resolution and strand transfer to the target DNA), resulting in the integration of the transposon into its new DNA site: this is what is known as “tagmentation”.

The invention is based on this tagmentation principle. By using the tagmentation properties of the transposases, it is possible to insert one sequence into another in a targeted manner, by virtue of the aforementioned complex.

Also in the context of the invention, when reference is made to a transposase, reference is being made to one of the aforementioned transposases, namely the transposases of transposons Tn5, Tn9, Tn10 or Tc1/mariner (or transposases mutated to increase their transposition or tagmentation activity).

In the invention, when several transposases are used simultaneously, one binding to the complex formed by the first molecule and the third molecule, and the other binding to the complex formed by the second molecule and the third molecule, the pairs of transposases resulting from transposons Tn5 and Tn10 are preferred.

In one advantageous embodiment, the invention relates to a kit comprising a vector allowing the expression of the first molecule of the aforementioned complex, and a vector allowing the expression of the aforementioned second molecule.

In the context of this kit, the vectors are preferentially circular molecules, of double-stranded DNA which have all the elements for allowing their replication in host cells (prokaryotic and or eukaryotic) and which have elements for allowing the expression of the first or the second molecule of the aforementioned complex.

In the event that a first molecule and a second molecule must be in the form of single-stranded DNA molecules, the sequence of each of said first and second molecules is under the control of a sequence enabling the synthesis of single-stranded DNA from double-stranded DNA. This is the case, for example, of the origin of replication sequence of the f1 bacteriophage contained in phagemid-type vectors. In the presence of an auxiliary phase M13, which carries all the genes necessary for activating the f1 sequence, the vector therefore produces single-stranded DNA from the double-stranded plasmid DNA.

The kit can therefore contain either two independent vectors each containing the sequence of one or the other of the first and second molecules forming the aforementioned complex, or a single vector comprising the two sequences, but isolated genetically from one another.

The aforementioned kit may also contain other elements such as a transposase allowing the transposition.

Advantageously, the aforementioned complex is such that the first and the second transposase recognition sequence is a sequence for recognizing the Tn5 transposase having one of the following sequences:

(SEQ ID NO: 1)
CTGtCTCTTataCAcAtcT,
(SEQ ID NO: 3)
CTGACTCTTataCACAagT,
and
(SEQ ID NO: 5)
CTGtCTCTTgatCAgATCT.

As a result, the corresponding complementary sequences are as follows:

(SEQ ID NO: 2)
AgaTgTGtatAAGAGaCAG,
(SEQ ID NO: 4)
ActTGTGtatAAGAGTCAG,
and
(SEQ ID NO: 6)
AGATcTGatcAAGAGaCAG.

Other transposase recognition sequences are as follows:

Tn5MErev,
(SEQ ID NO: 11)
5′-[phos]CTGTCTCTTATACACATCT-3′
Tn5ME-A (Illumina FC-121-1030),
(SEQ ID NO: 12)
5′-TCGTCGGCAGCGTCAGATGTGTATAAGAGACAG-3′;
and
Tn5ME-B (Illumina FC-121-1031),
(SEQ ID NO: 13)
5′-GTCTCGTGGGCTCGGAGATGTGTATAAGAGACAG-3′

Further sequences are as follows
sense sequence SEQ ID NO: i
antisense sequence SEQ ID NO: i+1,
wherein i ranges from 269 to 424.

This means for example that the following respective sense and antisense sequence pairs are considered: SEQ ID NO: 269 and SEQ ID NO: 270; SEQ ID NO: 271 and SEQ ID NO: 272; SEQ ID NO: 273 and SEQ ID NO: 274; SEQ ID NO: 275 and SEQ ID NO: 276; SEQ ID NO: 277 and SEQ ID NO: 278; SEQ ID NO: 279 and SEQ ID NO: 280; SEQ ID NO: 281 and SEQ ID NO: 282; SEQ ID NO: 283 and SEQ ID NO: 284; SEQ ID NO: 285 and SEQ ID NO: 286; SEQ ID NO: 287 and SEQ ID NO: 288; SEQ ID NO: 289 and SEQ ID NO: 290; SEQ ID NO: 291 and SEQ ID NO: 292; SEQ ID NO: 293 and SEQ ID NO: 294; SEQ ID NO: 295 and SEQ ID NO: 296; SEQ ID NO: 297 and SEQ ID NO: 298; SEQ ID NO: 299 and SEQ ID NO: 300; SEQ ID NO: 301 and SEQ ID NO: 302; SEQ ID NO: 303 and SEQ ID NO: 304; SEQ ID NO: 305 and SEQ ID NO: 306; SEQ ID NO: 307 and SEQ ID NO: 308; SEQ ID NO: 309 and SEQ ID NO: 310; SEQ ID NO: 311 and SEQ ID NO: 312; SEQ ID NO: 313 and SEQ ID NO: 314; SEQ ID NO: 315 and SEQ ID NO: 316; SEQ ID NO: 317 and SEQ ID NO: 318; SEQ ID NO: 319 and SEQ ID NO: 320; SEQ ID NO: 321 and SEQ ID NO: 322; SEQ ID NO: 323 and SEQ ID NO: 324; SEQ ID NO: 325 and SEQ ID NO: 326; SEQ ID NO: 327 and SEQ ID NO: 328; SEQ ID NO: 329 and SEQ ID NO: 330; SEQ ID NO: 331 and SEQ ID NO: 332; SEQ ID NO: 333 and SEQ ID NO: 334; SEQ ID NO: 335 and SEQ ID NO: 336; SEQ ID NO: 337 and SEQ ID NO: 338; SEQ ID NO: 339 and SEQ ID NO: 340; SEQ ID NO: 341 and SEQ ID NO: 342; SEQ ID NO: 343 and SEQ ID NO: 344; SEQ ID NO: 345 and SEQ ID NO: 346; SEQ ID NO: 347 and SEQ ID NO: 348; SEQ ID NO: 349 and SEQ ID NO: 350; SEQ ID NO: 351 and SEQ ID NO: 352; SEQ ID NO: 353 and SEQ ID NO: 354; SEQ ID NO: 355 and SEQ ID NO: 356; SEQ ID NO: 357 and SEQ ID NO: 358; SEQ ID NO: 359 and SEQ ID NO: 360; SEQ ID NO: 361 and SEQ ID NO: 362; SEQ ID NO: 363 and SEQ ID NO: 364; SEQ ID NO: 365 and SEQ ID NO: 366; SEQ ID NO: 367 and SEQ ID NO: 368, SEQ ID NO: 369 and SEQ ID NO: 370; SEQ ID NO: 371 and SEQ ID NO: 372; SEQ ID NO: 373 and SEQ ID NO: 374; SEQ ID NO: 375 and SEQ ID NO: 376; SEQ ID NO: 377 and SEQ ID NO: 378; SEQ ID NO: 379 and SEQ ID NO: 380; SEQ ID NO: 381 and SEQ ID NO: 382; SEQ ID NO: 383 and SEQ ID NO: 384; SEQ ID NO: 385 and SEQ ID NO: 386; SEQ ID NO: 387 and SEQ ID NO: 388; SEQ ID NO: 389 and SEQ ID NO: 390; SEQ ID NO: 391 and SEQ ID NO: 392; SEQ ID NO: 393 and SEQ ID NO: 394; SEQ ID NO: 395 and SEQ ID NO: 396; SEQ ID NO: 397 and SEQ ID NO: 398; SEQ ID NO: 399 and SEQ ID NO: 400; SEQ ID NO: 401 and SEQ ID NO: 402; SEQ ID NO: 403 and SEQ ID NO: 404; SEQ ID NO: 405 and SEQ ID NO: 406; SEQ ID NO: 407 and SEQ ID NO: 408; SEQ ID NO: 409 and SEQ ID NO: 410; SEQ ID NO: 411 and SEQ ID NO: 412; SEQ ID NO: 413 and SEQ ID NO: 414; SEQ ID NO: 415 and SEQ ID NO: 416; SEQ ID NO: 417 and SEQ ID NO: 418; SEQ ID NO: 419 and SEQ ID NO: 420; SEQ ID NO: 421 and SEQ ID NO: 422; SEQ ID NO: 423 and SEQ ID NO: 424;

Advantageously, the first G/C-rich domain of the first molecule corresponds to the following sequence GG CGATCGC (SEQ ID NO: 425) so that the second G/C-rich domain is the same. Indeed, due to the folding of the molecule onto itself, the second G/C-rich domain is in a complementary and antiparallel orientation with respect to the first G/C-rich domain, and the interaction takes place at the palindromic region (underlined in the sequence hereinbefore).

The first and second G/C-rich domains may also be the following sequence GCG GCGATCGGC (SEQ ID NO: 426). The explanations hereinbefore apply mutatis mutandis.

Other G/C-rich domain sequences may be as follows:
first G/C-rich domain of sequence GGTCGC (SEQ ID NO: 427) and the second C/C-rich domain of sequence GCGACC (SEQ ID NO: 428).

These examples are given only by way of illustration and cannot limit the scope of the invention.

In one advantageous embodiment, the A/T-rich sequences of the first molecule of said complex consist essentially, or are made up of A or T.

Even more advantageously, the A/T-rich sequence of the first molecule of said complex consists of T.

Even more advantageously, the aforementioned complex is such that it comprises the following sequence corresponding to the first molecule:

5′-TGCAGCTGCTGTCTCTTATACACATCTTTTTTTTTTTTTTTTTTTTTTTTTTTTGGCGATCGC
TTTTTTTTTTTTTTTTTTTTXTTTTTTTTTTTTTTTTTTTTGCGATCGCCTTTTTTTTTTTTTTTT
GATACATGTTT AGATGTGTATAAGAGACAGCTGTAAGC-3' SEQ ID NO: sumarized by
M-X-N
wherein M is:
(SEQ ID NO: 7)

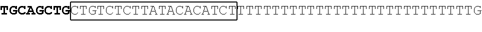

GCGATCGCTTTTTTTTTTTTTTTTTTTT,
N is
(SEQ ID NO: 442)
TTTTTTTTTTTTTTTTTTTTGCGATCGCCTTTTTTTTTTTTTTTTGATACATGTTT
AGATGTGTATAAGAGACAGCTGTAAGC

wherein X represents no nucleotide, two nucleotides or at least one restriction site.

The first transposase binding site is depicted flanked and the second transposase binding site is depicted underlined.

Even more advantageously, the aforementioned complex is such that it comprises the following sequence corresponding to the second molecule:

5′-AGATGTGTATAAGAGACAGCAGCTGCAGACAAAGCTTACAGCTGTCTCTTATACACATCTTTTTTT
TTTTTTTTTTTTTTTTcatatgccaagtY-3′ sumarized by O-YSEQ ID NO:
wherein O is:
(SEQ ID NO: 8)

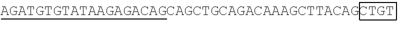

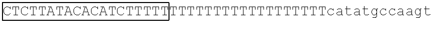

wherein Y represents no nucleotide, two nucleotides or at least one restriction site.

The first transposase binding site is depicted flanked and the second transposase binding site is depicted underlined.

Advantageously, the aforementioned complex is such that it comprises the following sequence corresponding to the first molecule:

5′-ATCATCCTGTCTCTTATACACATCTTTTTTTTTTTTTTTTTTGATAGTAGCTGTCTCTTATACACATCT
TTTTTTTTTTTTTTTTTTTTTTGGCGATCGCTTTTTTTTTTTTTTTTXTTTTTTTTTTTTTTTTGCGAT
CGCCTTTTTTTTTTTTTGATACATTT AGATGTGTATAAGAGACAG GATGAT-3′ SEQ ID NO:
sumarized by M-X-N
Wherein M is:
(SEQ ID NO:9)

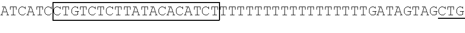

TCTCTTATACACATCTTTTTTTTTTTTTTTTTTTTTTTGGCGATCGCTTTTTTTTTTTTTTTT
N is:
(SEQ ID NO: 444)
TTTTTTTTTTTTTTTTGCGATCGCCTTTTTTTTTTTTTGATACATTTAGATGTGTATAAGAGACAGGATGAT

wherein X represents no nucleotide, two nucleotides or at least one restriction site.

The complementary sequence of the first transposase binding site and the second transposase binding site is depicted underlined, and the first transposase binding site is depicted in italics and underlined.

In this embodiment, the aforementioned complex is such that it comprises the following sequence corresponding to the second molecule:

(SEQ ID NO: 10)
5′-
YacttggTTAATTAATTTTTTTTTTTTTTTTTTTTTTAGATGTGTATAA
GAGACAGCTACTATC-3′ SEQ ID NO: sumarized by Y-O
wherein O is
acttggTTAATTAATTTTTTTTTTTTTTTTTTTTTTTTTAGATGTGTA
TAAGAGACAGCTACTATC

wherein Y represents no nucleotide, two nucleotides or at least one restriction site.

The complementary sequence of the second transposase binding site is depicted underlined.

Advantageously, the invention relates to the following complexes:
a first molecule of sequence

5′-TGCAGCTGR1TTTTTTTTTTTTTTTTTTTTTTTTTTTGGCGATCGCTTTTTTT
TTTTTTTTTTTTTXTTTTTTTTTTTTTTTTTTTTGCGATCGCCTTTTTTTTTTT
TTTTTGATACATGTTTR2CTGTAAGC-3′ SEQ ID NO: sumarized by M1R1M2-
X-M3R2M4
Wherein M1 is
(SEQ ID NO: 436)
TGCAGCTG
Wherein R1 is
(SEQ ID NO: 1)
5′-CTGtCTCTTataCAcAtcT,
wherein M2 is
(SEQ ID NO: 437)
TTTTTTTTTTTTTTTTTTTTTTTTTTTGGCGATCGCTTTTTTTTTTTTTTTTTTTT
M3 is
(SEQ ID NO: 438)
TTTTTTTTTTTTTTTTTTTTGCGATCGCCTTTTTTTTTTTTTTTTGATACATGTTT
R2 is
(SEQ ID NO: 2)
5′-AgaTgTGtatAAGAGaCAG,
and
M4 is
(SEQ ID NO: 439)
CTGTAAGC,

and

Wherein X corresponds to the sequence allowing the recognition of the target region
and
   a second molecule comprising the following sequence:

5′-
R2CAGCTGCAGACAAAGCTTACAGR1TTTTTTTTTTTTTTTTTTTTTTcatatg
ccaagtY-3′ SEQ ID NO: sumarized by R2M5R1M6-Y
wherein R2 is
(SEQ ID NO: 2)
5′-AgaTgTGtatAAGAGaCAG,
M5 is
(SEQ ID NO: 440)
CAGCTGCAGACAAAGCTTACAG
Wherein R1 is
(SEQ ID NO: 1)
5′-CTGtCTCTTataCAcAtcT,
R2 is
(SEQ ID NO: 2)
5′-AgaTgTGtatAAGAGaCAG,
M6 is
(SEQ ID NO: 441)
TTTTTTTTTTTTTTTTTTTTTTcatatgccaagt,

and wherein
Y corresponds to no nucleotide, or to the replacement sequence of the target region.

This means that the complex consists of molecules of the following sequence

5′-TGCAGCTGCTGtCTCTTataCAcAtcTTTTTTTTTTTTTTTTTTTTTTTTTTTTG
GCGATCGCTTTTTTTTTTTTTTTTTTTTXTTTTTTTTTTTTTTTTTTTTGCGA
TCGCCTTTTTTTTTTTTTTTTGATACATGTTTAgaTgTGtatAAGAGaCAG
CTGTAAGC-3′ SEQ ID NO: sumarized by M-X-N,
wherein M is SEQ ID NO: 7 and N is
(SEQ ID NO: 443)
TTTTTTTTTTTTTTTTTTTTGCGATCGCCTTTTTTTTTTTTTTTTGATACATGTTTAga
TgTGtatAAGAGaCAG CTGTAAGC
And
5′-AgaTgTGtatAAGAGaCAGCAGCTGCAGACAAAGCTTACAGCTGtCTCTTata
CAcAtcTTTTTTTTTTTTTTTTTTTTTTTcatatgccaagtY-3′ SEQ ID NO:
sumarized by Y-O

Wherein O is

(SEQ ID NO: 8)
AgaTgTGtatAAGAGaCAGCAGCTGCAGACAAAGCTTACAGCTGTCTCT
TataCAcAtcTTTTTTTTTTTTTTTTTTTTTTTcatatgccaagt

Or X and Y are as defined hereinbefore.

Advantageously, the invention relates to the following complexes:
a first molecule of sequence

(SEQ ID NO: 449)
5′-ATCATCR1TTTTTTTTTTTTTTTTTGATAGTAGR1TTTTTTTTTTTTTTTTTTT
TTTTGGCGATCGCTTTTTTTTTTTTTTTT
XTTTTTTTTTTTTTTTTGCGATCGCCTTTTTTTTTTTTTGATACATTTR2GAT
GAT-3′ SEQ ID NO:sumarized by X1R1X2R1X3-X-X4R2X5
wherein X1 is
(SEQ ID NO: 445)
ATCATC
Wherein R1 is
(SEQ ID NO: 1)
5′-CTGtCTCTTataCAcAtcT,
wherein X2 is
(SEQ ID NO: 446)
TTTTTTTTTTTTTTTTTGATAGTAG
wherein X3 est
(SEQ ID NO: 447)
TTTTTTTTTTTTTTTTTGGCGATCGCTTTTTTTTTTTTTTTT
wherein X4 is
(SEQ ID NO: 448)
TTTTTTTTTTTTTTTTGCGATCGCCTTTTTTTTTTTTTGATACATTT
R2 is 5′
(SEQ ID NO: 2)
AgaTgTGtatAAGAGaCAG,
and
X5 is
(SEQ ID NO: 449)
GATGAT

X corresponds to the sequence allowing the recognition of the target region
and
   a second molecule comprising the following sequence:

5′-YacttggTTAATTAATTTTTTTTTTTTTTTTTTTTTTR2CTACTATC-3′
SEQ ID NO:sumarized by Y-X6R2X7
Wherein X6 est
(SEQ ID NO: 450)
acttggTTAATTAATTTTTTTTTTTTTTTTTTTTTT
SEQ ID NO:
R2 is
(SEQ ID NO: 2)
5′-AgaTgTGtatAAGAGaCAG,
and
X6 is
(SEQ ID NO: 450)
acttggTTAATTAATTTTTTTTTTTTTTTTTTTTTT,

and
Y corresponds to no nucleotide, or to the replacement sequence of the target region.

Advantageously, the invention relates to the following complexes:
a first molecule of sequence

5′-
TGCAGCTGR2TTTTTTTTTTTTTTTTTTTTTTTTTTTGGCGATCGCTTTTTTT
TTTTTTTTTTTTTXTTTTTTTTTTTTTTTTTTTTGCGATCGCCTTTTTTTTTTTT
TTTTTGATACATGTTTR2CTGTAAGC-3′ SEQ ID NO: sumarized by X1R2X2-
X-X3R2X4
wherein X1 is
(SEQ ID NO: 436)
TGCAGCTG
R2 is
(SEQ ID NO: 2)
5′-AgaTgTGtatAAGAGaCAG,

X2 is SEQ ID NO: 437,
X3 is SEQ ID NO: 438, et
X4 is SEQ ID NO: 439
SEQ ID NO: SEQ ID NO: X corresponds to the sequence allowing the recognition of the target region
and
   a second molecule comprising the following sequence:

5′-
R1CAGCTGCAGACAAAGCTTACAGR1TTTTTTTTTTTTTTTTTTTTTTcatatg
ccaagtY-3′ SEQ ID NO:sumarized by X5R1X6-Y
wherein X5 is
(SEQ ID NO: 440)
CAGCTGCAGACAAAGCTTACAG
Wherein R1 is
(SEQ ID NO: 1)
5′-CTGtCTCTTataCAcAtcT,
X6 is
SEQ ID NO: 441
R2 is
(SEQ ID NO: 2)
5′-AgaTgTGtatAAGAGaCAG,

Y corresponds to no nucleotide, or to the replacement sequence of the target region.

Advantageously, the invention relates to the following complexes:
a first molecule of sequence

5′-
ATCATCR2TTTTTTTTTTTTTTTTTGATAGTAGR2TTTTTTTTTTTTTTTTTTT
TTTTGGCGATCGCTTTTTTTTTTTTTTTTXTTTTTTTTTTTTTTTTGCGATCG
CCTTTTTTTTTTTTTGATACATTTR1GATGAT-3′ SEQ ID NO:sumarized by
X1R2X2R2X3-X-X4R1X5
Wherein X1 is
SEQ D NO: 445
R2 is
(SEQ ID NO: 2)
5′-AgaTgTGtatAAGAGaCAG,

X2 is SEQ ID NO: 446
X3 is SEQ ID NO: 447
X4 is SEQ ID NO: 448

Wherein R1 is 5′-CTGtCTCTTataCAcAtcT (SEQ ID NO: 1),
X5 est SEQ ID NO: 449SEQ ID NO: X corresponds to the sequence allowing the recognition of the target region
and
   a second molecule comprising the following sequence:

5′-YacttggTTAATTAATTTTTTTTTTTTTTTTTTTTTTR1CTACTA
SEQ ID NO:sumarized by Y-X6R1X7

wherein X6 is SEQ ID NO: 450
Wherein R1 is 5′-CTGtCTCTTataCAcAtcT (SEQ ID NO: 1), and
X7 is SEQ ID NO: 451SEQ ID NO: Y corresponds to no nucleotide, or to the replacement sequence of the target region.

Advantageously, the aforementioned complex consists of the following pairs of sequences:

TABLE 2

| Molecule 1 | Molecule 2 | R1 | R2 |
|---|---|---|---|
| 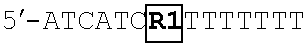 | 5′-YacttggTTAATTA | SEQ ID NO: n | SEQ ID NO: n − 1 |
| TTTTTTTTTTGATAGTA | ATTTTTTTTTTTTTT | n being an even | n being the same |
| GR1TTTTTTTTTTTTTT | TTTTTTTTR2CTACT | from 1 to 6 and | even number as |
| TTTTTTTTTGGCGATC | ATC-3′ | from 269 to 424 | R1 ranging from |
| GCTTTTTTTTTTTTTTT | sumarized by Y-SEQ | number ranging | 1 to 6 and from |
| TXTTTTTTTTTTTTTTT | ID NO: 450-R2-SEQ | | 269 to 424 |
| TGCGATCGCCTTTTTT | ID NO: 451 | | |
| TTTTTTTGATACATTTR | SEQ ID NO: | | |
| 2GATGAT-3′ sumarized | | | |
| by SEQ ID NO: 445-R1- | | | |
| SEQ ID NO: 446-R1- | | | |
| SEQ ID NO: 447-X-SEQ | | | |
| ID NO: 448-R2-SEQ | | | |
| ID NO: 449 SEQ ID | | | |
| NO: | | | |
| 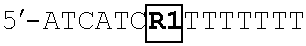 | 5′-YacttggTTAATTA | SEQ ID NO: n | SEQ ID NO: n + 1 |
| TTTTTTTTTTGATAGT | ATTTTTTTTTTTTTT | n being an odd | n being the same |
| AGR1TTTTTTTTTTTTT | TTTTTTTTR2CTACT | number ranging | odd number as |
| TTTTTTTTTTGGCGAT | ATC-3′ sumarized by | from 1 to 6 and | R1 ranging from |
| CGCTTTTTTTTTTTTTT | Y-SEQ ID NO: 450- | from 269 to 424 | 1 to 6 and from |
| TTXTTTTTTTTTTTTTT | R2-SEQ ID NO: 451 | | 269 to 424 |
| TTGCGATCGCCTTTTT | SEQ ID NO: | | |
| TTTTTTTTGATACATTT | | | |
| R2GATGAT-3′ | | | |
| Sumarized by SEQ ID | | | |
| NO: 445-R1-SEQ ID | | | |
| NO: 446-R1-SEQ ID | | | |
| NO: 447-X-SEQ ID NO: | | | |
| 448-R2-SEQ ID NO: 449 | | | |
| SEQ ID NO: | | | |
| 5′-TGCAGCTGR2TTTT | 5′-R1CAGCTGCAGA | SEQ ID NO: n | SEQ ID NO: n − 1 |
| TTTTTTTTTTTTTTTTT | CAAAGCTTACAGR1 | In being an even | n being the same |
| TTTTTTGGCGATCGCT | TTTTTTTTTTTTTTT | number ranging | even number as |
| TTTTTTTTTTTTTTTTT | TTTTTTTcatatgccaa | from 1 to 6 and | R1 ranging from |
| TTXTTTTTTTTTTTTTT | gtY-3′ | from 269 to 424 | 1 to 6 and from |
| TTTTTTGCGATCGCCT | Sumarized by SEQ ID | | 269 to 424 |
| TTTTTTTTTTTTTTTGA | NO: 440-R1-SEQ ID | | |
| TACATGTTTR2CTGTA | NO: 441 | | |
| AGC-3′ | SEQ ID NO: | | |
| Sumarized by SEQ ID | | | |
| NO: 436-R2-SEQ ID | | | |
| NO: 437-X-SEQ ID NO: | | | |
| 438-R2-SEQ ID NO: 439 | | | |
| SEQ ID NO: | | | |
| 5′-TGCAGCTGR2TTTT | 5′-R1CAGCTGCAGA | SEQ ID NO: n | SEQ ID NO: n + 1 |
| TTTTTTTTTTTTTTTTT | CAAAGCTTACAGR1 | n being an odd | n being the same |
| TTTTTTGGCGATCGCT | TTTTTTTTTTTTTTT | number ranging | odd number as |
| TTTTTTTTTTTTTTTTT | TTTTTTTcatatgccaa | from 1 to 6 and | R1 ranging from |
| TTXTTTTTTTTTTTTTT | gtY-3′ | from 269 to 424 | 1 to 6 and from |
| TTTTTTGCGATCGCCT | Sumarized by SEQ ID | | 269 to 424 |
| TTTTTTTTTTTTTTTGA | NO: 440-R1-SEQ ID | | |
| TACATGTTTR2CTGTA | NO: 441 | | |
| AGC-3′ | SEQ ID NO: | | |
| Sumarized by SEQ ID | | | |
| NO: 436-R2-SEQ ID | | | |
| NO: 437-X-SEQ ID NO: | | | |
| 438-R2-SEQ ID NO: 439 | | | |
| SEQ ID NO: | | | |
| 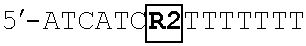 | 5′-YacttggTTAATTA | SEQ ID NO: n | SEQ ID NO: n − 1 |
| TTTTTTTTTTGATAGT | ATTTTTTTTTTTTTT | n being an even | n being the same |
| AGR2TTTTTTTTTTTTT | TTTTTTTTR1CTACT | number ranging | even number as |
| TTTTTTTTTTGGCGAT | ATC-3′ | from 1 to 6 and | R1 ranging from |
| CGCTTTTTTTTTTTTTT | Sumarized by Y-SEQ | from 269 to 424 | 1 to 6 and from |
| TTXTTTTTTTTTTTTTT | ID NO: 450-R1-SEQ | | 269 to 424 |
| TTGCGATCGCCTTTTT | ID NO: 451 | | |
| TTTTTTTTGATACATTT | SEQ ID NO: | | |
| R1GATGAT-3′ | | | |
| Sumarized by SEQ ID | | | |
| NO: 445 -R2- SEQ ID | | | |
| NO: 446-R2-SEQ ID | | | |
| NO: 447-X-SEQ ID NO: | | | |
| 448-R1-SEQ ID NO: 449 | | | |
| SEQ ID NO: | | | |
| 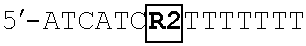 | 5′-YacttggTTAATTA | SEQ ID NO: n | SEQ ID NO: n + 1 |
| TTTTTTTTTTGATAGT | ATTTTTTTTTTTTTT | n being an odd | n being the same |
| AGR2TTTTTTTTTTTTT | TTTTTTTTR1CTACT | number ranging | odd number as |
| TTTTTTTTTTGGCGAT | ATC-3′ | from 1 to 6 and | R1 ranging from |
| CGCTTTTTTTTTTTTTT | Sumarized by Y-SEQ | from 269 to 424 | 1 to 6 and from |
| TTXTTTTTTTTTTTTTT | ID NO: 450-R1-SEQ | | 269 to 424 |
| TTGCGATCGCCTTTTT | ID NO: 451 | | |
| TTTTTTTTGATACATTT | SEQ ID NO: | | |
| R1GATGAT-3′ | | | |
| Sumarized by SEQ ID | | | |
| NO: 445-R2-SEQ ID | | | |
| NO: 446-R2-SEQ ID | | | |
| NO: 447-X-SEQ ID NO: | | | |
| 448-R1-SEQ ID NO: 449 | | | |
| SEQ ID NO: | | | |

5′ insertion-model 1

| | | | |
|---|---|---|---|
| 5′-TGCAGCTGR1TTTT | 5′-R2CAGCTGCAGA | SEQ ID NO: n | SEQ ID NO: n − 1 |
| TTTTTTTTTTTTTTTTT | CAAAGCTTACAGR1 | n being an even | n being the same |
| TTTTTTGGTCGCTTTT | TTTTTTTTTTTTTTT | number ranging | even number as |
| TTTTTTTTTTTTTTTTX | TTTTTTTYccaagt-3′ | from 1 to 6 and | R1 ranging from |
| TTTTTTTTTTTTTTTTT | Sumarized by R2- | from 269 to 424 | 1 to 6 and from |
| TTTGCGACCTTTTTTT | SEQ ID NO: 440-R1 | | 269 to 424 |
| TTTTTTTTTGATACAT | SEQ ID NO: 454-Y- | | |
| GTTTR2CTGTAAGC-3′ | ccaagt | | |
| Sumarized by SEQ ID | SEQ ID NO: | | |
| NO: 436-R1-SEQ ID | | | |
| NO: 452-X-SEQ ID NO: | | | |
| 453-R2-SEQ ID NO: 439 | | | |
| SEQ ID NO: | | | |
| 5′-TGCAGCTGR1TTTT | 5′-R2CAGCTGCAGA | SEQ ID NO: n | SEQ ID NO: n + 1 |
| TTTTTTTTTTTTTTTTT | CAAAGCTTACAGR1 | n being an odd | n being the same |
| TTTTTTGGTCGCTTTT | TTTTTTTTTTTTTTT | number ranging | odd number as |
| TTTTTTTTTTTTTTTTX | TTTTTTTYccaagt-3′ | from 1 to 6 and | R1 ranging from |
| TTTTTTTTTTTTTTTTT | Sumarized by R2- | from 269 to 424 | 1 to 6 and from |
| TTTGCGACCTTTTTTT | SEQ ID NO: 440-R1- | | 269 to 424 |
| TTTTTTTTTGATACAT | SEQ ID NO: 454-Y- | | |
| GTTTR2CTGTAAGC-3′ | ccaagt | | |
| Sumarized by SEQ ID | SEQ ID NO: | | |
| NO: 436-R1-SEQ ID | | | |
| NO: 452-X-SEQ ID NO: | | | |
| 453-R2-SEQ ID NO: 439 | | | |
| SEQ ID NO: | | | |
| 5′-TGCAGCTGR1TTTT | 5′-R2CAGCTGCAGA | SEQ ID NO: n | SEQ ID NO: n − 1 |
| TTTTTTTTTTTTTTTTT | CAAAGCTTACAGR1 | n being an even | n being the same |
| TTTTTTGGTCGCTTTT | TTTTTTTTTTTTTTT | number ranging | even number as |
| TTTTTTTTTTTTTTTTX | TTTTTTTccaagtY-3′ | from 1 to 6 and | R1 ranging from |
| TTTTTTTTTTTTTTTTT | Sumarized by R2- | from 269 to 424 | 1 to 6 and from |
| TTTGCGACCTTTTTTT | SEQ ID NO: 440-R1- | | 269 to 424 |
| TTTTTTTTTGATACAT | SEQ ID NO: 455-Y | | |
| GTTTR2CTGTAAGC-3′ | SEQ ID NO: | | |
| Sumarized by SEQ ID | | | |
| NO: 436-R1-SEQ ID | | | |
| NO: 452-X-SEQ ID NO: | | | |
| 453-R2-SEQ ID NO: 439 | | | |
| SEQ ID NO: | | | |
| 5′-TGCAGCTGR1TTTT | 5′-R2CAGCTGCAGA | SEQ ID NO: n | SEQ ID NO: n + 1 |
| TTTTTTTTTTTTTTTTT | CAAAGCTTACAGR1 | n being an odd | n being the same |
| TTTTTTGGTCGCTTTT | TTTTTTTTTTTTTTT | number ranging | odd number as |
| TTTTTTTTTTTTTTTTX | TTTTTTTccaagtY- | from 1 to 6 and | R1 ranging from |
| TTTTTTTTTTTTTTTTT | 3′Sumarized by R2- | from 269 to 424 | 1 to 6 and from |
| TTTGCGACCTTTTTTT | SEQ ID NO: 440-R1- | | 269 to 424 |
| TTTTTTTTTGATACAT | SEQ ID NO: 455-Y | | |
| GTTTR2CTGTAAGC-3′ | SEQ ID NO: | | |
| Sumarized by SEQ ID | | | |
| NO: 436-R1-SEQ ID | | | |
| NO: 452-X-SEQ ID NO: | | | |
| 453-R2-SEQ ID NO: 439 | | | |
| SEQ ID NO: | | | |

5′ insertion-model 2

| | | | |
|---|---|---|---|
| 5′-TGCAGCTGR1TTTT | 5′-R2CAGCTGCAGA | SEQ ID NO: n | SEQ ID NO: n − 1 |
| TTTTTTTTTTTTTTTTT | CAAAGCTTACAGR1 | n being an even | n being the same |
| TTTTTTGCGGCGATC | TTTTTTTTTTTTTTT | number ranging | even number as |
| GGCTTTTTTTTTTTTTT | TTTTTTTYccaagt-3′ | from 1 to 6 and | R1 ranging from |
| TTTTTTXTTTTTTTTTT | Sumarized by R2- | from 269 to 424 | 1 to 6 and from |
| TTTTTTTTTTGCCGAT | SEQ ID NO: 440-R1- | | 269 to 424 |
| CGCCGCTTTTTTTTTT | SEQ ID NO: 454-Y- | | |
| TTTTTTGATACATGTT | ccaagt | | |
| TR2CTGTAAGC-3′ | SEQ ID NO: | | |
| Sumarized by SEQ ID | | | |
| NO: 436-R1-SEQ ID | | | |
| NO: 456-X-SEQ ID NO: | | | |
| 457-R2-SEQ ID NO: 439 | | | |
| SEQ ID NO: | | | |
| 5′-TGCAGCTGR1TTTT | 5′-R2CAGCTGCAGA | SEQ ID NO: n | SEQ ID NO: n + 1 |
| TTTTTTTTTTTTTTTTT | CAAAGCTTACAGR1 | n being an odd | n being the same |
| TTTTTTGCGGCGATC | TTTTTTTTTTTTTTT | number ranging | odd number as |
| GGCTTTTTTTTTTTTTT | TTTTTTTYccaagt- | from 1 to 6 and | R1 ranging from |
| TTTTTTXTTTTTTTTTT | 3′Sumarized by R2- | from 269 to 424 | 1 to 6 and from |
| TTTTTTTTTTGCCGAT | SEQ ID NO: 440-R1- | | 269 to 424 |
| CGCCGCTTTTTTTTTT | SEQ ID NO: 454-Y- | | |
| TTTTTTGATACATGTT | ccaagt | | |
| TR2CTGTAAGC-3′ | SEQ ID NO: | | |
| Sumarized by SEQ ID | | | |
| NO: 436-R1-SEQ ID | | | |
| NO: 456-X-SEQ ID NO: | | | |
| 457-R2-SEQ ID NO: 439 | | | |
| SEQ ID NO: | | | |
| 5′-TGCAGCTGR1TTTT | 5′-R2CAGCTGCAGA | SEQ ID NO: n | SEQ ID NO: n − 1 |
| TTTTTTTTTTTTTTTTT | CAAAGCTTACAGR1 | n being an even | n being the same |
| TTTTTTGCGGCGATC | TTTTTTTTTTTTTTT | number ranging | even number as |
| GGCTTTTTTTTTTTTTT | TTTTTTTccaagtY-3′ | from 1 to 6 and | R1 ranging from |
| TTTTTTXTTTTTTTTTT | 3′Sumarized by R2- | from 269 to 424 | 1 to 6 and from |
| TTTTTTTTTTGCCGAT | SEQ ID NO: 440-R1- | | 269 to 424 |
| CGCCGCTTTTTTTTTT | SEQ ID NO: 455-Y | | |
| TTTTTTGATACATGTT | SEQ ID NO: | | |
| TR2CTGTAAGC-3′ | | | |
| Sumarized by SEQ ID | | | |
| NO: 436-R1-SEQ ID | | | |
| NO: 456-X-SEQ ID NO: | | | |
| 457-R2-SEQ ID NO: 439 | | | |
| SEQ ID NO: | | | |
| 5′-TGCAGCTGR1TTTT | 5′-R2CAGCTGCAGA | SEQ ID NO: n | SEQ ID NO: n + 1 |
| TTTTTTTTTTTTTTTTT | CAAAGCTTACAGR1 | n being an odd | n being the same |
| TTTTTTGCGGCGATC | TTTTTTTTTTTTTTT | number ranging | odd number as |
| GGCTTTTTTTTTTTTTT | TTTTTTTccaagtY-3′ | from 1 to 6 and | R1 ranging from |
| TTTTTTXTTTTTTTTTT | 3′Sumarized by R2- | from 269 to 424 | 1 to 6 and from |
| CGCCGCTTTTTTTTTT | SEQ ID NO: 440-R1- | | 269 to 424 |
| TTTTTTTTTTGCCGAT | SEQ ID NO: 455-Y | | |
| TTTTTTGATACATGTT | SEQ ID NO: | | |
| TR2CTGTAAGC-3′ | | | |
| Sumarized by SEQ ID | | | |
| NO: 436-R1-SEQ ID | | | |
| NO: 456-X-SEQ ID NO: | | | |
| 457-R2-SEQ ID NO: 439 | | | |
| SEQ ID NO: | | | |

3′ insertion - model 1

| | | | |
|---|---|---|---|
| 5′-GTGCCCAGR1TTTC | 5′-YtacaagTCCGGA | SEQ ID NO: n | SEQ ID NO: n − 1 |
| TCGATCATTTTTTTTT | TAATAATTTTTTTTT | n being an even | n being the same |
| TTTTTTTGGTCGCTTT | TTTTTTTTTTTTTR2 | number ranging | even number as |
| TTTTTTTTTTTTTTTTT | CTGGGCACGCGTA | from 1 to 6 and | R1 ranging from |
| XTTTTTTTTTTTTTTTT | TAAGCAGR1-3′ | from 269 to 424 | 1 to 6 and from |
| TTTTGCGACCTTTTTT | Sumarized by Y-SEQ | | 269 to 424 |
| TTTTTTTTTTTTTTT | ID NO: 462-R2-SEQ | | |
| (SEQ ID NO: | ID NO: 463-R1 | | |
| 485)TTTTTTR2CTGCT | SEQ ID NO: | | |
| TAT-3′ | | | |
| Sumarized by SEQ ID | | | |
| NO: 458-R1-SEQ ID | | | |
| NO: 459-X-SEQ ID NO: | | | |
| 460-R2-SEQ ID NO: 461 | | | |
| SEQ ID NO: | | | |
| 5′-GTGCCCAGR1TTTC | 5′-YtacaagTCCGGA | SEQ ID NO: n | SEQ ID NO: n + 1 |
| TCGATCATTTTTTTTT | TAATAATTTTTTTTT | n being an odd | n being the same |
| TTTTTTTGGTCGCTTT | TTTTTTTTTTTTTR2 | number ranging | odd number as |
| TTTTTTTTTTTTTTTTT | CTGGGCACGCGTA | from 1 to 6 and | R1 ranging from |
| XTTTTTTTTTTTTTTTT | TAAGCAGR1-3′ | from 269 to 424 | 1 to 6 and from |
| TTTTGCGACCTTTTTT | Sumarized by Y-SEQ | | 269 to 424 |
| TTTTTTTTTTTTTTT | ID NO: 462-R2-SEQ | | |
| (SEQ ID NO: | ID NO: 463-R1 | | |
| 485)TTTTTTR2CTGCT | SEQ ID NO: | | |
| TAT-3′ | | | |
| Sumarized by SEQ ID | | | |
| NO: 458-R1-SEQ ID | | | |
| NO: 459-X-SEQ ID NO: | | | |
| 460-R2-SEQ ID NO: 461 | | | |
| SEQ ID NO: | | | |
| 5′-GTGCCCAGR1TTTC | 5′-tacaagYTAATAAT | SEQ ID NO: n | SEQ ID NO: n − 1 |
| TCGATCATTTTTTTTT | TTTTTTTTTTTTTTT | n being an even | n being the same |
| TTTTTTTGGTCGCTTT | TTTTTTR2CTGGGC | number ranging | even number as |
| TTTTTTTTTTTTTTTTT | ACGCGTATAAGCA | from 1 to 6 and | R1 ranging from |
| XTTTTTTTTTTTTTTTT | GR1-3′ | from 269 to 424 | 1 to 6 and from |
| TTTTGCGACCTTTTTT | Sumarized by tacaag- | | 269 to 424 |
| TTTTTTTTTTTTTTT | Y-SEQ ID NO: 464)- | | |
| (SEQ ID NO: | R2-SEQ ID NO: 463- | | |
| 485)TTTTTTR2CTGCT | R1 | | |
| TAT-3′ | SEQ ID NO: | | |
| Sumarized by SEQ ID | | | |
| NO: 458-R1-SEQ ID | | | |
| NO: 459-X-SEQ ID NO: | | | |
| 460-R2-SEQ ID NO: 461 | | | |
| SEQ ID NO: | | | |
| 5′-GTGCCCAGR1TTTC | 5′-tacaagYTAATAAT | SEQ ID NO: n | SEQ ID NO: n + 1 |
| TCGATCATTTTTTTTT | TTTTTTTTTTTTTTT | n being an odd | n being the same |
| TTTTTTTGGTCGCTTT | TTTTTTR2CTGGGC | number ranging | odd number as |
| TTTTTTTTTTTTTTTTT | ACGCGTATAAGCA | from 1 to 6 and | R1 ranging from |
| XTTTTTTTTTTTTTTTT | GR1-3′ | from 269 to 424 | 1 to 6 and from |
| TTTTGCGACCTTTTTT | Sumarized by tacaag- | | 269 to 424 |
| TTTTTTTTTTTTTTT | Y-SEQ ID NO: 464)- | | |
| (SEQ ID NO: | R2-SEQ ID NO: 463- | | |
| 485)TTTTTTR2CTGCT | R1 | | |
| TAT-3′ | SEQ ID NO: | | |
| Sumarized by SEQ ID | | | |
| NO: 458-R1-SEQ ID | | | |
| NO: 459-X-SEQ ID NO: | | | |
| 460-R2-SEQ ID NO: 461 | | | |
| SEQ ID NO: | | | |
| 5′-GTGCCCAGR1TTTC | 5′-YtacaagTAATAAT | SEQ ID NO: n | SEQ ID NO: n − 1 |
| TCGATCATTTTTTTTT | TTTTTTTTTTTTTTT | n being an even | n being the same |
| TTTTTTTGGTCGCTTT | 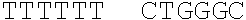 | number ranging | even number as |
| TTTTTTTTTTTTTTTTT | ACGCGTATAAGCA | from 1 to 6 and | R1 ranging from |
| XTTTTTTTTTTTTTTTT | 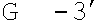 | from 269 to 424 | 1 to 6 and from |
| TTTTGCGACCTTTTTT | Répresenté par Y- | | 269 to 424 |
| TTTTTTTTTTTTTTT | SEQ ID NO: 465-R2- | | |
| (SEQ ID NO: | SEQ ID NO: 463-R1 | | |
| 485)TTTTTTR2CTGCT | SEQ ID NO: | | |
| TAT-3′Sumarized by | | | |
| SEQ ID NO: 458-R1- | | | |
| SEQ ID NO: 459-X-SEQ | | | |
| ID NO: 460-R2-SEQ ID | | | |
| NO: 461 | | | |
| SEQ ID NO: | | | |
| 5′-GTGCCCAGR1TTTC | 5′-YtacaagTAATAAT | SEQ ID NO: n | SEQ ID NO: n + 1 |
| TCGATCATTTTTTTTT | TTTTTTTTTTTTTTT | n being an odd | n being the same |
| TTTTTTTGGTCGCTTT | 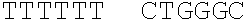 | number ranging | odd number as |
| TTTTTTTTTTTTTTTTT | ACGCGTATAAGCA | from 1 to 6 and | R1 ranging from |
| XTTTTTTTTTTTTTTTT | 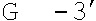 | from 269 to 424 | 1 to 6 and from |
| TTTTGCGACCTTTTTT | Represente par Y- | | 269 to 424 |
| TTTTTTTTTTTTTTTT | SEQ ID NO: 465-R2- | | |
| (SEQ ID NO: | SEQ ID NO: 463-R1 | | |
| 485)TTTTTTR2CTGCT | SEQ ID NO: | | |
| TAT-3′ | | | |
| Sumarized by SEQ ID | | | |
| NO: 458-R1-SEQ ID | | | |
| NO: 459-X-SEQ ID NO: | | | |
| 460-R2-SEQ ID NO: 461 | | | |
| SEQ ID NO: | | | |

3′ insertion-model 2

| | | | |
|---|---|---|---|
| 5′-GTGCCCAGR1TTTC | 5′-YtacaagTCCGGA | SEQ ID NO: n | SEQ ID NO: n − 1 |
| TCGATCATTTTTTTTT | TAATAATTTTTTTTT | n being an even | n being the same |
| TTTTTTTGGCGATCGC | TTTTTTTTTTTTTR2 | number ranging | even number as |
| TTTTTTTTTTTTTTTTT | CTGGGCACGCGTA | from 1 to 6 and | R1 ranging from |
| TTTXTTTTTTTTTTTTT | TAAGCAGR1-3′ | from 269 to 424 | 1 to 6 and from |
| TTTTTTTGCGATCGCC | Sumarized by Y-SEQ | | 269 to 424 |
| TTTTTTTTTTTTTTTTT | ID NO: 462-R2-SEQ | | |
| TTTTTTTTTTR2CTGCT | ID NO: 463-R1 | | |
| TAT-3′ | SEQ ID NO: | | |
| Sumarized by SEQ ID | | | |
| NO: 458-R1-SEQ ID | | | |
| NO: 466-X-SEQ ID NO: | | | |
| 467-R2-SEQ ID NO: 461 | | | |
| SEQ ID NO: | | | |
| 5′-GTGCCCAGR1TTTC | 5′-YtacaagTCCGGA | SEQ ID NO: n | SEQ ID NO: n + 1 |
| TCGATCATTTTTTTTT | TAATAATTTTTTTTT | n being an odd | n being the same |
| TTTTTTTGGCGATCGC | TTTTTTTTTTTTTR2 | number ranging | odd number as |
| TTTTTTTTTTTTTTTTT | CTGGGCACGCGTA | from 1 to 6 and | R1 ranging from |
| TTTXTTTTTTTTTTTTT | TAAGCAGR1-3′ | from 269 to 424 | 1 to 6 and from |
| TTTTTTTGCGATCGCC | Sumarized by Y-SEQ | | 269 to 424 |
| TTTTTTTTTTTTTTTTT | ID NO: 462-R2-SEQ | | |
| TTTTTTTTTTR2CTGCT | ID NO: 463-R1 | | |
| TAT-3′ | SEQ ID NO: | | |
| Sumarized by SEQ ID | | | |
| NO: 458-R1-SEQ ID | | | |
| NO: 466-X-SEQ ID NO: | | | |
| 467-R2-SEQ ID NO: 461 | | | |
| SEQ ID NO: | | | |
| 5′-GTGCCCAGR1TTTC | 5′-tacaagYTAATAAT | SEQ ID NO: n | SEQ ID NO: n − 1 |
| TCGATCATTTTTTTTTT | TTTTTTTTTTTTTTT | n being an even | n being the same |
| TTTTTTGGCGATCGCT | TTTTTTR2CTGGGC | number ranging | even number as |
| TTTTTTTTTTTTTTTTT | ACGCGTATAAGCA | from 1 to 6 and | R1 ranging from |
| TTXTTTTTTTTTTTTTT | GR1-3′ | from 269 to 424 | 1 to 6 and from |
| TTTTTTGCGATCGCCT | Sumarized by tacaag- | | 269 to 424 |
| TTTTTTTTTTTTTTTTT | Y-SEQ ID NO: 464)- | | |
| TTTTTTTTTR2CTGCTT | R2-SEQ ID NO: 463- | | |
| AT-3′ Sumarized by | R1 | | |
| SEQ ID NO: 458-R1- | SEQ ID NO: | | |
| SEQ ID NO: 466-X-SEQ | | | |
| ID NO: 467-R2-SEQ ID | | | |
| NO: 461 SEQ ID NO: | | | |
| 5′-GTGCCCAGR1TTTC | 5′-tacaagYTAATAAT | SEQ ID NO: n | SEQ ID NO: n + 1 |
| TCGATCATTTTTTTTT | TTTTTTTTTTTTTTT | n being an odd | n being the same |
| TTTTTTTGGCGATCGC | TTTTTTR2CTGGGC | number ranging | odd number as |
| TTTTTTTTTTTTTTTTT | ACGCGTATAAGCA | from 1 to 6 and | R1 ranging from |
| TTTXTTTTTTTTTTTTT | GR1-3′ | from 269 to 424 | 1 to 6 and from |
| TTTTTTTGCGATCGCC | Sumarized by tacaag- | | 269 to 424 |
| TTTTTTTTTTTTTTTTT | Y-SEQ ID NO: 464)- | | |
| TTTTTTTTTTR2CTGCT | R2-SEQ ID NO: 463- | | |
| TAT-3′ Sumarized by | R1 | | |
| SEQ ID NO: 458-R1- | SEQ ID NO: | | |
| SEQ ID NO: 466-X-SEQ | | | |
| ID NO: 467-R2-SEQ ID | | | |
| NO: 461 | | | |
| SEQ ID NO: | | | |
| 5′-GTGCCCAGR1TTTC | 5′-YtacaagTAATAAT | SEQ ID NO: n | SEQ ID NO: n − 1 |
| TCGATCATTTTTTTTT | TTTTTTTTTTTTTTT | n being an even | n being the same |
| TTTTTTTGGCGATCGC | TTTTTTR2CTGGGC | number ranging | even number as |
| TTTTTTTTTTTTTTTTT | ACGCGTATAAGCA | from 1 to 6 and | R1 ranging from |
| TTTXTTTTTTTTTTTTT | GR1-3′ | from 269 to 424 | 1 to 6 and from |
| TTTTTTTGCGATCGCC | Sumarized by Y-SEQ | | 269 to 424 |
| TTTTTTTTTTTTTTTTT | NO: 465-R2-SEQ | | |
| TTTTTTTTTTR2CTGCT | ID NO: 463-R1 | | |
| TAT-3′ | SEQ ID NO: | | |
| Sumarized by SEQ ID | | | |
| NO: 458-R1-SEQ ID | | | |
| NO: 466-X-SEQ ID NO: | | | |
| 467-R2-SEQ ID NO: 461 | | | |
| SEQ ID NO: | | | |
| 5′-GTGCCCAGR1TTTC | 5′-YtacaagTAATAAT | SEQ ID NO: n | SEQ ID NO: n + 1 |
| TCGATCATTTTTTTTT | TTTTTTTTTTTTTTT | n being an odd | n being the same |
| TTTTTTTGGCGATCGC | TTTTTTR2CTGGGC | number ranging | odd number as |
| TTTTTTTTTTTTTTTTT | ACGCGTATAAGCA | from 1 to 6 and | R1 ranging from |
| TTTXTTTTTTTTTTTTT | GR1-3′Sumarized by | from 269 to 424 | 1 to 6 and from |
| TTTTTTTGCGATCGCC | Y-SEQ ID NO: 465- | | 269 to 424 |
| TTTTTTTTTTTTTTTTT | R2-SEQ ID NO: 463- | | |
| TTTTTTTTR2CTGCT | R1 | | |
| TAT-3′ | SEQ ID NO: | | |
| Sumarized by SEQ ID | | | |
| NO: 458-R1-SEQ ID | | | |
| NO: 466-X-SEQ ID NO: | | | |
| 467-R2-SEQ ID NO: 461 | | | |
| SEQ ID NO: | | | |

3′ insertion-model 3

| | | | |
|---|---|---|---|
| 5′-GTGCCCAGR1TTTC | 5′-YtacaagTCCGGA | SEQ ID NO: n | SEQ ID NO: n − 1 |
| TCGATCATTTTTTTTT | TAATAATTTTTTTTT | n being an even | n being the same |
| TTTTTTTGCGGCGATC | TTTTTTTTTTTTTR2 | number ranging | even number as |
| GGCTTTTTTTTTTTTTT | CTGGGCACGCGTA | from 1 to 6 and | R1 ranging from |
| TTTTTTXTTTTTTTTTT | TAAGCAGR1- | from 269 to 424 | 1 to 6 and from |
| TTTTTTTTTTGCCGAT | 3′Sumarized by Y- | | 269 to 424 |
| CGCCGCTTTTTTTTTT | SEQ ID NO: 462-R2- | | |
| TTTTTTTTTTTTTTTTT | SEQ ID NO: 463-R1 | | |
| R2CTGCTTAT-3′ | SEQ ID NO: | | |
| Sumarized by SEQ ID | | | |
| NO: 458-R1-SEQ ID | | | |
| NO: 468-X-SEQ ID NO: | | | |
| 469-R2-SEQ ID NO: 461 | | | |
| SEQ ID NO: | | | |
| 5′-GTGCCCAGR1TTTC | 5′-YtacaagTCCGGA | SEQ ID NO: n | SEQ ID NO: n + 1 |
| TCGATCATTTTTTTTT | TAATAATTTTTTTTT | n being an odd | n being the same |
| TTTTTTTGCGGCGATC | TTTTTTTTTTTTTR2 | number ranging | odd number as |
| GGCTTTTTTTTTTTTTT | CTGGGCACGCGTA | from 1 to 6 and | R1 ranging from |
| TTTTTTXTTTTTTTTTT | TAAGCAGR1-3′ | from 269 to 424 | 1 to 6 and from |
| TTTTTTTTTTGCCGAT | Sumarized by Y- | | 269 to 424 |
| CGCCGCTTTTTTTTTT | SEQ ID NO: 462-R2- | | |
| TTTTTTTTTTTTTTTTT | SEQ ID NO: 463- | | |
| R2CTGCTTAT- | R1SEQ ID NO: | | |
| 3′Sumarized by SEQ ID | | | |
| NO: 458-R1-SEQ ID | | | |
| NO: 468-X-SEQ ID NO: | | | |
| 469-R2-SEQ ID NO: 461 | | | |
| SEQ ID NO: | | | |
| 5′-GTGCCCAGR1TTTC | 5′-tacaagYTAATAAT | SEQ ID NO: n | SEQ ID NO: n − 1 |
| TCGATCATTTTTTTTT | TTTTTTTTTTTTTTT | n being an even | n being the same |
| TTTTTTTGCGGCGATC | TTTTTTR2CTGGGC | number ranging | even number as |
| GGCTTTTTTTTTTTTTT | ACGCGTATAAGCA | from 1 to 6 and | R1 ranging from |
| TTTTTTXTTTTTTTTTT | GR1-3′Sumarized by | from 269 to 424 | 1 to 6 and from |
| TTTTTTTTTTGCCGAT | tacaag-Y-SEQ ID | | 269 to 424 |
| CGCCGCTTTTTTTTTT | NO: 464)-R2-SEQ ID | | |
| TTTTTTTTTTTTTTTTT | NO: 463 R1 | | |
| R2CTGCTTAT-3′ | SEQ ID NO: | | |
| Sumarized by SEQ ID | | | |
| NO: 458-R1-SEQ ID | | | |
| NO: 468-X-SEQ ID NO: | | | |
| 469-R2-SEQ ID NO: 461 | | | |
| SEQ ID NO: | | | |
| 5′-GTGCCCAGR1TTTC | 5′-tacaagYTAATAAT | SEQ ID NO: n | SEQ ID NO: n + 1 |
| TCGATCATTTTTTTTT | TTTTTTTTTTTTTTT | n being an odd | n being the same |
| TTTTTTTGCGGCGATC | TTTTTTR2CTGGGC | number ranging | odd number as |
| GGCTTTTTTTTTTTTTT | ACGCGTATAAGCA | from 1 to 6 and | R1 ranging from |
| TTTTTTXTTTTTTTTTT | GR1-3′ | from 269 to 424 | 1 to 6 and from |
| TTTTTTTTTTGCCGAT | Sumarized by tacaag- | | 269 to 424 |
| CGCCGCTTTTTTTTTT | Y-SEQ ID NO: 464)- | | |
| TTTTTTTTTTTTTTTTT | R2-SEQ ID NO: 463- | | |
| R2CTGCTTAT-3′ | R1 | | |
| Sumarized by SEQ ID | SEQ ID NO: | | |
| NO: 458-R1-SEQ ID | | | |
| NO: 468-X-SEQ ID NO: | | | |
| 469-R2-SEQ ID NO: 461 | | | |
| SEQ ID NO: | | | |
| 5′-GTGCCCAGR1TTTC | 5′-YtacaagTAATAAT | SEQ ID NO: n | SEQ ID NO: n − 1 |
| TCGATCATTTTTTTTT | TTTTTTTTTTTTTTT | n being an even | n being the same |
| TTTTTTTGCGGCGATC | TTTTTTR2CTGGGC | number ranging | even number as |
| GGCTTTTTTTTTTTTTT | ACGCGTATAAGCA | from 1 to 6 and | R1 ranging from |
| TTTTTTXTTTTTTTTTT | GR1-3′ | from 269 to 424 | 1 to 6 and from |
| TTTTTTTTTTGCCGAT | Sumarized by Y-SEQ | | 269 to 424 |
| CGCCGCTTTTTTTTTT | ID NO: 465-R2-SEQ | | |
| TTTTTTTTTTTTTTTTT | ID NO: 463-R1 | | |
| R2CTGCTTAT-3′ | SEQ ID NO: | | |
| Sumarized by SEQ ID | | | |
| NO: 458-R1-SEQ ID | | | |
| NO: 468-X-SEQ ID NO: | | | |
| 469-R2-SEQ ID NO: 461 | | | |
| SEQ ID NO: | | | |
| 5′-GTGCCCAGR1TTTC | 5′-YtacaagTAATAAT | SEQ ID NO: n | SEQ ID NO: n + 1 |
| TCGATCATTTTTTTTT | TTTTTTTTTTTTTTT | n being an odd | n being the same |
| TTTTTTTGCGGCGATC | TTTTTTR2CTGGGC | number ranging | odd number as |
| GGCTTTTTTTTTTTTTT | ACGCGTATAAGCA | from 1 to 6 and | R1 ranging from |
| TTTTTTXTTTTTTTTTT | GR1-3′Sumarized by | from 269 to 424 | 1 to 6 and from |
| TTTTTTTTTTGCCGAT | Y-SEQ ID NO: 465- | | 269 to 424 |
| CGCCGCTTTTTTTTTT | R2-SEQ ID NO: 463- | | |
| TTTTTTTTTTTTTTTTT | R1 | | |
| R2CTGCTTAT-3′ | SEQ ID NO: | | |
| Sumarized by SEQ ID | | | |
| NO: 458-R1-SEQ ID | | | |
| NO: 468-X-SEQ ID NO: | | | |
| 469-R2-SEQ ID NO: 461 | | | |
| SEQ ID NO: | | | |

Sequences with two transposase sequences
5′ insertion-model 1

| | | | |
|---|---|---|---|
| 5′-ATCATCR1TTTTTTT | 5′-YacttggTTAATTA | SEQ ID NO: n | SEQ ID NO: n − 1 |
| TTTTTTTTTTGATAGT | ATTTTTTTTTTTTTT | n being an even | n being the same |
| AGR1TTTTTTTTTTTTT | TTTTTTTTR2CTACT | number ranging | even number as |
| TTTTTTTTTGGTCGCT | ATC-3′ | from 1 to 6 and | R1 ranging from |
| TTTTTTTTTTTTTTTXT | Sumarized by Y-SEQ | from 269 to 424 | 1 to 6 and from |
| TTTTTTTTTTTTTTTGC | ID NO: 450-R2-SEQ | | 269 to 424 |
| GACCTTTTTTTTTTTTT | ID NO: 451 | | |
| GATACATTTR2GATGA | SEQ ID NO: | | |
| T-3′ | | | |
| Représenté par SEQ ID | | | |
| NO: 445-R1-SEQ ID | | | |
| NO: 446-R1-SEQ ID | | | |
| NO: 470-X-SEQ ID NO: | | | |
| 474-R2-SEQ ID NO: | | | |
| 449 SEQ ID NO: | | | |
| 5′-ATCATCR1TTTTTTT | 5′-YacttggTTAATTA | SEQ ID NO: n | SEQ ID NO: n + 1 |
| TTTTTTTTTTGATAGT | ATTTTTTTTTTTTTT | n being an odd | n being the same |
| AGR1TTTTTTTTTTTTT | TTTTTTTTR2CTACT | number ranging | odd number as |
| TTTTTTTTTGGTCGCT | ATC-3′Sumarized by | from 1 to 6 and | R1 ranging from |
| TTTTTTTTTTTTTTXT | Y-SEQ ID NO: 450- | from 269 to 424 | 1 to 6 and from |
| TTTTTTTTTTTTTTGC | R2-SEQ ID NO: 451 | | 269 to 424 |
| GACCTTTTTTTTTTTTT | SEQ ID NO: | | |
| GATACATTTR2GATGA | | | |
| T-3′ | | | |
| Représenté par SEQ ID | | | |
| NO: 445-R1-SEQ ID | | | |
| NO: 446-R1-SEQ ID | | | |
| NO: 470-X-SEQ ID NO: | | | |
| 474-R2-SEQ ID NO: | | | |
| 449 SEQ ID NO: | | | |
| 5′-ATCATCR1TTTTTTT | 5′-acttggYTTAATTA | SEQ ID NO: n | SEQ ID NO: n − 1 |
| TTTTTTTTTTGATAGT | ATTTTTTTTTTTTTT | n being an even | n being the same |
| AGR1TTTTTTTTTTTTT | TTTTTTTTR2CTACT | number ranging | even number as |
| TTTTTTTTTGGTCGCT | ATC-3′ | from 1 to 6 and | R1 ranging from |
| TTTTTTTTTTTTTTTXT | Représenté par | from 269 to 424 | 1 to 6 and from |
| TTTTTTTTTTTTTTTGC | acttgg-Y-SEQ ID NO: | | 269 to 424 |
| GACCTTTTTTTTTTTTT | 473-R2-SEQ ID NO: | | |
| GATACATTTR2GATGA | 451 | | |
| T-3′ | SEQ ID NO: | | |
| Représenté par SEQ ID | | | |
| NO: 445-R1-SEQ ID | | | |
| NO: 446-R1-SEQ ID | | | |
| NO: 470-X-SEQ ID NO: | | | |
| 474-R2-SEQ ID NO: | | | |
| 449 SEQ ID NO: | | | |
| 5′-ATCATCR1TTTTTTT | 5′-acttggYTTAATTA | SEQ ID NO: n | SEQ ID NO: n + 1 |
| TTTTTTTTTTGATAGT | ATTTTTTTTTTTTTT | n being an odd | n being the same |
| AGR1TTTTTTTTTTTTT | TTTTTTTTR2CTACT | number ranging | odd number as |
| TTTTTTTTTTGGTCGCT | ATC-3′ | from 1 to 6 and | R1 ranging from |
| TTTTTTTTTTTTTTTXT | Représenté par | from 269 to 424 | 1 to 6 and from |
| TTTTTTTTTTTTTTTGC | acttgg-Y-SEQ ID NO: | | 269 to 424 |
| GACCTTTTTTTTTTTTT | 473-R2-SEQ ID NO: | | |
| GATACATTTR2GATGA | 451 | | |
| T-3′ | SEQ ID NO: | | |
| Représenté par SEQ ID | | | |
| NO: 445-R1-SEQ ID | | | |
| NO: 446-R1-SEQ ID | | | |
| NO: 470-X-SEQ ID NO: | | | |
| 474-R2-SEQ ID NO: 449 | | | |
| SEQ ID NO: | | | |
| 5′-ATCATCR1TTTTTTT | 5′-YacttggcatatgTTA | SEQ ID NO: n | SEQ ID NO: n − 1 |
| TTTTTTTTTTGATAGT | ATTAATTTTTTTTTT | n being an even | n being the same |
| AGR1TTTTTTTTTTTTT | TTTTTTTTTTTTR2C | number ranging | even number as |
| TTTTTTTTTGGTCGCT | TACTATC-3′ | from 1 to 6 and | R1 ranging from |
| TTTTTTTTTTTTTTTXT | Représenté par Y- | from 269 to 424 | 1 to 6 and from |
| TTTTTTTTTTTTTTTGC | SEQ ID NO: 475-R2- | | 269 to 424 |
| GACCTTTTTTTTTTTTT | SEQ ID NO: 451 | | |
| GATACATTTR2GATGA | SEQ ID NO: | | |
| T-3′ | | | |
| Représenté par SEQ ID | | | |
| NO: 445-R1-SEQ ID | | | |
| NO: 446-R1-SEQ ID | | | |
| NO: 470-X-SEQ ID NO: | | | |
| 474-R2-SEQ ID NO: 449 | | | |
| SEQ ID NO: | | | |
| 5′-ATCATCR1TTTTTTT | 5′-YacttggcatatgTTA | SEQ ID NO: n | SEQ ID NO: n + 1 |
| TTTTTTTTTTGATAGT | ATTAATTTTTTTTTT | n being an odd | n being the same |
| AGR1TTTTTTTTTTTTT | TTTTTTTTTTTTR2C | number ranging | odd number as |
| TTTTTTTTTGGTCGCT | TACTATC-3′ | from 1 to 6 and | R1 ranging from |
| TTTTTTTTTTTTTTTXT | Représenté par Y- | from 269 to 424 | 1 to 6 and from |
| TTTTTTTTTTTTTTTGC | SEQ ID NO: 475-R2- | | 269 to 424 |
| GACCTTTTTTTTTTTTT | SEQ ID NO: 451 | | |
| GATACATTTR2GATGA | SEQ ID NO: | | |
| T-3′ | | | |
| Représenté par SEQ ID | | | |
| NO: 445-R1-SEQ ID | | | |
| NO: 446-R1-SEQ ID | | | |
| NO: 470-X-SEQ ID NO: | | | |
| 474-R2-SEQ ID NO: 449 | | | |
| SEQ ID NO: | | | |

Sequences with two transposase sequences
5′ insertion-model 2

| | | | |
|---|---|---|---|
| 5′-ATCATCR1TTTTTTT | 5′-YacttggTTAATTA | SEQ ID NO: n | SEQ ID NO: n − 1 |
| TTTTTTTTTTGATAGT | ATTTTTTTTTTTTTT | n being an even | n being the same |
| AGR1TTTTTTTTTTTTT | TTTTTTTTR2CTACT | number ranging | even number as |
| TTTTTTTTTGCGGCGA | ATC-3′ | from 1 to 6 and | R1 ranging from |
| TCGGCTTTTTTTTTTT | Sumarized by Y-SEQ | from 269 to 424 | 1 to 6 and from |
| TTTTTXTTTTTTTTTTT | ID NO: 450-R2-SEQ | | 269 to 424 |
| TTTTTGCCGATCGCC | ID NO: 451 | | |
| GCTTTTTTTTTTTTTG | SEQ ID NO: | | |
| ATACATTTR2GATGAT-3′ | | | |
| Sumarized by SEQ ID | | | |
| NO: 445-R1-SEQ ID | | | |
| NO: 446-R1-SEQ ID | | | |
| NO: 471-X-SEQ ID NO: | | | |
| 472-R2-SEQ ID NO: 449 | | | |
| SEQ ID NO: | | | |
| 5′-ATCATCR1TTTTTTT | 5′-YacttggTTAATTA | SEQ ID NO: n | SEQ ID NO: n + 1 |
| TTTTTTTTTTGATAGT | ATTTTTTTTTTTTTT | n being an odd | n being the same |
| AGR1TTTTTTTTTTTTT | TTTTTTTTR2CTACT | number ranging | odd number as |
| TTTTTTTTTGCGGCGA | ATC-3′ | from 1 to 6 and | R1 ranging from |
| TCGGCTTTTTTTTTTT | Sumarized by Y-SEQ | from 269 to 424 | 1 to 6 and from |
| TTTTTXTTTTTTTTTTT | ID NO: 450-R2-SEQ | | 269 to 424 |
| TTTTTGCCGATCGCC | ID NO: 451 | | |
| GCTTTTTTTTTTTTTG | SEQ ID NO: | | |
| ATACATTTR2GATGAT-3′ | | | |
| Sumarized by SEQ ID | | | |
| NO: 445-R1-SEQ ID | | | |
| NO: 446-R1-SEQ ID | | | |
| NO: 471-X-SEQ ID NO: | | | |
| 472-R2-SEQ ID NO: 449 | | | |
| SEQ ID NO: | | | |
| 5′-ATCATCR1TTTTTTT | 5′-acttggYTTAATTA | SEQ ID NO: n | SEQ ID NO: n − 1 |
| TTTTTTTTTTGATAGT | ATTTTTTTTTTTTTT | n being an even | n being the same |
| AGR1TTTTTTTTTTTTT | TTTTTTTTR2CTACT | number ranging | even number as |
| TTTTTTTTTGCGGCGA | ATC-3′ | from 1 to 6 and | R1 ranging from |
| TCGGCTTTTTTTTTTT | Représenté par | from 269 to 424 | 1 to 6 and from |
| TTTTTXTTTTTTTTTTT | acttgg-Y-SEQ ID NO: | | 269 to 424 |
| TTTTTGCCGATCGCC | 473-R2-SEQ ID NO: | | |
| GCTTTTTTTTTTTTTG | 451 | | |
| ATACATTTR2GATGAT-3′ | SEQ ID NO: | | |
| Sumarized by SEQ | | | |
| ID NO: 445-R1-SEQ ID | | | |
| NO: 446-R1-SEQ ID | | | |
| NO: 471-X-SEQ ID NO: | | | |
| 472-R2-SEQ ID NO: 449 | | | |
| SEQ ID NO: | | | |
| 5′-ATCATCR1TTTTTTT | 5′-acttggYTTAATTA | SEQ ID NO: n | SEQ ID NO: n + 1 |
| TTTTTTTTTTGATAGT | ATTTTTTTTTTTTTT | n being an odd | n being the same |
| AGR1TTTTTTTTTTTTT | TTTTTTTTR2CTACT | number ranging | odd number as |
| TTTTTTTTTGCGGCGA | ATC-3′ | from 1 to 6 and | R1 ranging from |
| TCGGCTTTTTTTTTTT | Représenté par | from 269 to 424 | 1 to 6 and from |
| TTTTTXTTTTTTTTTTT | acttgg-Y-SEQ ID NO: | | 269 to 424 |
| TTTTTGCCGATCGCC | 473-R2-SEQ ID NO: | | |
| GCTTTTTTTTTTTTTG | 451 | | |
| ATACATTTR2GATGAT-3′ | SEQ ID NO: | | |
| Sumarized by SEQ ID | | | |
| NO: 445-R1-SEQ ID | | | |
| NO: 446-R1-SEQ ID | | | |
| NO: 471-X-SEQ ID NO: | | | |
| 472-R2-SEQ ID NO: 449 | | | |
| SEQ ID NO: | | | |
| 5′-ATCATCR1TTTTTTT | 5′-YacttggcatatgTTA | SEQ ID NO: n | SEQ ID NO: n − 1 |
| TTTTTTTTTTGATAGT | ATTAATTTTTTTTTT | n being an even | n being the same |
| AGR1TTTTTTTTTTTTT | TTTTTTTTTTTTR2C | number ranging | even number as |
| TTTTTTTTTGCGGCGA | TACTATC-3′ | from 1 to 6 and | R1 ranging from |
| TCGGCTTTTTTTTTTT | Représenté par Y- | from 269 to 424 | 1 to 6 and from |
| TTTTTXTTTTTTTTTTT | SEQ ID NO: 475-R2- | | 269 to 424 |
| TTTTTGCCGATCGCC | SEQ ID NO: 451 | | |
| GCTTTTTTTTTTTTTG | SEQ ID NO: | | |
| ATACATTTR2GATGAT-3′ | | | |
| Sumarized by SEQ | | | |
| ID NO: 445-R1-SEQ ID | | | |
| NO: 446-R1-SEQ ID | | | |
| NO: 471-X-SEQ ID NO: | | | |
| 472-R2-SEQ ID NO: 449 | | | |
| SEQ ID NO: | | | |
| 5′-ATCATCR1TTTTTTT | 5′-YacttggcatatgTTA | SEQ ID NO: n | SEQ ID NO: n + 1 |
| TTTTTTTTTTGATAGT | ATTAATTTTTTTTTT | n being an odd | n being the same |
| AGR1TTTTTTTTTTTTT | TTTTTTTTTTTTR2C | number ranging | odd number as |
| TTTTTTTTTGCGGCGA | TACTATC-3′ | from 1 to 6 and | R1 ranging from |
| TCGGCTTTTTTTTTTT | Représenté par Y- | from 269 to 424 | 1 to 6 and from |
| TTTTTXTTTTTTTTTTT | SEQ ID NO: 475-R2- | | 269 to 424 |
| TTTTTGCCGATCGCC | SEQ ID NO: 451 | | |
| GCTTTTTTTTTTTTTG | SEQ ID NO: | | |
| ATACATTTR2GATGAT-3′ | | | |
| Sumarized by SEQ | | | |
| ID NO: 445-R1-SEQ ID | | | |
| NO: 446-R1-SEQ ID | | | |
| NO: 471-X-SEQ ID NO: | | | |
| 472-R2-SEQ ID NO: 449 | | | |
| SEQ ID NO: | | | |

Sequences with two transposase sequences
5′ insertion-model 3

| | | | |
|---|---|---|---|
| 5′-GATAGTAGR1TTTT | 5′-YacttggTTAATTA | SEQ ID NO: n | SEQ ID NO: n − 1 |
| TTTTTTTTTTTTTTTTT | ATTTTTTTTTTTTTT | n being an even | n being the same |
| TGGCGATCGCTTTTTT | TTTTTTTTR2CTACT | number ranging | even number as |
| TTTTTTTTTTXTTTTTT | ATC-3′ | from 1 to 6 and | R1 ranging from |
| TTTTTTTTTTGCGATC | Sumarized by Y-SEQ | from 269 to 424 | 1 to 6 and from |
| GCCTTTTTTTTTTTTT | ID NO: 450-R2-SEQ | | 269 to 424 |
| GATACATTTR2GATGA | ID NO: 451 | | |
| TTTTTTTTTTTTTTTTT | SEQ ID NO: | | |
| TATCATCR1-3′ | | | |
| Représenté par | | | |
| GATAGTAG-R1-SEQ ID | | | |
| NO: 476-X-SEQ ID NO: | | | |
| 448-R2-SEQ ID NO: | | | |
| 477-R1 | | | |
| SEQ ID NO: | | | |
| 5′-GATAGTAGR1TTTT | 5′-YacttggTTAATTA | SEQ ID NO: n | SEQ ID NO: n + 1 |
| TTTTTTTTTTTTTTTTT | ATTTTTTTTTTTTTT | n being an odd | n being the same |
| TGGCGATCGCTTTTTT | TTTTTTTTR2CTACT | number ranging | odd number as |
| TTTTTTTTTTXTTTTTT | ATC-3′ | from 1 to 6 and | R1 ranging from |
| TTTTTTTTTTGCGATC | Sumarized by Y-SEQ | from 269 to 424 | 1 to 6 and from |
| GCCTTTTTTTTTTTTT | ID NO: 450-R2-SEQ | | 269 to 424 |
| GATACATTTR2GATGA | ID NO: 451 | | |
| TTTTTTTTTTTTTTTTT | SEQ ID NO: | | |
| TATCATCR1-3′ | | | |
| Représenté par | | | |
| GATAGTAG-R1-SEQ ID | | | |
| NO: 476-X-SEQ ID NO: | | | |
| 448-R2-SEQ ID NO: | | | |
| 477-R1 | | | |
| SEQ ID NO: | | | |
| 5′-GATAGTAGR1TTTT | 5′-acttggYTTAATTA | SEQ ID NO: n | SEQ ID NO: n − 1 |
| TTTTTTTTTTTTTTTTT | ATTTTTTTTTTTTTT | n being an even | n being the same |
| TGGCGATCGCTTTTTT | TTTTTTTTR2CTACT | number ranging | even number as |
| TTTTTTTTTTXTTTTTT | ATC-3′ Représenté | from 1 to 6 and | R1 ranging from |
| TTTTTTTTTTGCGATC | par acttgg-Y-SEQ ID | from 269 to 424 | 1 to 6 and from |
| GCCTTTTTTTTTTTTT | NO: 473-R2-SEQ ID | | 269 to 424 |
| GATACATTTR2GATGA | NO: 451 | | |
| TTTTTTTTTTTTTTTTT | SEQ ID NO: | | |
| TATCATCR1-3′ | | | |
| Représenté par | | | |
| GATAGTAG-R1-SEQ ID | | | |
| NO: 476-X-SEQ ID NO: | | | |
| 448-R2-SEQ ID NO: | | | |
| 477-R1 | | | |
| SEQ ID NO: | | | |
| 5′-GATAGTAGR1TTTT | 5′-acttggYTTAATTA | SEQ ID NO: n | SEQ ID NO: n + 1 |
| TTTTTTTTTTTTTTTTT | ATTTTTTTTTTTTTT | n being an odd | n being the same |
| TGGCGATCGCTTTTTT | TTTTTTTTR2CTACT | number ranging | odd number as |
| TTTTTTTTTTXTTTTTT | ATC-3′ | from 1 to 6 and | R1 ranging from |
| TTTTTTTTTTGCGATC | Représenté par | from 269 to 424 | 1 to 6 and from |
| GCCTTTTTTTTTTTTT | acttgg-Y-SEQ ID NO: | | 269 to 424 |
| GATACATTTR2GATGA | 473-R2-SEQ ID NO: | | |
| TTTTTTTTTTTTTTTTT | 451 | | |
| TATCATCR1-3′ | SEQ ID NO: | | |
| Représenté par | | | |
| GATAGTAG-R1-SEQ ID | | | |
| NO: 476-X-SEQ ID NO: | | | |
| 448-R2-SEQ ID NO: | | | |
| 477-R1 | | | |
| SEQ ID NO: | | | |
| 5′-GATAGTAGR1TTTT | 5′-YacttggcatatgTTA | SEQ ID NO: n | SEQ ID NO: n − 1 |
| TTTTTTTTTTTTTTTTT | ATTAATTTTTTTTTT | n being an even | n being the same |
| TGGCGATCGCTTTTTT | TTTTTTTTTTTTR2C | number ranging | even number as |
| TTTTTTTTTTXTTTTTT | TACTATC-3′ | from 1 to 6 and | R1 ranging from |
| TTTTTTTTTTGCGATC | Représenté par Y- | from 269 to 424 | 1 to 6 and from |
| GCCTTTTTTTTTTTTT | SEQ ID NO: 475-R2- | | 269 to 424 |
| GATACATTTR2GATGA | SEQ ID NO: 451 | | |
| TTTTTTTTTTTTTTTTT | SEQ ID NO: | | |
| TATCATCR1-3′ | | | |
| Représenté par | | | |
| GATAGTAG-R1-SEQ ID | | | |
| NO: 476-X-SEQ ID NO: | | | |
| 448-R2-SEQ ID NO: | | | |
| 477-R1SEQ ID NO: | | | |
| 5′-GATAGTAGR1TTTT | 5′-YacttggcatatgTTA | SEQ ID NO: n | SEQ ID NO: n + 1 |
| TTTTTTTTTTTTTTTTT | ATTAATTTTTTTTTT | n being an odd | n being the same |
| TGGCGATCGCTTTTTT | TTTTTTTTTTTTR2C | number ranging | odd number as |
| TTTTTTTTTTXTTTTTT | TACTATC-3′ | from 1 to 6 and | R1 ranging from |
| TTTTTTTTTTGCGATC | Représenté par Y- | from 269 to 424 | 1 to 6 and from |
| GCCTTTTTTTTTTTTT | SEQ ID NO: 475-R2- | | 269 to 424 |
| GATACATTTR2GATGA | SEQ ID NO: 451 | | |
| TTTTTTTTTTTTTTTTT | SEQ ID NO: | | |
| TATCATCR1-3′ | | | |
| Représenté par | | | |
| GATAGTAG-R1-SEQ ID | | | |
| NO: 476-X-SEQ ID NO: | | | |
| 448-R2-SEQ ID NO: | | | |
| 477-R1 | | | |
| SEQ ID NO: | | | |

Sequences with two transposase sequences
5′ insertion-model 4

| | | | |
|---|---|---|---|
| 5′-GATAGTAGR1TTTT | 5′-YacttggTTAATTA | SEQ ID NO: n | SEQ ID NO: n − 1 |
| TTTTTTTTTTTTTTTTT | ATTTTTTTTTTTTTT | n being an even | n being the same |
| TGGTCGCTTTTTTTTT | TTTTTTTTR2CTACT | number ranging | even number as |
| TTTTTTTXTTTTTTTTT | ATC-3′ | from 1 to 6 and | R1 ranging from |
| TTTTTTTGCGACCTTT | Sumarized by Y-SEQ | from 269 to 424 | 1 to 6 and from |
| TTTTTTTTTGATACAT | ID NO: 450-R2-SEQ | | 269 to 424 |
| TTR2GATGATTTTTTT | ID NO: 451 | | |
| TTTTTTTTTTTATCATC | SEQ ID NO: | | |
| R1-3′ | | | |
| Représenté par | | | |
| GATAGTAG-R1-SEQ ID | | | |
| NO: 470-X-SEQ ID NO: | | | |
| 471-R2-SEQID NO 477- | | | |
| R1 | | | |
| SEQ ID NO: | | | |
| 5′-GATAGTAGR1TTTT | 5′-YacttggTTAATTA | SEQ ID NO: n | SEQ ID NO: n + 1 |
| TTTTTTTTTTTTTTTTT | ATTTTTTTTTTTTTT | n being an odd | n being the same |
| TGGTCGCTTTTTTTTT | TTTTTTTTR2CTACT | number ranging | odd number as |
| TTTTTTTXTTTTTTTTT | ATC-3′ | from 1 to 6 and | R1 ranging from |
| TTTTTTTGCGACCTTT | Sumarized by Y-SEQ | from 269 to 424 | 1 to 6 and from |
| TTTTTTTTTTGATACAT | ID NO: 450-R2-SEQ | | 269 to 424 |
| TTR2GATGATTTTTTT | ID NO: 451 | | |
| TTTTTTTTTTTATCATC | SEQ ID NO: | | |
| R1-3′ | | | |
| Représenté par | | | |
| GATAGTAG-R1-SEQ ID | | | |
| NO: 470-X-SEQ ID NO: | | | |
| 471-R2-SEQID NO 477- | | | |
| R1 | | | |
| SEQ ID NO: | | | |
| 5′-GATAGTAGR1TTTT | 5′-acttggYTTAATTA | SEQ ID NO: n | SEQ ID NO: n − 1 |
| TTTTTTTTTTTTTTTTT | ATTTTTTTTTTTTTT | n being an even | n being the same |
| TGGTCGCTTTTTTTTT | TTTTTTTTR2CTACT | number ranging | even number as |
| TTTTTTTXTTTTTTTTT | ATC-3′ | from 1 to 6 and | R1 ranging from |
| TTTTTTTGCGACCTTT | Représenté par | from 269 to 424 | 1 to 6 and from |
| TTTTTTTTTTGATACAT | acttgg-Y-SEQ ID NO: | | 269 to 424 |
| TTR2GATGATTTTTTT | 473-R2-SEQ ID NO: | | |
| TTTTTTTTTTTATCATC | 451 | | |
| R1-3′ Représenté par | SEQ ID NO: | | |
| GATAGTAG-R1-SEQ ID | | | |
| NO: 470-X-SEQ ID NO: | | | |
| 471-R2-SEQID NO 477- | | | |
| R1 | | | |
| SEQ ID NO: | | | |
| 5′-GATAGTAGR1TTTT | 5′-acttggYTTAATTA | SEQ ID NO: n | SEQ ID NO: n + 1 |
| TTTTTTTTTTTTTTTTT | ATTTTTTTTTTTTTT | n being an odd | n being the same |
| TGGTCGCTTTTTTTTT | TTTTTTTTR2CTACT | number ranging | odd number as |
| TTTTTTTXTTTTTTTTT | ATC-3′ | from 1 to 6 and | R1 ranging from |
| TTTTTTTGCGACCTTT | Représenté par | from 269 to 424 | 1 to 6 and from |
| TTTTTTTTTTGATACAT | acttgg-Y-SEQ ID NO: | | 269 to 424 |
| TTR2GATGATTTTTTT | 473-R2-SEQ ID NO: | | |
| TTTTTTTTTTTATCATC | 451 | | |
| R1-3′ Représenté par | SEQ ID NO: | | |
| GATAGTAG-R1-SEQ ID | | | |
| NO: 470-X-SEQ ID NO: | | | |
| 471-R2-SEQID NO 477- | | | |
| R1 | | | |
| SEQ ID NO: | | | |
| 5′-GATAGTAGR1TTTT | 5′-YacttggcatatgTTA | SEQ ID NO: n | SEQ ID NO: n − 1 |
| TTTTTTTTTTTTTTTTT | ATTAATTTTTTTTTT | n being an even | n being the same |
| TGGTCGCTTTTTTTTT | TTTTTTTTTTTTR2C | number ranging | even number as |
| TTTTTTTXTTTTTTTTT | TACTATC-3′ | from 1 to 6 and | R1 ranging from |
| TTTTTTTGCGACCTTT | Représenté par Y- | from 269 to 424 | 1 to 6 and from |
| TTTTTTTTTTGATACAT | SEQ ID NO: 475-R2- | | 269 to 424 |
| TTR2GATGATTTTTTT | SEQ ID NO: 451 | | |
| TTTTTTTTTTTATCATC | SEQ ID NO: | | |
| R1-3′ | | | |
| Représenté par | | | |
| GATAGTAG-R1-SEQ ID | | | |
| NO: 470-X-SEQ ID NO: | | | |
| 471-R2-SEQID NO 477- | | | |
| R1 | | | |
| SEQ ID NO: | | | |
| 5′-GATAGTAGR1TTTT | 5′-YacttggcatatgTTA | SEQ ID NO: n | SEQ ID NO: n + 1 |
| TTTTTTTTTTTTTTTTT | ATTAATTTTTTTTTT | n being an odd | n being the same |
| TGGTCGCTTTTTTTTT | TTTTTTTTTTTTR2C | number ranging | odd number as |
| TTTTTTTXTTTTTTTTT | TACTATC-3′ | from 1 to 6 and | R1 ranging from |
| TTTTTTTGCGACCTTT | Représenté par Y- | from 269 to 424 | 1 to 6 and from |
| TTTTTTTTTTGATACAT | SEQ ID NO: 475-R2- | | 269 to 424 |
| TTR2GATGATTTTTTT | SEQ ID NO: 451 | | |
| TTTTTTTTTTTATCATC | SEQ ID NO: | | |
| R1-3′ | | | |
| Représenté par | | | |
| GATAGTAG-R1-SEQ ID | | | |
| NO: 470-X-SEQ ID NO: | | | |
| 471-R2-SEQID NO 477- | | | |
| R1 | | | |
| SEQ ID NO: | | | |

Sequences with two transposase sequences
5′ insertion-model 5

| | | | |
|---|---|---|---|
| 5′-GATAGTAGR1TTTT | 5′-YacttggTTAATTA | SEQ ID NO: n | SEQ ID NO: n − 1 |
| TTTTTTTTTTTTTTTTT | ATTTTTTTTTTTTTT | n being an even | n being the same |
| TGCGGCGATCGGCTT | TTTTTTTTR2CTACT | number ranging | even number as |
| TTTTTTTTTTTTTTXTT | ATC-3′ | from 1 to 6 and | R1 ranging from |
| TTTTTTTTTTTTTTGCC | Sumarized by Y-SEQ | from 269 to 424 | 1 to 6 and from |
| GATCGCCGCTTTTTTT | ID NO: 450-R2-SEQ | | 269 to 424 |
| TTTTTTGATACATTTR | ID NO: 451 | | |
| 2GATGATTTTTTTTTTT | SEQ ID NO: | | |
| TTTTTTTATCATCR1-3′ | | | |
| Représenté par | | | |
| GATAGTAG-R1- SEQ | | | |
| ID NO: 471-X-SEQ ID | | | |
| NO: 472-R2-SEQ ID | | | |
| NO: 477-R1 | | | |
| SEQ ID NO: | | | |
| 5′-GATAGTAGR1TTTT | 5′-YacttggTTAATTA | SEQ ID NO: n | SEQ ID NO: n + 1 |
| TTTTTTTTTTTTTTTTT | ATTTTTTTTTTTTTT | n being an odd | n being the same |
| TGCGGCGATCGGCTT | TTTTTTTTR2CTACT | number ranging | odd number as |
| TTTTTTTTTTTTTTXTT | ATC-3′ | from 1 to 6 and | R1 ranging from |
| TTTTTTTTTTTTTTGCC | Sumarized by Y-SEQ | from 269 to 424 | 1 to 6 and from |
| GATCGCCGCTTTTTTT | ID NO: 450-R2-SEQ | | 269 to 424 |
| TTTTTTGATACATTTR | ID NO: 451 | | |
| 2GATGATTTTTTTTTTT | SEQ ID NO: | | |
| TTTTTTTATCATCR1-3′ | | | |
| Représenté par | | | |
| GATAGTAG-R1- SEQ | | | |
| ID NO: 471-X-SEQ ID | | | |
| NO: 472-R2-SEQ ID | | | |
| NO: 477-R1 | | | |
| SEQ ID NO: | | | |
| 5′-GATAGTAGR1TTTT | 5′-acttggYTTAATTA | SEQ ID NO: n | SEQ ID NO: n − 1 |
| TTTTTTTTTTTTTTTTT | ATTTTTTTTTTTTTT | n being an even | n being the same |
| TGCGGCGATCGGCTT | TTTTTTTTR2CTACT | number ranging | even number as |
| TTTTTTTTTTTTTTXTT | ATC-3′ Représenté | from 1 to 6 and | R1 ranging from |
| TTTTTTTTTTTTTTGCC | par acttgg-Y-SEQ ID | from 269 to 424 | 1 to 6 and from |
| GATCGCCGCTTTTTTT | NO: 473-R2-SEQ ID | | 269 to 424 |
| TTTTTTGATACATTTR | NO: 451 | | |
| 2GATGATTTTTTTTTTT | SEQ ID NO: | | |
| TTTTTTTATCATCR1-3′ | | | |
| Représenté par | | | |
| GATAGTAG-R1- SEQ | | | |
| ID NO: 471-X-SEQ ID | | | |
| NO: 472-R2-SEQ ID | | | |
| NO: 477-R1 | | | |
| SEQ ID NO: | | | |
| 5′-GATAGTAGR1TTTT | 5′-acttggYTTAATTA | SEQ ID NO: n | SEQ ID NO: n + 1 |
| TTTTTTTTTTTTTTTTT | ATTTTTTTTTTTTTT | n being an odd | n being the same |
| TGCGGCGATCGGCTT | TTTTTTTTR2CTACT | number ranging | odd number as |
| TTTTTTTTTTTTTTXTT | ATC-3′ | from 1 to 6 and | R1 ranging from |
| TTTTTTTTTTTTTTGCC | Représenté par | from 269 to 424 | 1 to 6 and from |
| GATCGCCGCTTTTTTT | acttgg-Y-SEQ ID NO: | | 269 to 424 |
| TTTTTTGATACATTTR | 473-R2-SEQ ID NO: | | |
| 2GATGATTTTTTTTTTT | 451 | | |
| TTTTTTTATCATCR1-3′ | SEQ ID NO: | | |
| Représenté par | | | |
| GATAGTAG-R1- SEQ | | | |
| ID NO: 471-X-SEQ ID | | | |
| NO: 472-R2-SEQ ID | | | |
| NO: 477-R1 | | | |
| SEQ ID NO: | | | |
| 5′-GATAGTAGR1TTTT | 5′-YacttggcatatgTTA | SEQ ID NO: n | SEQ ID NO: n − 1 |
| TTTTTTTTTTTTTTTTT | ATTAATTTTTTTTTT | n being an even | n being the same |
| TGCGGCGATCGGCTT | TTTTTTTTTTTTR2C | number ranging | even number as |
| TTTTTTTTTTTTTTXTT | TACTATC-3′ | from 1 to 6 and | R1 ranging from |
| TTTTTTTTTTTTTTGCC | Représenté par Y- | from 269 to 424 | 1 to 6 and from |
| GATCGCCGCTTTTTTT | SEQ ID NO: 475-R2- | | 269 to 424 |
| TTTTTTGATACATTTR | SEQ ID NO: 451 | | |
| 2GATGATTTTTTTTTTT | SEQ ID NO: | | |
| TTTTTTTATCATCR1-3′ | | | |
| Représenté par | | | |
| GATAGTAG-R1- SEQ | | | |
| ID NO: 471-X-SEQ ID | | | |
| NO: 472-R2-SEQ ID | | | |
| NO: 477-R1 | | | |
| SEQ ID NO: | | | |
| 5′-GATAGTAGR1TTTT | 5′-YacttggcatatgTTA | SEQ ID NO: n | SEQ ID NO: n + 1 |
| TTTTTTTTTTTTTTTTT | ATTAATTTTTTTTTT | n being an odd | n being the same |
| TGCGGCGATCGGCTT | TTTTTTTTTTTTR2C | number ranging | odd number as |
| TTTTTTTTTTTTTTXTT | TACTATC-3′ | from 1 to 6 and | R1 ranging from |
| TTTTTTTTTTTTTTGCC | Représenté par Y- | from 269 to 424 | 1 to 6 and from |
| GATCGCCGCTTTTTTT | SEQ ID NO: 475-R2- | | 269 to 424 |
| TTTTTTGATACATTTR | SEQ ID NO: 451 | | |
| 2GATGATTTTTTTTTTT | SEQ ID NO: | | |
| TTTTTTTATCATCR1-3′ | | | |
| Représenté par | | | |
| GATAGTAG-R1- SEQ | | | |
| ID NO: 471-X-SEQ ID | | | |
| NO: 472-R2-SEQ ID | | | |
| NO: 477-R1 | | | |
| SEQ ID NO: | | | |

Sequences with two transposase sequences
3′ insertion-model 1

| | | | |
|---|---|---|---|
| 5′-CACGTGR1TTTCTC | 5′-TATTCGTCR1TTT | SEQ ID NO: n | SEQ ID NO: n − 1 |
| GATCATTATTATTTTT | TTTTTTTTTTTTTTT | n being an even | n being the same |
| TGGCGATCGCTTTTTT | ATTAATTATTActtgta | number ranging | even number as |
| TTTTTTTTTTXTTTTTT | Y-3′ | from 1 to 6 and | R1 ranging from |
| TTTTTTTTTTGCGATC | Représenté par | from 269 to 424 | 1 to 6 and from |
| GCCTTTTTTTTTTTTTT | TATTCGTC-R1-SEQ | | 269 to 424 |
| TTTTTTTR2GACGAAT | ID NO: 481-Y | | |
| ATTTTTTTTTTTTTTTT | SEQ ID NO: | | |
| TTTTR2CACGTG-3′ | | | |
| CACGTG-R1-SEQ ID | | | |
| NO: 478-X-SEQ ID NO: | | | |
| 479-R2-SEQ ID NO: | | | |
| 480-R2-CACGTG | | | |
| SEQ ID NO: | | | |
| 5′-CACGTGR1TTTCTC | 5′-TATTCGTCR1TTT | SEQ ID NO: n | SEQ ID NO: n + 1 |
| GATCATTATTATTTTT | TTTTTTTTTTTTTTT | n being an odd | n being the same |
| TGGCGATCGCTTTTTT | ATTAATTATTActtgta | number ranging | odd number as |
| TTTTTTTTTTXTTTTTT | Y-3′ | from 1 to 6 and | R1 ranging from |
| TTTTTTTTTTGCGATC | Représenté par | from 269 to 424 | 1 to 6 and from |
| GCCTTTTTTTTTTTTTT | TATTCGTC-R1-SEQ | | 269 to 424 |
| TTTTTTTR2GACGAAT | ID NO: 481-Y | | |
| ATTTTTTTTTTTTTTTT | SEQ ID NO: | | |
| TTTTR2CACGTG-3′ | | | |
| CACGTG-R1-SEQ ID | | | |
| NO: 478-X-SEQ ID NO: | | | |
| 479-R2-SEQ ID NO: | | | |
| 480-R2-CACGTG | | | |
| SEQ ID NO: | | | |
| 5′-CACGTGR1TTTCTC | 5′-TATTCGTCR1TTT | SEQ ID NO: n | SEQ ID NO: n − 1 |
| GATCATTATTATTTTT | TTTTTTTTTTTTTTT | n being an even | n being the same |
| TGGCGATCGCTTTTTT | ATTAATTATTAYcttgt | number ranging | even number as |
| TTTTTTTTTT(SEQ ID | a-3′ | from 1 to 6 and | R1 ranging from |
| NO: | Représenté par | from 269 to 424 | 1 to 6 and from |
| 478)XTTTTTTTTTTTTT | TATTCGTC-R1-SEQ | | 269 to 424 |
| TTTGCGATCGCCTTTT | ID NO: 482-Y-cttgta | | |
| TTTTTTTTTTTTTTTTT | SEQ ID NO: | | |
| (SEQ ID NO: | | | |
| 479)2GACGAATATTTT | | | |
| TTTTTTTTTTTTTTTT | | | |
| (SEQ ID NO: | | | |
| 480)2CACGTG-3′ | | | |
| CACGTG-R1-SEQ ID | | | |
| NO: 478-X-SEQ ID NO: | | | |
| 479-R2-SEQ ID NO: | | | |
| 480-R2-CACGTG | | | |
| SEQ ID NO: | | | |
| 5′-CACGTGR1TTTCTC | 5′-TATTCGTCR1TTT | SEQ ID NO: n | SEQ ID NO: n + 1 |
| TGGCGATCGCTTTTTT | TTTTTTTTTTTTTTT | n being an odd | n being the same |
| GATCATTATTATTTTT | ATTAATTATTAYcttgt | number ranging | odd number as |
| TTTTTTTTTTXTTTTTT | a-3′ | from 1 to 6 and | R1 ranging from |
| TTTTTTTTTTGCGATC | Représenté par | from 269 to 424 | 1 to 6 and from |
| GCCTTTTTTTTTTTTTT | TATTCGTC-R1-SEQ | | 269 to 424 |
| TTTTTTTR2GACGAAT | ID NO: 482-Y-cttgta | | |
| ATTTTTTTTTTTTTTTT | SEQ ID NO: | | |
| TTTTR2CACGTG-3′ | | | |
| CACGTG-R1-SEQ ID | | | |
| NO: 478-X-SEQ ID NO: | | | |
| 479-R2-SEQ ID NO: | | | |
| 480-R2-CACGTG | | | |
| SEQ ID NO: | | | |
| 5′-CACGTGR1TTTCTC | 5′-TATTCGTCR1TTT | SEQ ID NO: n | SEQ ID NO: n − 1 |
| GATCATTATTATTTTT | TTTTTTTTTTTTTTT | n being an even | n being the same |
| TGGCGATCGCTTTTTT | ATTAATTATTATCC | number ranging | even number as |
| TTTTTTTTTTXTTTTTT | GGActtgtaY-3′ | from 1 to 6 and | R1 ranging from |
| TTTTTTTTGCGATC | Représenté par | from 269 to 424 | 1 to 6 and from |
| GCCTTTTTTTTTTTTTT | TATTCGTC-R1-SEQ | | 269 to 424 |
| TTTTTTTR2GACGAAT | ID NO: 483-Y | | |
| ATTTTTTTTTTTTTTTT | SEQ ID NO: | | |
| TTTTR2CACGTG-3′ | | | |
| CACGTG-R1-SEQ ID | | | |
| NO: 478-X-SEQ ID NO: | | | |
| 479-R2-SEQ ID NO: | | | |
| 480-R2-CACGTGSEQ | | | |
| ID NO: | | | |
| 5′-CACGTGR1TTTCTC | 5′-TATTCGTCR1TTT | SEQ ID NO: n | SEQ ID NO: n + 1 |
| GATCATTATTATTTTT | TTTTTTTTTTTTTTT | n being an odd | n being the same |
| TGGCGATCGCTTTTTT | ATTAATTATTATCC | number ranging | odd number as |
| TTTTTTTTTTXTTTTTT | GGActtgtaY-3′ | from 1 to 6 and | R1 ranging from |
| TTTTTTTTTTTGCGATC | Représenté par | from 269 to 424 | 1 to 6 and from |
| GCCTTTTTTTTTTTTTT | TATTCGTC-R1-SEQ | | 269 to 424 |
| TTTTTTTR2GACGAAT | ID NO: 483-Y | | |
| ATTTTTTTTTTTTTTTT | SEQ ID NO: | | |
| TTTTR2CACGTG-3′ | | | |
| CACGTG-R1-SEQ ID | | | |
| NO: 478-X-SEQ ID NO: | | | |
| 479-R2-SEQ ID NO: | | | |
| 480-R2-CACGTG | | | |
| SEQ ID NO: | | | |

Sequences with two transposase sequences
3′ insertion-model 2

| | | | |
|---|---|---|---|
| 5′-CACGTGR1TTTCTC | 5′-TATTCGTCR1TTT | SEQ ID NO: n | SEQ ID NO: n − 1 |
| GATCATTATTATTTTT | TTTTTTTTTTTTTTT | n being an even | n being the same |
| TGGTCGCTTTTTTTTT | ATTAATTATTActtgta | number ranging | even number as |
| TTTTTTTXTTTTTTTTT | Y-3′ | from 1 to 6 and | R1 ranging from |
| TTTTTTTGCGACCTTT | Représenté par | from 269 to 424 | 1 to 6 and from |
| TTTTTTTTTTTTTTTTT | TATTCGTC-R1-SEQ | | 269 to 424 |
| TR2GACGAATATTTTT | ID NO: 481-Y) | | |
| TTTTTTTTTTTTTTTR2 | SEQ ID NO: | | |
| CACGTG-3′ | | | |
| Représenté par | | | |
| CACGTG-R1-SEQ ID | | | |
| NO: 484-X-SEQ ID NO: | | | |
| 485-R2-SEQ ID NO: | | | |
| 480-R2-CACGTG | | | |
| SEQ ID NO: | | | |
| 5′-CACGTGR1TTTCTC | 5′-TATTCGTCR1TTT | SEQ ID NO: n | SEQ ID NO: n + 1 |
| GATCATTATTATTTTT | TTTTTTTTTTTTTTT | n being an odd | n being the same |
| TGGTCGCTTTTTTTTT | ATTAATTATTActtgta | number ranging | odd number as |
| TTTTTTTXTTTTTTTTT | Y-3′ | from 1 to 6 and | R1 ranging from |
| TTTTTTTGCGACCTTT | Représenté par | from 269 to 424 | 1 to 6 and from |
| TTTTTTTTTTTTTTTTT | TATTCGTC-R1-SEQ | | 269 to 424 |
| TR2GACGAATATTTTT | ID NO: 481-Y | | |
| TTTTTTTTTTTTTTTR2 | SEQ ID NO: | | |
| CACGTG-3′ | | | |
| Représenté par | | | |
| CACGTG-R1-SEQ ID | | | |
| NO: 484-X-SEQ ID NO: | | | |
| 485-R2-SEQ ID NO: | | | |
| 480-R2-CACGTG | | | |
| SEQ ID NO: | | | |
| 5′-CACGTGR1TTTCTC | 5′-TATTCGTCR1TTT | SEQ ID NO: n | SEQ ID NO: n − 1 |
| GATCATTATTATTTTT | TTTTTTTTTTTTTTT | n being an even | n being the same |
| TGGTCGCTTTTTTTTT | ATTAATTATTAYcttgt | number ranging | even number as |
| TTTTTTTXTTTTTTTTT | a-3′ | from 1 to 6 and | R1 ranging from |
| TTTTTTTGCGACCTTT | Représenté par | from 269 to 424 | 1 to 6 and from |
| TTTTTTTTTTTTTTTTT | TATTCGTC-R1-SEQ | | 269 to 424 |
| TR2GACGAATATTTTT | ID NO: 482-Y-cttgta | | |
| TTTTTTTTTTTTTTTR2 | SEQ ID NO: | | |
| CACGTG-3′ | | | |
| Représenté par | | | |
| CACGTG-R1-SEQ ID | | | |
| NO: 484-X-SEQ ID NO: | | | |
| 485-R2-SEQ ID NO: | | | |
| 480-R2-CACGTG | | | |
| SEQ ID NO: | | | |
| 5′-CACGTGR1TTTCTC | 5′-TATTCGTCR1TTT | SEQ ID NO: n | SEQ ID NO: n + 1 |
| GATCATTATTATTTTT | TTTTTTTTTTTTTTT | n being an odd | n being the same |
| TGGTCGCTTTTTTTTT | ATTAATTATTAYcttgt | number ranging | odd number as |
| TTTTTTTXTTTTTTTTT | a-3′ | from 1 to 6 and | R1 ranging from |
| TTTTTTTGCGACCTTT | Représenté par | from 269 to 424 | 1 to 6 and from |
| TTTTTTTTTTTTTTTTT | TATTCGTC-R1-SEQ | | 269 to 424 |
| TR2GACGAATATTTTT | ID NO: 482-Y-cttgta | | |
| TTTTTTTTTTTTTTTR2 | SEQ ID NO: | | |
| CACGTG-3′ | | | |
| Représenté par | | | |
| CACGTG-R1-SEQ ID | | | |
| NO: 484-X-SEQ ID NO: | | | |
| 485-R2-SEQ ID NO: | | | |
| 480-R2-CACGTG | | | |
| SEQ ID NO: | | | |
| 5′-CACGTGR1TTTCTC | 5′-TATTCGTCR1TTT | SEQ ID NO: n | SEQ ID NO: n − 1 |
| GATCATTATTATTTTT | TTTTTTTTTTTTTTT | n being an even | n being the same |
| TGGTCGCTTTTTTTTT | ATTAATTATTATCC | number ranging | even number as |
| TTTTTTTXTTTTTTTTT | GGActtgtaY-3′ | from 1 to 6 and | R1 ranging from |
| TTTTTTTGCGACCTTT | Représenté par | from 269 to 424 | 1 to 6 and from |
| TTTTTTTTTTTTTTTTT | TATTCGTC-R1-SEQ | | 269 to 424 |
| TR2GACGAATATTTTT | ID NO: 483-Y | | |
| TTTTTTTTTTTTTTTR2 | SEQ ID NO: | | |
| CACGTG-3′ | | | |
| Représenté par | | | |
| CACGTG-R1-SEQ ID | | | |
| NO: 484-X-SEQ ID NO: | | | |
| 485-R2-SEQ ID NO: | | | |
| 480-R2-CACGTG | | | |
| SEQ ID NO: | | | |
| 5′-CACGTGR1TTTCTC | 5′-TATTCGTCR1TTT | SEQ ID NO: n | SEQ ID NO: n + 1 |
| GATCATTATTATTTTT | TTTTTTTTTTTTTTT | n being an odd | n being the same |
| TGGTCGCTTTTTTTTT | ATTAATTATTATCC | number ranging | lodd number as |
| TTTTTTTXTTTTTTTTT | GGActtgtaY-3′ | from 1 to 6 and | R1 ranging from |
| TTTTTTTGCGACCTTT | Représenté par | from 269 to 424 | 1 to 6 and from |
| TTTTTTTTTTTTTTTTT | TATTCGTC-R1-SEQ | | 269 to 424 |
| TR2GACGAATATTTTT | ID NO: 483-Y | | |
| TTTTTTTTTTTTTTTR2 | SEQ ID NO: | | |
| CACGTG-3′ | | | |
| Représenté par | | | |
| CACGTG-R1-SEQ ID | | | |
| NO: 484-X-SEQ ID NO: | | | |
| 485-R2-SEQ ID NO: | | | |
| 480-R2-CACGTG | | | |
| SEQ ID NO: | | | |

Sequences with two transposase sequences
3′ insertion-model 3

| | | | |
|---|---|---|---|
| 5′-CACGTGR1TTTCTC | 5′-TATTCGTCR1TTT | SEQ ID NO: n | SEQ ID NO: n − 1 |
| GATCATTATTATTTTT | TTTTTTTTTTTTTTT | n being an even | n being the same |
| TGCGGCGATCGGCTT | ATTAATTATTActtgta | number ranging | even number as |
| TTTTTTTTTTTTTTXTT | Y-3′ | from 1 to 6 and | R1 ranging from |
| TTTTTTTTTTTTTTGCC | Représenté par | from 269 to 424 | 1 to 6 and from |
| GATCGCCGCTTTTTTT | TATTCGTC-R1-SEQ | | 269 to 424 |
| TTTTTTTTTTTTTTR2G | ID NO: 481-Y | | |
| ACGAATATTTTTTTTT | SEQ ID NO: | | |
| TTTTTTTTTTTR2CAC | | | |
| GTG-3′ | | | |
| Représenté par | | | |
| CACGTG-R1-SEQ ID | | | |
| NO: 486-X-SEQ ID NO: | | | |
| 487-R2-SEQ ID NO: | | | |
| 480-R2-CACGTG | | | |
| SEQ ID NO: | | | |
| 5′-CACGTGR1TTTCTC | 5′-TATTCGTCR1TTT | SEQ ID NO: n | SEQ ID NO: n + 1 |
| GATCATTATTATTTTT | TTTTTTTTTTTTTTT | n being an odd | n being the same |
| TGCGGCGATCGGCTT | ATTAATTATTActtgta | from 1 to 6 and | odd number as |
| TTTTTTTTTTTTTTXTT | Y-3′ | from 269 to 424 | R1 ranging from |
| TTTTTTTTTTTTTTGCC | Représenté par | number ranging | 1 to 6 and from |
| GATCGCCGCTTTTTTT | TATTCGTC-R1-SEQ | | 269 to 424 |
| TTTTTTTTTTTTTTR2G | ID NO: 481-Y | | |
| ACGAATATTTTTTTTT | SEQ ID NO: | | |
| TTTTTTTTTTTR2CAC | | | |
| GTG-3′ | | | |
| Représenté par | | | |
| CACGTG-R1-SEQ ID | | | |
| NO: 486-X-SEQ ID NO: | | | |
| 487-R2-SEQ ID NO: | | | |
| 480-R2-CACGTG | | | |
| SEQ ID NO: | | | |
| 5′-CACGTGR1TTTCTC | 5′-TATTCGTCR1TTT | SEQ ID NO: n | SEQ ID NO: n − 1 |
| GATCATTATTATTTTT | TTTTTTTTTTTTTTT | n being an even | n being the same |
| TGCGGCGATCGGCTT | ATTAATTATTAYcttgt | number ranging | even number as |
| TTTTTTTTTTTTTTXTT | a-3′ | from 1 to 6 and | R1 ranging from |
| TTTTTTTTTTTTTTGCC | Représenté par | from 269 to 424 | 1 to 6 and from |
| GATCGCCGCTTTTTTT | TATTCGTC-R1-SEQ | | 269 to 424 |
| TTTTTTTTTTTTTTR2G | ID NO: 482-Y-cttgta | | |
| ACGAATATTTTTTTTT | SEQ ID NO: | | |
| TTTTTTTTTTTR2CAC | | | |
| GTG-3′ | | | |
| Représenté par | | | |
| CACGTG-R1-SEQ ID | | | |
| NO: 486-X-SEQ ID NO: | | | |
| 487-R2-SEQ ID NO: | | | |
| 480-R2-CACGTG | | | |
| SEQ ID NO: | | | |
| 5′-CACGTGR1TTTCTC | 5′-TATTCGTCR1TTT | SEQ ID NO: n | SEQ ID NO: n + 1 |
| GATCATTATTATTTTT | TTTTTTTTTTTTTTT | n being an odd | n being the same |
| TGCGGCGATCGGCTT | ATTAATTATTAYcttgt | number ranging | odd number as |
| TTTTTTTTTTTTTTXTT | a-3′ | from 1 to 6 and | R1 ranging from |
| TTTTTTTTTTTTTTGCC | Représenté par | from 269 to 424 | 1 to 6 and from |
| GATCGCCGCTTTTTTT | TATTCGTC-R1-SEQ | | 269 to 424 |
| TTTTTTTTTTTTTTR2G | ID NO: 482-Y-cttgta | | |
| ACGAATATTTTTTTTT | SEQ ID NO: | | |
| TTTTTTTTTTTR2CAC | | | |
| GTG-3′ | | | |
| Représenté par | | | |
| CACGTG-R1-SEQ ID | | | |
| NO: 486-X-SEQ ID NO: | | | |
| 487-R2-SEQ ID NO: | | | |
| 480-R2-CACGTG | | | |
| SEQ ID NO: | | | |
| 5′-CACGTGR1TTTCTC | 5′-TATTCGTCR1TTT | SEQ ID NO: n | SEQ ID NO: n − 1 |
| GATCATTATTATTTTT | TTTTTTTTTTTTTTT | n being an even | n being the same |
| TGCGGCGATCGGCTT | ATTAATTATTATCC | number ranging | even number as |
| TTTTTTTTTTTTTTXTT | GGActtgtaY-3′ | from 1 to 6 and | R1 ranging from |
| TTTTTTTTTTTTTTGCC | Représenté par | from 269 to 424 | 1 to 6 and from |
| GATCGCCGCTTTTTTT | TATTCGTC-R1-SEQ | | 269 to 424 |
| TTTTTTTTTTTTTTR2G | ID NO: 483-Y | | |
| ACGAATATTTTTTTTT | SEQ ID NO: | | |
| TTTTTTTTTTTR2CAC | | | |
| GTG-3′ | | | |
| Représenté par | | | |
| CACGTG-R1-SEQ ID | | | |
| NO: 486-X-SEQ ID NO: | | | |
| 487-R2-SEQ ID NO: | | | |
| 480-R2-CACGTG | | | |
| SEQ ID NO: | | | |
| 5′-CACGTGR1TTTCTC | 5′-TATTCGTCR1TTT | SEQ ID NO: n | SEQ ID NO: n + 1 |
| GATCATTATTATTTTT | TTTTTTTTTTTTTTT | n being an odd | n being the same |
| TGCGGCGATCGGCTT | ATTAATTATTATCC | number ranging | odd number as |
| TTTTTTTTTTTTTTXTT | GGActtgtaY-3′ | from 1 to 6 and | R1 ranging from |
| TTTTTTTTTTTTTTGCC | Représenté par | from 269 to 424 | 1 to 6 and from |
| GATCGCCGCTTTTTTT | TATTCGTC-R1-SEQ | | 269 to 424 |
| TTTTTTTTTTTTTTR2G | ID NO: 483-Y | | |
| ACGAATATTTTTTTTT | SEQ ID NO: | | |
| TTTTTTTTTTTR2CAC | | | |
| GTG-3′ | | | |
| Représenté par | | | |
| CACGTG-R1-SEQ | | | |
| NO: 486-X-SEQ ID | | | |
| 487-R2-SEQ ID | | | |
| 480-R2-CACGTG | | | |
| SEQ ID NO: | | | |

Séquences avec deux séquences de transposase
Insertion 3′-modèle 4

| | | | |
|---|---|---|---|
| 5′-R2CACGTGTTTTTT | 5′-TATTCGTCR1TTT | SEQ ID NO: n | SEQ ID NO: n − 1 |
| TTTTTTTTTTTTTTCAC | TTTTTTTTTTTTTTT | n being an even | n being the same |
| GTGR1TTTCTCGATCA | ATTAATTATTActtgta | number ranging | even number as |
| TTATTATTTTTTGGCG | Y-3′ | from 1 to 6 and | R1 ranging from |
| ATCGCTTTTTTTTTTTT | Représenté par | from 269 to 424 | 1 to 6 and from |
| TTTTXTTTTTTTTTTTT | TATTCGTC-R1-SEQ | | 269 to 424 |
| TTTTGCGATCGCCTTT | ID NO: 481-Y | | |
| TTTTTTTTTTTTTTTTT | SEQ ID NO: | | |
| TR2GACGAATA-3′ | | | |
| Représenté par R2-SEQ | | | |
| ID NO: 488-R1-SEQ ID | | | |
| NO: 478-X-SEQ ID NO: | | | |
| 479-R2- GACGAATA | | | |
| SEQ ID NO: | | | |
| 5′-R2CACGTGTTTTTT | 5′-TATTCGTCR1TTT | SEQ ID NO: n | SEQ ID NO: n + 1 |
| TTTTTTTTTTTTTTCAC | TTTTTTTTTTTTTTT | n being an odd | n being the same |
| GTGR1TTTCTCGATCA | ATTAATTATTActtgta | number ranging | odd number as |
| TTATTATTTTTTGGCG | Y-3′ | from 1 to 6 and | R1 ranging from |
| ATCGCTTTTTTTTTTTT | Représenté par | from 269 to 424 | 1 to 6 and from |
| TTTTXTTTTTTTTTTTT | TATTCGTC-R1-SEQ | | 269 to 424 |
| TTTTGCGATCGCCTTT | ID NO: 481-Y | | |
| TTTTTTTTTTTTTTTTT | SEQ ID NO: | | |
| TR2GACGAATA-3′ | | | |
| Représenté par R2-SEQ | | | |
| ID NO: 488-R1-SEQ | | | |
| NO: 478-X-SEQ ID | | | |
| 479-R2- GACGAATA | | | |
| SEQ ID NO: | | | |
| 5′-R2CACGTGTTTTTT | 5′-TATTCGTCR1TTT | SEQ ID NO: n | SEQ ID NO: n − 1 |
| TTTTTTTTTTTTTTCAC | TTTTTTTTTTTTTTT | n being an even | n being the same |
| GTGR1TTTCTCGATCA | ATTAATTATTAYcttgt | number ranging | even number as |
| TTATTATTTTTTGGCG | a-3′ | from 1 to 6 and | R1 ranging from |
| ATCGCTTTTTTTTTTTT | Représenté par | from 269 to 424 | 1 to 6 and from |
| TTTTXTTTTTTTTTTTT | TATTCGTC-R1-SEQ | | 269 to 424 |
| TTTTGCGATCGCCTTT | ID NO: 482-Y-cttgta | | |
| TTTTTTTTTTTTTTTTT | SEQ ID NO: | | |
| TR2GACGAATA-3′ | | | |
| Représenté par R2-SEQ | | | |
| ID NO: 488-R1-SEQ ID | | | |
| NO: 478-X-SEQ ID NO: | | | |
| 479-R2- GACGAATA | | | |
| SEQ ID NO: | | | |
| 5′-R2CACGTGTTTTTT | 5′-TATTCGTCR1TTT | SEQ ID NO: n | SEQ ID NO: n + 1 |
| TTTTTTTTTTTTTTCAC | TTTTTTTTTTTTTTT | n being an odd | n being the same |
| GTGR1TTTCTCGATCA | ATTAATTATTAYcttgt | number ranging | odd number as |
| TTATTATTTTTTGGCG | a-3′ | from 1 to 6 and | R1 ranging from |
| ATCGCTTTTTTTTTTTT | Représenté par | from 269 to 424 | 1 to 6 and from |
| TTTTXTTTTTTTTTTTT | TATTCGTC-R1-SEQ | | 269 to 424 |
| TTTTGCGATCGCCTTT | ID NO: 482-Y-cttgta | | |
| TTTTTTTTTTTTTTTTT | SEQ ID NO: | | |
| TR2GACGAATA-3′ | | | |
| Représenté par R2-SEQ | | | |
| ID NO: 488-R1-SEQ ID | | | |
| NO: 478-X-SEQ ID NO: | | | |
| 479-R2- GACGAATA | | | |
| SEQ ID NO: | | | |
| 5′-R2CACGTGTTTTTT | 5′-TATTCGTCR1TTT | SEQ ID NO: n | SEQ ID NO: n − 1 |
| TTTTTTTTTTTTTTCAC | TTTTTTTTTTTTTTT | n being an even | n being the same |
| GTGR1TTTCTCGATCA | ATTAATTATTATCC | number ranging | even number as |
| TTATTATTTTTTGGCG | GGActtgtaY-3′ | from 1 to 6 and | R1 ranging from |
| ATCGCTTTTTTTTTTTT | Représenté par | from 269 to 424 | 1 to 6 and from |
| TTTTXTTTTTTTTTTTT | TATTCGTC-R1-SEQ | | 269 to 424 |
| TTTTGCGATCGCCTTT | ID NO: 483-Y | | |
| TTTTTTTTTTTTTTTTT | SEQ ID NO: | | |
| TR2GACGAATA-3′ | | | |
| Représenté par R2-SEQ | | | |
| ID NO: 488-R1-SEQ ID | | | |
| NO: 478-X-SEQ ID NO: | | | |
| 479-R2- GACGAATA | | | |
| SEQ ID NO: | | | |
| 5′-R2CACGTGTTTTTT | 5′-TATTCGTCR1TTT | SEQ ID NO: n | SEQ ID NO: n + 1 |
| TTTTTTTTTTTTTTCAC | TTTTTTTTTTTTTTT | n being an odd | n being the same |
| GTGR1TTTCTCGATCA | ATTAATTATTATCC | number ranging | odd number as |
| TTATTATTTTTTGGCG | GGActtgtaY-3′ | from 1 to 6 and | R1 ranging from |
| ATCGCTTTTTTTTTTTT | Représenté par | from 269 to 424 | 1 to 6 and from |
| TTTTXTTTTTTTTTTTT | TATTCGTC-R1-SEQ | | 269 to 424 |
| TTTTGCGATCGCCTTT | ID NO: 483-Y | | |
| TTTTTTTTTTTTTTTTT | SEQ ID NO: | | |
| TR2GACGAATA-3′ | | | |
| Représenté par R2-SEQ | | | |
| ID NO: 488-R1-SEQ ID | | | |
| NO: 478-X-SEQ ID NO: | | | |
| 479-R2- GACGAATA | | | |
| SEQ ID NO: | | | |

Sequences with two transposase sequences
3′ insertion-model 5

| | | | |
|---|---|---|---|
| 5′-R2CACGTGTTTTTT | 5′-TATTCGTCR1TTT | SEQ ID NO: n | SEQ ID NO: n − 1 |
| TTTTTTTTTTTTTTCAC | TTTTTTTTTTTTTTT | n being an even | n being the same |
| GTGR1TTTCTCGATCA | ATTAATTATTActtgta | number ranging | even number as |
| TTATTATTTTTTGGTC | Y-3′ | from 1 to 6 and | R1 ranging from |
| GCTTTTTTTTTTTTTTT | Représenté par | from 269 to 424 | 1 to 6 and from |
| TXTTTTTTTTTTTTTTT | TATTCGTC-R1-SEQ | | 269 to 424 |
| TGCGACCTTTTTTTTT | ID NO: 481-Y | | |
| TTTTTTTTTTTTR2GAC | SEQ ID NO: | | |
| GAATA-3′ | | | |
| Représenté par R2-SEQ | | | |
| ID NO: 488-R1-SEQ ID | | | |
| NO: 484-X-SEQ ID NO: | | | |
| 485-R2- GACGAATA | | | |
| SEQ ID NO: | | | |
| 5′-R2CACGTGTTTTTT | 5′-TATTCGTCR1TTT | SEQ ID NO: n | SEQ ID NO: n + 1 |
| TTTTTTTTTTTTTTCAC | TTTTTTTTTTTTTTT | n being an odd | n being the same |
| GTGR1TTTCTCGATCA | ATTAATTATTActtgta | number ranging | odd number as |
| TTATTATTTTTTGGTC | Y-3′ | from 1 to 6 and | R1 ranging from |
| GCTTTTTTTTTTTTTTT | Représenté par | from 269 to 424 | 1 to 6 and from |
| TXTTTTTTTTTTTTTTT | TATTCGTC-R1-SEQ | | 269 to 424 |
| TGCGACCTTTTTTTTT | ID NO: 481-Y | | |
| TTTTTTTTTTTTR2GAC | SEQ ID NO: | | |
| GAATA-3′ | | | |
| Représenté par R2-SEQ | | | |
| ID NO: 488-R1-SEQ ID | | | |
| NO: 484-X-SEQ ID NO: | | | |
| 485-R2- GACGAATA | | | |
| SEQ ID NO: | | | |
| 5′-R2CACGTGTTTTTT | 5′-TATTCGTCR1TTT | SEQ ID NO: n | SEQ ID NO: n − 1 |
| TTTTTTTTTTTTTTCAC | TTTTTTTTTTTTTTT | n being an even | n being the same |
| GTGR1TTTCTCGATCA | ATTAATTATTAYcttgt | number ranging | even number as |
| TTATTATTTTTTGGTC | a-3′ | from 1 to 6 and | R1 ranging from |
| GCTTTTTTTTTTTTTTT | Représenté par | from 269 to 424 | 1 to 6 and from |
| TXTTTTTTTTTTTTTTT | TATTCGTC-R1-SEQ | | 269 to 424 |
| TGCGACCTTTTTTTTT | ID NO: 482-Y-cttgta | | |
| TTTTTTTTTTTTR2GAC | SEQ ID NO: | | |
| GAATA-3′ | | | |
| Représenté par R2-SEQ | | | |
| ID NO: 488-R1-SEQ ID | | | |
| NO: 484-X-SEQ ID NO: | | | |
| 485-R2- GACGAATA | | | |
| SEQ ID NO: | | | |
| 5′-R2CACGTGTTTTTT | 5′-TATTCGTCR1TTT | SEQ ID NO: n | SEQ ID NO: n + 1 |
| TTTTTTTTTTTTTTCAC | TTTTTTTTTTTTTTT | n being an odd | n being the same |
| GTGR1TTTCTCGATCA | ATTAATTATTAYcttgt | number ranging | odd number as |
| TTATTATTTTTTGGTC | a-3′ | from 1 to 6 and | R1 ranging from |
| GCTTTTTTTTTTTTTTT | Représenté par | from 269 to 424 | 1 to 6 and from |
| TXTTTTTTTTTTTTTTT | TATTCGTC-R1-SEQ | | 269 to 424 |
| TGCGACCTTTTTTTTT | ID NO: 482-Y-cttgta | | |
| TTTTTTTTTTTTR2GAC | SEQ ID NO: | | |
| GAATA-3′ | | | |
| Représenté par R2-SEQ | | | |
| ID NO: 488-R1-SEQ ID | | | |
| NO: 484-X-SEQ ID NO: | | | |
| 485-R2- GACGAATA | | | |
| SEQ ID NO: | | | |
| 5′-R2CACGTGTTTTTT | 5′-TATTCGTCR1TTT | SEQ ID NO: n | SEQ ID NO: n − 1 |
| TTTTTTTTTTTTTTCAC | TTTTTTTTTTTTTTT | n being an even | n being the same |
| GTGR1TTTCTCGATCA | ATTAATTATTATCC | number ranging | even number as |
| TTATTATTTTTTGGTC | GGActtgtaY-3′ | from 1 to 6 and | R1 ranging from |
| GCTTTTTTTTTTTTTTT | Représenté par | from 269 to 424 | 1 to 6 and from |
| TXTTTTTTTTTTTTTTT | TATTCGTC-R1-SEQ | | 269 to 424 |
| TGCGACCTTTTTTTTT | ID NO: 483-Y | | |
| TTTTTTTTTTTTR2GAC | SEQ ID NO: | | |
| GAATA-3′ | | | |
| Représenté par R2-SEQ | | | |
| ID NO: 488-R1-SEQ ID | | | |
| NO: 484-X-SEQ ID NO: | | | |
| 485-R2- GACGAATA | | | |
| SEQ ID NO: | | | |
| 5′-R2CACGTGTTTTTT | 5′-TATTCGTCR1TTT | SEQ ID NO: n | SEQ ID NO: n + 1 |
| TTTTTTTTTTTTTTCAC | TTTTTTTTTTTTTTT | n being an odd | n being the same |
| GTGR1TTTCTCGATCA | ATTAATTATTATCC | number ranging | odd number as |
| TTATTATTTTTTGGTC | GGActtgtaY-3′ | from 1 to 6 and | R1 ranging from |
| GCTTTTTTTTTTTTTTT | Représenté par | from 269 to 424 | 1 to 6 and from |
| TXTTTTTTTTTTTTTTT | TATTCGTC-R1-SEQ | | 269 to 424 |
| TGCGACCTTTTTTTTT | ID NO: 483-Y | | |
| TTTTTTTTTTTTR2GAC | SEQ ID NO: | | |
| GAATA-3′ | | | |
| Représenté par R2-SEQ | | | |
| ID NO: 488-R1-SEQ ID | | | |
| NO: 484-X-SEQ ID NO: | | | |
| 485-R2- GACGAATA | | | |
| SEQ ID NO: | | | |

Sequences with two transposase sequences
3′ insertion-model 6

| | | | |
|---|---|---|---|
| 5′-R2CACGTGTTTTTT | 5′-TATTCGTCR1TTT | SEQ ID NO: n | SEQ ID NO: n − 1 |
| TTTTTTTTTTTTTTCAC | TTTTTTTTTTTTTTT | n being an even | n being the same |
| GTGR1TTTCTCGATCA | ATTAATTATTActtgta | number ranging | even number as |
| TTATTATTTTTTGCGG | Y-3′ | from 1 to 6 and | R1 ranging from |
| CGATCGGCTTTTTTTT | Représenté par | from 269 to 424 | 1 to 6 and from |
| TTTTTTTTXTTTTTTTT | TATTCGTC-R1-SEQ | | 269 to 424 |
| TTTTTTTTGCCGATCG | ID NO: 481-Y | | |
| CCGCTTTTTTTTTTTT | SEQ ID NO: | | |
| TTTTTTTTTR2GACGA | | | |
| ATA-3′ | | | |
| Représenté par R2-SEQ | | | |
| ID NO: 488-R1-SEQ ID | | | |
| NO: 486-X-SEQ ID NO: | | | |
| 487-R2- GACGAATA | | | |
| SEQ ID NO: | | | |
| 5′-R2CACGTGTTTTTT | 5′-TATTCGTCR1TTT | SEQ ID NO: n | SEQ ID NO: n + 1 |
| TTTTTTTTTTTTTTCAC | TTTTTTTTTTTTTTT | n being an odd | n being the same |
| GTGR1TTTCTCGATCA | ATTAATTATTActtgta | number ranging | odd number as |
| TTATTATTTTTTGCGG | Y-3′ | from 1 to 6 and | R1 ranging from |
| CGATCGGCTTTTTTTT | Représenté par | from 269 to 424 | 1 to 6 and from |
| TTTTTTTTXTTTTTTTT | TATTCGTC-R1-SEQ | | 269 to 424 |
| TTTTTTTTGCCGATCG | ID NO: 481-Y | | |
| CCGCTTTTTTTTTTTT | SEQ ID NO: | | |
| TTTTTTTTTR2GACGA | | | |
| ATA-3′ | | | |
| Représenté par R2-SEQ | | | |
| ID NO: 488-R1-SEQ ID | | | |
| NO: 486-X-SEQ ID NO: | | | |
| 487-R2- GACGAATA | | | |
| SEQ ID NO: | | | |
| 5′-R2CACGTGTTTTTT | 5′-TATTCGTCR1TTT | SEQ ID NO: n | SEQ ID NO: n − 1 |
| TTTTTTTTTTTTTTCAC | TTTTTTTTTTTTTTT | n being an even | n being the same |
| GTGR1TTTCTCGATCA | ATTAATTATTAYcttgt | number ranging | even number as |
| TTATTATTTTTTGCGG | a-3′ | from 1 to 6 and | R1 ranging from |
| CGATCGGCTTTTTTTT | Représenté par | from 269 to 424 | 1 to 6 and from |
| TTTTTTTTXTTTTTTTT | TATTCGTC-R1-SEQ | | 269 to 424 |
| TTTTTTTTGCCGATCG | ID NO: 482-Y-cttgta | | |
| CCGCTTTTTTTTTTTT | SEQ ID NO: | | |
| TTTTTTTTTR2GACGA | | | |
| ATA-3′ | | | |
| Représenté par R2-SEQ | | | |
| ID NO: 488-R1-SEQ ID | | | |
| NO: 486-X-SEQ ID NO: | | | |
| 487-R2- GACGAATA | | | |
| SEQ ID NO: | | | |
| 5′-R2CACGTGTTTTTT | 5′-TATTCGTCR1TTT | SEQ ID NO: n | SEQ ID NO: n + 1 |
| TTTTTTTTTTTTTTCAC | TTTTTTTTTTTTTTT | n being an odd | n being the same |
| GTGR1TTTCTCGATCA | ATTAATTATTAYcttgt | number ranging | odd number as |
| TTATTATTTTTTGCGG | a-3′ | from 1 to 6 and | R1 ranging from |
| CGATCGGCTTTTTTTT | Représenté par | from 269 to 424 | 1 to 6 and from |
| TTTTTTTTXTTTTTTTT | TATTCGTC-R1-SEQ | | 269 to 424 |
| TTTTTTTTGCCGATCG | ID NO: 482-Y-cttgta | | |
| CCGCTTTTTTTTTTTT | SEQ ID NO: | | |
| TTTTTTTTTR2GACGA | | | |
| ATA-3′ | | | |
| Représenté par R2-SEQ | | | |
| ID NO: 488-R1-SEQ ID | | | |
| NO: 486-X-SEQ ID NO: | | | |
| 487-R2- GACGAATA | | | |
| SEQ ID NO: | | | |
| 5′-R2CACGTGTTTTTT | 5′-TATTCGTCR1TTT | SEQ ID NO: n | SEQ ID NO: n − 1 |
| TTTTTTTTTTTTTTCAC | TTTTTTTTTTTTTTT | n being an even | n being the same |
| GTGR1TTTCTCGATCA | ATTAATTATTATCC | number ranging | even number as |
| TTATTATTTTTTGCGG | GGActtgtaY-3′ | from 1 to 6 and | R1 ranging from |
| CGATCGGCTTTTTTTT | Représenté par | from 269 to 424 | 1 to 6 and from |
| TTTTTTTTXTTTTTTTT | TATTCGTC-R1-SEQ | | 269 to 424 |
| TTTTTTTTGCCGATCG | ID NO: 483-Y | | |
| CCGCTTTTTTTTTTTT | SEQ ID NO: | | |
| TTTTTTTTTR2GACGA | | | |
| ATA-3′ | | | |
| Représenté par R2-SEQ | | | |
| ID NO: 488-R1-SEQ ID | | | |
| NO: 486-X-SEQ ID NO: | | | |
| 487-R2- GACGAATA | | | |
| SEQ ID NO: | | | |
| 5′-R2CACGTGTTTTTT | 5′-TATTCGTCR1TTT | SEQ ID NO: n | SEQ ID NO: n + 1 |
| TTTTTTTTTTTTTTCAC | TTTTTTTTTTTTTTT | n being an odd | n being the same |
| GTGR1TTTCTCGATCA | ATTAATTATTATCC | number ranging | odd number as |
| TTATTATTTTTTGCGG | GGActtgtaY-3′ | from 1 to 6 and | R1 ranging from |
| CGATCGGCTTTTTTTT | Représenté par | from 269 to 424 | 1 to 6 and from |
| TTTTTTTTXTTTTTTTT | TATTCGTC-R1-SEQ | | 269 to 424 |
| TTTTTTTTGCCGATCG | ID NO: 483-Y | | |
| CCGCTTTTTTTTTTTT | SEQ ID NO: | | |
| TTTTTTTTTR2GACGA | | | |
| ATA-3′ | | | |
| Représenté par R2-SEQ | | | |
| ID NO: 488-R1-SEQ ID | | | |
| NO: 486-X-SEQ ID NO: | | | |
| 487-R2- GACGAATA | | | |
| SEQ ID NO: | | | |

In other words, the invention advantageously relates to an aforementioned complex, the complex comprising the pairs of first and second molecules, said first and second molecules comprising the following respective sequences, wherein X and Y are as defined above:
SEQ ID NO: 436-R1-SEQ ID NO: 452-X-SEQ ID NO: 453-R2-SEQ ID NO: 439 and R2-SEQ ID NO: 440-R1-SEQ ID NO: 454-Y-ccaagtSEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
SEQ ID NO: 436-R1-SEQ ID NO: 452-X-SEQ ID NO: 453-R2-SEQ ID NO: 439 and R2-SEQ ID NO: 440-R1-SEQ ID NO: 454-Y-ccaagtSEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
SEQ ID NO: 436-R1-SEQ ID NO: 452-X-SEQ ID NO: 453-R2-SEQ ID NO: 439 and R2-SEQ ID NO: 440-R1-SEQ ID NO: 455-YSEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
SEQ ID NO: 436-R1-SEQ ID NO: 452-X-SEQ ID NO: 453-R2-SEQ ID NO: 439 and R2-SEQ ID NO: 440-R1-SEQ ID NO: 455-YSEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
SEQ ID NO: 436-R1-SEQ ID NO: 456-X-SEQ ID NO: 457-R2-SEQ ID NO: 439 and R2-SEQ ID NO: 440-R1-SEQ ID NO: 454-Y-ccaagtSEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
SEQ ID NO: 436-R1-SEQ ID NO: 456-X-SEQ ID NO: 457-R2-SEQ ID NO: 439 and R2-SEQ ID NO: 440-R1-SEQ ID NO: 454-Y-ccaagtSEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
SEQ ID NO: 436-R1-SEQ ID NO: 456-X-SEQ ID NO: 457-R2-SEQ ID NO: 439 and R2-SEQ ID NO: 440-R1-SEQ ID NO: 455-Y, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
SEQ ID NO: 436-R1-SEQ ID NO: 456-X-SEQ ID NO: 457-R2-SEQ ID NO: 439 and R2-SEQ ID NO: 440-R1-SEQ ID NO: 455-Y, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
SEQ ID NO: 458-R1-SEQ ID NO: 459-X-SEQ ID NO: 460-R2-SEQ ID NO: 461 and Y-SEQ ID NO: 462-R2-SEQ ID NO: 463-R1SEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
SEQ ID NO: 458-R1-SEQ ID NO: 459-X-SEQ ID NO: 460-R2-SEQ ID NO: 461 and Y-SEQ ID NO: 462-R2-SEQ ID NO: 463-R1SEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
SEQ ID NO: 458-R1-SEQ ID NO: 459-X-SEQ ID NO: 460-R2-SEQ ID NO: 461 and Y-SEQ ID NO: 464)-R2-SEQ ID NO: 463-R1SEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
SEQ ID NO: 458-R1-SEQ ID NO: 459-X-SEQ ID NO: 460-R2-SEQ ID NO: 461 and Y-SEQ ID NO: 464)-R2-SEQ ID NO: 463-R1SEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
SEQ ID NO: 458-R1-SEQ ID NO: 459-X-SEQ ID NO: 460-R2-SEQ ID NO: 461 and Y-SEQ ID NO: 465-R2-SEQ ID NO: 463-R1SEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
SEQ ID NO: 458-R1-SEQ ID NO: 459-X-SEQ ID NO: 460-R2-SEQ ID NO: 461 and Y-SEQ ID NO: 465-R2-SEQ ID NO: 463-R1SEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
SEQ ID NO: 458-R1-SEQ ID NO: 466-X-SEQ ID NO: 467-R2-SEQ ID NO: 461 and Y-SEQ ID NO: 462-R2-SEQ ID NO: 463-R1SEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
SEQ ID NO: 458-R1-SEQ ID NO: 466-X-SEQ ID NO: 467-R2-SEQ ID NO: 461 and Y-SEQ ID NO: 462-R2-SEQ ID NO: 463-R1SEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
SEQ ID NO: 458-R1-SEQ ID NO: 466-X-SEQ ID NO: 467-R2-SEQ ID NO: 461 and tacaag-Y-SEQ ID NO: 464)-R2-SEQ ID NO: 463-R1SEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
SEQ ID NO: 458-R1-SEQ ID NO: 466-X-SEQ ID NO: 467-R2-SEQ ID NO: 461 and tacaag-Y-SEQ ID NO: 464)-R2-SEQ ID NO: 463-R1SEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
SEQ ID NO: 458-R1-SEQ ID NO: 466-X-SEQ ID NO: 467-R2-SEQ ID NO: 461 and Y-SEQ ID NO: 465-R2-SEQ ID NO: 463-R1SEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
SEQ ID NO: 458-R1-SEQ ID NO: 466-X-SEQ ID NO: 467-R2-SEQ ID NO: 461 and Y-SEQ ID NO: 465-R2-SEQ ID NO: 463-R1SEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
SEQ ID NO: 458-R1-SEQ ID NO: 468-X-SEQ ID NO: 469-R2-SEQ ID NO: 461 and Y-SEQ ID NO: 462-R2-SEQ ID NO: 463-R1SEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
SEQ ID NO: 458-R1-SEQ ID NO: 468-X-SEQ ID NO: 469-R2-SEQ ID NO: 461 and Y-SEQ ID NO: 462-R2-SEQ ID NO: 463-R1SEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
SEQ ID NO: 458-R1-SEQ ID NO: 468-X-SEQ ID NO: 469-R2-SEQ ID NO: 461 and tacaag-Y-SEQ ID NO: 464)-R2-SEQ ID NO: 463-R1SEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
SEQ ID NO: 458-R1-SEQ ID NO: 468-X-SEQ ID NO: 469-R2-SEQ ID NO: 461 and tacaag-Y-SEQ ID NO: 464)-R2-SEQ ID NO: 463-R1SEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
SEQ ID NO: 458-R1-SEQ ID NO: 468-X-SEQ ID NO: 469-R2-SEQ ID NO: 461 and Y-SEQ ID NO: 465-R2-SEQ ID NO: 463-R1SEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
SEQ ID NO: 458-R1-SEQ ID NO: 468-X-SEQ ID NO: 469-R2-SEQ ID NO: 461 and Y-SEQ ID NO: 465-R2-SEQ ID NO: 463-R1SEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
SEQ ID NO: 445-R1-SEQ ID NO: 446-R1-SEQ ID NO: 470-X-SEQ ID NO: 474-R2-SEQ ID NO: 449 and Y-SEQ ID NO: 450-R2-SEQ ID NO: 451SEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
SEQ ID NO: 445-R1-SEQ ID NO: 446-R1-SEQ ID NO: 470-X-SEQ ID NO: 474-R2-SEQ ID NO: 449 et Y-SEQ ID NO: 450-R2-SEQ ID NO: 451SEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
SEQ ID NO: 445-R1-SEQ ID NO: 446-R1-SEQ ID NO: 470-X-SEQ ID NO: 474-R2-SEQ ID NO: 449 and ACTTGG-Y-SEQ ID NO: 473-R2-SEQ ID NO: 451SEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
SEQ ID NO: 445-R1-SEQ ID NO: 446-R1-SEQ ID NO: 470-X-SEQ ID NO: 474-R2-SEQ ID NO: 449 and ACTTGG-Y-SEQ ID NO: 473-R2-SEQ ID NO: 451SEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
SEQ ID NO: 445-R1-SEQ ID NO: 446-R1-SEQ ID NO: 470-X-SEQ ID NO: 474-R2-SEQ ID NO: 449 and Y-SEQ ID NO: 475-R2-SEQ ID NO: 451SEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
SEQ ID NO: 445-R1-SEQ ID NO: 446-R1-SEQ ID NO: 470-X-SEQ ID NO: 474-R2-SEQ ID NO: 449 and Y-SEQ ID NO: 475-R2-SEQ ID NO: 451SEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
SEQ ID NO: 445-R1-SEQ ID NO: 446-R1-SEQ ID NO: 471-X-SEQ ID NO: 472-R2-SEQ ID NO: 449 and Y-SEQ ID NO: 450-R2-SEQ ID NO: 451SEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
SEQ ID NO: 445-R1-SEQ ID NO: 446-R1-SEQ ID NO: 471-X-SEQ ID NO: 472-R2-SEQ ID NO: 449 and Y-SEQ ID NO: 450-R2-SEQ ID NO: 451SEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
SEQ ID NO: 445-R1-SEQ ID NO: 446-R1-SEQ ID NO: 471-X-SEQ ID NO: 472-R2-SEQ ID NO: 449 and ACTTGG-Y-SEQ ID NO: 473-R2-SEQ ID NO: 451SEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
SEQ ID NO: 445-R1-SEQ ID NO: 446-R1-SEQ ID NO: 471-X-SEQ ID NO: 472-R2-SEQ ID NO: 449 and ACTTGG-Y-SEQ ID NO: 473-R2-SEQ ID NO: 451SEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
SEQ ID NO: 445-R1-SEQ ID NO: 446-R1-SEQ ID NO: 471-X-SEQ ID NO: 472-R2-SEQ ID NO: 449 and Y-SEQ ID NO: 475-R2-SEQ ID NO: 451SEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
SEQ ID NO: 445-R1-SEQ ID NO: 446-R1-SEQ ID NO: 471-X-SEQ ID NO: 472-R2-SEQ ID NO: 449 and Y-SEQ ID NO: 475-R2-SEQ ID NO: 451SEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
GATAGTAG-R1-SEQ ID NO: 476-X-SEQ ID NO: 448-R2-SEQ ID NO: 477-R1 and Y-SEQ ID NO: 450-R2-SEQ ID NO: 451SEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
GATAGTAG-R1-SEQ ID NO: 476-X-SEQ ID NO: 448-R2-SEQ ID NO: 477-R1 and Y-SEQ ID NO: 450-R2-SEQ ID NO: 451SEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
GATAGTAG-R1-SEQ ID NO: 476-X-SEQ ID NO: 448-R2-SEQ ID NO: 477-R1 and ACTTGG-Y-SEQ ID NO: 473-R2-SEQ ID NO: 451SEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
GATAGTAG-R1-SEQ ID NO: 476-X-SEQ ID NO: 448-R2-SEQ ID NO: 477-R1 and ACTTGG-Y-SEQ ID NO: 473-R2-SEQ ID NO: 451SEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
GATAGTAG-R1-SEQ ID NO: 476-X-SEQ ID NO: 448-R2-SEQ ID NO: 477-R1 and Y-SEQ ID NO: 475-R2-SEQ ID NO: 451SEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
GATAGTAG-R1-SEQ ID NO: 476-X-SEQ ID NO: 448-R2-SEQ ID NO: 477-R1 and Y-SEQ ID NO: 475-R2-SEQ ID NO: 451SEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
GATAGTAG-R1-SEQ ID NO: 470-X-SEQ ID NO: 471-R2-SEQID NO 477-R1 and Y-SEQ ID NO: 450-R2-SEQ ID NO: 451SEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
GATAGTAG-R1-SEQ ID NO: 470-X-SEQ ID NO: 471-R2-SEQID NO 477-R1 and Y-SEQ ID NO: 450-R2-SEQ ID NO: 451SEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
GATAGTAG-R1-SEQ ID NO: 470-X-SEQ ID NO: 471-R2-SEQID NO 477-R1 and ACTTGG-Y-SEQ ID NO: 473-R2-SEQ ID NO: 451SEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
GATAGTAG-R1-SEQ ID NO: 470-X-SEQ ID NO: 471-R2-SEQID NO 477-R1 and ACTTGG-Y-SEQ ID NO: 473-R2-SEQ ID NO: 451SEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
GATAGTAG-R1-SEQ ID NO: 470-X-SEQ ID NO: 471-R2-SEQID NO 477-R1 and Y-SEQ ID NO: 475-R2-SEQ ID NO: 451SEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
GATAGTAG-R1-SEQ ID NO: 470-X-SEQ ID NO: 471-R2-SEQID NO 477-R1 and Y-SEQ ID NO: 475-R2-SEQ ID NO: 451SEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
SEQ ID NO: GATAGTAG-R1-SEQ ID NO: 471-X-SEQ ID NO: 472-R2-SEQ ID NO: 477-R1 and Y-SEQ ID NO: 450-R2-SEQ ID NO: 451SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
GATAGTAG-R1-SEQ ID NO: 471-X-SEQ ID NO: 472-R2-SEQ ID NO: 477-R1 and Y-SEQ ID NO: 450-R2-SEQ ID NO: 451SEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
GATAGTAG-R1-SEQ ID NO: 471-X-SEQ ID NO: 472-R2-SEQ ID NO: 477-R1 and ACTTGG-Y-SEQ ID NO: 473-R2-SEQ ID NO: 451SEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
GATAGTAG-R1-SEQ ID NO: 471-X-SEQ ID NO: 472-R2-SEQ ID NO: 477-R1 and ACTTGG-Y-SEQ ID NO: 473-R2-SEQ ID NO: 451SEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
GATAGTAG-R1-SEQ ID NO: 471-X-SEQ ID NO: 472-R2-SEQ ID NO: 477-R1 and Y-SEQ ID NO: 475-R2-SEQ ID NO: 451SEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
GATAGTAG-R1-SEQ ID NO: 471-X-SEQ ID NO: 472-R2-SEQ ID NO: 477-R1 and Y-SEQ ID NO: 475-R2-SEQ ID NO: 451SEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
CACGTG-R1-SEQ ID NO: 478-X-SEQ ID NO: 479-R2-SEQ ID NO: 480-R2-CACGTG and TATTCGTC-R1-SEQ ID NO: 481-YSEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
CACGTG-R1-SEQ ID NO: 478-X-SEQ ID NO: 479-R2-SEQ ID NO: 480-R2-CACGTG and TATTCGTC-R1-SEQ ID NO: 481-YSEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
CACGTG-R1-SEQ ID NO: 478-X-SEQ ID NO: 479-R2-SEQ ID NO: 480-R2-CACGTG and TATTCGTC-R1-SEQ ID NO: 482-Y-CTTGTASEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
CACGTG-R1-SEQ ID NO: 478-X-SEQ ID NO: 479-R2-SEQ ID NO: 480-R2-CACGTG and TATTCGTC-R1-SEQ ID NO: 482-Y-CTTGTASEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
CACGTG-R1-SEQ ID NO: 478-X-SEQ ID NO: 479-R2-SEQ ID NO: 480-R2-CACGTG and TATTCGTC-R1-SEQ ID NO: 483-YSEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
CACGTG-R1-SEQ ID NO: 478-X-SEQ ID NO: 479-R2-SEQ ID NO: 480-R2-CACGTG and TATTCGTC-R1-SEQ ID NO: 483-YSEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
CACGTG-R1-SEQ ID NO: 484-X-SEQ ID NO: 485-R2-SEQ ID NO: 480-R2-CACGTG and TATTCGTC-R1-SEQ ID NO: 481-YSEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
CACGTG-R1-SEQ ID NO: 484-X-SEQ ID NO: 485-R2-SEQ ID NO: 480-R2-CACGTG and TATTCGTC-R1-SEQ ID NO: 481-YSEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
CACGTG-R1-SEQ ID NO: 484-X-SEQ ID NO: 485-R2-SEQ ID NO: 480-R2-CACGTG and TATTCGTC-R1-SEQ ID NO: 482-Y-CTTGTASEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
CACGTG-R1-SEQ ID NO: 484-X-SEQ ID NO: 485-R2-SEQ ID NO: 480-R2-CACGTG and TATTCGTC-R1-SEQ ID NO: 482-Y-CTTGTASEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
CACGTG-R1-SEQ ID NO: 484-X-SEQ ID NO: 485-R2-SEQ ID NO: 480-R2-CACGTG and TATTCGTC-R1-SEQ ID NO: 483-YSEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
CACGTG-R1-SEQ ID NO: 484-X-SEQ ID NO: 485-R2-SEQ ID NO: 480-R2-CACGTG and TATTCGTC-R1-SEQ ID NO: 483-YSEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
CACGTG-R1-SEQ ID NO: 486-X-SEQ ID NO: 487-R2-SEQ ID NO: 480-R2-CACGTG et TATTCGTC-R1-SEQ ID NO: 481-YSEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
CACGTG-R1-SEQ ID NO: 486-X-SEQ ID NO: 487-R2-SEQ ID NO: 480-R2-CACGTG et TATTCGTC-R1-SEQ ID NO: 481-YSEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
CACGTG-R1-SEQ ID NO: 486-X-SEQ ID NO: 487-R2-SEQ ID NO: 480-R2-CACGTG and TATTCGTC-R1-SEQ ID NO: 482-Y-CTTGTASEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
CACGTG-R1-SEQ ID NO: 486-X-SEQ ID NO: 487-R2-SEQ ID NO: 480-R2-CACGTG and TATTCGTC-R1-SEQ ID NO: 482-Y-CTTGTASEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
CACGTG-R1-SEQ ID NO: 486-X-SEQ ID NO: 487-R2-SEQ ID NO: 480-R2-CACGTG and TATTCGTC-R1-SEQ ID NO: 483-YSEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
CACGTG-R1-SEQ ID NO: 486-X-SEQ ID NO: 487-R2-SEQ ID NO: 480-R2-CACGTG and TATTCGTC-R1-SEQ ID NO: 483-YSEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
R2-SEQ ID NO: 488-R1-SEQ ID NO: 478-X-SEQ ID NO: 479-R2-GACGAATA and TATTCGTC-R1-SEQ ID NO: 481-YSEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
R2-SEQ ID NO: 488-R1-SEQ ID NO: 478-X-SEQ ID NO: 479-R2-GACGAATA and TATTCGTC-R1-SEQ ID NO: 481-YSEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
R2-SEQ ID NO: 488-R1-SEQ ID NO: 478-X-SEQ ID NO: 479-R2-GACGAATA and TATTCGTC-R1-SEQ ID NO: 482-Y-CTTGTASEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
R2-SEQ ID NO: 488-R1-SEQ ID NO: 478-X-SEQ ID NO: 479-R2-GACGAATA and TATTCGTC-R1-SEQ ID NO: 482-Y-CTTGTASEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
R2-SEQ ID NO: 488-R1-SEQ ID NO: 478-X-SEQ ID NO: 479-R2-GACGAATA and TATTCGTC-R1-SEQ ID NO: 483-YSEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
R2-SEQ ID NO: 488-R1-SEQ ID NO: 478-X-SEQ ID NO: 479-R2-GACGAATA and TATTCGTC-R1-SEQ ID NO: 483-YSEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
R2-SEQ ID NO: 488-R1-SEQ ID NO: 484-X-SEQ ID NO: 485-R2-GACGAATA and TATTCGTC-R1-SEQ ID NO: 481-YSEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
R2-SEQ ID NO: 488-R1-SEQ ID NO: 484-X-SEQ ID NO: 485-R2-GACGAATA and TATTCGTC-R1-SEQ ID NO: 481-YSEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
R2-SEQ ID NO: 488-R1-SEQ ID NO: 484-X-SEQ ID NO: 485-R2-GACGAATA and TATTCGTC-R1-SEQ ID NO: 482-Y-CTTGTASEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
R2-SEQ ID NO: 488-R1-SEQ ID NO: 484-X-SEQ ID NO: 485-R2-GACGAATA and TATTCGTC-R1-SEQ ID NO: 482-Y-CTTGTASEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
R2-SEQ ID NO: 488-R1-SEQ ID NO: 484-X-SEQ ID NO: 485-R2-GACGAATA and TATTCGTC-R1-SEQ ID NO: 483-YSEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
R2-SEQ ID NO: 488-R1-SEQ ID NO: 484-X-SEQ ID NO: 485-R2-GACGAATA and TATTCGTC-R1-SEQ ID NO: 483-YSEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
R2-SEQ ID NO: 488-R1-SEQ ID NO: 486-X-SEQ ID NO: 487-R2-GACGAATA and TATTCGTC-R1-SEQ ID NO: 481-YSEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
R2-SEQ ID NO: 488-R1-SEQ ID NO: 486-X-SEQ ID NO: 487-R2-GACGAATA and TATTCGTC-R1-SEQ ID NO: 481-YSEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
R2-SEQ ID NO: 488-R1-SEQ ID NO: 486-X-SEQ ID NO: 487-R2-GACGAATA and TATTCGTC-R1-SEQ ID NO: 482-Y-CTTGTASEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424,
R2-SEQ ID NO: 488-R1-SEQ ID NO: 486-X-SEQ ID NO: 487-R2-GACGAATA and TATTCGTC-R1-SEQ ID NO: 482-Y-CTTGTASEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424,
R2-SEQ ID NO: 488-R1-SEQ ID NO: 486-X-SEQ ID NO: 487-R2-GACGAATA and TATTCGTC-R1-SEQ ID NO: 483-YSEQ ID NO: SEQ ID NO:, wherein R1 is any one of the sequences SEQ ID NO: n, n being an even number ranging from 269 to 424, and R2 is any one of the sequences SEQ ID NO: n+1, n being the same even number as that of R1 ranging from 269 to 424, and
R2-SEQ ID NO: 488-R1-SEQ ID NO: 486-X-SEQ ID NO: 487-R2-GACGAATA and TATTCGTC-R1-SEQ ID NO: 483-YSEQ ID NO: SEQ ID NO:, wherein R1 is any one of the sequences SEQ ID NO: n, n being an odd number ranging from 269 to 424, and R2 is any one of the sequences SEQ ID NO: n−1, n being the same odd number as that of R1 ranging from 269 to 424.

Advantageously, the invention relates to the aforementioned complex, said complex comprising one of the 300 pairs of first and second molecules of the following table 4:

TABLE 4
second molecule comprising

| | the first molecule comprising | the second molecule comprising |
|---|---|---|
| # | the sequence | the sequence |
| 1 | SEQ ID NO: 7-X-SEQ ID NO: | SEQ ID NO: 489-YSEQ ID NO: |
| | 442SEQ ID NO: | |
| 2 | SEQ ID NO: 490-X-SEQ ID | SEQ ID NO: 492-YSEQ ID NO: |
| | NO:491SEQ ID NO: | |
| 3 | SEQ ID NO: 493-X-SEQ ID NO: | SEQ ID NO: 489-YSEQ ID NO: |
| | 494SEQ ID NO: | |
| 4 | SEQ ID NO: 495-X-SEQ ID NO: | SEQ ID NO: 492-YSEQ ID NO: |
| | 496SEQ ID NO: | |
| 5 | SEQ ID NO: 493-X-SEQ ID NO: | SEQ ID NO: 499-Y-ccaagtSEQ ID |
| | 494SEQ ID NO: | NO: |
| 6 | SEQ ID NO: 495-X-SEQ ID NO: | SEQ ID NO: 500-Y-ccaagtSEQ ID |
| | 496SEQ ID NO: | NO: |
| 7 | SEQ ID NO: 497-X-SEQ ID NO: | SEQ ID NO: 489-YSEQ ID NO: |
| | 498SEQ ID NO: | |
| 8 | SEQ ID NO: 501-X-SEQ ID NO: | SEQ ID NO: 492-YSEQ ID NO: |
| | 502SEQ ID NO: | |
| 9 | SEQ ID NO: 497-X-SEQ ID NO: | SEQ ID NO: 499-Y-ccaagtSEQ ID |
| | 498SEQ ID NO: | NO: |
| 10 | SEQ ID NO: 501-X-SEQ ID NO: | SEQ ID NO: 500-Y-ccaagtSEQ ID |
| | 502SEQ ID NO: | NO: |
| 11 | SEQ ID NO: 503-X-SEQ ID NO: | Y-SEQ ID NO: 507, SEQ ID NO: |
| | 504SEQ ID NO: | |
| 12 | SEQ ID NO: 505-X-SEQ ID NO: | Y-SEQ ID NO: 508, SEQ ID NO: |
| | 506SEQ ID NO: | |
| 13 | SEQ ID NO: 503-X-SEQ ID NO: | Tacaag-Y-SEQ ID NO: 509, SEQ ID |
| | 504SEQ ID NO: | NO: |
| 14 | SEQ ID NO: 505-X-SEQ ID NO: | Tacaag-Y-SEQ ID NO: 510, SEQ ID |
| | 506SEQ ID NO: | NO: |
| 15 | SEQ ID NO: 503-X-SEQ ID NO: | Y-SEQ ID NO: 511, SEQ ID NO: |
| | 504SEQ ID NO: | |
| 16 | SEQ ID NO: 505-X-SEQ ID NO: | Y-SEQ ID NO: 512, SEQ ID NO: |
| | 506SEQ ID NO: | |
| 17 | SEQ ID NO: 525-X-SEQ ID NO: | Y-SEQ ID NO: 507, SEQ ID NO: |
| | 526SEQ ID NO: | |
| 18 | SEQ ID NO: 527-X-SEQ ID NO: | Y-SEQ ID NO: 508, SEQ ID NO: |
| | 528SEQ ID NO: | |
| 19 | SEQ ID NO: 525-X-SEQ ID NO: | Tacaag-Y-SEQ ID NO: 509, SEQ ID |
| | 526SEQ ID NO: | NO: |
| 20 | SEQ ID NO: 527-X-SEQ ID NO: | Tacaag-Y-SEQ ID NO: 510, SEQ ID |
| | 528SEQ ID NO: | NO: |
| 21 | SEQ ID NO: 525-X-SEQ ID NO: | Y-SEQ ID NO: 511, SEQ ID NO: |
| | 526SEQ ID NO: | |
| 22 | SEQ ID NO: 527-X-SEQ ID NO: | Y-SEQ ID NO: 512, SEQ ID NO: |
| | 528SEQ ID NO: | |
| 23 | SEQ ID NO: 529-X-SEQ ID NO: | Y-SEQ ID NO: 507, SEQ ID NO: |
| | 530SEQ ID NO: | |
| 24 | SEQ ID NO: 531-X-SEQ ID NO: | Y-SEQ ID NO: 508, SEQ ID NO: |
| | 532SEQ ID NO: | |
| 25 | SEQ ID NO: 529-X-SEQ ID NO: | Tacaag-Y-SEQ ID NO: 509, SEQ ID |
| | 530SEQ ID NO: | NO: |
| 26 | SEQ ID NO: 531-X-SEQ ID NO: | Tacaag-Y-SEQ ID NO: 510, SEQ ID |
| | 532SEQ ID NO: | NO: |
| 27 | SEQ ID NO: 529-X-SEQ ID NO: | Y-SEQ ID NO: 511, SEQ ID NO: |
| | 530SEQ ID NO: | |
| 28 | SEQ ID NO: 531-X-SEQ ID NO: | Y-SEQ ID NO: 512, SEQ ID NO: |
| | 532SEQ ID NO: | |
| 29 | SEQ ID NO: 533-X-SEQ ID NO: | Y-SEQ ID NO: 513, SEQ ID NO: |
| | 534SEQ ID NO: | |
| 30 | SEQ ID NO: 535-X-SEQ ID NO: | Y-SEQ ID NO: 514, SEQ ID NO: |
| | 536SEQ ID NO: | |
| 31 | SEQ ID NO: 533-X-SEQ ID NO: | Acttgg-Y-SEQ ID NO: 515, SEQ ID |
| | 534SEQ ID NO: | NO: |
| 32 | SEQ ID NO: 535-X-SEQ ID NO: | Acttgg-Y-SEQ ID NO: 516, SEQ ID |
| | 536SEQ ID NO: | NO: |
| 33 | SEQ ID NO: 533-X-SEQ ID NO: | Y-SEQ ID NO: 517, SEQ ID NO: |
| | 534SEQ ID NO: | |
| 34 | SEQ ID NO: 535-X-SEQ ID NO: | Y-SEQ ID NO: 518, SEQ ID NO: |
| | 536SEQ ID NO: | |
| 35 | SEQ ID NO: 537-X-SEQ ID NO: | Y-SEQ ID NO: 513,SEQ ID NO: |
| | 538SEQ ID NO: | |
| 36 | SEQ ID NO: 539-X-SEQ ID NO: | Y-SEQ ID NO: 514, SEQ ID NO: |
| | 540SEQ ID NO: | |
| 37 | SEQ ID NO: 537-X-SEQ ID NO: | Acttgg-Y-SEQ ID NO: 515, SEQ ID |
| | 538SEQ ID NO: | NO: |
| 38 | SEQ ID NO: 539-X-SEQ ID NO: | Acttgg-Y-SEQ ID NO: 516, SEQ ID |
| | 540SEQ ID NO: | NO: |
| 39 | SEQ ID NO: 537-X-SEQ ID NO: | Y-SEQ ID NO: 517, SEQ ID NO: |
| | 538SEQ ID NO: | |
| 40 | SEQ ID NO: 539-X-SEQ ID NO: | Y-SEQ ID NO: 518, SEQ ID NO: |
| | 540SEQ ID NO: | |
| 41 | SEQ ID NO: 541-X-SEQ ID NO: | Y-SEQ ID NO: 513, SEQ ID NO: |
| | 542SEQ ID NO: | |
| 42 | SEQ ID NO: 543-X-SEQ ID NO: | Y-SEQ ID NO: 514, SEQ ID NO: |
| | 544SEQ ID NO: | |
| 43 | SEQ ID NO: 541-X-SEQ ID NO: | Acttgg-Y-SEQ ID NO: 515, SEQ ID |
| | 542SEQ ID NO: | NO: |
| 44 | SEQ ID NO: 543-X-SEQ ID NO: | Acttgg-Y-SEQ ID NO: 516, SEQ ID |
| | 544SEQ ID NO: | NO: |
| 45 | SEQ ID NO: 541-X-SEQ ID NO: | Y-SEQ ID NO: 517, SEQ ID NO: |
| | 542SEQ ID NO: | |
| 46 | SEQ ID NO: 543-X-SEQ ID NO: | Y-SEQ ID NO: 518, SEQ ID NO: |
| | 544SEQ ID NO: | |
| 47 | SEQ ID NO: 545-X-SEQ ID NO: | Y-SEQ ID NO: 513,SEQ ID NO: |
| | 546SEQ ID NO: | |
| 48 | SEQ ID NO: 547-X-SEQ ID NO: | Y-SEQ ID NO: 514, SEQ ID NO: |
| | 548SEQ ID NO: | |
| 49 | SEQ ID NO: 545-X-SEQ ID NO: | Acttgg-Y-SEQ ID NO: 515, SEQ ID |
| | 546SEQ ID NO: | NO: |
| 50 | SEQ ID NO: 547-X-SEQ ID NO: | Acttgg-Y-SEQ ID NO: 516, SEQ ID |
| | 548SEQ ID NO: | NO: |
| 51 | SEQ ID NO: 545-X-SEQ ID NO: | Y-SEQ ID NO: 517, SEQ ID NO: |
| | 546SEQ ID NO: | |
| 52 | SEQ ID NO: 547-X-SEQ ID NO: | Y-SEQ ID NO: 518, SEQ ID NO: |
| | 548SEQ ID NO: | |
| 53 | SEQ ID NO: 549-X-SEQ ID NO: | Y-SEQ ID NO: 513, SEQ ID NO: |
| | 550SEQ ID NO: | |
| 54 | SEQ ID NO: 551-X-SEQ ID NO: | Y-SEQ ID NO: 514, SEQ ID NO: |
| | 552SEQ ID NO: | |
| 55 | SEQ ID NO: 549-X-SEQ ID NO: | Acttgg-Y-SEQ ID NO: 515, SEQ ID |
| | 550SEQ ID NO: | NO: |
| 56 | SEQ ID NO: 551-X-SEQ ID NO: | Acttgg-Y-SEQ ID NO:5 16, SEQ ID |
| | 552SEQ ID NO: | NO: |
| 57 | SEQ ID NO: 549-X-SEQ ID NO: | Y-SEQ ID NO: 517, SEQ ID NO: |
| | 550SEQ ID NO: | |
| 58 | SEQ ID NO: 551-X-SEQ ID NO: | Y-SEQ ID NO: 518, SEQ ID NO: |
| | 552SEQ ID NO: | |
| 59 | SEQ ID NO: 553-X-SEQ ID NO: | Y-SEQ ID NO: 513, SEQ ID NO: |
| | 554SEQ ID NO: | |
| 60 | SEQ ID NO: 555-X-SEQ ID NO: | Y-SEQ ID NO: 514, SEQ ID NO: |
| | 556SEQ ID NO: | |
| 61 | SEQ ID NO: 553-X-SEQ ID NO: | Acttgg-Y-SEQ ID NO: 515, SEQ ID |
| | 554SEQ ID NO: | INO: |
| 62 | SEQ ID NO: 555-X-SEQ ID NO: | Acttgg-Y-SEQ ID NO: 516, SEQ ID |
| | 556SEQ ID NO: | NO: |
| 63 | SEQ ID NO: 553-X-SEQ ID NO: | Y-SEQ ID NO: 517, SEQ ID NO: |
| | 554SEQ ID NO: | |
| 64 | SEQ ID NO: 555-X-SEQ ID NO: | Y-SEQ ID NO: 518, SEQ ID NO: |
| | 556SEQ ID NO: | |
| 65 | SEQ ID NO: 557-X-SEQ ID NO: | SEQ ID NO: 519-YSEQ ID NO: |
| | 558SEQ ID NO: | |
| 66 | SEQ ID NO: 559-X-SEQ ID NO: | SEQ ID NO: 520-YSEQ ID NO: |
| | 560SEQ ID NO: | |
| 67 | SEQ ID NO: 557-X-SEQ ID NO: | SEQ ID NO: 521-Y-cttgtaSEQ ID |
| | 558SEQ ID NO: | NO: |
| 68 | SEQ ID NO: 559-X-SEQ ID NO: | SEQ ID NO: 522, SEQ ID NO: |
| | 560SEQ ID NO: | |
| 69 | SEQ ID NO: 557-X-SEQ ID NO: | SEQ ID NO: 523, SEQ ID NO: |
| | 558SEQ ID NO: | |
| 70 | SEQ ID NO: 559-X-SEQ ID NO: | SEQ ID NO: 524, SEQ ID NO: |
| | 560SEQ ID NO: | |
| 71 | SEQ ID NO: 561-X-SEQ ID NO: | SEQ ID NO: 519-YSEQ ID NO: |
| | 562SEQ ID NO: | |
| 72 | SEQ ID NO: 563-X-SEQ ID NO: | SEQ ID NO: 520-YSEQ ID NO: |
| | 564SEQ ID NO: | |
| 73 | SEQ ID NO: 561-X-SEQ ID NO: | SEQ ID NO: 521-Y-cttgtaSEQ ID |
| | 562SEQ ID NO: | NO: |
| 74 | SEQ ID NO: 563-X-SEQ ID NO: | SEQ ID NO: 522, SEQ ID NO: |
| | 564SEQ ID NO: | |
| 75 | SEQ ID NO: 561-X-SEQ ID NO: | SEQ ID NO: 523, SEQ ID NO: |
| | 562SEQ ID NO: | |
| 76 | SEQ ID NO: 563-X-SEQ ID NO: | SEQ ID NO: 524, SEQ ID NO: |
| | 564SEQ ID NO: | |
| 77 | SEQ ID NO: 565-X-SEQ ID NO: | SEQ ID NO: 519-YSEQ ID NO: |
| | 566SEQ ID NO: | |
| 78 | SEQ ID NO: 567-X-SEQ ID NO: | SEQ ID NO: 520-YSEQ ID NO: |
| | 568SEQ ID NO: | |
| 79 | SEQ ID NO: 565-X-SEQ ID NO: | SEQ ID NO: 521-Y-cttgtaSEQ ID |
| | 566SEQ ID NO: | NO: |
| 80 | SEQ ID NO: 567-X-SEQ ID NO: | SEQ ID NO: 522, SEQ ID NO: |
| | 568SEQ ID NO: | |
| 81 | SEQ ID NO: 565-X-SEQ ID NO: | SEQ ID NO: 523, SEQ ID NO: |
| | 566SEQ ID NO: | |
| 82 | SEQ ID NO: 567-X-SEQ ID NO: | SEQ ID NO: 524, SEQ ID NO: |
| | 568SEQ ID NO: | |
| 83 | SEQ ID NO: 569-X-SEQ ID NO: | SEQ ID NO: 519-YSEQ ID NO: |
| | 570SEQ ID NO: | |
| 84 | SEQ ID NO: 571-X-SEQ ID NO: | SEQ ID NO: 520-YSEQ ID NO: |
| | 572SEQ ID NO: | |
| 85 | SEQ ID NO: 569-X-SEQ ID NO: | SEQ ID NO: 521-Y-cttgtaSEQ ID |
| | 570SEQ ID NO: | NO: |
| 86 | SEQ ID NO: 571-X-SEQ ID NO: | SEQ ID NO: 522-Y-cttgtaSEQ ID |
| | 572SEQ ID NO: | NO: |
| 87 | SEQ ID NO: 569-X-SEQ ID NO: | SEQ ID NO: 523-YSEQ ID NO: |
| | 570SEQ ID NO: | |
| 88 | SEQ ID NO: 571-X-SEQ ID NO: | SEQ ID NO: 524-YSEQ ID NO: |
| | 572SEQ ID NO: | |
| 89 | SEQ ID NO: 573-X-SEQ ID NO: | SEQ ID NO: 519-YSEQ ID NO: |
| | 574SEQ ID NO: | |
| 90 | SEQ ID NO: 575-X-SEQ ID NO: | SEQ ID NO: 520-YSEQ ID NO: |
| | 576SEQ ID NO: | |
| 91 | SEQ ID NO: 573-X-SEQ ID NO: | SEQ ID NO: 521-Y-cttgtaSEQ ID |
| | 574SEQ ID NO: | NO: |
| 92 | SEQ ID NO: 575-X-SEQ ID NO: | SEQ ID NO: 522-Y-cttgtaSEQ ID |
| | 576SEQ ID NO: | NO: |
| 93 | SEQ ID NO: 573-X-SEQ ID NO: | SEQ ID NO: 523-YSEQ ID NO: |
| | 574SEQ ID NO: | |
| 94 | SEQ ID NO: 575-X-SEQ ID NO: | SEQ ID NO: 524-YSEQ ID NO: |
| | 576SEQ ID NO: | |
| 95 | SEQ ID NO: 577-X-SEQ ID NO: | SEQ ID NO: 519-YSEQ ID NO: |
| | 578SEQ ID NO: | |
| 96 | SEQ ID NO: 579-X-SEQ ID NO: | SEQ ID NO: 520-YSEQ ID NO: |
| | 580SEQ ID NO: | |
| 97 | SEQ ID NO: 577-X-SEQ ID NO: | SEQ ID NO: 521-Y-cttgtaSEQ ID |
| | 578SEQ ID NO: | NO: |
| 98 | SEQ ID NO: 579-X-SEQ ID NO: | SEQ ID NO: 522-Y-cttgtaSEQ ID |
| | 580SEQ ID NO: | NO: |
| 99 | SEQ ID NO: 577-X-SEQ ID NO: | SEQ ID NO: 523-YSEQ ID NO: |
| | 578SEQ ID NO: | |
| 100 | SEQ ID NO: 579-X-SEQ ID NO: | SEQ ID NO: 524-YSEQ ID NO: |
| | 580SEQ ID NO: | |
| 101 | SEQ ID NO: 581-X-SEQ ID NO: | Y-SEQ ID NO: 645SEQ ID NO: |
| | 582SEQ ID NO: | |
| 102 | SEQ ID NO: 583-X-SEQ ID NO: | Y-SEQ ID NO: 646SEQ ID NO: |
| | 584SEQ ID NO: | |
| 103 | SEQ ID NO: 585-X-SEQ ID NO: | Y-SEQ ID NO: 645SEQ ID NO: |
| | 586SEQ ID NO: | |
| 104 | SEQ ID NO: 587-X-SEQ ID NO: | Y-SEQ ID NO: 646SEQ ID NO: |
| | 588SEQ ID NO: | |
| 105 | SEQ ID NO: 585-X-SEQ ID NO: | tacaag-Y-SEQ ID NO: 647SEQ ID |
| | 586SEQ ID NO: | NO: |
| 106 | SEQ ID NO: 587-X-SEQ ID NO: | tacaag-Y-SEQ ID NO: 648SEQ ID |
| | 588SEQ ID NO: | NO: |
| 107 | SEQ ID NO: 589-X-SEQ ID NO: | Y-SEQ ID NO: 645SEQ ID NO: |
| | 590SEQ ID NO: | |
| 108 | SEQ ID NO: 591-X-SEQ ID NO: | Y-SEQ ID NO: 646SEQ ID NO: |
| | 592SEQ ID NO: | |
| 109 | SEQ ID NO: 589-X-SEQ ID NO: | tacaag-Y-SEQ ID NO: 647SEQ ID |
| | 590SEQ ID NO: | NO: |
| 110 | SEQ ID NO: 591-X-SEQ ID NO: | tacaag-Y-SEQ ID NO: 648SEQ ID |
| | 592SEQ ID NO: | NO: |
| 111 | SEQ ID NO: 593-X-SEQ ID NO: | Y-SEQ ID NO: 649SEQ ID NO: |
| | 594SEQ ID NO: | |
| 112 | SEQ ID NO: 595-X-SEQ ID NO: | Y-SEQ ID NO: 650SEQ ID NO: |
| | 596SEQ ID NO: | |
| 113 | SEQ ID NO: 593-X-SEQ ID NO: | Y-SEQ ID NO: 651SEQ ID NO: |
| | 594SEQ ID NO: | |
| 114 | SEQ ID NO: 595-X-SEQ ID NO: | Y-SEQ ID NO: 652SEQ ID NO: |
| | 596SEQ ID NO: | |
| 115 | SEQ ID NO: 593-X-SEQ ID NO: | acttgg-Y-SEQ ID NO: 653SEQ ID |
| | 594SEQ ID NO: | NO: |
| 116 | SEQ ID NO: 595-X-SEQ ID NO: | acttgg-Y-SEQ ID NO: 654SEQ ID |
| | 596SEQ ID NO: | NO: |
| 117 | SEQ ID NO: 597-X-SEQ ID NO: | Y-SEQ ID NO: 649SEQ ID NO: |
| | 598SEQ ID NO: | |
| 118 | SEQ ID NO: 599-X-SEQ ID NO: | Y-SEQ ID NO: 650SEQ ID NO: |
| | 600SEQ ID NO: | |
| 119 | SEQ ID NO: 597-X-SEQ ID NO: | Y-SEQ ID NO: 651SEQ ID NO: |
| | 598SEQ ID NO: | |
| 120 | SEQ ID NO: 599-X-SEQ ID NO: | Y-SEQ ID NO: 652SEQ ID NO: |
| | 600SEQ ID NO: | |
| 121 | SEQ ID NO: 597-X-SEQ ID NO: | acttgg-Y-SEQ ID NO: 653SEQ ID |
| | 598SEQ ID NO: | NO: |
| 122 | SEQ ID NO: 599-X-SEQ ID NO: | acttgg-Y-SEQ ID NO: 654SEQ ID |
| | 600SEQ ID NO: | NO: |
| 123 | SEQ ID NO: 601-X-SEQ ID NO: | Y-SEQ ID NO: 649SEQ ID NO: |
| | 602SEQ ID NO: | |
| 124 | SEQ ID NO: 603-X-SEQ ID NO: | Y-SEQ ID NO: 650SEQ ID NO: |
| | 604SEQ ID NO: | |
| 125 | SEQ ID NO: 601-X-SEQ ID NO: | Y-SEQ ID NO: 651SEQ ID NO: |
| | 602SEQ ID NO: | |
| 126 | SEQ ID NO: 603-X-SEQ ID NO: | Y-SEQ ID NO: 652SEQ ID NO: |
| | 604SEQ ID NO: | |
| 127 | SEQ ID NO: 601-X-SEQ ID NO: | acttgg-Y-SEQ ID NO: 653SEQ ID |
| | 602SEQ ID NO: | NO: |
| 128 | SEQ ID NO: 603-X-SEQ ID NO: | acttgg-Y-SEQ ID NO: 654SEQ ID |
| | 604SEQ ID NO: | NO: |
| 129 | SEQ ID NO: 605-X-SEQ ID NO: | Y-SEQ ID NO: 655SEQ ID NO: |
| | 606SEQ ID NO: | |
| 130 | SEQ ID NO: 607-X-SEQ ID NO: | Y-SEQ ID NO: 656SEQ ID NO: |
| | 608SEQ ID NO: | |
| 131 | SEQ ID NO: 605-X-SEQ ID NO: | SEQ ID NO: 657-YSEQ ID NO: |
| | 606SEQ ID NO: | |
| 132 | SEQ ID NO: 607-X-SEQ ID NO: | SEQ ID NO: 658-YSEQ ID NO: |
| | 608SEQ ID NO: | |
| 133 | SEQ ID NO: 605-X-SEQ ID NO: | SEQ ID NO: 659-Y- cttgtaSEQ ID |
| | 606SEQ ID NO: | NO: |
| 134 | SEQ ID NO: 607-X-SEQ ID NO: | SEQ ID NO: 660-Y- cttgtaSEQ ID |
| | 608SEQ ID NO: | NO: |
| 135 | SEQ ID NO: 609-X-SEQ ID NO: | Y-SEQ ID NO: 655SEQ ID NO: |
| | 610SEQ ID NO: | |
| 136 | SEQ ID NO: 611-X-SEQ ID NO: | Y-SEQ ID NO: 656SEQ ID NO: |
| | 612SEQ ID NO: | |
| 137 | SEQ ID NO: 609-X-SEQ ID NO: | SEQ ID NO: 657-Y, SEQ ID NO: |
| | 610SEQ ID NO: | |
| 138 | SEQ ID NO: 611-X-SEQ ID NO: | SEQ ID NO: 658-YSEQ ID NO: |
| | 612SEQ ID NO: | |
| 139 | SEQ ID NO: 609-X-SEQ ID NO: | SEQ ID NO: 659-Y-cttgtaSEQ ID |
| | 610SEQ ID NO: | NO: |
| 140 | SEQ ID NO: 611-X-SEQ ID NO: | SEQ ID NO: 660-Y-cttgtaSEQ ID |
| | 612SEQ ID NO: | NO: |
| 141 | SEQ ID NO: 613-X-SEQ ID NO: | Y-SEQ ID NO: 655SEQ ID NO: |
| | 614SEQ ID NO: | |
| 142 | SEQ ID NO: 615-X-SEQ ID NO: | Y-SEQ ID NO: 656SEQ ID NO: |
| | 616SEQ ID NO: | |
| 143 | SEQ ID NO: 613-X-SEQ ID NO: | SEQ ID NO: 657-Y, SEQ ID NO: |
| | 614SEQ ID NO: | |
| 144 | SEQ ID NO: 615-X-SEQ ID NO: | SEQ ID NO: 658-YSEQ ID NO: |
| | 616SEQ ID NO: | |
| 145 | SEQ ID NO: 613-X-SEQ ID NO: | SEQ ID NO: 659-Y- cttgtaSEQ ID |
| | 614SEQ ID NO: | NO: |
| 146 | SEQ ID NO: 615-X-SEQ ID NO: | SEQ ID NO: 660-Y- cttgtaSEQ ID |
| | 616SEQ ID NO: | NO: |
| 147 | SEQ ID NO: 617-X-SEQ ID NO: | Y-SEQ ID NO: 655SEQ ID NO: |
| | 618SEQ ID NO: | |
| 148 | SEQ ID NO: 619-X-SEQ ID NO: | Y-SEQ ID NO: 656SEQ ID NO: |
| | 620SEQ ID NO: | |
| 149 | SEQ ID NO: 617-X-SEQ ID NO: | SEQ ID NO: 657-YSEQ ID NO: |
| | 618SEQ ID NO: | |
| 150 | SEQ ID NO: 619-X-SEQ ID NO: | SEQ ID NO: 658-YSEQ ID NO: |
| | 620SEQ ID NO: | |
| 151 | SEQ ID NO: 617-X-SEQ ID NO: | SEQ ID NO: 659-Y-cttgtaSEQ ID |
| | 618SEQ ID NO: | NO: |
| 152 | SEQ ID NO: 619-X-SEQ ID NO: | SEQ ID NO: 660-Y-cttgtaSEQ ID |
| | 620SEQ ID NO: | NO: |
| 153 | SEQ ID NO: 621-X-SEQ ID NO: | Y-SEQ ID NO: 655SEQ ID NO: |
| | 622SEQ ID NO: | |
| 154 | SEQ ID NO: 623-X-SEQ ID NO: | Y-SEQ ID NO: 656SEQ ID NO: |
| | 624SEQ ID NO: | |
| 155 | SEQ ID NO: 621-X-SEQ ID NO: | SEQ ID NO: 657-YSEQ ID NO: |
| | 622SEQ ID NO: | |
| 156 | 624SEQ ID NO: | SEQ ID NO: 658-YSEQ ID NO: |
| | SEQ ID NO: 623-X-SEQ ID NO: | |
| 157 | SEQ ID NO: 621-X-SEQ ID NO: | SEQ ID NO: 659-Y-cttgtaSEQ ID |
| | 622SEQ ID NO: | NO: |
| 158 | SEQ ID NO: 623-X-SEQ ID NO: | SEQ ID NO: 660-Y-cttgtaSEQ ID |
| | 624SEQ ID NO: | NO: |
| 159 | SEQ ID NO: 625-X-SEQ ID NO: | Y-SEQ ID NO: 655SEQ ID NO: |
| | 626SEQ ID NO: | |
| 160 | SEQ ID NO: 627-X-SEQ ID NO: | Y-SEQ ID NO: 656SEQ ID NO: |
| | 628SEQ ID NO: | |
| 161 | SEQ ID NO: 625-X-SEQ ID NO: | SEQ ID NO: 657-Y, SEQ ID NO: |
| | 626SEQ ID NO: | |
| 162 | SEQ ID NO: 627-X-SEQ ID NO: | SEQ ID NO: 658-YSEQ ID NO: |
| | 628SEQ ID NO: | |
| 163 | SEQ ID NO: 625-X-SEQ ID NO: | SEQ ID NO: 659-Y-cttgtaSEQ ID |
| | 626SEQ ID NO: | NO: |
| 164 | SEQ ID NO: 627-X-SEQ ID NO: | SEQ ID NO: 660-Y-cttgtaSEQ ID |
| | 628SEQ ID NO: | NO: |
| 165 | SEQ ID NO: 629-X-SEQ ID NO: | SEQ ID NO: 661-YSEQ ID NO: |
| | 630SEQ ID NO: | |
| 166 | 632SEQ ID NO: | SEQ ID NO: 662-YSEQ ID NO: |
| | SEQ ID NO: 631-X-SEQ ID NO: | |
| 1676 | SEQ ID NO: 629-X-SEQ ID NO: | SEQ ID NO: 749-X-SEQ ID NO: |
| | 30SEQ ID NO: | 750SEQ ID NO: |
| 168 | SEQ ID NO: 631-X-SEQ ID NO: | SEQ ID NO: 751-X-SEQ ID NO: |
| | 632SEQ ID NO: | 752SEQ ID NO: |
| 169 | SEQ ID NO: 629-X-SEQ ID NO: | SEQ ID NO: 753-X-SEQ ID NO: |
| | 630SEQ ID NO: | 754SEQ ID NO: |
| 170 | SEQ ID NO: 631-X-SEQ ID NO: | SEQ ID NO: 755-X-SEQ ID NO: |
| | 632SEQ ID NO: | 756SEQ ID NO: |
| 171 | SEQ ID NO: 633-X-SEQ ID NO: | SEQ ID NO: 661-YSEQ ID NO: |
| | 634SEQ ID NO: | |
| 172 | SEQ ID NO: 635-X-SEQ ID NO: | SEQ ID NO: 662-YSEQ ID NO: |
| | 636SEQ ID NO: | |
| 173 | SEQ ID NO: 633-X-SEQ ID NO: | SEQ ID NO: 749-X-SEQ ID NO: |
| | 634SEQ ID NO: | 750SEQ ID NO: |
| 174 | SEQ ID NO: 635-X-SEQ ID NO: | SEQ ID NO: 751-X-SEQ ID NO: |
| | 636SEQ ID NO: | 752SEQ ID NO: |
| 175 | SEQ ID NO: 633-X-SEQ ID NO: | SEQ ID NO: 753-X-SEQ ID NO: |
| | 634SEQ ID NO: | 754SEQ ID NO: |
| 176 | SEQ ID NO: 635-X-SEQ ID NO: | SEQ ID NO: 755-X-SEQ ID NO: |
| | 636SEQ ID NO: | 756SEQ ID NO: |
| 177 | SEQ ID NO: 637-X-SEQ ID NO: | SEQ ID NO: 661-YSEQ ID NO: |
| | 638SEQ ID NO: | |
| 178 | SEQ ID NO: 639-X-SEQ ID NO: | SEQ ID NO: 662-YSEQ ID NO: |
| | 640SEQ ID NO: | |
| 179 | SEQ ID NO: 637-X-SEQ ID NO: | SEQ ID NO: 749-X-SEQ ID NO: |
| | 638SEQ ID NO: | 750SEQ ID NO: |
| 180 | SEQ ID NO: 639-X-SEQ ID NO: | SEQ ID NO: 751-X-SEQ ID NO: |
| | 640SEQ ID NO: | 752SEQ ID NO: |
| 181 | SEQ ID NO: 637-X-SEQ ID NO: | SEQ ID NO: 753-X-SEQ ID NO: |
| | 638SEQ ID NO: | 754SEQ ID NO: |
| 182 | SEQ ID NO: 639-X-SEQ ID NO: | SEQ ID NO: 755-X-SEQ ID NO: |
| | 640SEQ ID NO: | 756SEQ ID NO: |
| 183 | SEQ ID NO: 641-X-SEQ ID NO: | SEQ ID NO: 661-YSEQ ID NO: |
| | 642SEQ ID NO: | |
| 184 | SEQ ID NO: 643-X-SEQ ID NO: | SEQ ID NO: 662-YSEQ ID NO: |
| | 644SEQ ID NO: | |
| 185 | SEQ ID NO: 641-X-SEQ ID NO: | SEQ ID NO: 749-X-SEQ ID NO: |
| | 642SEQ ID NO: | 750SEQ ID NO: |
| 186 | SEQ ID NO: 643-X-SEQ ID NO: | SEQ ID NO: 751-X-SEQ ID NO: |
| | 644SEQ ID NO: | 752SEQ ID NO: |
| 187 | SEQ ID NO: 641-X-SEQ ID NO: | SEQ ID NO: 753-X-SEQ ID NO: |
| | 642SEQ ID NO: | 754SEQ ID NO: |
| 188 | SEQ ID NO: 643-X-SEQ ID NO: | SEQ ID NO: 755-X-SEQ ID NO: |
| | 644SEQ ID NO: | 756SEQ ID NO: |
| 189 | SEQ ID NO: 757-YSEQ ID NO: | SEQ ID NO: 761-YSEQ ID NO: |
| 190 | SEQ ID NO: 758-YSEQ ID NO: | SEQ ID NO: 762-YSEQ ID NO: |
| 191 | SEQ ID NO: 757-YSEQ ID NO: | SEQ ID NO: 763-X-SEQ ID NO: |
| | | 764SEQ ID NO: |
| 192 | SEQ ID NO: 758-YSEQ ID NO: | SEQ ID NO: 765-X-SEQ ID NO: |
| | | 766SEQ ID NO: |
| 193 | SEQ ID NO: 757-YSEQ ID NO: | SEQ ID NO: 767-X-SEQ ID NO: |
| | | 768SEQ ID NO: |
| 194 | SEQ ID NO: 758-YSEQ ID NO: | SEQ ID NO: 769-X-SEQ ID NO: |
| | | 770SEQ ID NO: |
| 195 | SEQ ID NO: 759-Y- ccaagtSEQ ID | SEQ ID NO: 761-YSEQ ID NO: |
| | NO: | |
| 196 | SEQ ID NO: 760-Y- ccaagtSEQ ID | SEQ ID NO: 762-YSEQ ID NO: |
| | NO: | |
| 197 | SEQ ID NO: 759-Y- ccaagtSEQ ID | SEQ ID NO: 763-X-SEQ ID NO: |
| | NO: | 764SEQ ID NO: |
| 198 | SEQ ID NO: 760-Y- ccaagtSEQ ID | SEQ ID NO: 765-X-SEQ ID NO: |
| | NO: | 766SEQ ID NO: |
| 199 | SEQ ID NO: 759-Y- ccaagtSEQ ID | SEQ ID NO: 767-X-SEQ ID NO: |
| | NO: | 768SEQ ID NO: |
| 200 | SEQ ID NO: 760-Y-ccaagtSEQ ID | SEQ ID NO: 769-X-SEQ ID NO: |
| | NO: | 770SEQ ID NO: |
| 201 | SEQ ID NO: 663-X-SEQ ID NO: | Y-SEQ ID NO: 727SEQ ID NO: |
| | 664SEQ ID NO: | |
| 202 | SEQ ID NO: 665-X-SEQ ID NO: | Y-SEQ ID NO: 728SEQ ID NO: |
| | 666SEQ ID NO: | |
| 203 | SEQ ID NO: 667-X-SEQ ID NO: | Y-SEQ ID NO: 727SEQ ID NO: |
| | 668SEQ ID NO: | |
| 204 | SEQ ID NO: 669-X-SEQ ID NO: | Y-SEQ ID NO: 728SEQ ID NO: |
| | 670SEQ ID NO: | |
| 205 | SEQ ID NO: 667-X-SEQ ID NO: | tacaag-Y-SEQ ID NO: 729SEQ ID |
| | 668SEQ ID NO: | NO: |
| 206 | SEQ ID NO: 669-X-SEQ ID NO: | tacaag-Y-SEQ ID NO: 730SEQ ID |
| | 670SEQ ID NO: | NO: |
| 207 | SEQ ID NO: 671-X-SEQ ID NO: | Y-SEQ ID NO: 727SEQ ID NO: |
| | 672SEQ ID NO: | |
| 208 | SEQ ID NO: 673-X-SEQ ID NO: | Y-SEQ ID NO: 728SEQ ID NO: |
| | 674SEQ ID NO: | |
| 209 | SEQ ID NO: 671-X-SEQ ID NO: | tacaag-Y-SEQ ID NO: 729SEQ ID |
| | 672SEQ ID NO: | NO: |
| 210 | SEQ ID NO: 673-X-SEQ ID NO: | tacaag-Y-SEQ ID NO: 730SEQ ID |
| | 674SEQ ID NO: | NO: |
| 211 | SEQ ID NO: 675-X-SEQ ID NO: | Y-SEQ ID NO: 731SEQ ID NO: |
| | 676SEQ ID NO: | |
| 212 | SEQ ID NO: 677-X-SEQ ID NO: | Y-SEQ ID NO: 732SEQ ID NO: |
| | 678SEQ ID NO: | |
| 213 | SEQ ID NO: 675-X-SEQ ID NO: | Y-SEQ ID NO: 733SEQ ID NO: |
| | 676SEQ ID NO: | |
| 214 | SEQ ID NO: 677-X-SEQ ID NO: | Y-SEQ ID NO: 734SEQ ID NO: |
| | 678SEQ ID NO: | |
| 215 | SEQ ID NO: 675-X-SEQ ID NO: | acttgg-Y-SEQ ID NO: 735SEQ ID |
| | 676SEQ ID NO: | NO: |
| 216 | SEQ ID NO: 677-X-SEQ ID NO: | acttgg-Y-SEQ ID NO: 736SEQ ID |
| | 678SEQ ID NO: | NO: |
| 217 | SEQ ID NO: 679-X-SEQ ID NO: | Y-SEQ ID NO: 731SEQ ID NO: |
| | 680SEQ ID NO: | |
| 218 | SEQ ID NO: 681-X-SEQ ID NO: | Y-SEQ ID NO: 732SEQ ID NO: |
| | 682SEQ ID NO: | |
| 219 | SEQ ID NO: 679-X-SEQ ID NO: | Y-SEQ ID NO: 733SEQ ID NO: |
| | 680SEQ ID NO: | |
| 220 | SEQ ID NO: 681-X-SEQ ID NO: | Y-SEQ ID NO: 734SEQ ID NO: |
| | 682SEQ ID NO: | |
| 221 | SEQ ID NO: 679-X-SEQ ID NO: | acttgg-Y-SEQ ID NO: 735SEQ ID |
| | 680SEQ ID NO: | NO: |
| 222 | SEQ ID NO: 681-X-SEQ ID NO: | acttgg-Y-SEQ ID NO: 736SEQ ID |
| | 682SEQ ID NO: | NO: |
| 223 | SEQ ID NO: 683-X-SEQ ID NO: | Y-SEQ ID NO: 731SEQ ID NO: |
| | 684SEQ ID NO: | |
| 224 | SEQ ID NO: 685-X-SEQ ID NO: | Y-SEQ ID NO: 732SEQ ID NO: |
| | 686SEQ ID NO: | |
| 225 | SEQ ID NO: 683-X-SEQ ID NO: | Y-SEQ ID NO: 733SEQ ID NO: |
| | 684SEQ ID NO: | |
| 226 | SEQ ID NO: 685-X-SEQ ID NO: | Y-SEQ ID NO: 734SEQ ID NO: |
| | 686SEQ ID NO: | |
| 227 | SEQ ID NO: 683-X-SEQ ID NO: | acttgg-Y-SEQ ID NO: 735SEQ ID |
| | 684SEQ ID NO: | NO: |
| 228 | SEQ ID NO: 685-X-SEQ ID NO: | acttgg-Y-SEQ ID NO: 736SEQ ID |
| | 686SEQ ID NO: | NO: |
| 229 | SEQ ID NO: 687-X-SEQ ID NO: | Y-SEQ ID NO: 737SEQ ID NO: |
| | 688SEQ ID NO: | |
| 230 | SEQ ID NO: 689-X-SEQ ID NO: | Y-SEQ ID NO: 738SEQ ID NO: |
| | 690SEQ ID NO: | |
| 231 | SEQ ID NO: 687-X-SEQ ID NO: | SEQ ID NO: 739-YSEQ ID NO: |
| | 688SEQ ID NO: | |
| 232 | SEQ ID NO: 689-X-SEQ ID NO: | SEQ ID NO: 740-YSEQ ID NO: |
| | 690SEQ ID NO: | |
| 233 | SEQ ID NO: 687-X-SEQ ID NO: | SEQ ID NO: 741-Y-cttgtaSEQ ID |
| | 688SEQ ID NO: | INO: |
| 234 | SEQ ID NO: 689-X-SEQ ID NO: | SEQ ID NO: 742-Y-cttgtaSEQ ID |
| | 690SEQ ID NO: | NO: |
| 235 | SEQ ID NO: 691-X-SEQ ID NO: | Y-SEQ ID NO: 737SEQ ID NO: |
| | 692SEQ ID NO: | |
| 236 | SEQ ID NO:693-X-SEQ ID NO: | Y-SEQ ID NO: 738SEQ ID NO: |
| | 694SEQ ID NO: | |
| 237 | SEQ ID NO: 691-X-SEQ ID NO: | SEQ ID NO: 739-YSEQ ID NO: |
| | 692SEQ ID NO: | |
| 238 | SEQ ID NO: 693-X-SEQ ID NO: | SEQ ID NO: 740-YSEQ ID NO: |
| | 694SEQ ID NO: | |
| 239 | SEQ ID NO: 691-X-SEQ ID NO: | SEQ ID NO: 741-Y-cttgtaSEQ ID |
| | 692SEQ ID NO: | NO: |
| 240 | SEQ ID NO: 693-X-SEQ ID NO: | SEQ ID NO: 742-Y-cttgtaSEQ ID |
| | 694SEQ ID NO: | NO: |
| 241 | SEQ ID NO: 695-X-SEQ ID NO: | Y-SEQ ID NO: 737SEQ ID NO: |
| | 696SEQ ID NO: | |
| 242 | SEQ ID NO: 697-X-SEQ ID NO: | Y-SEQ ID NO: 738SEQ ID NO: |
| | 698SEQ ID NO: | |
| 243 | SEQ ID NO: 695-X-SEQ ID NO: | SEQ ID NO: 739-YSEQ ID NO: |
| | 696SEQ ID NO: | |
| 244 | SEQ ID NO: 697-X-SEQ ID NO: | SEQ ID NO: 740-YSEQ ID NO: |
| | 698SEQ ID NO: | |
| 245 | SEQ ID NO: 695-X-SEQ ID NO: | SEQ ID NO: 741-Y-cttgtaSEQ ID |
| | 696SEQ ID NO: | NO: |
| 246 | SEQ ID NO: 697-X-SEQ ID NO: | SEQ ID NO: 742-Y-cttgtaSEQ ID |
| | 698SEQ ID NO: | NO: |
| 247 | SEQ ID NO: 699-X-SEQ ID NO: | Y-SEQ ID NO: 737SEQ ID NO: |
| | 700SEQ ID NO: | |
| 248 | SEQ ID NO: 701-X-SEQ ID NO: | Y-SEQ ID NO: 738SEQ ID NO: |
| | 702SEQ ID NO: | |
| 249 | SEQ ID NO: 699-X-SEQ ID NO: | SEQ ID NO: 739-YSEQ ID NO: |
| | 700SEQ ID NO: | |
| 250 | SEQ ID NO: 701-X-SEQ ID NO: | SEQ ID NO: 740-YSEQ ID NO: |
| | 702SEQ ID NO: | |
| 251 | SEQ ID NO: 699-X-SEQ ID NO: | SEQ ID NO: 741-Y-cttgtaSEQ ID |
| | 700SEQ ID NO: | NO: |
| 252 | SEQ ID NO: 701-X-SEQ ID NO: | SEQ ID NO: 742-Y-cttgtaSEQ ID |
| | 702SEQ ID NO: | NO: |
| 253 | SEQ ID NO: 703-X-SEQ ID NO: | Y-SEQ ID NO: 737SEQ ID NO: |
| | 704SEQ ID NO: | |
| 254 | SEQ ID NO: 705-X-SEQ ID NO: | Y-SEQ ID NO: 738SEQ ID NO: |
| | 706SEQ ID NO: | |
| 255 | SEQ ID NO: 703-X-SEQ ID NO: | SEQ ID NO: 739-YSEQ ID NO: |
| | 704SEQ ID NO: | |
| 256 | SEQ ID NO: 705-X-SEQ ID NO: | SEQ ID NO: 740-YSEQ ID NO: |
| | 706SEQ ID NO: | |
| 257 | SEQ ID NO: 703-X-SEQ ID NO: | SEQ ID NO: 741-Y-cttgtaSEQ ID |
| | 704SEQ ID NO: | NO: |
| 258 | SEQ ID NO: 705-X-SEQ ID NO: | SEQ ID NO: 742-Y-cttgtaSEQ ID |
| | 706SEQ ID NO: | NO: |
| 259 | SEQ ID NO: 707-X-SEQ ID NO: | Y-SEQ ID NO: 737SEQ ID NO: |
| | 708SEQ ID NO: | |
| 260 | SEQ ID NO: 709-X-SEQ ID NO: | Y-SEQ ID NO: 738SEQ ID NO: |
| | 710SEQ ID NO: | |
| 261 | SEQ ID NO: 707-X-SEQ ID NO: | SEQ ID NO: 739-YSEQ ID NO: |
| | 708SEQ ID NO: | |
| 262 | SEQ ID NO: 709-X-SEQ ID NO: | SEQ ID NO: 740-YSEQ ID NO: |
| | 710SEQ ID NO: | |
| 263 | SEQ ID NO: 707-X-SEQ ID NO: | SEQ ID NO: 741-Y-cttgtaSEQ ID |
| | 708SEQ ID NO: | NO: |
| 264 | SEQ ID NO: 709-X-SEQ ID NO: | SEQ ID NO: 742-Y-cttgtaSEQ ID |
| | 710SEQ ID NO: | NO: |
| 265 | SEQ ID NO: 711-X-SEQ ID NO: | SEQ ID NO: 743-YSEQ ID NO: |
| | 712SEQ ID NO: | |
| 266 | SEQ ID NO: 713-X-SEQ ID NO: | SEQ ID NO: 744-YSEQ ID NO: |
| | 714SEQ ID NO: | |
| 267 | SEQ ID NO: 711-X-SEQ ID NO: | SEQ ID NO: 745-Y-cttgtaSEQ ID |
| | 712SEQ ID NO: | NO: |
| 268 | SEQ ID NO: 713-X-SEQ ID NO: | SEQ ID NO: 746-Y-cttgtaSEQ ID |
| | 714SEQ ID NO: | NO: |
| 269 | SEQ ID NO: 711-X-SEQ ID NO: | SEQ ID NO: 747-YSEQ ID NO: |
| | 712SEQ ID NO: | |
| 270 | SEQ ID NO: 713-X-SEQ ID NO: | SEQ ID NO: 748-YSEQ ID NO: |
| | 714SEQ ID NO: | |
| 271 | SEQ ID NO: 715-X-SEQ ID NO: | SEQ ID NO: 743-YSEQ ID NO: |
| | 716SEQ ID NO: | |
| 272 | SEQ ID NO: 717-SEQ ID NO: | SEQ ID NO: 744-YSEQ ID NO: |
| | 718SEQ ID NO: | |
| 273 | SEQ ID NO: 715-X-SEQ ID NO: | SEQ ID NO: 745-Y-cttgtaSEQ ID |
| | 716SEQ ID NO: | NO: |
| 274 | SEQ ID NO: 717-SEQ ID NO: | SEQ ID NO: 746-Y-cttgtaSEQ ID |
| | 718SEQ ID NO: | NO: |
| 275 | SEQ ID NO: 715-X-SEQ ID NO: | SEQ ID NO: 747-YSEQ ID NO: |
| | 716SEQ ID NO: | |
| 276 | SEQ ID NO: 717-SEQ ID NO: | SEQ ID NO: 748-YSEQ ID NO: |
| | 718SEQ ID NO: | |
| 277 | SEQ ID NO: 719-X-SEQ ID NO: | SEQ ID NO: 743-YSEQ ID NO: |
| | 720SEQ ID NO: | |
| 278 | SEQ ID NO: 721-X-SEQ ID NO: | SEQ ID NO: 744-YSEQ ID NO: |
| | 722SEQ ID NO: | |
| 279 | SEQ ID NO: 719-X-SEQ ID NO: | SEQ ID NO: 745-Y-cttgtaSEQ ID |
| | 720SEQ ID NO: | NO: |
| 280 | SEQ ID NO: 721-X-SEQ ID NO: | SEQ ID NO: 746-Y-cttgtaSEQ ID |
| | 722SEQ ID NO: | NO: |
| 281 | SEQ ID NO: 719-X-SEQ ID NO: | SEQ ID NO: 747-YSEQ ID NO: |
| | 720SEQ ID NO: | |
| 282 | SEQ ID NO: 721-X-SEQ ID NO: | SEQ ID NO: 748-YSEQ ID NO: |
| | 722SEQ ID NO: | |
| 283 | SEQ ID NO: 723-X-SEQ ID NO: | SEQ ID NO: 743-YSEQ ID NO: |
| | 724SEQ ID NO: | |
| 284 | SEQ IDNO: 725-X-SEQ ID NO: | SEQ ID NO: 744-YSEQ ID NO: |
| | 726SEQ ID NO: | |
| 285 | SEQ ID NO: 723-X-SEQ ID NO: | SEQ ID NO: 745-Y-cttgtaSEQ ID |
| | 724SEQ ID NO: | NO: |
| 286 | SEQ ID NO: 725-X-SEQ ID NO: | SEQ ID NO: 746-Y-cttgtaSEQ ID |
| | 726SEQ ID NO: | NO: |
| 287 | SEQ ID NO: 723-X-SEQ ID NO: | SEQ ID NO: 747-YSEQ ID NO: |
| | 724SEQ ID NO: | |
| 288 | SEQ ID NO: 725-X-SEQ ID NO: | SEQ ID NO: 748-YSEQ ID NO: |
| | 726SEQ ID NO: | |
| 289 | SEQ ID NO: 771-YSEQ ID NO: | SEQ ID NO: 743-YSEQ ID NO: |
| 290 | SEQ ID NO: 772-YSEQ ID NO: | SEQ ID NO: 744-YSEQ ID NO: |
| 291 | SEQ ID NO: 771-YSEQ ID NO: | SEQ ID NO: 745-Y-cttgtaSEQ ID |
| | | NO: |
| 292 | SEQ ID NO: 772-YSEQ ID NO: | SEQ ID NO: 746-Y-cttgtaSEQ ID |
| | | NO: |
| 293 | SEQ ID NO: 771-YSEQ ID NO: | SEQ ID NO: 747-YSEQ ID NO: |
| 294 | SEQ ID NO: 772-YSEQ ID NO: | SEQ ID NO: 748-YSEQ ID NO: |
| 295 | SEQ ID NO: 773-Y- ccaagtSEQ ID | SEQ ID NO: 743-YSEQ ID NO: |
| | NO: | |
| 296 | SEQ ID NO: 774-Y- ccaagtSEQ ID | SEQ ID NO: 744-YSEQ ID NO: |
| | NO: | |
| 297 | SEQ ID NO: 773-Y- ccaagtSEQ ID | SEQ ID NO: 745-Y-cttgtaSEQ ID |
| | NO: | NO: |
| 298 | SEQ ID NO: 774-Y- ccaagtSEQ ID | SEQ ID NO: 746-Y-cttgtaSEQ ID |
| | NO: | NO: |
| 299 | SEQ ID NO: 773-Y- ccaagtSEQ ID | SEQ ID NO: 747-YSEQ ID NO: |
| | NO: | |
| 300 | SEQ ID NO: 774-Y- ccaagtSEQ ID | SEQ ID NO: 748-YSEQ ID NO: |
| | NO: | |

In another aspect, the invention relates to an ensemble comprising
a first single-stranded nucleic acid molecule comprising or consisting essentially of an A sequence allowing the insertion of a complementary sequence of a nucleic acid of interest, or comprising a complementary sequence of a nucleic acid of interest, said complementary A sequence binding at 5′ to a first A/T-rich, especially T-rich, sequence of 40 to 60 nucleotides in length and at 3′ to a second A/T-rich, especially T-rich, sequence of 40 to 60 nucleotides in length, said first and second A/T-rich, especially T-rich, sequences respectively comprising a first and a second domain of 6 to 12 G/C-rich nucleotides, the sequence of the first domain being complementary to the sequence of the second domain, said first and second domains being positioned 15 to 52 nucleotides from said A sequence, said first molecule comprising at its 5′ end a first sequence oriented 5′-to-3′ for recognizing a transposase and at its 3′ end at least one second sequence for recognizing said transposase,
a second single-stranded nucleic acid molecule comprising or consisting essentially of a B sequence allowing the insertion of a complementary sequence of a nucleic acid of interest, or comprising a complementary sequence of a nucleic acid of interest, said complementary B sequence binding at 5′ to a third T-rich sequence of 40 to 60 nucleotides in length and at 3′ to a fourth T-rich sequence of 40 to 60 nucleotides in length, said third and fourth T-rich sequence respectively comprising a third and a fourth domain of 6 to 12 G/C-rich nucleotides, the sequence of the third domain being complementary to the sequence of the fourth domain, said third and fourth domain being positioned 15 to 52 nucleotides from said B sequence, said second molecule comprising at its 5′ end at least one first sequence oriented 5′-to-3′ for recognizing said transposase and at its 3′ end the second sequence for recognizing said transposase,
said B sequence being a complementary sequence of said nucleic acid of interest, the A sequence being positioned at 5′ of a region of interest of said nucleic acid of interest and the B sequence positioned at 3′ of the region of interest of said nucleic acid of interest, and
a third single-stranded molecule comprising
in its 5′ part, at least one complementary sequence of said second sequence for recognizing said transposase (of the first molecule),
in its 3′ part, at least one complementary sequence of said first sequence for recognizing said transposase (of the second molecule), and
a region located between complementary sequence of said second sequence for recognizing said transposase (of the first molecule) and the complementary sequence of said first sequence for recognizing said transposase (of the second molecule) allowing the insertion of a replacement nucleic acid molecule (single-stranded),
the first and third single-stranded nucleic acid molecules being paired according to the base complementarity defined by Watson and Crick so as to define two double-stranded binding sites of said transposase, and the second and third single-stranded nucleic acid molecules being paired according to the base complementarity defined by Watson and Crick so as to define two double-stranded binding sites of said transposase.

The aforementioned ensemble therefore comprises the molecular complex described hereinbefore. Also, all the embodiments and technical details described hereinbefore for the molecular complex apply, mutatis mutandis, to the aforementioned ensemble.

In this aspect of the invention, an ensemble of three molecules is described making it possible to precisely replace a sequence contained in a nucleic acid of interest with a selected sequence.

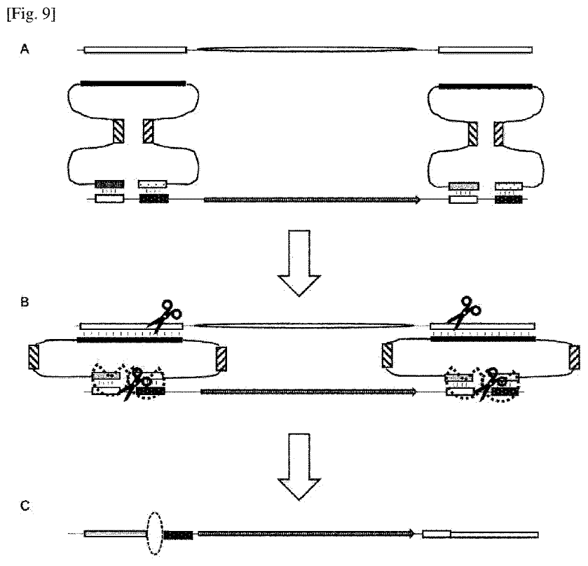

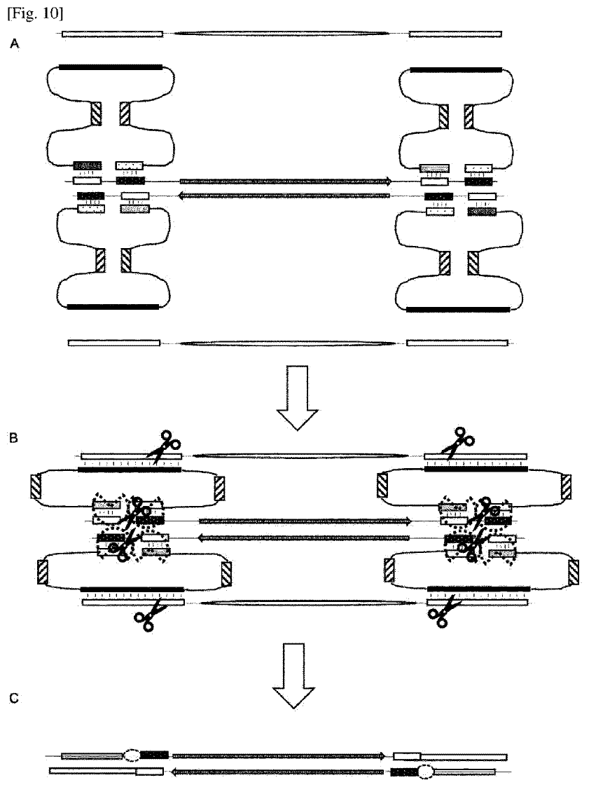

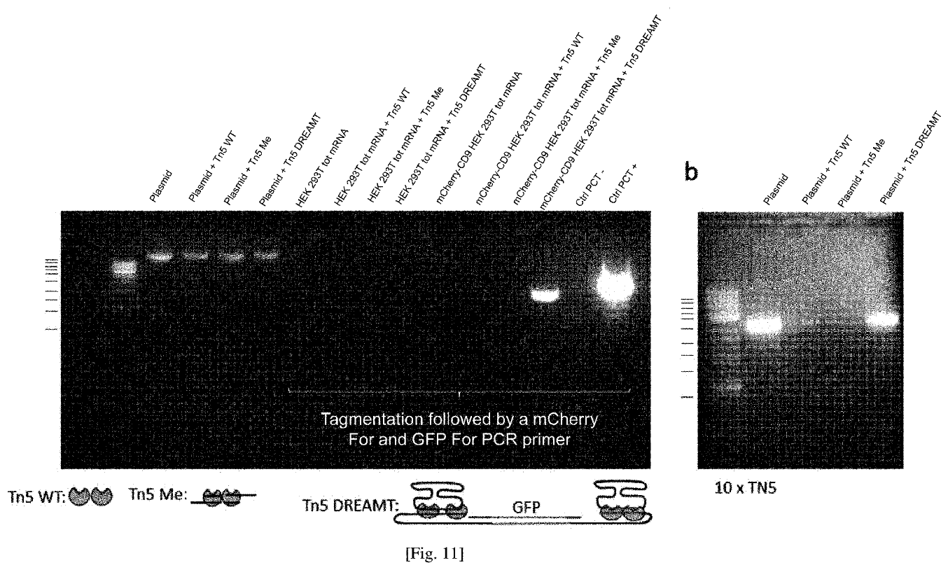

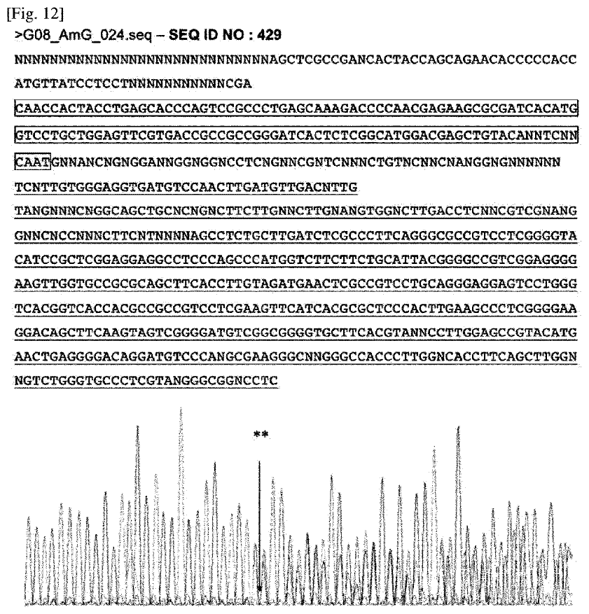

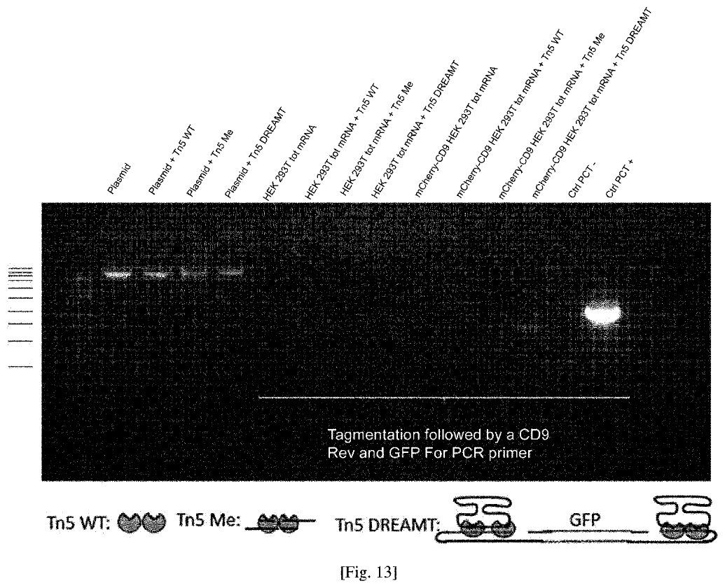

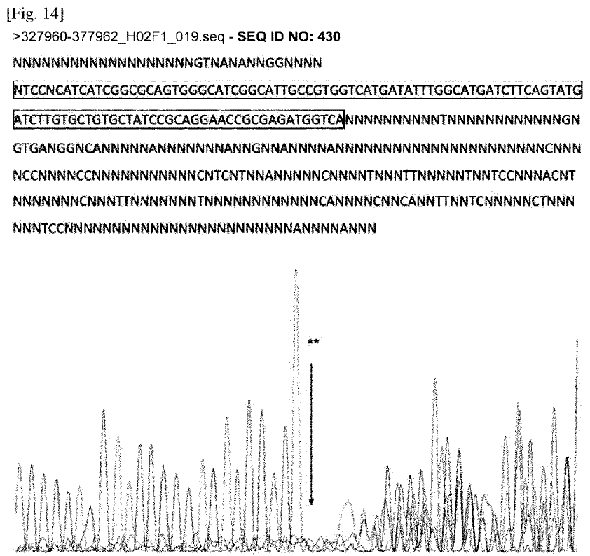

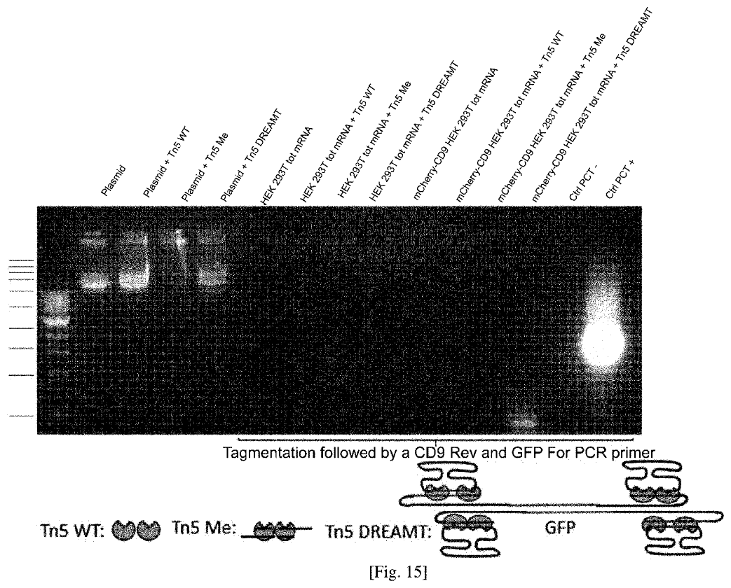

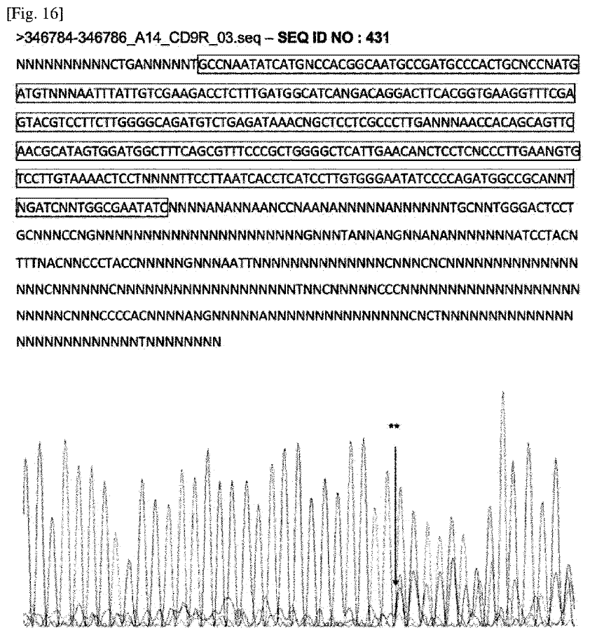

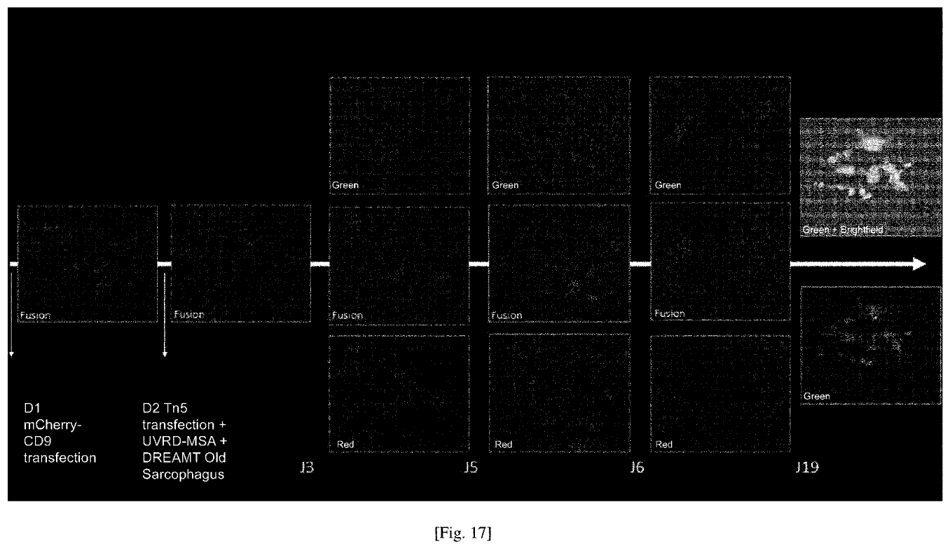

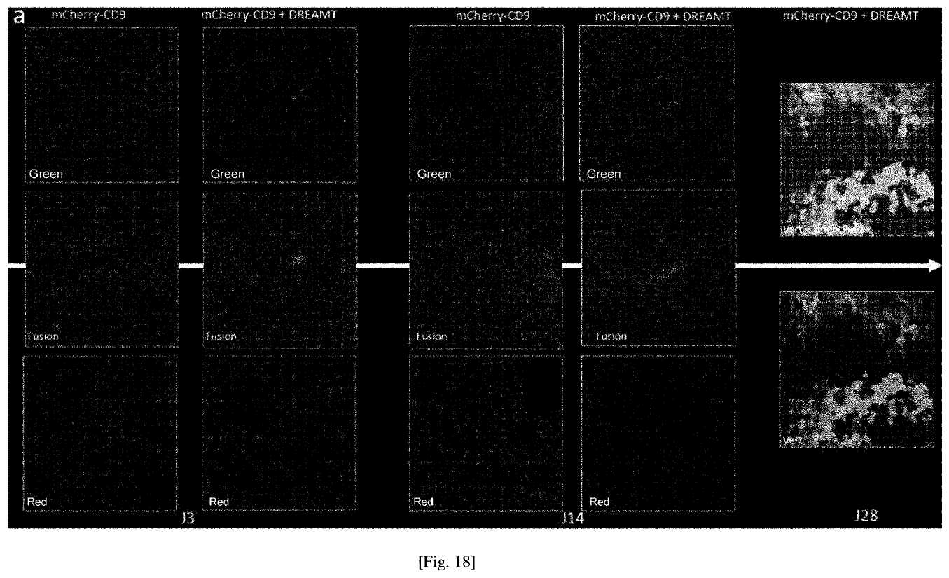

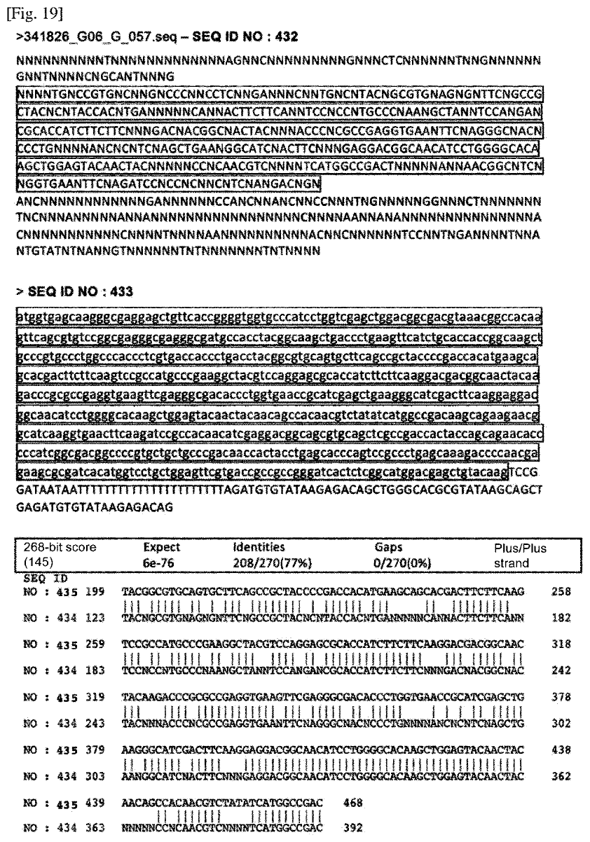

The ensemble according to the invention is based on the above-described complex, this complex being supplemented with a third molecule similar to the first molecule. [FIG. 8] schematically illustrates the ensemble according to the invention

The first molecule of the complex corresponds to the first molecule of the ensemble, the second molecule of the complex corresponds to the third molecule of the ensemble and the third molecule of the ensemble is added and is structurally similar to the first molecule of the ensemble or of the complex.

The ensemble according to the invention, when the three molecules are correctly paired, comprises two pairs of transposase recognition double-stranded binding sites:
the first pair being obtained by the hybridization of the first molecule with the third molecule, and
the second pair being obtained by the hybridization of the second molecule with the third molecule.

The A sequence contained in the first molecule of the ensemble is complementary to the same strand of the nucleic acid which is complementary to the B sequence contained in the second molecule. In other words, the A sequence contained in the first molecule and the B sequence contained in the second molecule are capable of hybridizing with the same nucleic acid simultaneously, since the two sequences A and B do not recognize the same sequence.

In order to further clarify the remarks, the interest of the ensemble defined in the present invention is to propose a first molecule and a second molecule, both as defined hereinbefore, the respective sequences A and B being such that they are capable of recognizing, for one, a sequence located at 5′ of the target sequence of the nucleic acid of interest and, for the other, a sequence located at 3′ of the same target sequence of the nucleic acid of interest. The sequences A and B are therefore complementary to regions bordering the sequence of interest, which it is sought to replace, of the nucleic acid molecule of interest.

The first and second molecules of interest are therefore essential for targeting specifically the molecule of interest, in order to flank the sequence to be replaced.

The third molecule of the ensemble, in turn, is the one that provides the nucleic acid molecule that contains the replacement sequence.

From a mechanistic point of view, the ensemble according to the invention is such that it consists of its three molecules, the first and second molecules being structurally organized in space so that their G/C-rich regions are paired.

On either side of the third molecule, that is to say at 5′ and at 3′, due to the hybridization with the first and second molecules, two pairs of sites for binding to a transposase allow, when transposase is present, transposase dimers to bind to the ensemble.

It will be noted that the transposase binding sites of the first molecule can be the same as those of the second molecule, or different. In the case where the binding sequences are the same, the transposases at 5′ of the third molecule (by hybridization of the 5′ part of the third molecule with the first molecule), and at 3′ of the third molecule (by hybridization of the 3′ part of the third molecule with the second molecule) are the same. Also, by way of example, if the binding sites are all binding sites for the Tn5 transposase, the ensemble is associated with two Tn5 transposase dimers.

It is also possible for the binding sites of the first molecule and of the second molecule not to recognize the same transposase. In this case, and according to the definition of the ensemble given hereinbefore, the 5′ part of the third molecule forms, by hybridization with the first molecule, two double-stranded sites for binding to a first transposase, and the 3′ part of the third molecule forms, by hybridization with the second molecule, two double-stranded sites for binding to a second transposase.

If the aforementioned ensemble, linked to two transposase dimers, is next brought into the presence of a nucleic acid molecule of interest, the 5′ part of which is complementary to the A sequence of the first molecule of the ensemble and the 3′ part of which is complementary to the B sequence of the second molecule of the ensemble, then the nucleic acid molecule of interest pairs with the ensemble at the above-described regions A and B. This interaction has the consequence of breaking the interaction of the two G/C-rich sequences of each of the first and second molecules of the ensemble. Therefore, the third molecule and the nucleic acid molecule of interest are moved closer together in space and the transposases can exercise their tagmentation activity, the result of which is the replacement of the sequence of the molecule of interest, bordered by the complementary sequences of the sequences A and B, by the sequence of the third molecule of the ensemble, which is located between the half-sites for binding to the transposase(s) at 5′ and at 3′.

In the invention, the first molecules, second molecules and third molecule of the ensemble are advantageously molecules consisting of deoxyribonucleotides, in order to form single-stranded DNA molecules.

Even more advantageously, the first molecules, second molecules and third molecule of the ensemble are hybrid DNA/RNA molecules, wherein the “backbone” of the molecules is DNA, and the recognition sequences A and B of the nucleic acid molecule of interest and the central region of the third molecule are RNA. This is particularly advantageous when the sequence replacement that enables the invention is to be carried out directly on an RNA molecule.

[FIG. 9]-A illustrates the interaction between the molecule of interest and the ensemble according to the invention.

Advantageously, the invention relates to the aforementioned complex, wherein said first molecule or said second molecule, or both molecules, are coupled with an enzyme, especially via a modified nucleotide. This enzyme is intended to promote the replacement of the molecule of interest. It can be
a helicase, an enzyme capable of opening a double-stranded molecule that is supercoiled or even associated with proteins such as histones,
a topoisomerase, an enzyme acting on the topological structure of the DNA by generating transient breaks,
a ligase that makes it possible to form a phosphodiester linkage between a phosphate 5′ end of a nucleotide and the OH 3′ end of another nucleotide,—a polymerase, which synthesizes a nucleic acid molecule from an initiation site, free OH 3′, in the 5′-to-3′ direction, especially by copying an antiparallel complementary strand according to the Watson and Crick model.

It is also possible to combine two or more of said enzymes in order to have all the enzymatic material necessary to enable the sequence replacement contemplated in the scope of the invention.

In one particular aspect, the first molecule of the ensemble can contain in its 5′ part, more precisely between the first transposase binding site and the A region, one or more modified nucleotides. In the same way, the third molecule of the ensemble can contain in its 3′ part, more precisely between the first transposase binding site and the 3 region, one or more modified nucleotides.

This modified nucleotide is especially modified by grafting a substituted carbon chain with a protein tag, or else a molecule enabling a specific interaction such as streptavidin or biotin.

Such modifications then make it possible to specifically bind, to the first molecule of the ensemble, enzymes which can be useful for promoting tagmentation, and sequence replacement. It is particularly advantageous to have, for example, streptavidin grafting, which makes it possible to graft a biotinylated helicase (or conversely a helicase grafted to the streptavidin and a biotinylated nucleotide) useful for opening a double-stranded molecule. It is also possible to contemplate grafting with a biotinylated ligase (or a ligase coupled with streptavidin), in order to connect the recombinant strand on the 3′ side.

It is also possible to associate with the first molecule or with the second molecule of the ensemble an oligonucleotide, so that said oligonucleotide pairs on a predetermined region of said first or second molecule. This oligonucleotide is then advantageously coupled with a grafting molecule as explained hereinbefore.

The above-described ensemble is suitable for a single-stranded sequence replacement, such as for example the replacement of a sequence on a single-stranded DNA molecule or an RNA.

To replace the two strands of a double-stranded molecule, it is then useful to use two in the aforementioned ensemble, these two ensembles being such that
the first ensemble comprises the first, second, and third molecules as defined hereinbefore,
the second ensemble comprises a fourth, a fifth, and a sixth sequence, so that:
the third and the sixth sequences each comprise a strand of the replacement sequence, these sequences being complementary according to Watson and Crick pairing;
the first and the fourth sequences comprise, in the A region and the A′ region (the A′ region being the equivalent of the fourth molecule of the A region of the first molecule) a sequence recognizing the 5′ part of the region to be replaced and the 3′ part of the complementary strand of the region to be replaced, respectively, and
the second and the fifth sequences comprise, in the B region and the B′ region (the B′ region being the equivalent of the fifth molecule of the B region of the second molecule) a sequence recognizing the 3′ part of the region to be replaced and the 5′ part of the complementary strand of the region to be replaced, respectively,

This double complex or this double ensemble is then depicted in [FIG. 10]-A.

Advantageously, the invention relates to the aforementioned ensemble, said ensemble comprising one of the 300 pairs of first and third molecules of the following table 5:

TABLE 5

| | the first molecule comprising the | the third molecule comprising the |
|---|---|---|
| # | sequence | sequence |
| 1 | SEQ ID NO: 7-X-SEQ ID NO: 442SEQ | SEQ ID NO: 489-YSEQ ID NO: |
| | ID NO: | |
| 2 | SEQ ID NO: 490-X-SEQ ID | SEQ ID NO: 492-YSEQ ID NO: |
| | NO:491SEQ ID NO: | |
| 3 | SEQ ID NO: 493-X-SEQ ID NO: | SEQ ID NO: 489-YSEQ ID NO: |
| | 494SEQ ID NO: | |
| 4 | SEQ ID NO: 495-X-SEQ ID NO: | SEQ ID NO: 492-YSEQ ID NO: |
| | 496SEQ ID NO: | |
| 5 | SEQ ID NO: 493-X-SEQ ID NO: | SEQ ID NO: 499-Y- ccaagtSEQ ID |
| | 494SEQ ID NO: | NO: |
| 6 | SEQ ID NO: 495-X-SEQ ID NO: | SEQ ID NO: 500-Y- ccaagtSEQ ID |
| | 496SEQ ID NO: | NO: |
| 7 | SEQ ID NO: 497-X-SEQ ID NO: | SEQ ID NO: 489-YSEQ ID NO: |
| | 498SEQ ID NO: | |
| 8 | SEQ ID NO: 501-X-SEQ ID NO: | SEQ ID NO: 492-YSEQ ID NO: |
| | 502SEQ ID NO: | |
| 9 | SEQ ID NO: 497-X-SEQ ID NO: | SEQ ID NO: 499-Y- ccaagtSEQ ID |
| | 498SEQ ID NO: | NO: |
| 10 | SEQ ID NO: 501-X-SEQ ID NO: | SEQ ID NO: 500-Y- ccaagtSEQ ID |
| | 502SEQ ID NO: | NO: |
| 11 | SEQ ID NO: 503-X-SEQ ID NO: | Y-SEQ ID NO: 507SEQ ID NO: |
| | 504SEQ ID NO: | |
| 12 | SEQ ID NO: 505-X-SEQ ID NO: | Y-SEQ ID NO: 508SEQ ID NO: |
| | 506SEQ ID NO: | |
| 13 | SEQ ID NO: 503-X-SEQ ID NO: | Tacaag-Y-SEQ ID NO: 509SEQ ID |
| | 504SEQ ID NO: | NO: |
| 14 | SEQ ID NO: 505-X-SEQ ID NO: | Tacaag-Y-SEQ ID NO: 510, SEQ ID |
| | 506SEQ ID NO: | NO: |
| 15 | SEQ ID NO: 503-X-SEQ ID NO: | Y-SEQ ID NO: 511, SEQ ID NO: |
| | 504SEQ ID NO: | |
| 16 | SEQ ID NO: 505-X-SEQ ID NO: | Y-SEQ ID NO: 512, SEQ ID NO: |
| | 506SEQ ID NO: | |
| 17 | SEQ ID NO: 525-X-SEQ ID NO: | Y-SEQ ID NO: 507SEQ ID NO: |
| | 526SEQ ID NO: | |
| 18 | SEQ ID NO: 527-X-SEQ ID NO: | Y-SEQ ID NO: 508SEQ ID NO: |
| | 528SEQ ID NO: | |
| 19 | SEQ ID NO: 525-X-SEQ ID NO: | Tacaag-Y-SEQ ID NO: 509SEQ ID |
| | 526SEQ ID NO: | NO: |
| 20 | SEQ ID NO: 527-X-SEQ ID NO: | Tacaag-Y-SEQ ID NO: 510, SEQ ID |
| | 528SEQ ID NO: | NO: |
| 21 | SEQ ID NO: 525-X-SEQ ID NO: | Y-SEQ ID NO: 511, SEQ ID NO: |
| | 526SEQ ID NO: | |
| 22 | SEQ ID NO: 527-X-SEQ ID NO: | Y-SEQ ID NO: 512, SEQ ID NO: |
| | 528SEQ ID NO: | |
| 23 | SEQ ID NO: 529-X-SEQ ID NO: | Y-SEQ ID NO: 507SEQ ID NO: |
| | 530SEQ ID NO: | |
| 24 | SEQ ID NO: 531-X-SEQ ID NO: | Y-SEQ ID NO: 508SEQ ID NO: |
| | 532SEQ ID NO: | |
| 25 | SEQ ID NO: 529-X-SEQ ID NO: | Tacaag-Y-SEQ ID NO: 509SEQ ID |
| | 530SEQ ID NO: | NO: |
| 26 | SEQ ID NO: 531-X-SEQ ID NO: | Tacaag-Y-SEQ ID NO: 510, SEQ ID |
| | 532SEQ ID NO: | NO: |
| 27 | SEQ ID NO: 529-X-SEQ ID NO: | Y-SEQ ID NO: 511, SEQ ID NO: |
| | 530SEQ ID NO: | |
| 28 | SEQ ID NO: 531-X-SEQ ID NO: | Y-SEQ ID NO: 512, SEQ ID NO: |
| | 532SEQ ID NO: | |
| 29 | SEQ ID NO: 533-X-SEQ ID NO: | Y-SEQ ID NO: 513SEQ ID NO: |
| | 534SEQ ID NO: | |
| 30 | SEQ ID NO: 535-X-SEQ ID NO: | Y-SEQ ID NO: 514SEQ ID NO: |
| | 536SEQ ID NO: | |
| 31 | SEQ ID NO: 533-X-SEQ ID NO: | Acttgg-Y-SEQ ID NO: 515SEQ ID NO: |
| | 534SEQ ID NO: | |
| 32 | SEQ ID NO: 535-X-SEQ ID NO: | Acttgg-Y-SEQ ID NO: 516SEQ ID NO: |
| | 536SEQ ID NO: | |
| 33 | SEQ ID NO: 533-X-SEQ ID NO: | Y-SEQ ID NO: 517SEQ ID NO: |
| | 534SEQ ID NO: | |
| 34 | SEQ ID NO: 535-X-SEQ ID NO: | Y-SEQ ID NO: 518SEQ ID NO: |
| | 536SEQ ID NO: | |
| 35 | SEQ ID NO: 537-X-SEQ ID NO: | Y-SEQ ID NO: 513SEQ ID NO: |
| | 538SEQ ID NO: | |
| 36 | SEQ ID NO: 539-X-SEQ ID NO: | Y-SEQ ID NO: 514SEQ ID NO: |
| | 540SEQ ID NO: | |
| 37 | SEQ ID NO: 537-X-SEQ ID NO: | Acttgg-Y-SEQ ID NO: 515SEQ ID NO: |
| | 538SEQ ID NO: | |
| 38 | SEQ ID NO: 539-X-SEQ ID NO: | Acttgg-Y-SEQ ID NO: 516SEQ ID NO: |
| | 540SEQ ID NO: | |
| 39 | SEQ ID NO: 537-X-SEQ ID NO: | Y-SEQ ID NO: 517SEQ ID NO: |
| | 538SEQ ID NO: | |
| 40 | SEQ ID NO: 539-X-SEQ ID NO: | Y-SEQ ID NO: 518SEQ ID NO: |
| | 540SEQ ID NO: | |
| 41 | SEQ ID NO: 541-X-SEQ ID NO: | Y-SEQ ID NO: 513SEQ ID NO: |
| | 542SEQ ID NO: | |
| 42 | SEQ ID NO: 543-X-SEQ ID NO: | Y-SEQ ID NO: 514SEQ ID NO: |
| | 544SEQ ID NO: | |
| 43 | SEQ ID NO: 541-X-SEQ ID NO: | Acttgg-Y-SEQ ID NO: 515SEQ ID NO: |
| | 542SEQ ID NO: | |
| 44 | SEQ ID NO: 543-X-SEQ ID NO: | Acttgg-Y-SEQ ID NO: 516SEQ ID NO: |
| | 544SEQ ID NO: | |
| 45 | SEQ ID NO: 541-X-SEQ ID NO: | Y-SEQ ID NO: 517SEQ ID NO: |
| | 542SEQ ID NO: | |
| 46 | SEQ ID NO: 543-X-SEQ ID NO: | Y-SEQ ID NO: 518SEQ ID NO: |
| | 544SEQ ID NO: | |
| 47 | SEQ ID NO: 545-X-SEQ ID NO: | Y-SEQ ID NO: 513SEQ ID NO: |
| | 546SEQ ID NO: | |
| 48 | SEQ ID NO: 547-X-SEQ ID NO: | Y-SEQ ID NO: 514SEQ ID NO: |
| | 548SEQ ID NO: | |
| 49 | SEQ ID NO: 545-X-SEQ ID NO: | Acttgg-Y-SEQ ID NO: 515SEQ ID NO: |
| | 546SEQ ID NO: | |
| 50 | SEQ ID NO: 547-X-SEQ ID NO: | Acttgg-Y-SEQ ID NO: 516SEQ ID NO: |
| | 548SEQ ID NO: | |
| 51 | SEQ ID NO: 545-X-SEQ ID NO: | Y-SEQ ID NO: 517SEQ ID NO: |
| | 546SEQ ID NO: | |
| 52 | SEQ ID NO: 547-X-SEQ ID NO: | Y-SEQ ID NO: 518SEQ ID NO: |
| | 548SEQ ID NO: | |
| 53 | SEQ ID NO: 549-X-SEQ ID NO: | Y-SEQ ID NO: 513SEQ ID NO: |
| | 550SEQ ID NO: | |
| 54 | SEQ ID NO: 551-X-SEQ ID NO: | Y-SEQ ID NO: 514SEQ ID NO: |
| | 552SEQ ID NO: | |
| 55 | SEQ ID NO: 549-X-SEQ ID NO: | Acttgg-Y-SEQ ID NO: 515SEQ ID NO: |
| | 550SEQ ID NO: | |
| 56 | SEQ ID NO: 551-X-SEQ ID NO: | Acttgg-Y-SEQ ID NO: 516SEQ ID NO: |
| | 552SEQ ID NO: | |
| 57 | SEQ ID NO: 549-X-SEQ ID NO: | Y-SEQ ID NO: 517SEQ ID NO: |
| | 550SEQ ID NO: | |
| 58 | SEQ ID NO: 551-X-SEQ ID NO: | Y-SEQ ID NO: 518SEQ ID NO: |
| | 552SEQ ID NO: | |
| 59 | SEQ ID NO: 553-X-SEQ ID NO: | Y-SEQ ID NO: 513SEQ ID NO: |
| | 554SEQ ID NO: | |
| 60 | SEQ ID NO: 555-X-SEQ ID NO: | Y-SEQ ID NO: 514SEQ ID NO: |
| | 556SEQ ID NO: | |
| 61 | SEQ ID NO: 553-X-SEQ ID NO: | Acttgg-Y-SEQ ID NO: 515SEQ ID NO: |
| | 554SEQ ID NO: | |
| 62 | SEQ ID NO: 555-X-SEQ ID NO: | Acttgg-Y-SEQ ID NO: 516SEQ ID NO: |
| | 556SEQ ID NO: | |
| 63 | SEQ ID NO: 553-X-SEQ ID NO: | Y-SEQ ID NO: 517SEQ ID NO: |
| | 554SEQ ID NO: | |
| 64 | SEQ ID NO: 555-X-SEQ ID NO: | Y-SEQ ID NO: 518, SEQ ID NO: |
| | 556SEQ ID NO: | |
| 65 | SEQ ID NO: 557-X-SEQ ID NO: | SEQ ID NO: 519-YSEQ ID NO: |
| | 558SEQ ID NO: | |
| 66 | SEQ ID NO: 559-X-SEQ ID NO: | SEQ ID NO: 520-YSEQ ID NO: |
| | 560SEQ ID NO: | |
| 67 | SEQ ID NO: 557-X-SEQ ID NO: | SEQ ID NO: 521-Y- cttgtaSEQ ID NO: |
| | 558SEQ ID NO: | |
| 68 | SEQ ID NO: 559-X-SEQ ID NO: | SEQ ID NO: 522, SEQ ID NO: |
| | 560SEQ ID NO: | |
| 69 | SEQ ID NO: 557-X-SEQ ID NO: | SEQ ID NO: 523, SEQ ID NO: |
| | 558SEQ ID NO: | |
| 70 | SEQ ID NO: 559-X-SEQ ID NO: | SEQ ID NO: 524, SEQ ID NO: |
| | 560SEQ ID NO: | |
| 71 | SEQ ID NO: 561-X-SEQ ID NO: | SEQ ID NO:519-YSEQ ID NO: |
| | 562SEQ ID NO: | |
| 72 | SEQ ID NO: 563-X-SEQ ID NO: | SEQ ID NO:520-YSEQ ID NO: |
| | 564SEQ ID NO: | |
| 73 | SEQ ID NO: 561-X-SEQ ID NO: | SEQ ID NO:521-Y- cttgtaSEQ ID NO: |
| | 562SEQ ID NO: | |
| 74 | SEQ ID NO: 563-X-SEQ ID NO: | SEQ ID NO:522, SEQ ID NO: |
| | 564SEQ ID NO: | |
| 75 | SEQ ID NO: 561-X-SEQ ID NO: | SEQ ID NO:523, SEQ ID NO: |
| | 562SEQ ID NO: | |
| 76 | SEQ ID NO: 563-X-SEQ ID NO: | SEQ ID NO:524, SEQ ID NO: |
| | 564SEQ ID NO: | |
| 77 | SEQ ID NO: 565-X-SEQ ID NO: | SEQ ID NO:519-YSEQ ID NO: |
| | 566SEQ ID NO: | |
| 78 | SEQ ID NO: 567-X-SEQ ID NO: | SEQ ID NO:520-YSEQ ID NO: |
| | 568SEQ ID NO: | |
| 79 | SEQ ID NO: 565-X-SEQ ID NO: | SEQ ID NO:521-Y- cttgtaSEQ ID NO: |
| | 566SEQ ID NO: | |
| 80 | SEQ ID NO: 567-X-SEQ ID NO: | SEQ ID NO:522, SEQ ID NO: |
| | 568SEQ ID NO: | |
| 81 | SEQ ID NO: 565-X-SEQ ID NO: | SEQ ID NO:523, SEQ ID NO: |
| | 566SEQ ID NO: | |
| 82 | SEQ ID NO: 567-X-SEQ ID NO: | SEQ ID NO:524, SEQ ID NO: |
| | 568SEQ ID NO: | |
| 83 | SEQ ID NO: 569-X-SEQ ID NO: | SEQ ID NO:519-YSEQ ID NO: |
| | 570SEQ ID NO: | |
| 84 | SEQ ID NO: 571-X-SEQ ID NO: | SEQ ID NO:520-YSEQ ID NO: |
| | 572SEQ ID NO: | |
| 85 | SEQ ID NO: 569-X-SEQ ID NO: | SEQ ID NO:521-Y- cttgtaSEQ ID NO: |
| | 570SEQ ID NO: | |
| 86 | SEQ ID NO: 571-X-SEQ ID NO: | SEQ ID NO:522-Y- cttgtaSEQ ID NO: |
| | 572SEQ ID NO: | |
| 87 | SEQ ID NO: 569-X-SEQ ID NO: | SEQ ID NO:523-YSEQ ID NO: |
| | 570SEQ ID NO: | |
| 88 | SEQ ID NO: 571-X-SEQ ID NO: | SEQ ID NO:524-YSEQ ID NO: |
| | 572SEQ ID NO: | |
| 89 | SEQ ID NO: 573-X-SEQ ID NO: | SEQ ID NO:519-YSEQ ID NO: |
| | 574SEQ ID NO: | |
| 90 | SEQ ID NO: 575-X-SEQ ID NO: | SEQ ID NO:520-YSEQ ID NO: |
| | 576SEQ ID NO: | |
| 91 | SEQ ID NO: 573-X-SEQ ID NO: | SEQ ID NO:521-Y- cttgtaSEQ ID NO: |
| | 574SEQ ID NO: | |
| 92 | SEQ ID NO: 575-X-SEQ ID NO: | SEQ ID NO:522-Y- cttgtaSEQ ID NO: |
| | 576SEQ ID NO: | |
| 93 | SEQ ID NO: 573-X-SEQ ID NO: | SEQ ID NO:523-YSEQ ID NO: |
| | 574SEQ ID NO: | |
| 94 | SEQ ID NO: 575-X-SEQ ID NO: | SEQ ID NO:524-YSEQ ID NO: |
| | 576SEQ ID NO: | |
| 95 | SEQ ID NO: 577-X-SEQ ID NO: | SEQ ID NO:519-YSEQ ID NO: |
| | 578SEQ ID NO: | |
| 96 | SEQ ID NO: 579-X-SEQ ID NO: | SEQ ID NO:520-YSEQ ID NO: |
| | 580SEQ ID NO: | |
| 97 | SEQ ID NO: 577-X-SEQ ID NO: | SEQ ID NO:521-Y- cttgtaSEQ ID NO: |
| | 578SEQ ID NO: | |
| 98 | SEQ ID NO: 579-X-SEQ ID NO: | SEQ ID NO:522-Y- cttgtaSEQ ID NO: |
| | 580SEQ ID NO: | |
| 99 | SEQ ID NO: 577-X-SEQ ID NO: | SEQ ID NO:523-YSEQ ID NO: |
| | 578SEQ ID NO: | |
| 100 | SEQ ID NO: 579-X-SEQ ID NO: | SEQ ID NO:524-YSEQ ID NO: |
| | 580SEQ ID NO: | |
| 101 | SEQ ID NO: 581-X-SEQ ID NO: | Y-SEQ ID NO: 645SEQ ID NO: |
| | 582SEQ ID NO: | |
| 102 | SEQ ID NO: 583-X-SEQ ID NO: | Y-SEQ ID NO: 646SEQ ID NO: |
| | 584SEQ ID NO: | |
| 103 | SEQ ID NO: 585-X-SEQ ID NO: | Y-SEQ ID NO: 645SEQ ID NO: |
| | 586SEQ ID NO: | |
| 104 | SEQ ID NO: 587-X-SEQ ID NO: | Y-SEQ ID NO: 646SEQ ID NO: |
| | 588SEQ ID NO: | |
| 105 | SEQ ID NO: 585-X-SEQ ID NO: | tacaag-Y-SEQ ID NO: 647SEQ ID |
| | 586SEQ ID NO: | NO: |
| 106 | SEQ ID NO: 587-X-SEQ ID NO: | tacaag-Y-SEQ ID NO: 648SEQ ID NO: |
| | 588SEQ ID NO: | |
| 107 | SEQ ID NO: 589-X-SEQ ID NO: | Y-SEQ ID NO: 645SEQ ID NO: |
| | 590SEQ ID NO: | |
| 108 | SEQ ID NO: 591-X-SEQ ID NO: | Y-SEQ ID NO: 646SEQ ID NO: |
| | 592SEQ ID NO: | |
| 109 | SEQ ID NO: 589-X-SEQ ID NO: | tacaag-Y-SEQ ID NO: 647SEQ ID NO: |
| | 590SEQ ID NO: | |
| 110 | SEQ ID NO: 591-X-SEQ ID NO: | tacaag-Y-SEQ ID NO: 648SEQ ID NO: |
| | 592SEQ ID NO: | |
| 111 | SEQ ID NO: 593-X-SEQ ID NO: | Y-SEQ ID NO: 649SEQ ID NO: |
| | 594SEQ ID NO: | |
| 112 | SEQ ID NO: 595-X-SEQ ID NO: | Y-SEQ ID NO: 650SEQ ID NO: |
| | 596SEQ ID NO: | |
| 113 | SEQ ID NO: 593-X-SEQ ID NO: | Y-SEQ ID NO: 651SEQ ID NO: |
| | 594SEQ ID NO: | |
| 114 | SEQ ID NO: 595-X-SEQ ID NO: | Y- SEQ ID NO: 652SEQ ID NO: |
| | 596SEQ ID NO: | |
| 115 | SEQ ID NO: 593-X-SEQ ID NO: | acttgg-Y-SEQ ID NO: 653SEQ ID NO: |
| | 594SEQ ID NO: | |
| 116 | SEQ ID NO: 595-X-SEQ ID NO: | acttgg-Y-SEQ ID NO: 654SEQ ID NO: |
| | 596SEQ ID NO: | |
| 117 | SEQ ID NO: 597-X-SEQ ID NO: | Y-SEQ ID NO: 649SEQ ID NO: |
| | 598SEQ ID NO: | |
| 118 | SEQ ID NO: 599-X-SEQ ID NO: | Y-SEQ ID NO: 650SEQ ID NO: |
| | 600SEQ ID NO: | |
| 119 | SEQ ID NO: 597-X-SEQ ID NO: | Y-SEQ ID NO: 651SEQ ID NO: |
| | 598SEQ ID NO: | |
| 120 | SEQ ID NO: 599-X-SEQ ID NO: | Y- SEQ ID NO: 652SEQ ID NO: |
| | 600SEQ ID NO: | |
| 121 | SEQ ID NO: 597-X-SEQ ID NO: | acttgg-Y-SEQ ID NO: 653SEQ ID NO: |
| | 598SEQ ID NO: | |
| 122 | SEQ ID NO: 599-X-SEQ ID NO: | acttgg-Y-SEQ ID NO: 654SEQ ID NO: |
| | 600SEQ ID NO: | |
| 123 | SEQ ID NO: 601-X-SEQ ID NO: | Y-SEQ ID NO: 649SEQ ID NO: |
| | 602SEQ ID NO: | |
| 124 | SEQ ID NO: 603-X-SEQ ID NO: | Y-SEQ ID NO: 650SEQ ID NO: |
| | 604SEQ ID NO: | |
| 125 | SEQ ID NO: 601-X-SEQ ID NO: | Y-SEQ ID NO: 651SEQ ID NO: |
| | 602SEQ ID NO: | |
| 126 | SEQ ID NO: 603-X-SEQ ID NO: | Y- SEQ ID NO: 652SEQ ID NO: |
| | 604SEQ ID NO: | |
| 127 | SEQ ID NO: 601-X-SEQ ID NO: | acttgg-Y-SEQ ID NO: 653SEQ ID NO: |
| | 602SEQ ID NO: | |
| 128 | SEQ ID NO: 603-X-SEQ ID NO: | acttgg-Y-SEQ ID NO: 654SEQ ID NO: |
| | 604SEQ ID NO: | |
| 129 | SEQ ID NO: 605-X-SEQ ID NO: | Y-SEQ ID NO: 655SEQ ID NO: |
| | 606SEQ ID NO: | |
| 130 | SEQ ID NO: 607-X-SEQ ID NO: | Y-SEQ ID NO: 656SEQ ID NO: |
| | 608SEQ ID NO: | |
| 131 | SEQ ID NO: 605-X-SEQ ID NO: | SEQ ID NO: 657-YSEQ ID NO: |
| | 606SEQ ID NO: | |
| 132 | SEQ ID NO: 607-X-SEQ ID NO: | , SEQ ID NO: 658-YSEQ ID NO: |
| | 608SEQ ID NO: | |
| 133 | SEQ ID NO: 605-X-SEQ ID NO: | SEQ ID NO: 659-Y- cttgtaSEQ ID NO: |
| | 606SEQ ID NO: | |
| 134 | SEQ ID NO: 607-X-SEQ ID NO: | SEQ ID NO: 660-Y- cttgtaSEQ ID NO: |
| | 608SEQ ID NO: | |
| 135 | SEQ ID NO: 609-X-SEQ ID NO: | Y-SEQ ID NO: 655SEQ ID NO: |
| | 610SEQ ID NO: | |
| 136 | SEQ ID NO: 611-X-SEQ ID NO: | Y-SEQ ID NO: 656SEQ ID NO: |
| | 612SEQ ID NO: | |
| 137 | SEQ ID NO: 609-X-SEQ ID NO: | SEQ ID NO: 657-Y, SEQ ID NO: |
| | 610SEQ ID NO: | |
| 138 | SEQ ID NO: 611-X-SEQ ID NO: | SEQ ID NO: 658-YSEQ ID NO: |
| | 612SEQ ID NO: | |
| 139 | SEQ ID NO: 609-X-SEQ ID NO: | SEQ ID NO: 659-Y- cttgtaSEQ ID NO: |
| | 610SEQ ID NO: | |
| 140 | SEQ ID NO: 611-X-SEQ ID NO: | SEQ ID NO: 660-Y- cttgtaSEQ ID NO: |
| | 612SEQ ID NO: | |
| 141 | SEQ ID NO: 613-X-SEQ ID NO: | Y-SEQ ID NO: 655SEQ ID NO: |
| | 614SEQ ID NO: | |
| 142 | SEQ ID NO: 615-X-SEQ ID NO: | Y-SEQ ID NO: 656SEQ ID NO: |
| | 616SEQ ID NO: | |
| 143 | SEQ ID NO: 613-X-SEQ ID NO: | SEQ ID NO: 657-Y, SEQ ID NO: |
| | 614SEQ ID NO: | |
| 144 | SEQ ID NO: 615-X-SEQ ID NO: | SEQ ID NO: 658-YSEQ ID NO: |
| | 616SEQ ID NO: | |
| 145 | SEQ ID NO: 613-X-SEQ ID NO: | SEQ ID NO: 659-Y- cttgtaSEQ ID NO: |
| | 614SEQ ID NO: | |
| 146 | SEQ ID NO: 615-X-SEQ ID NO: | SEQ ID NO: 660-Y- cttgtaSEQ ID NO: |
| | 616SEQ ID NO: | |
| 147 | SEQ ID NO: 617-X-SEQ ID NO: | Y-SEQ ID NO: 655SEQ ID NO: |
| | 618SEQ ID NO: | |
| 148 | SEQ ID NO: 619-X-SEQ ID NO: | Y-SEQ ID NO: 656SEQ ID NO: |
| | 620SEQ ID NO: | |
| 149 | SEQ ID NO: 617-X-SEQ ID NO: | SEQ ID NO: 657-YSEQ ID NO: |
| | 618SEQ ID NO: | |
| 150 | SEQ ID NO: 619-X-SEQ ID NO: | SEQ ID NO: 658-YSEQ ID NO: |
| | 620SEQ ID NO: | |
| 151 | SEQ ID NO: 617-X-SEQ ID NO: | SEQ ID NO: 659-Y- cttgtaSEQ ID NO: |
| | 618SEQ ID NO: | |
| 152 | SEQ ID NO: 619-X-SEQ ID NO: | SEQ ID NO: 660-Y- cttgtaSEQ ID NO: |
| | 620SEQ ID NO: | |
| 153 | SEQ ID NO: 621-X-SEQ ID NO: | Y-SEQ ID NO: 655SEQ ID NO: |
| | 622SEQ ID NO: | |
| 154 | SEQ ID NO: 623-X-SEQ ID NO: | Y-SEQ ID NO: 656SEQ ID NO: |
| | 624SEQ ID NO: | |
| 155 | SEQ ID NO: 621-X-SEQ ID NO: | SEQ ID NO: 657-YSEQ ID NO: |
| | 622SEQ ID NO: | |
| 156 | SEQ ID NO: 623-X-SEQ ID NO: | SEQ ID NO: 658-YSEQ ID NO: |
| | 624SEQ ID NO: | |
| 157 | SEQ ID NO: 621-X-SEQ ID NO: | SEQ ID NO: 659-Y- cttgtaSEQ ID NO: |
| | 622SEQ ID NO: | |
| 158 | SEQ ID NO: 623-X-SEQ ID NO: | SEQ ID NO: 660-Y- cttgtaSEQ ID NO: |
| | 624SEQ ID NO: | |
| 159 | SEQ ID NO: 625-X-SEQ ID NO: | Y-SEQ ID NO: 655SEQ ID NO: |
| | 626SEQ ID NO: | |
| 160 | SEQ ID NO: 627-X-SEQ ID NO: | Y-SEQ ID NO: 656SEQ ID NO: |
| | 628SEQ ID NO: | |
| 161 | SEQ ID NO: 625-X-SEQ ID NO: | SEQ ID NO: 657-Y, SEQ ID NO: |
| | 626SEQ ID NO: | |
| 162 | SEQ ID NO: 627-X-SEQ ID NO: | SEQ ID NO: 658-YSEQ ID NO: |
| | 628SEQ ID NO: | |
| 163 | SEQ ID NO: 625-X-SEQ ID NO: | SEQ ID NO: 659-Y- cttgtaSEQ ID NO: |
| | 626SEQ ID NO: | |
| 164 | SEQ ID NO: 627-X-SEQ ID NO: | SEQ ID NO: 660-Y- cttgtaSEQ ID NO: |
| | 628SEQ ID NO: | |
| 165 | SEQ ID NO: 629-X-SEQ ID NO: | SEQ ID NO: 661-YSEQ ID NO: |
| | 630SEQ ID NO: | |
| 166 | SEQ ID NO: 631-X-SEQ ID NO: | SEQ ID NO: 662-Y, SEQ ID NO: |
| | 632SEQ ID NO: | |
| 167 | SEQ ID NO: 629-X-SEQ ID NO: | SEQ ID NO: 749-X-SEQ ID NO: |
| | 630SEQ ID NO: | 750SEQ ID NO: |
| 168 | SEQ ID NO: 631-X-SEQ ID NO: | SEQ ID NO: 751-X-SEQ ID NO: |
| | 632SEQ ID NO: | 752SEQ ID NO: |
| 169 | SEQ ID NO: 629-X-SEQ ID NO: | SEQ ID NO: 753-X-SEQ ID NO: |
| | 630SEQ ID NO: | 754SEQ ID NO: |
| 170 | SEQ ID NO: 631-X-SEQ ID NO: | SEQ ID NO: 755-X-SEQ ID NO: |
| | 632SEQ ID NO: | 756SEQ ID NO: |
| 171 | SEQ ID NO: 633-X-SEQ ID NO: | SEQ ID NO: 661-YSEQ ID NO: |
| | 634SEQ ID NO: | |
| 172 | SEQ ID NO: 635-X-SEQ ID NO: | SEQ ID NO: 662-YSEQ ID NO: |
| | 636SEQ ID NO: | |
| 173 | SEQ ID NO: 633-X-SEQ ID NO: | SEQ ID NO: 749-X-SEQ ID NO: |
| | 634SEQ ID NO: | 750SEQ ID NO: |
| 174 | SEQ ID NO: 635-X-SEQ ID NO: | SEQ ID NO: 751-X-SEQ ID NO: |
| | 636SEQ ID NO: | 752SEQ ID NO: |
| 175 | SEQ ID NO: 633-X-SEQ ID NO: | SEQ ID NO: 753-X-SEQ ID NO: |
| | 634SEQ ID NO: | 754SEQ ID NO: |
| 176 | SEQ ID NO: 635-X-SEQ ID NO: | SEQ ID NO: 755-X-SEQ ID NO: |
| | 636SEQ ID NO: | 756SEQ ID NO: |
| 177 | SEQ ID NO: 637-X-SEQ ID NO: | SEQ ID NO: 661-YSEQ ID NO: |
| | 638SEQ ID NO: | |
| 178 | SEQ ID NO: 639-X-SEQ ID NO: | SEQ ID NO: 662-YSEQ ID NO: |
| | 640SEQ ID NO: | |
| 179 | SEQ ID NO: 637-X-SEQ ID NO: | SEQ ID NO: 749-X-SEQ ID NO: |
| | 638SEQ ID NO: | 750SEQ ID NO: |
| 180 | SEQ ID NO: 639-X-SEQ ID NO: | SEQ ID NO: 751-X-SEQ ID NO: |
| | 640SEQ ID NO: | 752SEQ ID NO: |
| 181 | SEQ ID NO: 637-X-SEQ ID NO: | SEQ ID NO: 753-X-SEQ ID NO: |
| | 638SEQ ID NO: | 754SEQ ID NO: |
| 182 | SEQ ID NO: 639-X-SEQ ID NO: | SEQ ID NO: 755-X-SEQ ID NO: |
| | 640SEQ ID NO: | 756SEQ ID NO: |
| 183 | SEQ ID NO: 641-X-SEQ ID NO: | SEQ ID NO: 661-YSEQ ID NO: |
| | 642SEQ ID NO: | |
| 184 | SEQ ID NO: 643-X-SEQ ID NO: | SEQ ID NO: 662-YSEQ ID NO: |
| | 644SEQ ID NO: | |
| 185 | SEQ ID NO: 641-X-SEQ ID NO: | SEQ ID NO: 749-X-SEQ ID NO: |
| | 642SEQ ID NO: | 750SEQ ID NO: |
| 186 | SEQ ID NO: 643-X-SEQ ID NO: | SEQ ID NO: 751-X-SEQ ID NO: |
| | 644SEQ ID NO: | 752SEQ ID NO: |
| 187 | SEQ ID NO: 641-X-SEQ ID NO: | SEQ ID NO: 753-X-SEQ ID NO: |
| | 642SEQ ID NO: | 754SEQ ID NO: |
| 188 | SEQ ID NO: 643-X-SEQ ID NO: | SEQ ID NO: 755-X-SEQ ID NO: |
| | 644SEQ ID NO: | 756SEQ ID NO: |
| 189 | SEQ ID NO: 757-YSEQ ID NO: | SEQ ID NO: 761-YSEQ ID NO: |
| 190 | SEQ ID NO: 758-YSEQ ID NO: | SEQ ID NO: 762-YSEQ ID NO: |
| 191 | SEQ ID NO: 757-YSEQ ID NO: | SEQ ID NO: 763-X-SEQ ID NO: |
| | | 764SEQ ID NO: |
| 192 | SEQ ID NO: 758-YSEQ ID NO: | SEQ ID NO: 765-X-SEQ ID NO: |
| | | 766SEQ ID NO: |
| 193 | SEQ ID NO: 757-YSEQ ID NO: | SEQ ID NO: 767-X-SEQ ID NO: |
| | | 768SEQ ID NO: |
| 194 | SEQ ID NO: 758-YSEQ ID NO: | SEQ ID NO: 769-X-SEQ ID NO: |
| | | 770SEQ ID NO: |
| 195 | SEQ ID NO: 759-Y- ccaagtSEQ ID | SEQ ID NO: 761-YSEQ ID NO: |
| | NO: | |
| 196 | SEQ ID NO: 760-Y- ccaagtSEQ ID | SEQ ID NO: 762-YSEQ ID NO: |
| | NO: | |
| 197 | SEQ ID NO: 759-Y- ccaagtSEQ ID | SEQ ID NO: 763-X-SEQ ID NO: |
| | NO: | 764SEQ ID NO: |
| 198 | SEQ ID NO: 760-Y- ccaagtSEQ ID | SEQ ID NO: 765-X-SEQ ID NO: |
| | NO: | 766SEQ ID NO: |
| 199 | SEQ ID NO: 759-Y- ccaagtSEQ ID | SEQ ID NO: 767-X-SEQ ID NO: |
| | NO: | 768SEQ ID NO: |
| 200 | SEQ ID NO: 760-Y- ccaagtSEQ ID | SEQ ID NO: 769-X-SEQ ID NO: |
| | NO: | 770SEQ ID NO: |
| 201 | SEQ ID NO: 663-X-SEQ ID NO: | Y-SEQ ID NO: 727SEQ ID NO: |
| | 664SEQ ID NO: | |
| 202 | SEQ ID NO: 665-X-SEQ ID NO: | Y-SEQ ID NO: 728SEQ ID NO: |
| | 666SEQ ID NO: | |
| 203 | SEQ ID NO: 667-X-SEQ ID NO: | Y-SEQ ID NO: 727SEQ ID NO: |
| | 668SEQ ID NO: | |
| 204 | SEQ ID NO: 669-X-SEQ ID NO: | Y-SEQ ID NO: 728SEQ ID NO: |
| | 670SEQ ID NO: | |
| 205 | SEQ ID NO: 667-X-SEQ ID NO: | tacaag-Y-SEQ ID NO: 729SEQ ID NO: |
| | 668SEQ ID NO: | |
| 206 | SEQ ID NO: 669-X-SEQ ID NO: | tacaag-Y-SEQ ID NO: 730SEQ ID NO: |
| | 670SEQ ID NO: | |
| 207 | SEQ ID NO: 671-X-SEQ ID NO: | Y-SEQ ID NO: 727SEQ ID NO: |
| | 672SEQ ID NO: | |
| 208 | SEQ ID NO: 673-X-SEQ ID NO: | Y-SEQ ID NO: 728SEQ ID NO: |
| | 674SEQ ID NO: | |
| 209 | SEQ ID NO: 671-X-SEQ ID NO: | tacaag-Y-SEQ ID NO: 729SEQ ID NO: |
| | 672SEQ ID NO: | |
| 210 | SEQ ID NO: 673-X-SEQ ID NO: | tacaag-Y-SEQ ID NO: 730SEQ ID NO: |
| | 674SEQ ID NO: | |
| 211 | SEQ ID NO: 675-X-SEQ ID NO: | Y-SEQ ID NO: 731SEQ ID NO: |
| | 676SEQ ID NO: | |
| 212 | SEQ ID NO: 677-X-SEQ ID NO: | Y-SEQ ID NO: 732SEQ ID NO: |
| | 678SEQ ID NO: | |
| 213 | SEQ ID NO: 675-X-SEQ ID NO: | Y-SEQ ID NO: 733SEQ ID NO: |
| | 676SEQ ID NO: | |
| 214 | SEQ ID NO: 677-X-SEQ ID NO: | Y-SEQ ID NO: 734SEQ ID NO: |
| | 678SEQ ID NO: | |
| 215 | SEQ ID NO: 675-X-SEQ ID NO: | acttgg-Y-SEQ ID NO: 735SEQ ID NO: |
| | 676SEQ ID NO: | |
| 216 | SEQ ID NO: 677-X-SEQ ID NO: | acttgg-Y-SEQ ID NO: 736SEQ ID NO: |
| | 678SEQ ID NO: | |
| 217 | SEQ ID NO: 679-X-SEQ ID NO: | Y-SEQ ID NO: 731SEQ ID NO: |
| | 680SEQ ID NO: | |
| 218 | SEQ ID NO: 681-X-SEQ ID NO: | Y-SEQ ID NO: 732SEQ ID NO: |
| | 682SEQ ID NO: | |
| 219 | SEQ ID NO: 679-X-SEQ ID NO: | Y-SEQ ID NO: 733SEQ ID NO: |
| | 680SEQ ID NO: | |
| 220 | SEQ ID NO: 681-X-SEQ ID NO: | Y-SEQ ID NO: 734SEQ ID NO: |
| | 682SEQ ID NO: | |
| 221 | SEQ ID NO: 679-X-SEQ ID NO: | acttgg-Y-SEQ ID NO: 735SEQ ID NO: |
| | 680SEQ ID NO: | |
| 222 | SEQ ID NO: 681-X-SEQ ID NO: | acttgg-Y-SEQ ID NO: 736SEQ ID NO: |
| | 682SEQ ID NO: | |
| 223 | SEQ ID NO: 683-X-SEQ ID NO: | Y-SEQ ID NO: 731SEQ ID NO: |
| | 684SEQ ID NO: | |
| 224 | SEQ ID NO: 685-X-SEQ ID NO: | Y-SEQ ID NO: 732SEQ ID NO: |
| | 686SEQ ID NO: | |
| 225 | SEQ ID NO: 683-X-SEQ ID NO: | Y-SEQ ID NO: 733SEQ ID NO: |
| | 684SEQ ID NO: | |
| 226 | SEQ ID NO: 685-X-SEQ ID NO: | Y-SEQ ID NO: 734SEQ ID NO: |
| | 686SEQ ID NO: | |
| 227 | SEQ ID NO: 683-X-SEQ ID NO: | acttgg-Y-SEQ ID NO: 735SEQ ID NO: |
| | 684SEQ ID NO: | |
| 228 | SEQ ID NO: 685-X-SEQ ID NO: | acttgg-Y-SEQ ID NO: 736SEQ ID NO: |
| | 686SEQ ID NO: | |
| 229 | SEQ ID NO: 687-X-SEQ ID NO: | Y-SEQ ID NO: 737SEQ ID NO: |
| | 688SEQ ID NO: | |
| 230 | SEQ ID NO: 689-X-SEQ ID NO: | Y-SEQ ID NO: 738SEQ ID NO: |
| | 690SEQ ID NO: | |
| 231 | SEQ ID NO: 687-X-SEQ ID NO: | SEQ ID NO: 739-YSEQ ID NO: |
| | 688SEQ ID NO: | |
| 232 | SEQ ID NO: 689-X-SEQ ID NO: | SEQ ID NO: 740-YSEQ ID NO: |
| | 690SEQ ID NO: | |
| 233 | SEQ ID NO: 687-X-SEQ ID NO: | SEQ ID NO: 741-Y- cttgtaSEQ ID NO: |
| | 688SEQ ID NO: | |
| 234 | SEQ ID NO: 689-X-SEQ ID NO: | SEQ ID NO: 742-Y- cttgtaSEQ ID NO: |
| | 690SEQ ID NO: | |
| 235 | SEQ ID NO: 691-X-SEQ ID NO: | Y-SEQ ID NO: 737SEQ ID NO: |
| | 692SEQ ID NO: | |
| 236 | SEQ ID NO:693-X-SEQ ID NO: | Y-SEQ ID NO: 738SEQ ID NO: |
| | 694SEQ ID NO: | |
| 237 | SEQ ID NO: 691-X-SEQ ID NO: | SEQ ID NO: 739-YSEQ ID NO: |
| | 692SEQ ID NO: | |
| 238 | SEQ ID NO:693-X-SEQ ID NO: | SEQ ID NO: 740-YSEQ ID NO: |
| | 694SEQ ID NO: | |
| 239 | SEQ ID NO: 691-X-SEQ ID NO: | SEQ ID NO: 741-Y- cttgtaSEQ ID NO: |
| | 692SEQ ID NO: | |
| 240 | SEQ ID NO:693-X-SEQ ID NO: | SEQ ID NO: 742-Y- cttgtaSEQ ID NO: |
| | 694SEQ ID NO: | |
| 241 | SEQ ID NO: 695-X-SEQ ID NO: | Y-SEQ ID NO: 737SEQ ID NO: |
| | 696SEQ ID NO: | |
| 242 | SEQ ID NO: 697-X-SEQ ID NO: | Y-SEQ ID NO: 738SEQ ID NO: |
| | 698SEQ ID NO: | |
| 243 | SEQ ID NO: 695-X-SEQ ID NO: | SEQ ID NO: 739-YSEQ ID NO: |
| | 696SEQ ID NO: | |
| 244 | SEQ ID NO: 697-X-SEQ ID NO: | SEQ ID NO: 740-YSEQ ID NO: |
| | 698SEQ ID NO: | |
| 245 | SEQ ID NO: 695-X-SEQ ID NO: | SEQ ID NO: 741-Y- cttgtaSEQ ID NO: |
| | 696SEQ ID NO: | |
| 246 | SEQ ID NO: 697-X-SEQ ID NO: | SEQ ID NO: 742-Y- cttgtaSEQ ID NO: |
| | 698SEQ ID NO: | |
| 247 | SEQ ID NO: 699-X-SEQ ID NO: | Y-SEQ ID NO: 737SEQ ID NO: |
| | 700SEQ ID NO: | |
| 248 | SEQ ID NO: 701-X-SEQ ID NO: | Y-SEQ ID NO: 738SEQ ID NO: |
| | 702SEQ ID NO: | |
| 249 | SEQ ID NO: 699-X-SEQ ID NO: | SEQ ID NO: 739-YSEQ ID NO: |
| | 700SEQ ID NO: | |
| 250 | SEQ ID NO: 701-X-SEQ ID NO: | SEQ ID NO: 740-YSEQ ID NO: |
| | 702SEQ ID NO: | |
| 251 | SEQ ID NO: 699-X-SEQ ID NO: | SEQ ID NO: 741-Y- cttgtaSEQ ID NO: |
| | 700SEQ ID NO: | |
| 252 | SEQ ID NO: 701-X-SEQ ID NO: | SEQ ID NO: 742-Y- cttgtaSEQ ID NO: |
| | 702SEQ ID NO: | |
| 253 | SEQ ID NO: 703-X-SEQ ID NO: | Y-SEQ ID NO: 737SEQ ID NO: |
| | 704SEQ ID NO: | |
| 254 | SEQ ID NO: 705-X-SEQ ID NO: | Y-SEQ ID NO: 738SEQ ID NO: |
| | 706SEQ ID NO: | |
| 255 | SEQ ID NO: 703-X-SEQ ID NO: | SEQ ID NO: 739-YSEQ ID NO: |
| | 704SEQ ID NO: | |
| 256 | SEQ ID NO: 705-X-SEQ ID NO: | SEQ ID NO: 740-YSEQ ID NO: |
| | 706SEQ ID NO: | |
| 257 | SEQ ID NO: 703-X-SEQ ID NO: | SEQ ID NO: 741-Y- cttgtaSEQ ID NO: |
| | 704SEQ ID NO: | |
| 258 | SEQ ID NO: 705-X-SEQ ID NO: | SEQ ID NO: 742-Y- cttgtaSEQ ID NO: |
| | 706SEQ ID NO: | |
| 259 | SEQ ID NO: 707-X-SEQ ID NO: | Y-SEQ ID NO: 737SEQ ID NO: |
| | 708SEQ ID NO: | |
| 260 | SEQ ID NO: 709-X-SEQ ID NO: | Y-SEQ ID NO: 738SEQ ID NO: |
| | 710SEQ ID NO: | |
| 261 | SEQ ID NO: 707-X-SEQ ID NO: | SEQ ID NO: 739-YSEQ ID NO: |
| | 708SEQ ID NO: | |
| 262 | SEQ ID NO: 709-X-SEQ ID NO: | SEQ ID NO: 740-YSEQ ID NO: |
| | 710SEQ ID NO: | |
| 263 | SEQ ID NO: 707-X-SEQ ID NO: | SEQ ID NO: 741-Y- cttgtaSEQ ID NO: |
| | 708SEQ ID NO: | |
| 264 | SEQ ID NO: 709-X-SEQ ID NO: | SEQ ID NO: 742-Y- cttgtaSEQ ID NO: |
| | 710SEQ ID NO: | |
| 265 | SEQ ID NO: 711-X-SEQ ID NO: | SEQ ID NO: 743-YSEQ ID NO: |
| | 712SEQ ID NO: | |
| 266 | SEQ ID NO: 713-X-SEQ ID NO: | SEQ ID NO: 744-YSEQ ID NO: |
| | 714SEQ ID NO: | |
| 267 | SEQ ID NO: 711-X-SEQ ID NO: | SEQ ID NO: 745-Y- cttgtaSEQ ID NO: |
| | 712SEQ ID NO: | |
| 268 | SEQ ID NO: 713-X-SEQ ID NO: | SEQ ID NO: 746-Y- cttgtaSEQ ID NO: |
| | 714SEQ ID NO: | |
| 269 | SEQ ID NO: 711-X-SEQ ID NO: | SEQ ID NO: 747-YSEQ ID NO: |
| | 712SEQ ID NO: | |
| 270 | SEQ ID NO: 713-X-SEQ ID NO: | SEQ ID NO: 748-YSEQ ID NO: |
| | 714SEQ ID NO: | |
| 271 | SEQ ID NO: 715-X-SEQ ID NO: | SEQ ID NO: 743-YSEQ ID NO: |
| | 716SEQ ID NO: | |
| 272 | SEQ ID NO: 717-SEQ ID NO: 718SEQ | SEQ ID NO: 744-YSEQ ID NO: |
| | ID NO: | |
| 273 | SEQ ID NO: 715-X-SEQ ID NO: | SEQ ID NO: 745-Y- cttgtaSEQ ID NO: |
| | 716SEQ ID NO: | |
| 274 | SEQ ID NO: 717-SEQ ID NO: 718SEQ | SEQ ID NO: 746-Y- cttgtaSEQ ID NO: |
| | ID NO: | |
| 275 | SEQ ID NO: 715-X-SEQ ID NO: | SEQ ID NO: 747-YSEQ ID NO: |
| | 716SEQ ID NO: | |
| 276 | SEQ ID NO: 717-SEQ ID NO: 718SEQ | SEQ ID NO: 748-YSEQ ID NO: |
| | ID NO: | |
| 277 | SEQ ID NO: 719-X-SEQ ID NO: | SEQ ID NO: 743-YSEQ ID NO: |
| | 720SEQ ID NO: | |
| 278 | SEQ ID NO: 721-X-SEQ ID NO: | SEQ ID NO: 744-YSEQ ID NO: |
| | 722SEQ ID NO: | |
| 279 | SEQ ID NO: 719-X-SEQ ID NO: | SEQ ID NO: 745-Y- cttgtaSEQ ID NO: |
| | 720SEQ ID NO: | |
| 280 | SEQ ID NO: 721-X-SEQ ID NO: | SEQ ID NO: 746-Y- cttgtaSEQ ID NO: |
| | 722SEQ ID NO: | |
| 281 | SEQ ID NO: 719-X-SEQ ID NO: | SEQ ID NO: 747-YSEQ ID NO: |
| | 720SEQ ID NO: | |
| 282 | SEQ ID NO: 721-X-SEQ ID NO: | SEQ ID NO: 748-YSEQ ID NO: |
| | 722SEQ ID NO: | |
| 283 | SEQ ID NO: 723-X-SEQ ID NO: | SEQ ID NO: 743-YSEQ ID NO: |
| | 724SEQ ID NO: | |
| 284 | SEQ ID NO: 725-X-SEQ ID NO: | SEQ ID NO: 744-YSEQ ID NO: |
| | 726SEQ ID NO: | |
| 285 | SEQ ID NO: 723-X-SEQ ID NO: | SEQ ID NO: 745-Y- cttgtaSEQ ID NO: |
| | 724SEQ ID NO: | |
| 286 | SEQ ID NO: 725-X-SEQ ID NO: | SEQ ID NO: 746-Y- cttgtaSEQ ID NO: |
| | 726SEQ ID NO: | |
| 287 | SEQ ID NO: 723-X-SEQ ID NO: | SEQ ID NO: 747-YSEQ ID NO: |
| | 724SEQ ID NO: | |
| 288 | SEQ ID NO: 725-X-SEQ ID NO: | SEQ ID NO: 748-YSEQ ID NO: |
| | 726SEQ ID NO: | |
| 289 | SEQ ID NO: 771-YSEQ ID NO: | SEQ ID NO: 743-YSEQ ID NO: |
| 290 | SEQ ID NO: 772-YSEQ ID NO: | SEQ ID NO: 744-YSEQ ID NO: |
| 291 | SEQ ID NO: 771-YSEQ ID NO: | SEQ ID NO: 745-Y- cttgtaSEQ ID NO: |
| 292 | SEQ ID NO: 772-YSEQ ID NO: | SEQ ID NO: 746-Y- cttgtaSEQ ID NO: |
| 293 | SEQ ID NO: 771-YSEQ ID NO: | SEQ ID NO: 747-YSEQ ID NO: |
| 294 | SEQ ID NO: 772-YSEQ ID NO: | SEQ ID NO: 748-YSEQ ID NO: |
| 295 | SEQ ID NO: 773-Y- ccaagtSEQ ID | SEQ ID NO: 743-YSEQ ID NO: |
| | NO: | |
| 296 | SEQ ID NO: 774-Y- ccaagtSEQ ID | SEQ ID NO: 744-YSEQ ID NO: |
| | NO: | |
| 297 | SEQ ID NO: 773-Y- ccaagtSEQ ID | SEQ ID NO: 745-Y- cttgtaSEQ ID NO: |
| | NO: | |
| 298 | SEQ ID NO: 774-Y- ccaagtSEQ ID | SEQ ID NO: 746-Y- cttgtaSEQ ID NO: |
| | NO: | |
| 299 | SEQ ID NO: 773-Y- ccaagtSEQ ID | SEQ ID NO: 747-YSEQ ID NO: |
| | NO: | |
| 300 | SEQ ID NO: 774-Y- ccaagtSEQ ID | SEQ ID NO: 748-YSEQ ID NO: |
| | NO: | |

In yet another aspect, the invention relates to a kit, or a set, comprising at least one vector allowing the expression of a recombinase and the first, second and third molecules of the ensemble as defined hereinbefore.

The kit consists either of one container comprising the three molecules of the ensemble, or several separate containers.

The transposase contained in the aforementioned kit is a transposase capable of recognizing the binding sites formed by the interaction between the first molecule and the third molecule of the complex, and/or the second molecule and the third molecule of the complex.

In the event that the complex is such that it contains two binding sites for two different transposases, the kit then comprises two different vectors each coding one of the transposases.

The kit may also further contain, as discussed hereinbefore, other enzymes, such as for example a helicase, or a ligase.

The kit according to the invention advantageously comprises what is needed to form two ensembles as defined hereinbefore.

The invention also relates to the use of the ensemble as defined hereinbefore, for nucleic acid engineering, especially for the replacement of a target sequence with a sequence of interest. The aforementioned use is especially subject to it not comprising a method for modifying the germline genetic identity of human beings and/or said use not being a method for treating the human or animal body by surgery or therapy.

As explained hereinbefore, the aforementioned ensemble makes it possible to target specifically a target region and to replace it with a sequence of interest, by using the transposase tagmentation properties.

The ensemble according to the invention is especially advantageous for carrying out targeted gene modifications (and in particular replacements of genes, or non-coding sequences) in different living organisms, for example in plants for obtaining genetically modified plants, or in animals for producing models of human diseases, or living therapeutic tools, making it possible to test drugs.

Furthermore, the invention relates to the aforementioned ensemble, for use as a drug, more particularly as a gene therapy drug, especially for treating or preventing a disease associated with a nucleic acid modification

Since the aforementioned ensemble allows the substitution of specific sequences, it is possible to design first and second molecules that specifically recognize a mutated gene, by a modification such as a substitution, a deletion or an insertion, and to design a third molecule comprising the reference wild-type sequence of said mutated gene.

Also, in the presence of the appropriate transposase, it is possible to replace the sequence of the mutated gene with the wild-type sequence, and thus to treat an individual suffering from a disease caused by the mutation of said gene.

The invention also relates to the use of the aforementioned complex, for manufacturing a drug for treating or preventing a disease associated with a nucleic acid modification.

In some aspects, these drugs are not intended to modify the germline identity of human beings, and are also not intended to create unjustified suffering in animals.

The invention further relates to a method, especially in vitro, for replacing a target region of a nucleic acid molecule with a region of interest of another nucleic acid molecule, so as to obtain a hybrid nucleic acid molecule, said method comprising:
bringing an ensemble as defined hereinbefore into contact with the nucleic acid comprising the target region,
said ensemble being such that the A sequence of said first molecule comprises a complementary sequence of the region immediately at 5′ of the target region,
the B sequence of said first molecule comprises a complementary sequence of the region immediately at 3′ of the target region, and
the third molecule comprises said region of interest in the region located between complementary sequence of said second sequence for recognizing said transposase of the first molecule and the complementary sequence of said first sequence for recognizing said transposase of the second molecule, in order to obtain a replacement complex,
placing the replacement complex in the presence of a transposase recognizing the double-stranded binding sites of said transposase contained in said ensemble, to obtain a recombination complex, and
recombining the combination complex in order to obtain the hybrid nucleic acid molecule comprising the region of interest instead of the target region.

It should be noted that during the replacement, that is to say the tagmentation by the transposase, the 3′ end of the replacement fragment binds by phosphodiester bond to the 5′ end of the sequence which immediately follows the sequence that has been replaced. This ligation takes place during the tagmentation.

Conversely, the 3′ end of the sequence located just before the replaced sequence and the 5′ end of the replacement strand do not bind to each other. Also, to finalize the replacement, it is necessary, using a ligase, to connect these two ends.

It should also be noted that at the replacement site, the replacement molecule is bordered at 3′ and 5′ by a transposase recognition sequence.

The replacement mechanism for a single-stranded target is depicted in [FIG. 9].

In a first step, the ensemble consisting of the first, second, and third molecule comprising the complementary region of the 5′ part of the target sequence, the complementary region of the 3′ part of the target sequence, and the sequence of interest, respectively, are organized in space so that the G/C-rich complementary regions of the first molecule are paired with one another and those of the second molecule are paired together ([FIG. 9])-A).

In the presence of the target sequence, the complementary regions of the 5′ part and of the 3′ part of the target sequence, contained respectively in the first and second molecules pair with their respective complementary region so as to form a four-molecule complex containing the target molecule and the three molecules of the ensemble. The interaction (or pairing) between the first and second molecules and their targets has the effect of breaking the interaction of the G/C-rich regions, so that the transposase binding sites, on which the transposase dimers are positioned, are located spatially close to the target molecule. The transposases can then exercise their activity and cleave both the target molecule and the molecule of interest (between the two binding sites) as shown in [FIG. 9]-B.

Therefore, the tagmentation takes place so that
the 3′ end of the third molecule binds to the free 5′ end generated by the transposase in the region at 3′ of the target sequence. This bond is covalent by ligation of the two ends,
the 5′ end of the third molecule is brought opposite the free 3′ end generated by the transposase in the region at 5′ of the target sequence. A deletion of nine base pairs is generated by the transposase in the region at 5′ of the target sequence (depicted by the dotted circle in [FIG. 9]-C). In order to recover an entire molecule with a sequence of interest located between the regions at 5′ and 3′ of the initial target sequence, a ligation is necessary.

The final molecule resulting from the tagmentation consists, in the 5′-to-3′ direction, of a part of the 5′ region of the initial target sequence, partially deleted of nine base pairs, followed by the second transposase binding sequence, followed by the sequence of interest itself followed by the first transposase binding sequence and finally a part of the 5′ region of the initial target sequence.

In yet another aspect, the invention relates to a method, especially in vitro or ex vivo, for editing the genome of a cell, making it possible to replace a specific fragment of the double-stranded DNA of said genome of said cell with another double-stranded DNA fragment of interest, in order to obtain a recombinant hybrid genome comprising the other double-stranded DNA fragment of interest instead of the specific fragment of the double-stranded DNA, said method comprising:
preparing a first ensemble as defined hereinbefore,
wherein the A sequence of the first molecule comprises a complementary sequence of the adjacent region at 5′ of the specific fragment;
wherein the B sequence of the second molecule comprises a complementary sequence of the adjacent region at 3′ of the specific fragment; and
wherein the third molecule comprises, between the region located between complementary sequence of said second sequence for recognizing said transposase of the first molecule and the complementary sequence of said first sequence for recognizing said transposase of the second molecule, a sequence of one of the strands of said specific fragment;
and optionally preparing a second ensemble as defined hereinbefore,
wherein the A sequence of said complementary region of the first molecule of the second ensemble comprises a complementary sequence of the adjacent region at 5′ of the specific fragment;
wherein the B sequence of said complementary region of the second molecule of the second ensemble comprises a complementary sequence of the adjacent region at 3′ of the specific fragment; and
wherein the third molecule of the second ensemble comprises, between the region located between complementary sequence of said second sequence for recognizing said transposase of the first molecule of the second ensemble and the complementary sequence of said first sequence for recognizing said transposase of the second molecule of the second ensemble, the sequence of the complementary strand of said specific fragment contained in the third sequence of the first ensemble;
wherein the complementary sequence of the adjacent region at 5′ of the specific fragment contained in the A region of the first molecule of the first ensemble is at most 95% complementary to the complementary sequence of the adjacent region at 5′ of the specific fragment contained in the A region of the first molecule of the second ensemble; and
wherein the complementary sequence of the adjacent region at 3′ of the
specific fragment contained in the B region of the first molecule of the first
ensemble is at most 95% complementary to the complementary sequence of the adjacent region at 3′ of the specific fragment contained in the B region of the first molecule of the second ensemble;
in order to obtain a recombination complex;
bringing said cell into contact with said recombination complex, in order to obtain a cell ready to be edited,
expressing said transposase in said cell ready to be edited, in order to obtain an edited cell,
selecting the edited cell, wherein the genome of said edited cell comprises, instead of the specific double-stranded DNA fragment, the other double-stranded DNA fragment of interest,
subject to said method not being a method for modifying the germline genetic identity of human beings and to said method not being a method for treating the human or animal body by surgery or therapy.

Advantageously, the invention relates to a method for editing the genome of a cell, making it possible to replace a specific fragment of the double-stranded DNA of said genome of said cell with another double-stranded DNA fragment of interest, in order to obtain a recombinant hybrid genome comprising the other double-stranded DNA fragment of interest instead of the specific fragment of the double-stranded DNA, said method comprising:
preparing a first ensemble as defined hereinbefore,
wherein the A sequence of the first molecule comprises a complementary sequence of the adjacent region at 5′ of the specific fragment;
wherein the B sequence of the second molecule comprises a complementary sequence of the adjacent region at 3′ of the specific fragment; and
wherein the third molecule comprises, between the region located between complementary sequence of said second sequence for recognizing said transposase of the first molecule and the complementary sequence of said first sequence for recognizing said transposase of the second molecule, a sequence of one of the strands of said specific fragment;
and preparing a second ensemble as defined hereinbefore,
wherein the A sequence of said complementary region of the first molecule of the second ensemble comprises a complementary sequence of the adjacent region at 5′ of the specific fragment;
wherein the B sequence of said complementary region of the second molecule of the second ensemble comprises a complementary sequence of the adjacent region at 3′ of the specific fragment; and wherein the third molecule of the second ensemble comprises, between
the region located between complementary sequence of said second sequence for recognizing said transposase of the first molecule of the second ensemble and the complementary sequence of said first sequence for recognizing said transposase of the second molecule of the second ensemble, the sequence of the complementary strand of said specific fragment contained in the third sequence of the first ensemble;
wherein the complementary sequence of the adjacent region at 5′ of the specific fragment contained in the A region of the first molecule of the first ensemble is at most 95% complementary to the complementary sequence of the adjacent region at 5′ of the specific fragment contained in the A region of the first molecule of the second ensemble; and wherein the complementary sequence of the adjacent region at 3′ of the specific fragment contained in the B region of the first molecule of the first ensemble is at most 95% complementary to the complementary sequence of the adjacent region at 3′ of the specific fragment contained in the B region of the first molecule of the second ensemble;
in order to obtain a recombination complex;
bringing said cell into contact with said recombination complex, in order to obtain a cell ready to be edited,
expressing said transposase in said cell ready to be edited, in order to obtain an edited cell,
selecting the edited cell, wherein the genome of said edited cell comprises, instead of the specific double-stranded DNA fragment, the other double-stranded DNA fragment of interest,
subject to said method not being a method for modifying the germline genetic identity of human beings and to said method not being a method for treating the human or animal body by surgery or therapy.

To the extent that this genome editing method is based on the method for replacing a single molecule described hereinbefore, and using an ensemble described hereinbefore, all the previously described features and all the variants apply herein, mutatis mutandis.

In this aspect of the invention, it should be noted that the first molecule of the ensemble considered (first or second) is always positioned at 5′ of the third molecule of said ensemble considered.

Also, the first molecule of the first ensemble is located “above” the second molecule of the second ensemble, and vice versa. Also, the aforementioned 5′-to-3′ orientations make it possible to correctly position the sequence of interest with respect to the target sequence.

Genome editing consists of modifying the genome of a cell with high precision. It is possible to inactivate a gene, to introduce a targeted mutation, to correct a particular mutation or to insert a new gene. This genetic engineering technique involves nucleases, herein transposase, capable of cleaving nucleic acids at the phosphodiester bonds.

In the context of the invention, and as previously explained, the first and second molecules of the ensemble according to the invention make it possible to position the ensemble at the target region so as to flank the target region in order for it to be replaced with the sequence of interest contained in the third sequence.

In the context of editing a double-stranded molecule, it is necessary to have, in order to simultaneously replace the two strands of the target molecule to have two ensembles according to the invention, each ensemble specifically targeting one of the two strands of the target molecule.

It should be noted that the complementary sequence contained in the A sequence of the first molecule of the first ensemble is not complementary to the complementary sequence contained in the A sequence of the first molecule of the second ensemble. Likewise, the B sequence of the second molecule of the first ensemble is not complementary to the complementary sequence contained in the B sequence of the second molecule of the second ensemble.

Furthermore, it is advantageous for the complementary sequence contained in the A sequence of the first molecule of the first ensemble to be offset with respect to the complementary sequence contained in the B sequence of the second molecule of the first ensemble, these two molecules being one “above” the other. This means that the region for recognizing these complementary sequences is at most 95%. In other words, if the complementary sequence contained in said A sequence and said B sequence contains twenty nucleotides, the complementary sequences are only complementary to each other for up to 19 nucleotides.

However, it is preferable for the mutual complementarity of these complementary regions to be low as possible, or even for them not to be mutually complementary.

In order to clearly illustrate these remarks, if the 5′ part of the target sequence is considered to comprise 40 nucleotides, then it would be appropriate for the complementary region contained in the A sequence of the first molecule of the first ensemble to be complementary to the first 20 nucleotides, whereas the complementary region contained in the B sequence of the second molecule of the second ensemble is complementary to the last 20 nucleotides of the complementary sequence of the 5′ part of the target sequence.

This offset, even if the sequence orientations do not make it possible, avoid any incorrect orientation of the sequence of interest.

The sequence of the tagmentation steps and the resultant are described in [FIG. 10].

In order to obtain the final molecule, and due to the deletion of nine base pairs at 5′ following the tagmentation, it is necessary for the cell to mobilize the repair system, in order to fill the hole, especially by using a DNA polymerase which copies the complementary strand, the 3′ end of which was bonded during the tagmentation.

### EXAMPLES

### Example 1—Implementation of the Invention In Vitro

The aim of this example is to show that the ensemble according to the invention makes it possible to easily and specifically replace a sequence of a single-stranded nucleic acid molecule (RNA or cDNA) with a sequence of interest.

In this example, the aim is to replace the sequence of mCherry-CD9 with the sequence of GFP.
Preparing the recombination ensemble targeting mCherry-CD9.
Preparing two separate tubes containing:
for Tube 1 (10 μL):
+10 μM of the loop A oligonucleotide (first molecule) comprising in the A region a sequence for recognizing the sequence of mCherry;
+10 μM of the oligonucleotide A (third molecule) comprising a restriction half-site at 3′
+the reverse BSPEi Frag oligo (10 μM)
for Tube 2 (10 μL),
+10 μM of the loop B oligonucleotide (second molecule) comprising in the B region a sequence for recognizing the sequence of CD9;
+10 μM of the oligonucleotide A (third molecule) comprising a restriction half-site at 3′
+the Ndel Frag oligo (10 μM).

The two tubes are then heated to 95° C. for 5 min and then left for 1 hour at room temperature.

Once at room temperature, the content of the tubes is placed in the presence either of the enzyme Ndel or of the enzyme BSPEi in order to allow the digestion of the restriction sites.

The digestion products are then purified with a PCR purification kit (elution 20 μL).

In parallel, a sequence coding GFP is amplified in order to have, at 5′ and 3′, Nde I and BSPEi restriction sites. The PCR product is then subjected to digestion with the two restriction enzymes and the digestion product is purified with a PCR purification kit (elution 20 μL).

The contents of Tubes 1 and 2 are then combined with the GFP fragment (amplified and digested) in the presence of ligase of the T4 phage (4 μL, i.e. 100U) with 5 μL of T4 buffer (10×) and 1 μL of ddH20, for a total reaction volume of 50 μL.

The solution is then left for 1 hour at room temperature, and then gel purification is carried out to isolate the largest GFP fragment, greater than 1 kilobases (kb), and comprising the two single-stranded molecules (first and second molecules of the ensemble) at its 5′ and 3′ ends, in order to form a recombination complex.

The recombination complex is then ready to be incubated with transposase dimers.

In control, MeA and MeB oligonucleotides at 10 μM each were prepared with the MERev fragment (previously described by S. Picelli et al Genome Res 2014):

Tn5MErev,
(SEQ ID NO: 11)
5′-[phos]CTGTCTCTTATACACATCT-3′
Tn5ME-A (Illumina FC-121-1030),
(SEQ ID NO: 12)
5′-TCGTCGGCAGCGTCAGATGTGTATAAGAGACAG-3′;
and
Tn5ME-B (Illumina FC-121-1031),
(SEQ ID NO: 13)
5′-GTCTCGTGGGCTCGGAGATGTGTATAAGAGACAG-3′

by pairing at 95° C. for 5 min then left for 1 hour at ambient temperature. These oligos are useful as positive control for the tagmentation reaction.

Regarding the production of Tn5 transposase, it is produced using the recommendations of the following publication: S. Picelli et al. Genome res 2014.

The two previous preparations, recombination complex and positive control oligos, were then mixed with the production of Tn5 transposase via the following protocol:

0.125 vol. of the 10 μM solution of recombination complex or control oligos (or just the dialysis buffer (cf. S. Picelli et al. Genome, 2014) for so-called WT wild-type Tn5)+0.4 vol. of 100% glycerol solution+0.24 vol. of dialysis buffer (cf. S. Picelli et al. Genome res 2014)+0.36 vol. of Tn5 solution.

The reaction is then brought from 45° C. to 37° C. over 30 min, decreasing by 1° C. every 4 minutes in a thermocycler.

At the same time, several targets were produced in order to test the technology according to the invention.

An mCherry-CD9 plasmid was used as negative control as well as a production of a library of mRNA and cDNA originating from HEK 293T cells previously transfected by lipofectamine 3000 with the mCherry-CD9 vector.

Forty-eight hours after transfection and visual inspection (red membrane due to the expression of the mCherry) of the HEK cells, the total population of mRNAs is then extracted and reverse transcribed into cDNA.

These mRNA or cDNA libraries are then used for our “in-tube” test of the DREAMT technology.

In order to characterize and test the technology according to the invention, different mRNA/cDNA and plasmid targets were brought into contact with
Tn5 WT dimers: without oligo,
Tn5 Me: with Me oligo (positive control) and
Tn5 complex: with the ensemble according to the invention.

Three types of targets were tested, the mCherry-CD9 plasmid and the mRNA or cDNA library with or without transfection of mCherry-CD9.

The double- or single-stranded DNA/RNA solutions were brought into contact with the different solutions of Tn5 activated via the following reaction:
X or 10× concentration of Tn5 WT, Tn5 Me, Tn5 complex solution or the same volume of ddH20+500 ng of mCherry-CD9 plasmid or 2 μL of cDNA or mRNA
+5× tagmentation buffer (100 mM HEPES, 50 mM MgCl2, 40% PEG 3500)+
ddH20 Q.S. 20 μL.

Each tagmentation reaction is carried out in a thermocycler at 55° C. for 7 min, then 0.5 μL of proteinase K solution (20 μg/μL) is added before a second incubation temp at 55° C. for 7 min in order to inactivate the transposase.

Thus, as presented in [FIG. 11], a sedimentation (ligation) of the 3′ end of the GFP was carried out towards the mCherry target sequence.

On the agarose gel in [FIG. 11], the mCherry-CD9 plasmid (Davidson Lab) was brought into contact with two concentrations (X and 10×) of Tn5 solution. As expected, at high concentration (10X), the Tn5 WT mix and the Tn5 Me positive control carried out their tagmentation, illustrated by a degradation of the plasmid (smear, disappearance of the plasmid band).

In addition, as expected, the ensemble alone was not able to carry out tagmentation (plasmid degradation), illustrated by the preservation of the DNA band on the agarose gel ([FIG. 11]) [because it does not comprise a molecule for opening the double-stranded DNA of the plasmid (helicases) in order to pair with the mCherry-CD9 target which would release the Tn5 molecules and allow the tagmentation]. This data makes it possible to conclude by confining the Tn5 complex dimers which can only carry out their tagmentation activity by pairing the strand to be recombined with the target single strand, herein the mCherry-CD9 mRNA fragment.

The same concentration of Tn5 solution (X) was used with 2 μL of the mRNA library (originating from HEK 293T cells, normal or transfected with mCherry-CD9). Then a PCR with the mCherry Forward and GFP Forward primers was carried out (cf. table 3).

TABLE 3
Oligos used for the PCR reactions and plasmid
constructions [Table 3]

| Names | Sequences 5′ to 3′ |
|---|---|
| GFP For | CTGGTCGAGCTGGACGGCGACG |
| | (SEQ ID NO: 14) |
| GFP Rev | CACGAACTCCAGCAGGACCATG |
| | (SEQ ID NO: 15) |
| mCherry | AAGGGCGAGGAGGATAACATG |
| For | (SEQ ID NO: 16) |
| CD9 Rev | GACCATCTCGCGGTTCCT |
| | (SEQ ID NO: 17) |
| GFP Nde1 | ACTTGGCATATGATGGTGAGCAAGGGCGAGGA |
| For | (SEQ ID NO: 18) |
| GFP Nde1 | ACTTGGCATATGCTTGTACAGCTCGTCCAT |
| Rev | (SEQ ID NO: 19) |
| GFP bspei | TACAAGTCCGGAATGGTGAGCAAGGGCGAGGA |
| For | (SEQ ID NO: 20) |
| GFP bspei | TACAAGTCCGGACTTGTACAGCTCGTCCAT |
| Rev | (SEQ ID NO: 21) |
| CMV Nde1 | ACTTGGCATATGCCAAGTACGCCCCCTATTGA |
| For | (SEQ ID NO: 22) |
| UVRD For | CTCTTCGCTAGCTGCCACCATGACGCGTGGCCCCAA |
| | GAAAAAGCGGAAAGTGGGACCGGCCACCATGGACG |
| | TTTCTTACCTGCTCG (SEQ ID NO: 23) |
| UVRD Rev | AACAACACGTGCCGGTGATACCCGCTTCCGCGCCTG |
| | ATCCACCACCACCTGACCCACCACCAGCCACCGACT |
| | CCAGCCGGG (SEQ ID NO: 24) |
| mSA For | CTCTTCGCTAGCGCGGAAGCGGGTATCAC |
| | (SEQ ID NO: 25) |
| mSA Rev | AACAAGAATTCTTATTATTTAACTTTGGTGAAGGT |
| | (SEQ ID NO: 26) |
| Tn5 For | CTCTTCGCTAGCATGATTACCAGTGCACTGCAT |
| | (SEQ ID NO: 27) |
| Tn5 Rev | AACAAGAATTCTTATTAGATTTTAATGCCCTGCGCCA |
| | (SEQ ID NO: 28) |

As expected, no signal was detected for the mRNA library obtained from cells without transfection of the mCherry-CD9 vector. In addition, no amplification was likewise detected for the negative controls (mRNA fragments alone, mRNA+Tn5 WT, mRNA+Tn5 Me) and the samples of the mRNA library with transfection of the mCherry-CD9. Nevertheless, remaining with this same batch of samples, a clear band was detected at the right molecular weight ˜ 1 kb ([FIG. 11]) for the sample corresponding to a replacement of the mCherry-CD9 with the GFP sequence (Tn5 complex). This band underlines a cleavage of the mCherry 3′ end (antisense mRNA) with insertion of the GFP 3′ end (sense strand) or, in other words, ligation of the mCherry mRNA antisense strand to the GFP sense strand in a 3′-to-5′ direction.

The amplification product was sequenced and the sequence obtained is presented in [FIG. 12]. As expected, an amplification of mCherry towards the GFP 3′ side was detected with a detection of the two sequences connected by ligation. The chromatogram of this sequencing clearly shows the sedimentation zone (tagmentation) at 10 bp from the target sequence ([FIG. 12]).

The same analysis was repeated but this time by changing the design for a ligation to the 3′ side: CD9 of the cDNA sense strand ([FIG. 12]). Thus, the same results were obtained with an amplification gel showing an expected band towards ˜ 1 kb ([FIG. 13]) for the cDNA library with mCherry-CD9 transfection and brought into contact with the ensemble according to the invention (Tn5 complex), and sequencing of the obtained fragment was carried out ([FIG. 14]). As expected, this sequencing made it possible to identify the sequence of the CD9 sense strand of sedimented cDNA with multiple sequences of the GFP 5′ side sense strand (ligation at 3′ of the GFP sense sequence; [FIG. 14]), at 5 bp from the target sequence.

### Example 2—Implementation of the Invention in Cellulo

Strengthened by these encouraging results, the design was modified in order to obtain a version compatible with a direct transfection of the ensemble, of the Tn5 plasmid and the strand opening protein, the plasmid of the UVRD bacterial helicase, which fuses with the monomeric streptavidin (UVRD-mSA plasmid) so that the latter is attached in situ to one of the molecules of the ensemble via previously biotinylated oligos. To do this, a new version of the ensemble was developed for easier placement and allowing control of close to 100% of the design (reduction of the Tn5 dimers not confined by the Beacon sarcophagus system and thus possible associated “off targets”). Thus, for this “New Design Ensemble”, the same type of protocol as before was carried out with certain major changes. In terms of actual design, the Loop nDREAMT A, B, C and D oligos (one per Tn5 dimer and one per target for a tagmentation of the four target areas—complete double-stranded replacement) are longer with three sequences for attachment to the transposase. This allows fast and controlled pairing with the simple addition of the last sequence for attachment to the transposase linked to the GFP fragments for replacing the mCherry-CD9 target fragment (nDREAMT A, B, C and D oligos). Furthermore, other nDREAMT Bio A and B oligos (biotinylated oligos) are added to allow in cellulo assembly of the ensemble with the UVRD-mSA fusion protein.

The following oligomers were mixed in two separate tubes 1 and 2: for tube 1 (10 μL), nDREAMT A and C loop (10 μM)+nDREAMT A and C oligos (10 μM)+nDREAMT Bio A oligo (10 μM); for tube 2 10 μl), nDREAMT B and D loop (10 μM)+nDREAMT B and D oligos (10 μM)+nDREAMT Bio B oligo (10 μM). The two tubes are then heated to 95° C. for 5 min and then left for 1 hour at room temperature, then each tube is digested with the corresponding restriction enzymes and purified by PCR purification kit (elution 20 μL).

At the same time, an amplified sequence of GFP comprising as ends flanking the sequences of restriction enzymes Ndel at 5′ and BSPEi at 3′ (oligos with restriction site architecture digested after PCR) was amplified and then digested with the corresponding restriction enzymes and purified by PCR purification kit eluted with a volume of 20 μL.

The contents of Tubes 1 and 2 are then mixed with the amplified and digested GFP neo fragment, a solution of T4 ligase (4 μL or 100U), 5 μL of T4 buffer (10×) and 1 μL of ddH20, for a total reaction volume of 50 μL. The solution is left for 1 hour at room temperature, then gel purification is carried out to isolate the largest GFP fragment, greater than 1 kb, and comprising the four sarcophagi A, B, C and D at its 5′ and 3′ ends. The GFP replacement fragment with its four sarcophagi positioned at the 5′ and 3′ ends is then ready to receive the transposase dimers ([FIG. 15]).

Before testing this system at the cell level, the new design was tested via the same series of experiments as previously with a GFP 3′ side ligation (sense strand) to the CD9 side of mCherry-CD9 target cDNA (sense strand). Thus, the same type of result as previously was obtained (all the remaining negative controls are undetectable) with, after amplification via the GFP Forward and CD9 Reverse PCR primers, an expected band towards 0.5 kb ([FIG. 15]). After Sanger sequencing of this band, as expected, a detection of the CD9 fragment in the 5′-to-3′ direction was carried out with ligation of a multiple sequence of GFP at its 3′ end ([FIG. 16]), within two bases of the original target sequence ([FIG. 16]).

After this positive verification of this new design, an in situ test in HEK 293T cells expressing mCherry-CD9 was carried out. To this end, two intracellular protein expression plasmids for the Tn5 transposase and UVRD-mSA proteins were created. For the Tn5 plasmid, the Tn5 sequence was amplified by PCR via the PTXB1-Tn5 plasmid (S. Picelli et al Genome Res 2014). It should be noted that the intein tail tag allowing purification of the Tn5 transposase by binding with chitin beads and self-cleavage, leaving no element of the tag on the Tn5 protein (S. Picelli et al Genome Res 2014), was replaced with a stop codon. The whole was cloned instead of the mCherry-CD9 fragment of the mCherry-CD9 plasmid (Davidson Lab) via the BMT1 and EcoR1 restriction enzymes, to finally obtain a CMV-Tn5 promoter plasmid. PCR amplification of the mSA fragment was carried out (table 1) via the pRSET-mSA plasmid (Sheldon Park Lab) with an addition of BMT1 restriction site at 5′ and EcoR1 restriction site at 3′ (table 1). A first cloning step was carried out in the mCherry-CD9 plasmid via the BM1 and EcoR1 enzymes, replacing the mCherry-CD9 fragment with the mSA fragment obtaining a CMV-mSA promoter plasmid. For the UVRD fragment, a cDNA library of E. coli bacteria was generated (DH5α). PCR amplification of the UVRD cDNA was carried out using precise oligonucleotides (table 1). On the 5′ side, the usual elements such as the BMT1 sequence for cloning and the Kozac sequence are found, but also the NLS import sequence to allow a complete Complex+Tn5+UVRD-mSA complex to be imported into the cell nucleus. At 3′, a flexible part was added (GGGSx3-type polyglycine tail), one end of the mSA 5′ sequence to a precise restriction enzyme contained in the PMiI mSA 5′ sequence. A second cloning step was then carried out using the BMT1 and PMiI restriction enzymes allowing an addition at 5′ of the UVRD sequence and obtaining a final plasmid of the CMV-UVRD-mSA promoter type.

The transfection of the HEK 293T cells was carried out by lipofectamine 3000 with the mCherry-CD9 plasmid. After waiting for 24 hours and verification by confocal microscope of the red signal on the cytoplasmic membrane, the cells were transfected with the three compounds: the CMV-Tn5 and CMV-UVRD-mSA plasmids and the New design complex. This complete complex allows double-stranded replacement of the sequence located between the four targets (quadruplet of targets; two mCherry sides and two CD9 sides) with a CMV-GFP sequence. Thus, by this experiment, a decrease or even a replacement of the red membrane signal with a green cytoplasmic signal was expected.

As expected and presented in [FIG. 17], five days after the second transfection with the technology according to the invention, green cells were detected.

These cells were isolated by cell sorting by cytometry and knowing that the CMV-GFP fragment replaced the mCherry-CD9 part of the CMV-mCherry-CD9 plasmid, this new CMV-GFP plasmid should contain an antibiotic selection sequence for creating a NeoR/KanR cell line. After more than 6 days of transfection, these still-green cells were placed in a medium comprising 2 mg/ml of G418, changed every day for 14 days and then kept at a concentration of 0.5 mg/ml for 3 months.

As expected, after 19 days post-transfection, a new GFP+ green cell line was obtained, which was very stable despite repeated freeze and thaw cycles (cf. [FIG. 17]).

In order to reconfirm this result in situ with greater certainty, a stable HEK 293T red membrane cell line was created by transfection with the CMV-mCherry-CD9 plasmid and treatment with 2 mg/ml of G418 as previously. After two weeks of treatment, a HEK 293T red membrane cell line was obtained.

This cell line was transfected as previously with the aforementioned technology. After three days of transfection, the first green cells began to appear (cf. [FIG. 18]). After 14 days post-transfection, certain groups of green cells were identified and isolated for further analysis (cf. [FIG. 18]).

After isolating these cells, a reverse transcription was carried out using these cells 18 days post-transfection, in order to obtain a cDNA library.

The GFP sequence was then amplified by PCR and the amplified fragment was then sequenced according to the Sanger method.

As expected, after analysis by comparison with the sequence databases (NCBI BLAST), a very close similarity is found with the theoretical GFP fragment replacing the mCherry-CD9 sequence [FIG. 19]. In addition, after almost one month, the green cells obtained remain stable with a strong green signal ([FIG. 18]). These results confirm the in cellulo efficacy of the technology according to the invention, allowing replacement of the mCherry-CD9 target sequence with the GFP replacement sequence illustrated by the change in color from the membrane red to the cytoplasmic green. This change in color makes it possible to validate the nuclear import of the new design+Tn5+UVRD-mSA complex (with the NLS on the N-Term sequence of the UVRD), the opening of the DNA strands carried out by the two 5′ and 3′ side helicases, the pairing at the four desired target points (two on the mCherry side and two on the CD9 side) and the replacement of the mCherry-CD9 sequence with the CMV-GFP fragment by the technology according to the invention.

1-15. (canceled)

16. A complex comprising a first and a second single-stranded nucleic acid molecules, the complex being such that the first and second single-stranded nucleic acid molecules are partially paired according to the base complementarity defined by Watson and Crick so as to define a first and a second double-stranded binding sites of a transposase,
wherein the first double-stranded binding site of the transposase is constituted by a first sequence oriented 5′-to-3′ for recognizing the transposase and a complementary sequence of the first sequence for recognizing the transposase,
wherein the second double-stranded binding site of the transposase is constituted by a second sequence oriented 5′-to-3′ for recognizing the transposase and complementary sequence of said second sequence for recognizing said transposase,
wherein
the first single-stranded nucleic acid molecule comprising or consisting essentially of an A sequence allowing the insertion of a complementary sequence of a nucleic acid of interest,
the A sequence being linked at its 5′-end to a first A/T-rich, sequence of 40 to 60 nucleotides in length and at its 3′-end to a second A/T-rich sequence of 40 to 60 nucleotides in length, the first and second A/T-rich sequences respectively comprising a first and a second G/C-rich domain of 6 to 12 nucleotides, the sequence of the first domain being complementary to the sequence of the second domain, the first and the second domains being positioned 15 to 52 nucleotides from said A sequence, the first molecule comprising at its 5′-end the first sequence oriented 5′-to-3′ for recognizing a transposase and at its 3′-end at least the second sequence for recognizing the transposase; and
the second single-stranded nucleic acid molecule comprising or consisting essentially at its 5′ end of at least the complementary sequence of the second sequence for recognizing the transposase,

17. The complex according to claim 16, wherein the A sequence comprises a complementary sequence of the nucleic acid of interest.

18. The complex according to claim 16, wherein the first molecule comprises at its 5′ end the first sequence oriented 5′-to-3′ for recognizing the transposase and at its 3′ end the second sequence oriented 5′-to-3′ for recognizing the transposase and
wherein the second molecule comprises its 5′ end the first complementary sequence of the first sequence for recognizing the transposase followed by the second complementary sequence of the second sequence for recognizing the transposase.

19. The complex according to claim 16, wherein said the molecule comprises at its 5′ end the first sequence oriented 5′-to-3′ for recognizing the transposase and at its 3′ end the second sequence for recognizing the transposase, followed by the first complementary sequence of the first sequence for recognizing the transposase and
wherein the second molecule comprises at its 5′ end the complementary sequence of the second sequence for recognizing the transposase.

20. The complex according to claim 16, wherein the transposase is a bacterial transposase.

21. The complex according to claim 16, wherein the first molecule is coupled with an enzyme.

22. The complex according to claim 16, wherein the first molecule comprises one of the following sequences
SEQ ID NO: 1-SEQ ID NO: 437-X-SEQ ID NO: 437-SEQ ID NO: 2-SEQ ID NO: 438,
ATCATC-SEQ ID NO: 1-SEQ ID NO: 446-SEQ ID NO: 1-SEQ ID NO: 447-X-SEQ ID NO: 448-SEQ ID NO: 2-SEQ ID NO: 449,
SEQ ID NO: 436-R1-SEQ ID NO: 452-X-SEQ ID NO: 453-R2-SEQ ID NO: 439,
SEQ ID NO: 436-R1-SEQ ID NO: 456-X-SEQ ID NO: 457-R2-SEQ ID NO: 439,
SEQ ID NO: 458-R1-SEQ ID NO: 459-X-SEQ ID NO: 460-R2-SEQ ID NO: 461,
SEQ ID NO: 458-R1-SEQ ID NO: 466-X-SEQ ID NO: 467-R2-SEQ ID NO: 461,
SEQ ID NO: 458-R1-SEQ ID NO: 468-X-SEQ ID NO: 469-R2-SEQ ID NO: 461,
SEQ ID NO: 445-R1-SEQ ID NO: 446-R1-SEQ ID NO: 470-X-SEQ ID NO: 474-R2-SEQ ID NO: 449,
SEQ ID NO: 445-R1-SEQ ID NO: 446-R1-SEQ ID NO: 471-X-SEQ ID NO: 472-R2-SEQ ID NO: 449,
GATAGTAG-R1-SEQ ID NO: 476-X-SEQ ID NO: 448-R2-SEQ ID NO: 477-R1,
GATAGTAG-R1-SEQ ID NO: 470-X-SEQ ID NO: 471-R2-SEQ ID NO: 477-R1,
GATAGTAG-R1-SEQ ID NO: 471-X-SEQ ID NO: 472-R2-SEQ ID NO: 477-R1,
CACGTG-R1-SEQ ID NO: 478-X-SEQ ID NO: 479-R2-SEQ ID NO: 480-R2-CACGTG,
CACGTG-R1-SEQ ID NO 484-X-SEQ ID NO 485-R2-SEQ ID NO 480-R2-CACGTG,
CACGTG-R1-SEQ ID NO 486-X-SEQ ID NO 487-R2-SEQ ID NO 480-R2-CACGTG,
R2-SEQ ID NO 488-R1-SEQ ID NO 478-X-SEQ ID NO 479-R2-GACGAATA,
R2-SEQ ID NO 488-R1-SEQ ID NO 484-X-SEQ ID NO 485-R2-GACGAATA, and
R2-SEQ ID NO 488-R1-SEQ ID NO 486-X-SEQ ID NO 487-R2-GACGAATA,
wherein R1 and R2 are such that
R1 is SEQ ID NO: n and R2 is SEQ ID NO: n+1, wherein n is an even number ranging from 1 to 6 and from 269 to 424, and
R1 is SEQ ID NO: n and R2 is SEQ ID NO: n+1, wherein n is an odd number ranging from 1 to 6 and from 269 to 424.

23. The complex according to claim 16, the complex comprising a pair of first and second molecules, the first and the second molecules comprising the sequences as defined in table 2.

24. The complex according to claim 16, the complex comprising one of the pairs of the first and the second molecules as defined in lines 1-166, 171-172, 177-178, 183-184 et 201 à 288 of table 4.

25. An ensemble comprising a first, a second and a third single-stranded nucleic acid molecules, the ensemble being such that
the first and third single-stranded nucleic acid molecules are partially paired according to the base complementarity defined by Watson and Crick so as to define a first and a second double-stranded binding sites of a transposase,
the second and third single-stranded nucleic acid molecules are partially paired according to the base complementarity defined by Watson and Crick so as to define the first and the second double-stranded binding sites of a transposase,
wherein the first double-stranded binding site of the transposase is constituted by a first sequence oriented 5′-to-3′ for recognizing the transposase and a complementary sequence of the first sequence for recognizing the transposase,
wherein the second double-stranded binding site of the transposase is constituted by a second sequence oriented 5′-to-3′ for recognizing the transposase and complementary sequence of said second sequence for recognizing said transposase,
wherein the third double-stranded binding site of the transposase is constituted by a third sequence oriented 5′-to-3′ for recognizing the transposase and a complementary sequence of the third sequence for recognizing the transposase,
wherein the fourth double-stranded binding site of the transposase is constituted by a fourth sequence oriented 5′-to-3′ for recognizing the transposase and complementary sequence of said fourth sequence for recognizing said transposase, wherein
the first single-stranded nucleic acid molecule comprising or consisting essentially of the A sequence allowing the insertion of a complementary sequence of a nucleic acid of interest, or comprising a complementary sequence of a nucleic acid of interest, the complementary sequence binding at 5′ to a first T-rich sequence of 40 to 60 nucleotides in length and at 3′ to a second T-rich sequence of 40 to 60 nucleotides in length, the first and second T-rich sequences respectively comprising a first and a second G/C-rich domain of 6 to 12 nucleotides, the sequence of the first domain being complementary to the sequence of the second domain, the first and second domains being positioned 15 to 52 nucleotides from the A sequence, the first molecule comprising at its 5′ end at least the first sequence oriented 5′-to-3′ for recognizing the transposase and at its 3′ end the second sequence for recognizing the transposase,
the second single-stranded nucleic acid molecule comprising or consisting essentially of a B sequence allowing the insertion of a complementary sequence of the nucleic acid of interest, or comprising a complementary sequence of a nucleic acid of interest, the complementary B sequence binding at 5′ to a third T-rich sequence of 40 to 60 nucleotides in length and at 3′ to a fourth T-rich sequence of 40 to 60 nucleotides in length, the third and fourth T-rich sequence respectively comprising a third and a fourth G/C-rich domain of 6 to 12 nucleotides, the sequence of the third domain being complementary to the sequence of the fourth domain, the third and fourth domains being positioned 15 to 52 nucleotides from the B sequence, the second molecule comprising at its 5′ end at least the first sequence oriented 5′-to-3′ for recognizing the transposase and at its 3′ end the second sequence for recognizing said transposase,
the B sequence being a complementary sequence of the nucleic acid of interest, the A sequence being positioned at 5′ end of a region of interest of the nucleic acid of interest and the B sequence being positioned at 3′ end of the region of interest of the nucleic acid of interest, and
a third single-stranded molecule comprising
in its 5′ part, at least the complementary sequence of said second sequence for recognizing the transposase of the first molecule,
in its 3′ part, at least the complementary sequence of said first sequence for recognizing the transposase of the second molecule, and
a region located between complementary sequence of the second sequence for recognizing the transposase of the first molecule and the complementary sequence of the first sequence for recognizing the transposase of the second molecule allowing the insertion of a single-stranded replacement nucleic acid molecule.

26. The ensemble according to claim 25, the ensemble comprising one of the pairs of first and third molecules as defined in lines 1-166, 171-172, 177-178, 183-184 et 201 à 288 of table 5.

27. A kit comprising at least one vector allowing the expression of a recombinase and the first, second, and third molecules of the ensemble as defined in claim 25.

28. A method for replacement of a target region of a nucleic acid molecule with a region of interest of another nucleic acid molecule, so as to obtain a hybrid nucleic acid molecule, said method comprising:
bringing an ensemble as defined in claim 25 into contact with the nucleic acid comprising the target region,
said ensemble being such that
the A sequence of the first molecule comprises a complementary sequence of the region immediately at 5′ end of the target region,
the B sequence of said second molecule comprises a complementary sequence of the region immediately at 3′ end of the target region, and
the third molecule comprises the region of interest in the region located between complementary sequence of the second sequence for recognizing the transposase of the first molecule and the complementary sequence of the first sequence for recognizing the transposase of the second molecule, in order to obtain a replacement complex,
placing the replacement complex in the presence of a transposase recognizing the double-stranded binding sites the said transposase contained in the ensemble, to obtain a recombination complex, and
recombining the combination complex in order to obtain a hybrid nucleic acid molecule comprising the region of interest instead of the target region.

29. A method for editing the genome of a cell, making it possible to replace a specific fragment of the double-stranded DNA of said genome of said cell with another double-stranded DNA fragment of interest, in order to obtain a recombinant hybrid genome comprising the other double-stranded DNA fragment of interest instead of the specific fragment of double-stranded DNA, said method comprising:
preparing a first ensemble as defined in claim 25,
wherein the A sequence of the first molecule comprises a complementary sequence of the adjacent region at 5′ of the specific fragment;
wherein the B sequence of the second molecule comprises a complementary sequence of the adjacent region at 3′ of the specific fragment; and
wherein the third molecule comprises, between the region located between complementary sequence of said second sequence for recognizing said transposase of the first molecule and the complementary sequence of said first sequence for recognizing said transposase of the second molecule, a sequence of one of the strands of said specific fragment;
and preparing a second ensemble,
wherein the A sequence of said complementary region of the first molecule of the second ensemble comprises a complementary sequence of the adjacent region at 5′ of the specific fragment;
wherein the B sequence of said complementary region of the second molecule of the second ensemble comprises a complementary sequence of the adjacent region at 3′ of the specific fragment; and
wherein the third molecule of the second ensemble comprises, between the region located between complementary sequence of said second sequence for recognizing said transposase of the first molecule of the second ensemble and the complementary sequence of said first sequence for recognizing said transposase of the second molecule of the second ensemble, the sequence of the complementary strand of said specific fragment contained in the third sequence of the first ensemble;
wherein the complementary sequence of the adjacent region at 5′ of the specific fragment contained in the A region of the first molecule of the first ensemble is at most 95% complementary to the complementary sequence of the adjacent region at 5′ of the specific fragment contained in the A region of the first molecule of the second ensemble; and
wherein the complementary sequence of the adjacent region at 3′ of the specific fragment contained in the B region of the first molecule of the first ensemble is at most 95% complementary to the complementary sequence of the adjacent region at 3′ of the specific fragment contained in the B region of the first molecule of the second ensemble;
in order to obtain a recombination complex;
bringing said cell into contact with said recombination complex, in order to obtain a cell ready to be edited,
expressing the transposase in the cell ready to be edited, in order to obtain an edited cell,
selecting the edited cell, wherein the genome of the edited cell comprises, instead of the specific double-stranded DNA fragment, the other double-stranded DNA fragment of interest

30. The complex according to claim 20, wherein the transposase is selected from the group consisting of Tn5, Tn9, Tn10 or Tc1/mariner.


SEQUENCE LISTING (774 sequences)

SEQ ID NO: 1 - DNA, 19 bp, Artificial sequence
  molecule of the complex
ctgtctcttatacacatct

SEQ ID NO: 2 - DNA, 19 bp, Artificial sequence
  transposase binding site
agatgtgtataagagacag

SEQ ID NO: 3 - DNA, 19 bp, Artificial sequence
  transposase binding site
ctgactcttatacacaagt

SEQ ID NO: 4 - DNA, 19 bp, Artificial sequence
  transposase binding site
acttgtgtataagagtcag

SEQ ID NO: 5 - DNA, 19 bp, Artificial sequence
  transposase binding site
ctgtctcttgatcagatct

SEQ ID NO: 6 - DNA, 19 bp, Artificial sequence
  transposase binding site
agatctgatcaagagacag

SEQ ID NO: 7 - DNA, 83 bp, Artificial sequence
  first molecule of the complex
tgcagctgctgtctcttatacacatcttttttttttttttttttttttttttttggcgatcgctttttttttttttttttttt

SEQ ID NO: 8 - DNA, 94 bp, Artificial sequence
  third molecule of the complex
agatgtgtataagagacagcagctgcagacaaagcttacagctgtctcttatacacatctttttttttttttttttttttttcatatgccaagt

SEQ ID NO: 9 - DNA, 116 bp, Artificial sequence
  second molecule of the complex
atcatcctgtctcttatacacatcttttttttttttttttttgatagtagctgtctcttatacacatctttttttttttttttttttttttggcgatcgctttttttttttttttt

SEQ ID NO: 10 - DNA, 63 bp, Artificial sequence
  third molecule of the complex
acttggttaattaattttttttttttttttttttttagatgtgtataagagacagctactatc

SEQ ID NO: 11 - DNA, 19 bp, Artificial sequence
  Tn5 binding sequence
ctgtctcttatacacatct

SEQ ID NO: 12 - DNA, 33 bp, Artificial sequence
  Tn5 binding sequence
tcgtcggcagcgtcagatgtgtataagagacag

SEQ ID NO: 13 - DNA, 34 bp, Artificial sequence
  Tn5 binding sequence
gtctcgtgggctcggagatgtgtataagagacag

SEQ ID NO: 14 - DNA, 22 bp, Artificial sequence
  GFP sense oligo
ctggtcgagctggacggcgacg

SEQ ID NO: 15 - DNA, 22 bp, Artificial sequence
  GFP reverse oligo
cacgaactccagcaggaccatg

SEQ ID NO: 16 - DNA, 21 bp, Artificial sequence
  mCherry sense oligo
aagggcgaggaggataacatg

SEQ ID NO: 17 - DNA, 18 bp, Artificial sequence
  CD9 reverse oligo
gaccatctcgcggttcct

SEQ ID NO: 18 - DNA, 32 bp, Artificial sequence
  GFP Nde1 sense oligo
acttggcatatgatggtgagcaagggcgagga

SEQ ID NO: 19 - DNA, 30 bp, Artificial sequence
  GFP Nde1 reverse oligo
acttggcatatgcttgtacagctcgtccat

SEQ ID NO: 20 - DNA, 32 bp, Artificial sequence
  GFP BSPEI sense oligo
tacaagtccggaatggtgagcaagggcgagga

SEQ ID NO: 21 - DNA, 30 bp, Artificial sequence
  GFP BSPEI reverse oligo
tacaagtccggacttgtacagctcgtccat

SEQ ID NO: 22 - DNA, 32 bp, Artificial sequence
  CMV Nde1 sense oligo
acttggcatatgccaagtacgccccctattga

SEQ ID NO: 23 - DNA, 86 bp, Artificial sequence
  UVRD sense oligo
ctcttcgctagctgccaccatgacgcgtggccccaagaaaaagcggaaagtgggaccggccaccatggacgtttcttacctgctcg

SEQ ID NO: 24 - DNA, 81 bp, Artificial sequence
  UVRD reverse oligo
aacaacacgtgccggtgatacccgcttccgcgcctgatccaccaccacctgacccaccaccagccaccgactccagccggg

SEQ ID NO: 25 - DNA, 29 bp, Artificial sequence
  mSA sense oligo
ctcttcgctagcgcggaagcgggtatcac

SEQ ID NO: 26 - DNA, 35 bp, Artificial sequence
  mSA reverse oligo
aacaagaattcttattatttaactttggtgaaggt

SEQ ID NO: 27 - DNA, 33 bp, Artificial sequence
  Tn5 sense oligo
ctcttcgctagcatgattaccagtgcactgcat

SEQ ID NO: 28 - DNA, 37 bp, Artificial sequence
  Tn5 reverse oligo
aacaagaattcttattagattttaatgccctgcgcca

SEQ ID NO: 29 - DNA, 6 bp, Artificial sequence
  restriction site
aacgtt

SEQ ID NO: 30 - DNA, 6 bp, Artificial sequence
  restriction site
aagctt

SEQ ID NO: 31 - DNA, 6 bp, Artificial sequence
  restriction site
aatatt

SEQ ID NO: 32 - DNA, 4 bp, Artificial sequence
  restriction site
aatt

SEQ ID NO: 33 - DNA, 6 bp, Artificial sequence
  restriction site
acatgt

SEQ ID NO: 34 - DNA, 6 bp, Artificial sequence
  restriction site
accggt

SEQ ID NO: 35 - DNA, 6 bp, Artificial sequence
  restriction site
acctgc

SEQ ID NO: 36 - DNA, 7 bp, Artificial sequence
  restriction site
accwggt

SEQ ID NO: 37 - DNA, 6 bp, Artificial sequence
  restriction site
acgcgt

SEQ ID NO: 38 - DNA, 5 bp, Artificial sequence
  restriction site
acggc

SEQ ID NO: 39 - DNA, 4 bp, Artificial sequence
  restriction site
acgt

SEQ ID NO: 40 - DNA, 5 bp, Artificial sequence
  restriction site
  misc_feature (3)..(3): n is a, c, g, or t
acngt

SEQ ID NO: 41 - DNA, 11 bp, Artificial sequence
  restriction site
  misc_feature (3)..(6): n is a, c, g, or t
acnnnngtayc

SEQ ID NO: 42 - DNA, 11 bp, Artificial sequence
  restriction site
  misc_feature (3)..(7): n is a, c, g, or t
acnnnnnctcc

SEQ ID NO: 43 - DNA, 6 bp, Artificial sequence
  restriction site
acrygt

SEQ ID NO: 44 - DNA, 6 bp, Artificial sequence
  restriction site
actagt

SEQ ID NO: 45 - DNA, 5 bp, Artificial sequence
  restriction site
actgg

SEQ ID NO: 46 - DNA, 6 bp, Artificial sequence
  restriction site
actggg

SEQ ID NO: 47 - DNA, 6 bp, Artificial sequence
  restriction site
agatct

SEQ ID NO: 48 - DNA, 6 bp, Artificial sequence
  restriction site
agcgct

SEQ ID NO: 49 - DNA, 4 bp, Artificial sequence
  restriction site
agct

SEQ ID NO: 50 - DNA, 6 bp, Artificial sequence
  restriction site
aggcct

SEQ ID NO: 51 - DNA, 6 bp, Artificial sequence
  restriction site
agtact

SEQ ID NO: 52 - DNA, 6 bp, Artificial sequence
  restriction site
atcgat

SEQ ID NO: 53 - DNA, 30 bp, Artificial sequence
  restriction site
atctatgtcgggtgcggagaaagaggtaat

SEQ ID NO: 54 - DNA, 6 bp, Artificial sequence
  restriction site
atgcat

SEQ ID NO: 55 - DNA, 6 bp, Artificial sequence
  restriction site
attaat

SEQ ID NO: 56 - DNA, 8 bp, Artificial sequence
  restriction site
atttaaat

SEQ ID NO: 57 - DNA, 12 bp, Artificial sequence
  restriction site
  misc_feature (4)..(8): n is a, c, g, or t
caannnnngtgg

SEQ ID NO: 58 - DNA, 6 bp, Artificial sequence
  restriction site
caattg

SEQ ID NO: 59 - DNA, 7 bp, Artificial sequence
  restriction site
cacctgc

SEQ ID NO: 60 - DNA, 6 bp, Artificial sequence
  restriction site
cacgag

SEQ ID NO: 61 - DNA, 6 bp, Artificial sequence
  restriction site
cacgag

SEQ ID NO: 62 - DNA, 6 bp, Artificial sequence
  restriction site
cacgtc

SEQ ID NO: 63 - DNA, 6 bp, Artificial sequence
  restriction site
cacgtg

SEQ ID NO: 64 - DNA, 9 bp, Artificial sequence
  restriction site
  misc_feature (4)..(6): n is a, c, g, or t
cacnnngtg

SEQ ID NO: 65 - DNA, 10 bp, Artificial sequence
  restriction site
  misc_feature (4)..(7): n is a, c, g, or t
cacnnnngtg

SEQ ID NO: 66 - DNA, 6 bp, Artificial sequence
  restriction site
cagcag

SEQ ID NO: 67 - DNA, 6 bp, Artificial sequence
  restriction site
cagctg

SEQ ID NO: 68 - DNA, 9 bp, Artificial sequence
  restriction site
  misc_feature (4)..(6): n is a, c, g, or t
cagnnnctg

SEQ ID NO: 69 - DNA, 5 bp, Artificial sequence
  restriction site
cagtg

SEQ ID NO: 70 - DNA, 6 bp, Artificial sequence
  restriction site
catatg

SEQ ID NO: 71 - DNA, 4 bp, Artificial sequence
  restriction site
catg

SEQ ID NO: 72 - DNA, 4 bp, Artificial sequence
  restriction site
catg

SEQ ID NO: 73 - DNA, 4 bp, Artificial sequence
  restriction site
catg

SEQ ID NO: 74 - DNA, 10 bp, Artificial sequence
  restriction site
  misc_feature (4)..(7): n is a, c, g, or t
caynnnnrtg

SEQ ID NO: 75 - DNA, 15 bp, Artificial sequence
  restriction site
  misc_feature (4)..(12): n is a, c, g, or t
ccannnnnnnnntgg

SEQ ID NO: 76 - DNA, 12 bp, Artificial sequence
  restriction site
  misc_feature (4)..(9): n is a, c, g, or t
ccannnnnntgg

SEQ ID NO: 77 - DNA, 11 bp, Artificial sequence
  restriction site
  misc_feature (4)..(8): n is a, c, g, or t
ccannnnntgg

SEQ ID NO: 78 - DNA, 5 bp, Artificial sequence
  restriction site
ccatc

SEQ ID NO: 79 - DNA, 6 bp, Artificial sequence
  restriction site
ccatgg

SEQ ID NO: 80 - DNA, 6 bp, Artificial sequence
  restriction site
cccagc

SEQ ID NO: 81 - DNA, 5 bp, Artificial sequence
  restriction site
cccgc

SEQ ID NO: 82 - DNA, 6 bp, Artificial sequence
  restriction site
cccggg

SEQ ID NO: 83 - DNA, 6 bp, Artificial sequence
  restriction site
cccggg

SEQ ID NO: 84 - DNA, 4 bp, Artificial sequence
  restriction site
ccdg

SEQ ID NO: 85 - DNA, 4 bp, Artificial sequence
  restriction site
ccgc

SEQ ID NO: 86 - DNA, 6 bp, Artificial sequence
  restriction site
ccgcgg

SEQ ID NO: 87 - DNA, 6 bp, Artificial sequence
  restriction site
ccgctc

SEQ ID NO: 88 - DNA, 4 bp, Artificial sequence
  restriction site
ccgg

SEQ ID NO: 89 - DNA, 5 bp, Artificial sequence
  restriction site
  misc_feature (3)..(3): n is a, c, g, or t
ccngg

SEQ ID NO: 90 - DNA, 5 bp, Artificial sequence
  restriction site
  misc_feature (3)..(3): n is a, c, g, or t
ccngg

SEQ ID NO: 91 - DNA, 6 bp, Artificial sequence
  restriction site
  misc_feature (3)..(4): n is a, c, g, or t
ccnngg

SEQ ID NO: 92 - DNA, 11 bp, Artificial sequence
  restriction site
  misc_feature (3)..(9): n is a, c, g, or t
ccnnnnnnngg

SEQ ID NO: 93 - DNA, 6 bp, Artificial sequence
  restriction site
ccrygg

SEQ ID NO: 94 - DNA, 5 bp, Artificial sequence
  restriction site
ccsgg

SEQ ID NO: 95 - DNA, 6 bp, Artificial sequence
  restriction site
cctagg

SEQ ID NO: 96 - DNA, 4 bp, Artificial sequence
  restriction site
cctc

SEQ ID NO: 97 - DNA, 7 bp, Artificial sequence
  restriction site
cctcagc

SEQ ID NO: 98 - DNA, 7 bp, Artificial sequence
  restriction site
cctcagc

SEQ ID NO: 99 - DNA, 7 bp, Artificial sequence
  restriction site
cctcagc

SEQ ID NO: 100 - DNA, 8 bp, Artificial sequence
  restriction site
cctgcagg

SEQ ID NO: 101 - DNA, 7 bp, Artificial sequence
  restriction site
  misc_feature (4)..(4): n is a, c, g, or t
cctnagc

SEQ ID NO: 102 - DNA, 7 bp, Artificial sequence
  restriction site
  misc_feature (4)..(4): n is a, c, g, or t
cctnagg

SEQ ID NO: 103 - DNA, 11 bp, Artificial sequence
  restriction site
  misc_feature (4)..(8): n is a, c, g, or t
cctnnnnnagg

SEQ ID NO: 104 - DNA, 5 bp, Artificial sequence
  restriction site
ccttc

SEQ ID NO: 105 - DNA, 5 bp, Artificial sequence
  restriction site
ccwgg

SEQ ID NO: 106 - DNA, 5 bp, Artificial sequence
  restriction site
ccwgg

SEQ ID NO: 107 - DNA, 6 bp, Artificial sequence
  restriction site
ccwwgg

SEQ ID NO: 108 - DNA, 12 bp, Artificial sequence
  restriction site
  misc_feature (4)..(9): n is a, c, g, or t
cgannnnnntgc

SEQ ID NO: 109 - DNA, 6 bp, Artificial sequence
  restriction site
cgatcg

SEQ ID NO: 110 - DNA, 4 bp, Artificial sequence
  restriction site
cgcg

SEQ ID NO: 111 - DNA, 6 bp, Artificial sequence
  restriction site
cggccg

SEQ ID NO: 112 - DNA, 7 bp, Artificial sequence
  restriction site
cggwccg

SEQ ID NO: 113 - DNA, 6 bp, Artificial sequence
  restriction site
cgrycg

SEQ ID NO: 114 - DNA, 6 bp, Artificial sequence
  restriction site
cgtacg

SEQ ID NO: 115 - DNA, 6 bp, Artificial sequence
  restriction site
cgtctc

SEQ ID NO: 116 - DNA, 6 bp, Artificial sequence
  restriction site
cgtctc

SEQ ID NO: 117 - DNA, 5 bp, Artificial sequence
  restriction site
cgwcg

SEQ ID NO: 118 - DNA, 6 bp, Artificial sequence
  restriction site
cmgckg

SEQ ID NO: 119 - DNA, 22 bp, Artificial sequence
  restriction site
  misc_feature (2)..(21): n is a, c, g, or t
cnnnnnnnnnnnnnnnnnnnng

SEQ ID NO: 120 - DNA, 4 bp, Artificial sequence
  restriction site
  misc_feature (2)..(3): n is a, c, g, or t
cnnr

SEQ ID NO: 121 - DNA, 8 bp, Artificial sequence
  restriction site
crccggyg

SEQ ID NO: 122 - DNA, 4 bp, Artificial sequence
  restriction site
ctag

SEQ ID NO: 123 - DNA, 5 bp, Artificial sequence
  restriction site
ctcag

SEQ ID NO: 124 - DNA, 6 bp, Artificial sequence
  restriction site
ctcgag

SEQ ID NO: 125 - DNA, 6 bp, Artificial sequence
  restriction site
ctcttc

SEQ ID NO: 126 - DNA, 6 bp, Artificial sequence
  restriction site
ctgaag

SEQ ID NO: 127 - DNA, 6 bp, Artificial sequence
  restriction site
ctgcag

SEQ ID NO: 128 - DNA, 6 bp, Artificial sequence
  restriction site
ctggag

SEQ ID NO: 129 - DNA, 5 bp, Artificial sequence
  restriction site
  misc_feature (3)..(3): n is a, c, g, or t
ctnag

SEQ ID NO: 130 - DNA, 6 bp, Artificial sequence
  restriction site
ctryag

SEQ ID NO: 131 - DNA, 6 bp, Artificial sequence
  restriction site
cttaag

SEQ ID NO: 132 - DNA, 6 bp, Artificial sequence
  restriction site
cttgag

SEQ ID NO: 133 - DNA, 6 bp, Artificial sequence
  restriction site
ctyrag

SEQ ID NO: 134 - DNA, 6 bp, Artificial sequence
  restriction site
cycgrg

SEQ ID NO: 135 - DNA, 5 bp, Artificial sequence
  restriction site
gaaga

SEQ ID NO: 136 - DNA, 6 bp, Artificial sequence
  restriction site
gaagac

SEQ ID NO: 137 - DNA, 10 bp, Artificial sequence
  restriction site
  misc_feature (4)..(7): n is a, c, g, or t
gaannnnttc

SEQ ID NO: 138 - DNA, 6 bp, Artificial sequence
  restriction site
gaatgc

SEQ ID NO: 139 - DNA, 6 bp, Artificial sequence
  restriction site
gaatgc

SEQ ID NO: 140 - DNA, 6 bp, Artificial sequence
  restriction site
gaattc

SEQ ID NO: 141 - DNA, 5 bp, Artificial sequence
  restriction site
gacgc

SEQ ID NO: 142 - DNA, 6 bp, Artificial sequence
  restriction site
gacgtc

SEQ ID NO: 143 - DNA, 6 bp, Artificial sequence
  restriction site
gacgtc

SEQ ID NO: 144 - DNA, 9 bp, Artificial sequence
  restriction site
  misc_feature (4)..(6): n is a, c, g, or t
gacnnngtc

SEQ ID NO: 145 - DNA, 10 bp, Artificial sequence
  restriction site
  misc_feature (4)..(7): n is a, c, g, or t
gacnnnngtc

SEQ ID NO: 146 - DNA, 11 bp, Artificial sequence
  restriction site
  misc_feature (4)..(8): n is a, c, g, or t
gacnnnnngtc

SEQ ID NO: 147 - DNA, 12 bp, Artificial sequence
  restriction site
  misc_feature (4)..(9): n is a, c, g, or t
gacnnnnnngtc

SEQ ID NO: 148 - DNA, 6 bp, Artificial sequence
  restriction site
gagctc

SEQ ID NO: 149 - DNA, 6 bp, Artificial sequence
  restriction site
gagctc

SEQ ID NO: 150 - DNA, 6 bp, Artificial sequence
  restriction site
gaggag

SEQ ID NO: 151 - DNA, 5 bp, Artificial sequence
  restriction site
gagtc

SEQ ID NO: 152 - DNA, 5 bp, Artificial sequence
  restriction site
gagtc

SEQ ID NO: 153 - DNA, 5 bp, Artificial sequence
  restriction site
gagtc

SEQ ID NO: 154 - DNA, 5 bp, Artificial sequence
  restriction site
  misc_feature (3)..(3): n is a, c, g, or t
gantc

SEQ ID NO: 155 - DNA, 6 bp, Artificial sequence
  restriction site
gatatc

SEQ ID NO: 156 - DNA, 4 bp, Artificial sequence
  restriction site
gatc

SEQ ID NO: 157 - DNA, 4 bp, Artificial sequence
  restriction site
gatc

SEQ ID NO: 158 - DNA, 10 bp, Artificial sequence
  restriction site
  misc_feature (4)..(7): n is a, c, g, or t
gatnnnnatc

SEQ ID NO: 159 - DNA, 5 bp, Artificial sequence
  restriction site
gawtc

SEQ ID NO: 160 - DNA, 6 bp, Artificial sequence
  restriction site
gcaatg

SEQ ID NO: 161 - DNA, 6 bp, Artificial sequence
  restriction site
gcaatg

SEQ ID NO: 162 - DNA, 5 bp, Artificial sequence
  restriction site
gcagc

SEQ ID NO: 163 - DNA, 6 bp, Artificial sequence
  restriction site
gcagtg

SEQ ID NO: 164 - DNA, 6 bp, Artificial sequence
  restriction site
gcagtg

SEQ ID NO: 165 - DNA, 11 bp, Artificial sequence
  restriction site
  misc_feature (4)..(8): n is a, c, g, or t
gcannnnntgc

SEQ ID NO: 166 - DNA, 5 bp, Artificial sequence
  restriction site
gcatc

SEQ ID NO: 167 - DNA, 6 bp, Artificial sequence
  restriction site
gcatgc

SEQ ID NO: 168 - DNA, 8 bp, Artificial sequence
  restriction site
gcccgggc

SEQ ID NO: 169 - DNA, 6 bp, Artificial sequence
  restriction site
gccgag

SEQ ID NO: 170 - DNA, 6 bp, Artificial sequence
  restriction site
gccggc

SEQ ID NO: 171 - DNA, 6 bp, Artificial sequence
  restriction site
gccggc

SEQ ID NO: 172 - DNA, 11 bp, Artificial sequence
  restriction site
  misc_feature (4)..(8): n is a, c, g, or t
gccnnnnnggc

SEQ ID NO: 173 - DNA, 8 bp, Artificial sequence
  restriction site
gcgatcgc

SEQ ID NO: 174 - DNA, 6 bp, Artificial sequence
  restriction site
gcgatg

SEQ ID NO: 175 - DNA, 4 bp, Artificial sequence
  restriction site
gcgc

SEQ ID NO: 176 - DNA, 4 bp, Artificial sequence
  restriction site
gcgc

SEQ ID NO: 177 - DNA, 6 bp, Artificial sequence
  restriction site
gcgcgc

SEQ ID NO: 178 - DNA, 8 bp, Artificial sequence
  restriction site
gcggccgc

SEQ ID NO: 179 - DNA, 5 bp, Artificial sequence
  restriction site
  misc_feature (3)..(3): n is a, c, g, or t
gcngc

SEQ ID NO: 180 - DNA, 6 bp, Artificial sequence
  restriction site
  misc_feature (3)..(4): n is a, c, g, or t
gcnngc

SEQ ID NO: 181 - DNA, 11 bp, Artificial sequence
  restriction site
  misc_feature (3)..(9): n is a, c, g, or t
gcnnnnnnngc

SEQ ID NO: 182 - DNA, 6 bp, Artificial sequence
  restriction site
gctagc

SEQ ID NO: 183 - DNA, 6 bp, Artificial sequence
  restriction site
gctagc

SEQ ID NO: 184 - DNA, 7 bp, Artificial sequence
  restriction site
gctcttc

SEQ ID NO: 185 - DNA, 7 bp, Artificial sequence
  restriction site
gctcttc

SEQ ID NO: 186 - DNA, 7 bp, Artificial sequence
  restriction site
  misc_feature (4)..(4): n is a, c, g, or t
gctnagc

SEQ ID NO: 187 - DNA, 5 bp, Artificial sequence
  restriction site
gcwgc

SEQ ID NO: 188 - DNA, 6 bp, Artificial sequence
  restriction site
gdgchc

SEQ ID NO: 189 - DNA, 5 bp, Artificial sequence
  restriction site
ggatc

SEQ ID NO: 190 - DNA, 5 bp, Artificial sequence
  restriction site
ggatc

SEQ ID NO: 191 - DNA, 6 bp, Artificial sequence
  restriction site
ggatcc

SEQ ID NO: 192 - DNA, 5 bp, Artificial sequence
  restriction site
ggatg

SEQ ID NO: 193 - DNA, 5 bp, Artificial sequence
  restriction site
ggatg

SEQ ID NO: 194 - DNA, 4 bp, Artificial sequence
  restriction site
ggcc

SEQ ID NO: 195 - DNA, 8 bp, Artificial sequence
  restriction site
ggccggcc

SEQ ID NO: 196 - DNA, 13 bp, Artificial sequence
  restriction site
  misc_feature (5)..(9): n is a, c, g, or t
ggccnnnnnggcc

SEQ ID NO: 197 - DNA, 6 bp, Artificial sequence
  restriction site
ggcgcc

SEQ ID NO: 198 - DNA, 6 bp, Artificial sequence
  restriction site
ggcgcc

SEQ ID NO: 199 - DNA, 6 bp, Artificial sequence
  restriction site
ggcgcc

SEQ ID NO: 200 - DNA, 6 bp, Artificial sequence
  restriction site
ggcgcc

SEQ ID NO: 201 - DNA, 8 bp, Artificial sequence
  restriction site
ggcgcgcc

SEQ ID NO: 202 - DNA, 6 bp, Artificial sequence
  restriction site
ggcgga

SEQ ID NO: 203 - DNA, 5 bp, Artificial sequence
  restriction site
gggac

SEQ ID NO: 204 - DNA, 6 bp, Artificial sequence
  restriction site
gggccc

SEQ ID NO: 205 - DNA, 6 bp, Artificial sequence
  restriction site
gggccc

SEQ ID NO: 206 - DNA, 5 bp, Artificial sequence
  restriction site
  misc_feature (3)..(3): n is a, c, g, or t
ggncc

SEQ ID NO: 207 - DNA, 6 bp, Artificial sequence
  restriction site
  misc_feature (3)..(4): n is a, c, g, or t
ggnncc

SEQ ID NO: 208 - DNA, 6 bp, Artificial sequence
  restriction site
ggtacc

SEQ ID NO: 209 - DNA, 6 bp, Artificial sequence
  restriction site
ggtacc

SEQ ID NO: 210 - DNA, 6 bp, Artificial sequence
  restriction site
ggtctc

SEQ ID NO: 211 - DNA, 5 bp, Artificial sequence
  restriction site
ggtga

SEQ ID NO: 212 - DNA, 7 bp, Artificial sequence
  restriction site
  misc_feature (4)..(4): n is a, c, g, or t
ggtnacc

SEQ ID NO: 213 - DNA, 5 bp, Artificial sequence
  restriction site
ggwcc

SEQ ID NO: 214 - DNA, 6 bp, Artificial sequence
  restriction site
ggyrcc

SEQ ID NO: 215 - DNA, 6 bp, Artificial sequence
  restriction site
gkgcmc

SEQ ID NO: 216 - DNA, 6 bp, Artificial sequence
  restriction site
grcgyc

SEQ ID NO: 217 - DNA, 6 bp, Artificial sequence
  restriction site
grgcyc

SEQ ID NO: 218 - DNA, 4 bp, Artificial sequence
  restriction site
gtac

SEQ ID NO: 219 - DNA, 4 bp, Artificial sequence
  restriction site
gtac

SEQ ID NO: 220 - DNA, 6 bp, Artificial sequence
  restriction site
gtatac

SEQ ID NO: 221 - DNA, 6 bp, Artificial sequence
  restriction site
gtatcc

SEQ ID NO: 222 - DNA, 6 bp, Artificial sequence
  restriction site
gtcgac

SEQ ID NO: 223 - DNA, 5 bp, Artificial sequence
  restriction site
gtctc

SEQ ID NO: 224 - DNA, 5 bp, Artificial sequence
  restriction site
gtctc

SEQ ID NO: 225 - DNA, 6 bp, Artificial sequence
  restriction site
gtgcac

SEQ ID NO: 226 - DNA, 6 bp, Artificial sequence
  restriction site
gtgcag

SEQ ID NO: 227 - DNA, 6 bp, Artificial sequence
  restriction site
gtmkac

SEQ ID NO: 228 - DNA, 6 bp, Artificial sequence
  restriction site
  misc_feature (3)..(4): n is a, c, g, or t
gtnnac

SEQ ID NO: 229 - DNA, 5 bp, Artificial sequence
  restriction site
gtsac

SEQ ID NO: 230 - DNA, 6 bp, Artificial sequence
  restriction site
gttaac

SEQ ID NO: 231 - DNA, 8 bp, Artificial sequence
  restriction site
gtttaaac

SEQ ID NO: 232 - DNA, 6 bp, Artificial sequence
  restriction site
gtyrac

SEQ ID NO: 233 - DNA, 6 bp, Artificial sequence
  restriction site
gwgcwc

SEQ ID NO: 234 - DNA, 9 bp, Artificial sequence
  restriction site
  misc_feature (1)..(2): n is a, c, g, or t
  misc_feature (8)..(9): n is a, c, g, or t
nncastgnn

SEQ ID NO: 235 - DNA, 6 bp, Artificial sequence
  restriction site
raatty

SEQ ID NO: 236 - DNA, 6 bp, Artificial sequence
  restriction site
rcatgy

SEQ ID NO: 237 - DNA, 6 bp, Artificial sequence
  restriction site
rccggy

SEQ ID NO: 238 - DNA, 6 bp, Artificial sequence
  restriction site
rgatcy

SEQ ID NO: 239 - DNA, 6 bp, Artificial sequence
  restriction site
rgcgcy

SEQ ID NO: 240 - DNA, 4 bp, Artificial sequence
  restriction site
rgcy

SEQ ID NO: 241 - DNA, 7 bp, Artificial sequence
  restriction site
  misc_feature (4)..(4): n is a, c, g, or t
rggnccy

SEQ ID NO: 242 - DNA, 7 bp, Artificial sequence
  restriction site
rggwccy

SEQ ID NO: 243 - DNA, 27 bp, Artificial sequence
  restriction site
taactataacggtcctaaggtagcgaa

SEQ ID NO: 244 - DNA, 6 bp, Artificial sequence
  restriction site
tacgta

SEQ ID NO: 245 - DNA, 18 bp, Artificial sequence
  restriction site
tagggataacagggtaat

SEQ ID NO: 246 - DNA, 6 bp, Artificial sequence
  restriction site
tcatga

SEQ ID NO: 247 - DNA, 6 bp, Artificial sequence
  restriction site
tccgga

SEQ ID NO: 248 - DNA, 6 bp, Artificial sequence
  restriction site
tccrac

SEQ ID NO: 249 - DNA, 4 bp, Artificial sequence
  restriction site
tcga

SEQ ID NO: 250 - DNA, 6 bp, Artificial sequence
  restriction site
tcgcga

SEQ ID NO: 251 - DNA, 5 bp, Artificial sequence
  restriction site
  misc_feature (3)..(3): n is a, c, g, or t
tcnga

SEQ ID NO: 252 - DNA, 6 bp, Artificial sequence
  restriction site
  misc_feature (3)..(4): n is a, c, g, or t
tcnnga

SEQ ID NO: 253 - DNA, 6 bp, Artificial sequence
  restriction site
tctaga

SEQ ID NO: 254 - DNA, 6 bp, Artificial sequence
  restriction site
tgatca

SEQ ID NO: 255 - DNA, 4 bp, Artificial sequence
  restriction site
tgca

SEQ ID NO: 256 - DNA, 6 bp, Artificial sequence
  restriction site
tgcgca

SEQ ID NO: 257 - DNA, 30 bp, Artificial sequence
  restriction site
tggcaaacagctattatgggtattatgggt

SEQ ID NO: 258 - DNA, 6 bp, Artificial sequence
  restriction site
tggcca

SEQ ID NO: 259 - DNA, 6 bp, Artificial sequence
  restriction site
tgtaca

SEQ ID NO: 260 - DNA, 4 bp, Artificial sequence
  restriction site
ttaa

SEQ ID NO: 261 - DNA, 8 bp, Artificial sequence
  restriction site
ttaattaa

SEQ ID NO: 262 - DNA, 6 bp, Artificial sequence
  restriction site
ttataa

SEQ ID NO: 263 - DNA, 6 bp, Artificial sequence
  restriction site
ttcgaa

SEQ ID NO: 264 - DNA, 6 bp, Artificial sequence
  restriction site
tttaaa

SEQ ID NO: 265 - DNA, 8 bp, Artificial sequence
  restriction site
vctcgagb

SEQ ID NO: 266 - DNA, 6 bp, Artificial sequence
  restriction site
wccggw

SEQ ID NO: 267 - DNA, 6 bp, Artificial sequence
  restriction site
yacgtr

SEQ ID NO: 268 - DNA, 6 bp, Artificial sequence
  restriction site
yggccr

SEQ ID NO: 269 - DNA, 20 bp, Artificial sequence
  transposase binding site
ctgagagatcccctcataat

SEQ ID NO: 270 - DNA, 20 bp, Artificial sequence
  transposase binding site
attatgaggggatctctcag

SEQ ID NO: 271 - DNA, 20 bp, Artificial sequence
  transposase binding site
gactctctaggggagtatta

SEQ ID NO: 272 - DNA, 20 bp, Artificial sequence
  transposase binding site
taatactcccctagagagtc

SEQ ID NO: 273 - DNA, 20 bp, Artificial sequence
  transposase binding site
ctgagagatcccctcataat

SEQ ID NO: 274 - DNA, 20 bp, Artificial sequence
  transposase binding site
attatgaggggatctctcag

SEQ ID NO: 275 - DNA, 20 bp, Artificial sequence
  transposase binding site
gactctctaggggagtatta

SEQ ID NO: 276 - DNA, 20 bp, Artificial sequence
  transposase binding site
taatactcccctagagagtc

SEQ ID NO: 277 - DNA, 20 bp, Artificial sequence
  transposase binding site
ctgagagatcccctcataat

SEQ ID NO: 278 - DNA, 20 bp, Artificial sequence
  transposase binding site
attatgaggggatctctcag

SEQ ID NO: 279 - DNA, 20 bp, Artificial sequence
  transposase binding site
gactctctaggggagtatta

SEQ ID NO: 280 - DNA, 20 bp, Artificial sequence
  transposase binding site
taatactcccctagagagtc

SEQ ID NO: 281 - DNA, 20 bp, Artificial sequence
  transposase binding site
ctgagagatcccctcataat

SEQ ID NO: 282 - DNA, 20 bp, Artificial sequence
  transposase binding site
attatgaggggatctctcag

SEQ ID NO: 283 - DNA, 20 bp, Artificial sequence
  transposase binding site
gactctctaggggagtatta

SEQ ID NO: 284 - DNA, 20 bp, Artificial sequence
  transposase binding site
taatactcccctagagagtc

SEQ ID NO: 285 - DNA, 29 bp, Artificial sequence
  transposase binding site
ctgatgaatcccctaatgattttggtaaa

SEQ ID NO: 286 - DNA, 29 bp, Artificial sequence
  transposase binding site
tttaccaaaatcattaggggattcatcag

SEQ ID NO: 287 - DNA, 29 bp, Artificial sequence
  transposase binding site
ctgagagatcccctcataatttccccaaa

SEQ ID NO: 288 - DNA, 29 bp, Artificial sequence
  transposase binding site
tttggggaaattatgaggggatctctcag

SEQ ID NO: 289 - DNA, 29 bp, Artificial sequence
  transposase binding site
catggccgtcaacctaagaagccttattt

SEQ ID NO: 290 - DNA, 29 bp, Artificial sequence
  transposase binding site
aaataaggcttcttaggttgacggccatg

SEQ ID NO: 291 - DNA, 29 bp, Artificial sequence
  transposase binding site
catggccgtcaacctaagaaggcataaaa

SEQ ID NO: 292 - DNA, 29 bp, Artificial sequence
  transposase binding site
ttttatgccttcttaggttgacggccatg

SEQ ID NO: 293 - DNA, 29 bp, Artificial sequence
  transposase binding site
cactacagttgcattttgtgttgagttct

SEQ ID NO: 294 - DNA, 29 bp, Artificial sequence
  transposase binding site
agaactcaacacaaaatgcaactgtagtg

SEQ ID NO: 295 - DNA, 29 bp, Artificial sequence
  transposase binding site
cactacagttgcattttgtgtcgtgagtg

SEQ ID NO: 296 - DNA, 29 bp, Artificial sequence
  transposase binding site
cactcacgacacaaaatgcaactgtagtg

SEQ ID NO: 297 - DNA, 29 bp, Artificial sequence
  transposase binding site
cataagcgctaacttaagggttgtggtat

SEQ ID NO: 298 - DNA, 29 bp, Artificial sequence
  transposase binding site
ataccacaacccttaagttagcgcttatg

SEQ ID NO: 299 - DNA, 29 bp, Artificial sequence
  transposase binding site
cataagcgctaacttaagggttgaaccat

SEQ ID NO: 300 - DNA, 29 bp, Artificial sequence
  transposase binding site
atggttcaacccttaagttagcgcttatg

SEQ ID NO: 301 - DNA, 29 bp, Artificial sequence
  transposase binding site
catgcccatcaacttaagaatctagacta

SEQ ID NO: 302 - DNA, 29 bp, Artificial sequence
  transposase binding site
tagtctagattcttaagttgatgggcatg

SEQ ID NO: 303 - DNA, 29 bp, Artificial sequence
  transposase binding site
catgcccatcaacttaagaacaaaaataa

SEQ ID NO: 304 - DNA, 29 bp, Artificial sequence
  transposase binding site
ttatttttgttcttaagttgatgggcatg

SEQ ID NO: 305 - DNA, 29 bp, Artificial sequence
  transposase binding site
catcgccatcaagctaaggaaaaggagat

SEQ ID NO: 306 - DNA, 29 bp, Artificial sequence
  transposase binding site
atctccttttccttagcttgatggcgatg

SEQ ID NO: 307 - DNA, 29 bp, Artificial sequence
  transposase binding site
catcgccatcaagctaacgaaattgaact

SEQ ID NO: 308 - DNA, 29 bp, Artificial sequence
  transposase binding site
agttcaatttcgttagcttgatggcgatg

SEQ ID NO: 309 - DNA, 29 bp, Artificial sequence
  transposase binding site
taatgccgatcagttaaggatcagttcac

SEQ ID NO: 310 - DNA, 29 bp, Artificial sequence
  transposase binding site
gtgaactgatccttaactgatcggcatta

SEQ ID NO: 311 - DNA, 29 bp, Artificial sequence
  transposase binding site
taatgccagtcagttaagcaactgactgg

SEQ ID NO: 312 - DNA, 29 bp, Artificial sequence
  transposase binding site
ccagtcagttgcttaactgactggcatta

SEQ ID NO: 313 - DNA, 29 bp, Artificial sequence
  transposase binding site
taagagagttaatttataatctattttca

SEQ ID NO: 314 - DNA, 29 bp, Artificial sequence
  transposase binding site
tgaaaatagattataaattaactctctta

SEQ ID NO: 315 - DNA, 29 bp, Artificial sequence
  transposase binding site
taaggatgttaatttattaatttaaaagg

SEQ ID NO: 316 - DNA, 29 bp, Artificial sequence
  transposase binding site
ccttttaaattaataaattaacatcctta

SEQ ID NO: 317 - DNA, 29 bp, Artificial sequence
  transposase binding site
ctgactcttatacacaagtagcgtcctga

SEQ ID NO: 318 - DNA, 29 bp, Artificial sequence
  transposase binding site
tcaggacgctacttgtgtataagagtcag

SEQ ID NO: 319 - DNA, 30 bp, Artificial sequence
  transposase binding site
caggcgtgttgattatccaataaaatccca

SEQ ID NO: 320 - DNA, 29 bp, Artificial sequence
  transposase binding site
caggggatcaagatctgatcaagagacag

SEQ ID NO: 321 - DNA, 30 bp, Artificial sequence; identical to SEQ ID NO: 319
  transposase binding site

SEQ ID NO: 322 - DNA, 30 bp, Artificial sequence
  transposase binding site
tgggattttattggataatcaacacgcctg

SEQ ID NO: 323 - DNA, 30 bp, Artificial sequence
  transposase binding site
cacgagtgttgattatccaaaattataaca

SEQ ID NO: 324 - DNA, 30 bp, Artificial sequence
  transposase binding site
tgttataattttggataatcaacactcgtg

SEQ ID NO: 325 - DNA, 30 bp, Artificial sequence
  transposase binding site
cttaacgggtgacaaggcatcctctagcct

SEQ ID NO: 326 - DNA, 30 bp, Artificial sequence
  transposase binding site
aggctagaggatgccttgtcacccgttaag

SEQ ID NO: 327 - DNA, 30 bp, Artificial sequence
  transposase binding site
cttaacgggtgacaacgtagcctacgaagc

SEQ ID NO: 328 - DNA, 30 bp, Artificial sequence
  transposase binding site
gcttcgtaggctacgttgtcacccgttaag

SEQ ID NO: 329 - DNA, 29 bp, Artificial sequence
  transposase binding site
ccttaattccgcaacactatagtttaaca

SEQ ID NO: 330 - DNA, 29 bp, Artificial sequence
  transposase binding site
tgttaaactatagtgttgcggaattaagg

SEQ ID NO: 331 - DNA, 29 bp, Artificial sequence
  transposase binding site
cctcaaatccgcaacctatagggtttagt

SEQ ID NO: 332 - DNA, 29 bp, Artificial sequence
  transposase binding site
actaaaccctataggttgcggatttgagg

SEQ ID NO: 333 - DNA, 29 bp, Artificial sequence
  transposase binding site
cagtacctcacaaagccggttagttgaga

SEQ ID NO: 334 - DNA, 29 bp, Artificial sequence
  transposase binding site
tctcaactaaccggctttgtgaggtactg

SEQ ID NO: 335 - DNA, 29 bp, Artificial sequence
  transposase binding site
cagtacctcacaaagcattctcggctagc

SEQ ID NO: 336 - DNA, 29 bp, Artificial sequence
  transposase binding site
gctagccgagaatgctttgtgaggtactg

SEQ ID NO: 337 - DNA, 37 bp, Artificial sequence
  transposase binding site
ggccgtgaacccataaactgggtaccggattactgat

SEQ ID NO: 338 - DNA, 37 bp, Artificial sequence
  transposase binding site
atcagtaatccggtacccagtttatgggttcacggcc

SEQ ID NO: 339 - DNA, 37 bp, Artificial sequence
  transposase binding site
ggccgtagactcataagcccgcagccacaggcgcatt

SEQ ID NO: 340 - DNA, 37 bp, Artificial sequence
  transposase binding site
aatgcgcctgtggctgcgggcttatgagtctacggcc

SEQ ID NO: 341 - DNA, 37 bp, Artificial sequence
  transposase binding site
gggcttgtctgcattcaaggattcccttttgtacgaa

SEQ ID NO: 342 - DNA, 37 bp, Artificial sequence
  transposase binding site
ttcgtacaaaagggaatccttgaatgcagacaagccc

SEQ ID NO: 343 - DNA, 37 bp, Artificial sequence
  transposase binding site
gggcgtgtctgcattcaacgtaaccagatcatagcgc

SEQ ID NO: 344 - DNA, 37 bp, Artificial sequence
  transposase binding site
gcgctatgatctggttacgttgaatgcagacacgccc

SEQ ID NO: 345 - DNA, 37 bp, Artificial sequence
  transposase binding site
ggctttgttgaataaatcgaacttttgctgagttgaa

SEQ ID NO: 346 - DNA, 37 bp, Artificial sequence
  transposase binding site
ttcaactcagcaaaagttcgatttattcaacaaagcc

SEQ ID NO: 347 - DNA, 37 bp, Artificial sequence
  transposase binding site
ggctttgttgaataaatcagatttcgggtaagtctcc

SEQ ID NO: 348 - DNA, 37 bp, Artificial sequence
  transposase binding site
ggagacttacccgaaatctgatttattcaacaaagcc

SEQ ID NO: 349 - DNA, 28 bp, Artificial sequence
  transposase binding site
ggtgatgctgccaacttactgatttagt

SEQ ID NO: 350 - DNA, 28 bp, Artificial sequence
  transposase binding site
actaaatcagtaagttggcagcatcacc

SEQ ID NO: 351 - DNA, 28 bp, Artificial sequence
  transposase binding site
ggtaatgactccaacttattgatagtgt

SEQ ID NO: 352 - DNA, 28 bp, Artificial sequence
  transposase binding site
acactatcaataagttggagtcattacc

SEQ ID NO: 353 - DNA, 28 bp, Artificial sequence
  transposase binding site
ggtgatgcctctaattagttgaatctga

SEQ ID NO: 354 - DNA, 28 bp, Artificial sequence
  transposase binding site
tcagattcaactaattagaggcatcacc

SEQ ID NO: 355 - DNA, 28 bp, Artificial sequence
  transposase binding site
ggtgatgcttccaattagtagaacatgt

SEQ ID NO: 356 - DNA, 28 bp, Artificial sequence
  transposase binding site
acatgttctactaattggaagcatcacc

SEQ ID NO: 357 - DNA, 28 bp, Artificial sequence
  transposase binding site
ggtcatgcattcaatgcggagcggagat

SEQ ID NO: 358 - DNA, 28 bp, Artificial sequence
  transposase binding site
atctccgctccgcattgaatgcatgacc

SEQ ID NO: 359 - DNA, 28 bp, Artificial sequence
  transposase binding site
ggtcatgtattcaatctggttattagta

SEQ ID NO: 360 - DNA, 28 bp, Artificial sequence
  transposase binding site
tactaataaccagattgaatacatgacc

SEQ ID NO: 361 - DNA, 28 bp, Artificial sequence
  transposase binding site
gagagtttcaaatagataatgatgtggg

SEQ ID NO: 362 - DNA, 28 bp, Artificial sequence
  transposase binding site
cccacatcattatctatttgaaactctc

SEQ ID NO: 363 - DNA, 28 bp, Artificial sequence
  transposase binding site
gagagtttcaagaaagtaatgagaggcg

SEQ ID NO: 364 - DNA, 28 bp, Artificial sequence
  transposase binding site
cgcctctcattactttcttgaaactctc

SEQ ID NO: 365 - DNA, 28 bp, Artificial sequence
  transposase binding site
ggcattggtagttaattatagttatata

SEQ ID NO: 366 - DNA, 28 bp, Artificial sequence
  transposase binding site
tatataactataattaactaccaatgcc

SEQ ID NO: 367 - DNA, 28 bp, Artificial sequence
  transposase binding site
ggtattgttaattaaatatagataactc

SEQ ID NO: 368 - DNA, 28 bp, Artificial sequence
  transposase binding site
gagttatctatatttaattaacaatacc

SEQ ID NO: 369 - DNA, 28 bp, Artificial sequence
  transposase binding site
gatagtgccgtcattaagctatttatat

SEQ ID NO: 370 - DNA, 28 bp, Artificial sequence
  transposase binding site
atataaatagcttaatgacggcactatc

SEQ ID NO: 371 - DNA, 28 bp, Artificial sequence
  transposase binding site
gatagtgtcgtcattaagctacaaattc

SEQ ID NO: 372 - DNA, 28 bp, Artificial sequence
  transposase binding site
gaatttgtagcttaatgacgacactatc

SEQ ID NO: 373 - DNA, 55 bp, Artificial sequence
  transposase binding site
  misc_feature (23)..(46): n is a, c, g, or t
gggagtgttctcatgcagtcatnnnnnnnnnnnnnnnnnnnnnnnnttatattag

SEQ ID NO: 374 - DNA, 55 bp, Artificial sequence
  transposase binding site
  misc_feature (10)..(33): n is a, c, g, or t
ctaatataannnnnnnnnnnnnnnnnnnnnnnnatgactgcatgagaacactccc

SEQ ID NO: 375 - DNA, 55 bp, Artificial sequence
  transposase binding site
  misc_feature (23)..(46): n is a, c, g, or t
gggagtgttctcatgcagtcatnnnnnnnnnnnnnnnnnnnnnnnnatattaagg

SEQ ID NO: 376 - DNA, 55 bp, Artificial sequence
  transposase binding site
  misc_feature (10)..(33): n is a, c, g, or t
ccttaatatnnnnnnnnnnnnnnnnnnnnnnnnatgactgcatgagaacactccc

SEQ ID NO: 377 - DNA, 49 bp, Artificial sequence
  transposase binding site
  misc_feature (23)..(40): n is a, c, g, or t
gggactgttctcaaacagtcatnnnnnnnnnnnnnnnnnngtaatatta

SEQ ID NO: 378 - DNA, 49 bp, Artificial sequence
  transposase binding site
  misc_feature (10)..(27): n is a, c, g, or t
taatattacnnnnnnnnnnnnnnnnnnatgactgtttgagaacagtccc

SEQ ID NO: 379 - DNA, 49 bp, Artificial sequence
  transposase binding site
  misc_feature (23)..(40): n is a, c, g, or t
gggactgttctcaaacagtcatnnnnnnnnnnnnnnnnnngtaaaaata

SEQ ID NO: 380 - DNA, 49 bp, Artificial sequence
  transposase binding site
  misc_feature (10)..(27): n is a, c, g, or t
tatttttacnnnnnnnnnnnnnnnnnnatgactgtttgagaacagtccc

SEQ ID NO: 381 - DNA, 54 bp, Artificial sequence
  transposase binding site
cagtgctggccaaaaagatatccacttttggttttttgtgtgtaacttttttct

SEQ ID NO: 382 - DNA, 54 bp, Artificial sequence
  transposase binding site
agaaaaaagttacacacaaaaaaccaaaagtggatatctttttggccagcactg

SEQ ID NO: 383 - DNA, 54 bp, Artificial sequence
  transposase binding site
gtcacgaccggtttttctataggtgaaaaccaaaaaacacacattgaaaaaaga

SEQ ID NO: 384 - DNA, 54 bp, Artificial sequence
  transposase binding site
tcttttttcaatgtgtgttttttggttttcacctatagaaaaaccggtcgtgac

SEQ ID NO: 385 - DNA, 32 bp, Artificial sequence
  transposase binding site
  misc_feature (1)..(1): N= T, C ou A
nagttgaagtcggaagtttacatacacttaag

SEQ ID NO: 386 - DNA, 32 bp, Artificial sequence
  transposase binding site
  misc_feature (32)..(32): N=G ou A
cttaagtgtatgtaaacttccgacttcaactn

SEQ ID NO: 387 - DNA, 32 bp, Artificial sequence
  transposase binding site
  misc_feature (1)..(1): N = T ou C
nagttgaagtcggaagtttacatacaccttag

SEQ ID NO: 388 - DNA, 32 bp, Artificial sequence
  transposase binding site
  misc_feature (32)..(32): N= G ou A
ctaaggtgtatgtaaacttccgacttcaactn

SEQ ID NO: 389 - DNA, 22 bp, Artificial sequence
  transposase binding site
ctttcgcgtttttcgtgcgccg

SEQ ID NO: 390 - DNA, 22 bp, Artificial sequence
  transposase binding site
cggcgcacgaaaaacgcgaaag

SEQ ID NO: 391 - DNA, 22 bp, Artificial sequence
  transposase binding site
ttttcgcatttatcgtgaaacg

SEQ ID NO: 392 - DNA, 22 bp, Artificial sequence
  transposase binding site
cgtttcacgataaatgcgaaaa

SEQ ID NO: 393 - DNA, 22 bp, Artificial sequence
  transposase binding site
ctttcgcgtttttcgtgcgccg

SEQ ID NO: 394 - DNA, 22 bp, Artificial sequence
  transposase binding site
cggcgcacgaaaaacgcgaaag

SEQ ID NO: 395 - DNA, 23 bp, Artificial sequence
  transposase binding site
gttttcgcatttatcgtgaaacg

SEQ ID NO: 396 - DNA, 23 bp, Artificial sequence
  transposase binding site
cgtttcacgataaatgcgaaaac

SEQ ID NO: 397 - DNA, 22 bp, Artificial sequence
  transposase binding site
ctttcgcgtttttcgtgcgccg

SEQ ID NO: 398 - DNA, 22 bp, Artificial sequence
  transposase binding site
cggcgcacgaaaaacgcgaaag

SEQ ID NO: 399 - DNA, 22 bp, Artificial sequence
  transposase binding site
ttttcgcatttatcgtgaaacg

SEQ ID NO: 400 - DNA, 22 bp, Artificial sequence
  transposase binding site
cgtttcacgataaatgcgaaaa

SEQ ID NO: 401 - DNA, 38 bp, Artificial sequence
  transposase binding site
gaaagcgcaaaaagcacgcggcgcaaaaagcacgcggc

SEQ ID NO: 402 - DNA, 44 bp, Artificial sequence
  transposase binding site
gccgcgtgctttttgcgctttcgccgcgtgctttttgcgctttc

SEQ ID NO: 403 - DNA, 22 bp, Artificial sequence
  transposase binding site
ctttcgcgtttttcgtgcgccg

SEQ ID NO: 404 - DNA, 22 bp, Artificial sequence
  transposase binding site
cggcgcacgaaaaacgcgaaag

SEQ ID NO: 405 - DNA, 41 bp, Artificial sequence
  transposase binding site
tgtggggggacaaaaaagtctcaaactggacaaaaaagatc

SEQ ID NO: 406 - DNA, 41 bp, Artificial sequence
  transposase binding site
gatcttttttgtccagtttgagacttttttgtccgcccaca

SEQ ID NO: 407 - DNA, 41 bp, Artificial sequence
  transposase binding site
gatctattttgttcagttcaagactttattgtccgcccaca

SEQ ID NO: 408 - DNA, 41 bp, Artificial sequence
  transposase binding site
tgtgggcggacaataaagtcttgaactgaacaaaatagatc

SEQ ID NO: 409 - DNA, 50 bp, Artificial sequence
  transposase binding site
ggggtttggggagcaacggaaccaaaaaccaacgtaaggcttagctagag

SEQ ID NO: 410 - DNA, 50 bp, Artificial sequence
  transposase binding site
ctctagctaagccttacgttggtttttggttccgttgctccccaaacccc

SEQ ID NO: 411 - DNA, 50 bp, Artificial sequence
  transposase binding site
ggggtttggggagcaacggaacagaaagtgcacttaagcccagcgcctgc

SEQ ID NO: 412 - DNA, 50 bp, Artificial sequence
  transposase binding site
gcaggcgctgggcttaagtgcactttctgttccgttgctccccaaacccc

SEQ ID NO: 413 - DNA, 50 bp, Artificial sequence
  transposase binding site
ggggtttggggagcaacggaaccaaaaaccaacttaaggcttagctagag

SEQ ID NO: 414 - DNA, 50 bp, Artificial sequence
  transposase binding site
ctctagctaagccttaagttggtttttggttccgttgctccccaaacccc

SEQ ID NO: 415 - DNA, 50 bp, Artificial sequence; identical to SEQ ID NO: 411
  transposase binding site

SEQ ID NO: 416 - DNA, 50 bp, Artificial sequence; identical to SEQ ID NO: 412
  transposase binding site

SEQ ID NO: 417 - DNA, 50 bp, Artificial sequence
  transposase binding site
ggggtttggggagcaacggaaccaaaaaccaacgtaaggcttaccaacgc

SEQ ID NO: 418 - DNA, 50 bp, Artificial sequence
  transposase binding site
gcgttggtaagccttacgttggtttttggttccgttgctccccaaacccc

SEQ ID NO: 419 - DNA, 49 bp, Artificial sequence
  transposase binding site
ggggtttggggagcaatggaaccaaaaaccaacgtaagctctgaatccc

SEQ ID NO: 420 - DNA, 49 bp, Artificial sequence
  transposase binding site
gggattcagagcttacgttggtttttggttccattgctccccaaacccc

SEQ ID NO: 421 - DNA, 50 bp, Artificial sequence
  transposase binding site
ggggtttggggagcaacggaaccaaaaaccaacgtaaggcttaccacctg

SEQ ID NO: 422 - DNA, 50 bp, Artificial sequence
  transposase binding site
caggtggtaagccttacgttggtttttggttccgttgctccccaaacccc

SEQ ID NO: 423 - DNA, 49 bp, Artificial sequence
  transposase binding site
ggggtttggggagcaaggaaccaaaaaccaacgtaagccctaccagcgc

SEQ ID NO: 424 - DNA, 49 bp, Artificial sequence
  transposase binding site
gcgctggtagggcttacgttggtttttggttccttgctccccaaacccc

SEQ ID NO: 425 - DNA, 9 bp, Artificial sequence
  domaine riche en G/C
ggcgatcgc

SEQ ID NO: 426 - DNA, 12 bp, Artificial sequence
  domaine riche en G/C
gcggcgatcggc

SEQ ID NO: 427 - DNA, 6 bp, Artificial sequence
  domaine riche en G/C
ggtcgc

SEQ ID NO: 428 - DNA, 6 bp, Artificial sequence
  domaine riche en G/C
gcgacc

SEQ ID NO: 429 - DNA, 798 bp, Artificial sequence
  figure 12 sequence
  misc_feature (1)..(29): n is a, c, g, or t
  misc_feature (40)..(40): n is a, c, g, or t
  misc_feature (78)..(88): n is a, c, g, or t
  misc_feature (214)..(215): n is a, c, g, or t
  misc_feature (218)..(219): n is a, c, g, or t
  misc_feature (225)..(226): n is a, c, g, or t
  misc_feature (228)..(228): n is a, c, g, or t
  misc_feature (230)..(230): n is a, c, g, or t
  misc_feature (232)..(232): n is a, c, g, or t
  misc_feature (236)..(237): n is a, c, g, or t
  misc_feature (240)..(240): n is a, c, g, or t
  misc_feature (243)..(243): n is a, c, g, or t
  misc_feature (248)..(248): n is a, c, g, or t
  misc_feature (250)..(251): n is a, c, g, or t
  misc_feature (254)..(254): n is a, c, g, or t
  misc_feature (257)..(259): n is a, c, g, or t
  misc_feature (264)..(264): n is a, c, g, or t
  misc_feature (266)..(267): n is a, c, g, or t
  misc_feature (269)..(269): n is a, c, g, or t
  misc_feature (271)..(271): n is a, c, g, or t
  misc_feature (274)..(274): n is a, c, g, or t
  misc_feature (276)..(281): n is a, c, g, or t
  misc_feature (284)..(284): n is a, c, g, or t
  misc_feature (317)..(317): n is a, c, g, or t
  misc_feature (323)..(323): n is a, c, g, or t
  misc_feature (325)..(327): n is a, c, g, or t
  misc_feature (329)..(329): n is a, c, g, or t
  misc_feature (339)..(339): n is a, c, g, or t
  misc_feature (341)..(341): n is a, c, g, or t
  misc_feature (343)..(343): n is a, c, g, or t
  misc_feature (351)..(352): n is a, c, g, or t
  misc_feature (357)..(357): n is a, c, g, or t
  misc_feature (359)..(359): n is a, c, g, or t
  misc_feature (364)..(364): n is a, c, g, or t
  misc_feature (374)..(375): n is a, c, g, or t
  misc_feature (381)..(381): n is a, c, g, or t
  misc_feature (383)..(383): n is a, c, g, or t
  misc_feature (386)..(387): n is a, c, g, or t
  misc_feature (389)..(389): n is a, c, g, or t
  misc_feature (392)..(394): n is a, c, g, or t
  misc_feature (399)..(399): n is a, c, g, or t
  misc_feature (401)..(404): n is a, c, g, or t
  misc_feature (686)..(687): n is a, c, g, or t
  misc_feature (728)..(728): n is a, c, g, or t
  misc_feature (738)..(739): n is a, c, g, or t
  misc_feature (753)..(753): n is a, c, g, or t
  misc_feature (768)..(769): n is a, c, g, or t
  misc_feature (787)..(787): n is a, c, g, or t
  misc_feature (794)..(794): n is a, c, g, or t
nnnnnnnnnnnnnnnnnnnnnnnnnnnnnagctcgccgancactaccagcagaacacccccaccatgttatcctcctnnnnnnnnnnncgacaaccactacctgagcacccagtccgccctgagcaaagaccccaacgagaagcgcgatcacatggtcctgctggagttcgtgaccgccgccgggatcactctcggcatggacgagctgtacanntcnncaatgnnancngnggannggnggncctcngnncgntcnnnctgtncnncnanggngnnnnnntcnttgtgggaggtgatgtccaacttgatgttgacnttgtangnnncnggcagctgcncngncttcttgnncttgnangtggncttgacctcnncgtcgnanggnncnccnnncttcntnnnnagcctctgcttgatctcgcccttcagggcgccgtcctcggggtacatccgctcggaggaggcctcccagcccatggtcttcttctgcattacggggccgtcggaggggaagttggtgccgcgcagcttcaccttgtagatgaactcgccgtcctgcagggaggagtcctgggtcacggtcaccacgccgccgtcctcgaagttcatcacgcgctcccacttgaagccctcggggaaggacagcttcaagtagtcggggatgtcggcggggtgcttcacgtannccttggagccgtacatgaactgaggggacaggatgtcccangcgaagggcnngggccacccttggncaccttcagcttggnngtctgggtgccctcgtangggcggncctc

SEQ ID NO: 430 - DNA, 392 bp, Artificial sequence
  figure 14 sequence
  misc_feature (1)..(19): n is a, c, g, or t
  misc_feature (22)..(22): n is a, c, g, or t
  misc_feature (24)..(24): n is a, c, g, or t
  misc_feature (26)..(27): n is a, c, g, or t
  misc_feature (30)..(34): n is a, c, g, or t
  misc_feature (38)..(38): n is a, c, g, or t
  misc_feature (143)..(152): n is a, c, g, or t
  misc_feature (154)..(165): n is a, c, g, or t
  misc_feature (167)..(167): n is a, c, g, or t
  misc_feature (172)..(172): n is a, c, g, or t
  misc_feature (175)..(175): n is a, c, g, or t
  misc_feature (178)..(182): n is a, c, g, or t
  misc_feature (184)..(190): n is a, c, g, or t
  misc_feature (192)..(193): n is a, c, g, or t
  misc_feature (195)..(196): n is a, c, g, or t
  misc_feature (198)..(201): n is a, c, g, or t
  misc_feature (203)..(224): n is a, c, g, or t
  misc_feature (226)..(229): n is a, c, g, or t
  misc_feature (232)..(235): n is a, c, g, or t
  misc_feature (238)..(248): n is a, c, g, or t
  misc_feature (250)..(250): n is a, c, g, or t
  misc_feature (253)..(253): n is a, c, g, or t
  misc_feature (255)..(256): n is a, c, g, or t
  misc_feature (258)..(262): n is a, c, g, or t
  misc_feature (264)..(267): n is a, c, g, or t
  misc_feature (269)..(271): n is a, c, g, or t
  misc_feature (274)..(278): n is a, c, g, or t
  misc_feature (280)..(281): n is a, c, g, or t
  misc_feature (285)..(287): n is a, c, g, or t
  misc_feature (290)..(290): n is a, c, g, or t
  misc_feature (292)..(298): n is a, c, g, or t
  misc_feature (300)..(302): n is a, c, g, or t
  misc_feature (305)..(311): n is a, c, g, or t
  misc_feature (313)..(324): n is a, c, g, or t
  misc_feature (327)..(330): n is a, c, g, or t
  misc_feature (332)..(333): n is a, c, g, or t
  misc_feature (336)..(337): n is a, c, g, or t
  misc_feature (340)..(341): n is a, c, g, or t
  misc_feature (344)..(348): n is a, c, g, or t
  misc_feature (351)..(356): n is a, c, g, or t
  misc_feature (360)..(383): n is a, c, g, or t
  misc_feature (385)..(388): n is a, c, g, or t
  misc_feature (390)..(392): n is a, c, g, or t
nnnnnnnnnnnnnnnnnnngtnanannggnnnnntccncatcatcggcgcagtgggcatcggcattgccgtggtcatgatatttggcatgatcttcagtatgatcttgtgctgtgctatccgcaggaaccgcgagatggtcannnnnnnnnntnnnnnnnnnnnngngtganggncannnnnannnnnnnanngnnannnnannnnnnnnnnnnnnnnnnnnnncnnnnccnnnnccnnnnnnnnnnncntcntnnannnnncnnnntnnnttnnnnntnntccnnnacntnnnnnnncnnnttnnnnnnntnnnnnnnnnnnncannnncnncannttnntcnnnnnctnnnnnntccnnnnnnnnnnnnnnnnnnnnnnnnannnnannn

SEQ ID NO: 431 - DNA, 673 bp, Artificial sequence
  figure 16 sequence
  misc_feature (1)..(10): n is a, c, g, or t
  misc_feature (15)..(19): n is a, c, g, or t
  misc_feature (24)..(24): n is a, c, g, or t
  misc_feature (34)..(34): n is a, c, g, or t
  misc_feature (60)..(60): n is a, c, g, or t
  misc_feature (63)..(63): n is a, c, g, or t
  misc_feature (71)..(73): n is a, c, g, or t
  misc_feature (107)..(107): n is a, c, g, or t
  misc_feature (170)..(170): n is a, c, g, or t
  misc_feature (186)..(188): n is a, c, g, or t
  misc_feature (252)..(252): n is a, c, g, or t
  misc_feature (259)..(259): n is a, c, g, or t
  misc_feature (268)..(268): n is a, c, g, or t
  misc_feature (288)..(291): n is a, c, g, or t
  misc_feature (338)..(339): n is a, c, g, or t
  misc_feature (341)..(341): n is a, c, g, or t
  misc_feature (346)..(347): n is a, c, g, or t
  misc_feature (359)..(362): n is a, c, g, or t
  misc_feature (364)..(364): n is a, c, g, or t
  misc_feature (366)..(367): n is a, c, g, or t
  misc_feature (370)..(370): n is a, c, g, or t
  misc_feature (373)..(373): n is a, c, g, or t
  misc_feature (376)..(376): n is a, c, g, or t
  misc_feature (378)..(382): n is a, c, g, or t
  misc_feature (384)..(389): n is a, c, g, or t
  misc_feature (393)..(394): n is a, c, g, or t
  misc_feature (407)..(409): n is a, c, g, or t
  misc_feature (412)..(412): n is a, c, g, or t
  misc_feature (414)..(435): n is a, c, g, or t
  misc_feature (437)..(439): n is a, c, g, or t
  misc_feature (442)..(443): n is a, c, g, or t
  misc_feature (445)..(445): n is a, c, g, or t
  misc_feature (447)..(448): n is a, c, g, or t
  misc_feature (450)..(450): n is a, c, g, or t
  misc_feature (452)..(458): n is a, c, g, or t
  misc_feature (466)..(466): n is a, c, g, or t
  misc_feature (470)..(470): n is a, c, g, or t
  misc_feature (473)..(474): n is a, c, g, or t
  misc_feature (482)..(486): n is a, c, g, or t
  misc_feature (488)..(490): n is a, c, g, or t
  misc_feature (495)..(508): n is a, c, g, or t
  misc_feature (510)..(512): n is a, c, g, or t
  misc_feature (514)..(514): n is a, c, g, or t
  misc_feature (516)..(532): n is a, c, g, or t
  misc_feature (534)..(539): n is a, c, g, or t
  misc_feature (541)..(559): n is a, c, g, or t
  misc_feature (561)..(562): n is a, c, g, or t
  misc_feature (564)..(568): n is a, c, g, or t
  misc_feature (572)..(595): n is a, c, g, or t
  misc_feature (597)..(599): n is a, c, g, or t
  misc_feature (606)..(609): n is a, c, g, or t
  misc_feature (611)..(611): n is a, c, g, or t
  misc_feature (613)..(617): n is a, c, g, or t
  misc_feature (619)..(633): n is a, c, g, or t
  misc_feature (635)..(635): n is a, c, g, or t
  misc_feature (638)..(664): n is a, c, g, or t
  misc_feature (666)..(673): n is a, c, g, or t
nnnnnnnnnnctgannnnntgccnaatatcatgnccacggcaatgccgatgcccactgcnccnatgatgtnnnaatttattgtcgaagacctctttgatggcatcangacaggacttcacggtgaaggtttcgagtacgtccttcttggggcagatgtctgagataaacngctcctcgcccttgannnaaccacagcagttcaacgcatagtggatggctttcagcgtttcccgctggggctcattgaacanctcctcncccttgaangtgtccttgtaaaactcctnnnnttccttaatcacctcatccttgtgggaatatccccagatggccgcanntngatcnntggcgaatatcnnnnanannaanccnaanannnnnannnnnntgcnntgggactcctgcnnnccngnnnnnnnnnnnnnnnnnnnnnngnnntannangnnanannnnnnnatcctacntttnacnnccctaccnnnnngnnnaattnnnnnnnnnnnnnncnnncncnnnnnnnnnnnnnnnnncnnnnnncnnnnnnnnnnnnnnnnnnntnncnnnnncccnnnnnnnnnnnnnnnnnnnnnnnncnnnccccacnnnnangnnnnnannnnnnnnnnnnnnncnctnnnnnnnnnnnnnnnnnnnnnnnnnnntnnnnnnnn

SEQ ID NO: 432 - DNA, 671 bp, Artificial sequence
  figure 19 sequence
  misc_feature (1)..(10): n is a, c, g, or t
  misc_feature (12)..(24): n is a, c, g, or t
  misc_feature (27)..(28): n is a, c, g, or t
  misc_feature (30)..(38): n is a, c, g, or t
  misc_feature (40)..(42): n is a, c, g, or t
  misc_feature (46)..(51): n is a, c, g, or t
  misc_feature (53)..(54): n is a, c, g, or t
  misc_feature (56)..(61): n is a, c, g, or t
  misc_feature (63)..(64): n is a, c, g, or t
  misc_feature (66)..(69): n is a, c, g, or t
  misc_feature (71)..(71): n is a, c, g, or t
  misc_feature (75)..(75): n is a, c, g, or t
  misc_feature (77)..(79): n is a, c, g, or t
  misc_feature (81)..(84): n is a, c, g, or t
  misc_feature (87)..(87): n is a, c, g, or t
  misc_feature (93)..(93): n is a, c, g, or t
  misc_feature (95)..(96): n is a, c, g, or t
  misc_feature (98)..(98): n is a, c, g, or t
  misc_feature (102)..(103): n is a, c, g, or t
  misc_feature (108)..(109): n is a, c, g, or t
  misc_feature (112)..(114): n is a, c, g, or t
  misc_feature (116)..(117): n is a, c, g, or t
  misc_feature (120)..(120): n is a, c, g, or t
  misc_feature (122)..(122): n is a, c, g, or t
  misc_feature (126)..(126): n is a, c, g, or t
  misc_feature (132)..(132): n is a, c, g, or t
  misc_feature (135)..(135): n is a, c, g, or t
  misc_feature (137)..(137): n is a, c, g, or t
  misc_feature (141)..(141): n is a, c, g, or t
  misc_feature (150)..(150): n is a, c, g, or t
  misc_feature (152)..(152): n is a, c, g, or t
  misc_feature (159)..(159): n is a, c, g, or t
  misc_feature (163)..(167): n is a, c, g, or t
  misc_feature (170)..(171): n is a, c, g, or t
  misc_feature (181)..(182): n is a, c, g, or t
  misc_feature (186)..(186): n is a, c, g, or t
  misc_feature (189)..(189): n is a, c, g, or t
  misc_feature (195)..(195): n is a, c, g, or t
  misc_feature (198)..(198): n is a, c, g, or t
  misc_feature (203)..(204): n is a, c, g, or t
  misc_feature (209)..(209): n is a, c, g, or t
  misc_feature (212)..(212): n is a, c, g, or t
  misc_feature (228)..(230): n is a, c, g, or t
  misc_feature (234)..(234): n is a, c, g, or t
  misc_feature (240)..(240): n is a, c, g, or t
  misc_feature (246)..(248): n is a, c, g, or t
  misc_feature (253)..(253): n is a, c, g, or t
  misc_feature (266)..(266): n is a, c, g, or t
  misc_feature (270)..(270): n is a, c, g, or t
  misc_feature (276)..(276): n is a, c, g, or t
  misc_feature (279)..(279): n is a, c, g, or t
  misc_feature (285)..(288): n is a, c, g, or t
  misc_feature (290)..(290): n is a, c, g, or t
  misc_feature (292)..(292): n is a, c, g, or t
  misc_feature (294)..(294): n is a, c, g, or t
  misc_feature (297)..(297): n is a, c, g, or t
  misc_feature (305)..(305): n is a, c, g, or t
  misc_feature (312)..(312): n is a, c, g, or t
  misc_feature (318)..(320): n is a, c, g, or t
  misc_feature (363)..(367): n is a, c, g, or t
  misc_feature (370)..(370): n is a, c, g, or t
  misc_feature (378)..(381): n is a, c, g, or t
  misc_feature (394)..(398): n is a, c, g, or t
  misc_feature (400)..(401): n is a, c, g, or t
  misc_feature (408)..(408): n is a, c, g, or t
  misc_feature (411)..(412): n is a, c, g, or t
  misc_feature (419)..(419): n is a, c, g, or t
  misc_feature (423)..(423): n is a, c, g, or t
  misc_feature (430)..(430): n is a, c, g, or t
  misc_feature (433)..(433): n is a, c, g, or t
  misc_feature (435)..(436): n is a, c, g, or t
  misc_feature (438)..(438): n is a, c, g, or t
  misc_feature (441)..(441): n is a, c, g, or t
  misc_feature (443)..(443): n is a, c, g, or t
  misc_feature (447)..(447): n is a, c, g, or t
  misc_feature (449)..(449): n is a, c, g, or t
  misc_feature (451)..(451): n is a, c, g, or t
  misc_feature (453)..(464): n is a, c, g, or t
  misc_feature (467)..(472): n is a, c, g, or t
  misc_feature (476)..(476): n is a, c, g, or t
  misc_feature (478)..(479): n is a, c, g, or t
  misc_feature (481)..(481): n is a, c, g, or t
  misc_feature (483)..(484): n is a, c, g, or t
  misc_feature (487)..(489): n is a, c, g, or t
  misc_feature (491)..(491): n is a, c, g, or t
  misc_feature (493)..(497): n is a, c, g, or t
  misc_feature (500)..(502): n is a, c, g, or t
  misc_feature (505)..(511): n is a, c, g, or t
  misc_feature (513)..(513): n is a, c, g, or t
  misc_feature (515)..(517): n is a, c, g, or t
  misc_feature (519)..(523): n is a, c, g, or t
  misc_feature (525)..(526): n is a, c, g, or t
  misc_feature (528)..(544): n is a, c, g, or t
  misc_feature (546)..(549): n is a, c, g, or t
  misc_feature (552)..(553): n is a, c, g, or t
  misc_feature (555)..(555): n is a, c, g, or t
  misc_feature (557)..(571): n is a, c, g, or t
  misc_feature (574)..(584): n is a, c, g, or t
  misc_feature (586)..(589): n is a, c, g, or t
  misc_feature (591)..(594): n is a, c, g, or t
  misc_feature (597)..(606): n is a, c, g, or t
  misc_feature (609)..(610): n is a, c, g, or t
  misc_feature (612)..(617): n is a, c, g, or t
  misc_feature (621)..(622): n is a, c, g, or t
  misc_feature (624)..(624): n is a, c, g, or t
  misc_feature (627)..(630): n is a, c, g, or t
  misc_feature (632)..(633): n is a, c, g, or t
  misc_feature (635)..(635): n is a, c, g, or t
  misc_feature (641)..(641): n is a, c, g, or t
  misc_feature (643)..(643): n is a, c, g, or t
  misc_feature (645)..(646): n is a, c, g, or t
  misc_feature (649)..(654): n is a, c, g, or t
  misc_feature (656)..(656): n is a, c, g, or t
  misc_feature (658)..(664): n is a, c, g, or t
  misc_feature (666)..(666): n is a, c, g, or t
  misc_feature (668)..(671): n is a, c, g, or t
nnnnnnnnnntnnnnnnnnnnnnnagnncnnnnnnnnngnnnctcnnnnnntnngnnnnnngnntnnnncngcantnnngnnnntgnccgtgncnngncccnncctcnngannncnntgncntacngcgtgnagngnttcngccgctacncntaccacntgannnnncannacttcttcanntccnccntgcccnaangctanntccangancgcaccatcttcttcnnngacnacggcnactacnnnacccncgccgaggtgaanttcnagggcnacnccctgnnnnancncntcnagctgaanggcatcnacttcnnngaggacggcaacatcctggggcacaagctggagtacaactacnnnnnccncaacgtcnnnntcatggccgactnnnnnannaacggcntcnnggtgaanttcnagatccnccncnncntcnangacngnancnnnnnnnnnnnngannnnnnccancnnancnnccnnntngnnnnnggnnnctnnnnnnntncnnnannnnnannannnnnnnnnnnnnnnnncnnnnaannanannnnnnnnnnnnnnnacnnnnnnnnnnncnnnntnnnnaannnnnnnnnnacnncnnnnnntccnntngannnntnnantgtatntnanngtnnnnnntntnnnnnnntntnnnn

SEQ ID NO: 433 - DNA, 812 bp, Artificial sequence
  figure 19 sequence
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggataataattttttttttttttttttttttagatgtgtataagagacagctgggcacgcgtataagcagctgagatgtgtataagagacag

SEQ ID NO: 434 - DNA, 270 bp, Artificial sequence
  figure 19 sequence
  misc_feature (4)..(4): n is a, c, g, or t
  misc_feature (10)..(10): n is a, c, g, or t
  misc_feature (13)..(13): n is a, c, g, or t
  misc_feature (15)..(15): n is a, c, g, or t
  misc_feature (19)..(19): n is a, c, g, or t
  misc_feature (28)..(28): n is a, c, g, or t
  misc_feature (30)..(30): n is a, c, g, or t
  misc_feature (37)..(37): n is a, c, g, or t
  misc_feature (41)..(45): n is a, c, g, or t
  misc_feature (48)..(49): n is a, c, g, or t
  misc_feature (59)..(60): n is a, c, g, or t
  misc_feature (64)..(64): n is a, c, g, or t
  misc_feature (67)..(67): n is a, c, g, or t
  misc_feature (73)..(73): n is a, c, g, or t
  misc_feature (76)..(76): n is a, c, g, or t
  misc_feature (81)..(82): n is a, c, g, or t
  misc_feature (87)..(87): n is a, c, g, or t
  misc_feature (90)..(90): n is a, c, g, or t
  misc_feature (106)..(108): n is a, c, g, or t
  misc_feature (112)..(112): n is a, c, g, or t
  misc_feature (118)..(118): n is a, c, g, or t
  misc_feature (124)..(126): n is a, c, g, or t
  misc_feature (131)..(131): n is a, c, g, or t
  misc_feature (144)..(144): n is a, c, g, or t
  misc_feature (148)..(148): n is a, c, g, or t
  misc_feature (154)..(154): n is a, c, g, or t
  misc_feature (157)..(157): n is a, c, g, or t
  misc_feature (163)..(166): n is a, c, g, or t
  misc_feature (168)..(168): n is a, c, g, or t
  misc_feature (170)..(170): n is a, c, g, or t
  misc_feature (172)..(172): n is a, c, g, or t
  misc_feature (175)..(175): n is a, c, g, or t
  misc_feature (183)..(183): n is a, c, g, or t
  misc_feature (190)..(190): n is a, c, g, or t
  misc_feature (196)..(198): n is a, c, g, or t
  misc_feature (241)..(245): n is a, c, g, or t
  misc_feature (248)..(248): n is a, c, g, or t
  misc_feature (256)..(259): n is a, c, g, or t
tacngcgtgnagngnttcngccgctacncntaccacntgannnnncannacttcttcanntccnccntgcccnaangctanntccangancgcaccatcttcttcnnngacnacggcnactacnnnacccncgccgaggtgaanttcnagggcnacnccctgnnnnancncntcnagctgaanggcatcnacttcnnngaggacggcaacatcctggggcacaagctggagtacaactacnnnnnccncaacgtcnnnntcatggccgac

SEQ ID NO: 435 - DNA, 270 bp, Artificial sequence
  figure 19 sequence
tacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgac

SEQ ID NO: 436 - DNA, 8 bp, Artificial sequence
  molecule of the complex
tgcagctg

SEQ ID NO: 437 - DNA, 56 bp, Artificial sequence
  molecule of the complex
tttttttttttttttttttttttttttggcgatcgctttttttttttttttttttt

SEQ ID NO: 438 - DNA, 56 bp, Artificial sequence
  molecule of the complex
ttttttttttttttttttttgcgatcgccttttttttttttttttgatacatgttt

SEQ ID NO: 439 - DNA, 8 bp, Artificial sequence
  molecule of the complex
ctgtaagc

SEQ ID NO: 440 - DNA, 22 bp, Artificial sequence
  molecule of the complex
cagctgcagacaaagcttacag

SEQ ID NO: 441 - DNA, 34 bp, Artificial sequence
  molecule of the complex
ttttttttttttttttttttttcatatgccaagt

SEQ ID NO: 442 - DNA, 83 bp, Artificial sequence
  molecule of the complex
ttttttttttttttttttttgcgatcgccttttttttttttttttgatacatgtttagatgtgtataagagacagctgtaagc

SEQ ID NO: 443 - DNA, 83 bp, Artificial sequence; identical to SEQ ID NO: 442
  molecule of the complex

SEQ ID NO: 444 - DNA, 72 bp, Artificial sequence
  molecule of the complex
ttttttttttttttttgcgatcgcctttttttttttttgatacatttagatgtgtataagagacaggatgat

SEQ ID NO: 445 - DNA, 6 bp, Artificial sequence
  molecule of the complex
atcatc

SEQ ID NO: 446 - DNA, 25 bp, Artificial sequence
  molecule of the complex
tttttttttttttttttgatagtag

SEQ ID NO: 447 - DNA, 48 bp, Artificial sequence
  molecule of the complex
tttttttttttttttttttttttggcgatcgctttttttttttttttt

SEQ ID NO: 448 - DNA, 47 bp, Artificial sequence
  molecule of the complex
ttttttttttttttttgcgatcgcctttttttttttttgatacattt

SEQ ID NO: 449 - DNA, 6 bp, Artificial sequence
  molecule of the complex
gatgat

SEQ ID NO: 450 - DNA, 36 bp, Artificial sequence
  molecule of the complex
acttggttaattaatttttttttttttttttttttt

SEQ ID NO: 451 - DNA, 8 bp, Artificial sequence
  molecule of the complex
ctactatc

SEQ ID NO: 452 - DNA, 53 bp, Artificial sequence
  molecule of the complex
tttttttttttttttttttttttttttggtcgctttttttttttttttttttt

SEQ ID NO: 453 - DNA, 53 bp, Artificial sequence
  molecule of the complex
ttttttttttttttttttttgcgaccttttttttttttttttgatacatgttt

SEQ ID NO: 454 - DNA, 22 bp, Artificial sequence
  molecule of the complex
tttttttttttttttttttttt

SEQ ID NO: 455 - DNA, 28 bp, Artificial sequence
  molecule of the complex
ttttttttttttttttttttttccaagt

SEQ ID NO: 456 - DNA, 59 bp, Artificial sequence
  molecule of the complex
tttttttttttttttttttttttttttgcggcgatcggctttttttttttttttttttt

SEQ ID NO: 457 - DNA, 59 bp, Artificial sequence
  molecule of the complex
ttttttttttttttttttttgccgatcgccgcttttttttttttttttgatacatgttt

SEQ ID NO: 458 - DNA, 8 bp, Artificial sequence
  molecule of the complex
gtgcccag

SEQ ID NO: 459 - DNA, 53 bp, Artificial sequence
  molecule of the complex
tttctcgatcattttttttttttttttggtcgctttttttttttttttttttt

SEQ ID NO: 460 - DNA, 53 bp, Artificial sequence
  molecule of the complex
ttttttttttttttttttttgcgaccttttttttttttttttttttttttttt

SEQ ID NO: 461 - DNA, 8 bp, Artificial sequence
  molecule of the complex
ctgcttat

SEQ ID NO: 462 - DNA, 40 bp, Artificial sequence
  molecule of the complex
tacaagtccggataataatttttttttttttttttttttt

SEQ ID NO: 463 - DNA, 20 bp, Artificial sequence
  molecule of the complex
ctgggcacgcgtataagcag

SEQ ID NO: 464 - DNA, 28 bp, Artificial sequence
  molecule of the complex
taataatttttttttttttttttttttt

SEQ ID NO: 465 - DNA, 34 bp, Artificial sequence
  molecule of the complex
tacaagtaataatttttttttttttttttttttt

SEQ ID NO: 466 - DNA, 56 bp, Artificial sequence
  molecule of the complex
tttctcgatcattttttttttttttttggcgatcgctttttttttttttttttttt

SEQ ID NO: 467 - DNA, 56 bp, Artificial sequence
  molecule of the complex
ttttttttttttttttttttgcgatcgccttttttttttttttttttttttttttt

SEQ ID NO: 468 - DNA, 59 bp, Artificial sequence
  molecule of the complex
tttctcgatcattttttttttttttttgcggcgatcggctttttttttttttttttttt

SEQ ID NO: 469 - DNA, 59 bp, Artificial sequence
  molecule of the complex
ttttttttttttttttttttgccgatcgccgcttttttttttttttttttttttttttt

SEQ ID NO: 470 - DNA, 44 bp, Artificial sequence
  molecule of the complex
ttttttttttttttttttttttggtcgctttttttttttttttt

SEQ ID NO: 471 - DNA, 50 bp, Artificial sequence
  molecule of the complex
ttttttttttttttttttttttgcggcgatcggctttttttttttttttt

SEQ ID NO: 472 - DNA, 50 bp, Artificial sequence
  molecule of the complex
ttttttttttttttttgccgatcgccgctttttttttttttgatacattt

SEQ ID NO: 473 - DNA, 30 bp, Artificial sequence
  molecule of the complex
ttaattaatttttttttttttttttttttt

SEQ ID NO: 474 - DNA, 44 bp, Artificial sequence
  molecule of the complex
ttttttttttttttttgcgacctttttttttttttgatacattt

SEQ ID NO: 475 - DNA, 42 bp, Artificial sequence
  molecule of the complex
acttggcatatgttaattaatttttttttttttttttttttt

SEQ ID NO: 476 - DNA, 47 bp, Artificial sequence
  molecule of the complex
ttttttttttttttttttttttggcgatcgctttttttttttttttt

SEQ ID NO: 477 - DNA, 29 bp, Artificial sequence
  molecule of the complex
gatgattttttttttttttttttatcatc

SEQ ID NO: 478 - DNA, 48 bp, Artificial sequence
  molecule of the complex
tttctcgatcattattattttttggcgatcgctttttttttttttttt

SEQ ID NO: 479 - DNA, 46 bp, Artificial sequence
  molecule of the complex
ttttttttttttttttgcgatcgccttttttttttttttttttttt

SEQ ID NO: 480 - DNA, 28 bp, Artificial sequence
  molecule of the complex
gacgaatatttttttttttttttttttt

SEQ ID NO: 481 - DNA, 35 bp, Artificial sequence
  molecule of the complex
ttttttttttttttttttattaattattacttgta

SEQ ID NO: 482 - DNA, 29 bp, Artificial sequence
  molecule of the complex
ttttttttttttttttttattaattatta

SEQ ID NO: 483 - DNA, 41 bp, Artificial sequence
  molecule of the complex
ttttttttttttttttttattaattattatccggacttgta

SEQ ID NO: 484 - DNA, 45 bp, Artificial sequence
  molecule of the complex
tttctcgatcattattattttttggtcgctttttttttttttttt

SEQ ID NO: 485 - DNA, 43 bp, Artificial sequence
  molecule of the complex
ttttttttttttttttgcgaccttttttttttttttttttttt

SEQ ID NO: 486 - DNA, 51 bp, Artificial sequence
  molecule of the complex
tttctcgatcattattattttttgcggcgatcggctttttttttttttttt

SEQ ID NO: 487 - DNA, 49 bp, Artificial sequence
  molecule of the complex
ttttttttttttttttgccgatcgccgcttttttttttttttttttttt

SEQ ID NO: 488 - DNA, 32 bp, Artificial sequence
  molecule of the complex
cacgtgttttttttttttttttttttcacgtg

SEQ ID NO: 489 - DNA, 94 bp, Artificial sequence; identical to SEQ ID NO: 8
  molecule of the complex

SEQ ID NO: 490 - DNA, 83 bp, Artificial sequence
  molecule of the complex
tgcagctgagatgtgtataagagacagtttttttttttttttttttttttttttggcgatcgctttttttttttttttttttt

SEQ ID NO: 491 - DNA, 83 bp, Artificial sequence
  molecule of the complex
ttttttttttttttttttttgcgatcgccttttttttttttttttgatacatgtttctgtctcttatacacatctctgtaagc

SEQ ID NO: 492 - DNA, 94 bp, Artificial sequence
  molecule of the complex
ctgtctcttatacacatctcagctgcagacaaagcttacagagatgtgtataagagacagttttttttttttttttttttttcatatgccaagt

SEQ ID NO: 493 - DNA, 80 bp, Artificial sequence
  molecule of the complex
tgcagctgctgtctcttatacacatcttttttttttttttttttttttttttttggtcgctttttttttttttttttttt

SEQ ID NO: 494 - DNA, 80 bp, Artificial sequence
  molecule of the complex
ttttttttttttttttttttgcgaccttttttttttttttttgatacatgtttagatgtgtataagagacagctgtaagc

SEQ ID NO: 495 - DNA, 80 bp, Artificial sequence
  molecule of the complex
tgcagctgagatgtgtataagagacagtttttttttttttttttttttttttttggtcgctttttttttttttttttttt

SEQ ID NO: 496 - DNA, 80 bp, Artificial sequence
  molecule of the complex
ttttttttttttttttttttgcgaccttttttttttttttttgatacatgtttctgtctcttatacacatctctgtaagc

SEQ ID NO: 497 - DNA, 86 bp, Artificial sequence
  molecule of the complex
tgcagctgctgtctcttatacacatcttttttttttttttttttttttttttttgcggcgatcggctttttttttttttttttttt

SEQ ID NO: 498 - DNA, 86 bp, Artificial sequence
  molecule of the complex
ttttttttttttttttttttgccgatcgccgcttttttttttttttttgatacatgtttagatgtgtataagagacagctgtaagc

SEQ ID NO: 499 - DNA, 82 bp, Artificial sequence
  molecule of the complex
agatgtgtataagagacagcagctgcagacaaagcttacagctgtctcttatacacatcttttttttttttttttttttttt

SEQ ID NO: 500 - DNA, 82 bp, Artificial sequence
  molecule of the complex
ctgtctcttatacacatctcagctgcagacaaagcttacagagatgtgtataagagacagtttttttttttttttttttttt

SEQ ID NO: 501 - DNA, 86 bp, Artificial sequence
  molecule of the complex
tgcagctgagatgtgtataagagacagtttttttttttttttttttttttttttgcggcgatcggctttttttttttttttttttt

SEQ ID NO: 502 - DNA, 86 bp, Artificial sequence
  molecule of the complex
ttttttttttttttttttttgccgatcgccgcttttttttttttttttgatacatgtttctgtctcttatacacatctctgtaagc

SEQ ID NO: 503 - DNA, 80 bp, Artificial sequence
  molecule of the complex
gtgcccagctgtctcttatacacatcttttctcgatcattttttttttttttttggtcgctttttttttttttttttttt

SEQ ID NO: 504 - DNA, 80 bp, Artificial sequence
  molecule of the complex
ttttttttttttttttttttgcgacctttttttttttttttttttttttttttagatgtgtataagagacagctgcttat

SEQ ID NO: 505 - DNA, 80 bp, Artificial sequence
  molecule of the complex
gtgcccagagatgtgtataagagacagtttctcgatcattttttttttttttttggtcgctttttttttttttttttttt

SEQ ID NO: 506 - DNA, 80 bp, Artificial sequence
  molecule of the complex
ttttttttttttttttttttgcgacctttttttttttttttttttttttttttctgtctcttatacacatctctgcttat

SEQ ID NO: 507 - DNA, 98 bp, Artificial sequence
  molecule of the complex
tacaagtccggataataattttttttttttttttttttttagatgtgtataagagacagctgggcacgcgtataagcagctgtctcttatacacatct

SEQ ID NO: 508 - DNA, 98 bp, Artificial sequence
  molecule of the complex
tacaagtccggataataattttttttttttttttttttttctgtctcttatacacatctctgggcacgcgtataagcagagatgtgtataagagacag

SEQ ID NO: 509 - DNA, 86 bp, Artificial sequence
  molecule of the complex
taataattttttttttttttttttttttagatgtgtataagagacagctgggcacgcgtataagcagctgtctcttatacacatct

SEQ ID NO: 510 - DNA, 86 bp, Artificial sequence
  molecule of the complex
taataattttttttttttttttttttttctgtctcttatacacatctctgggcacgcgtataagcagagatgtgtataagagacag

SEQ ID NO: 511 - DNA, 92 bp, Artificial sequence
  molecule of the complex
tacaagtaataattttttttttttttttttttttagatgtgtataagagacagctgggcacgcgtataagcagctgtctcttatacacatct

SEQ ID NO: 512 - DNA, 92 bp, Artificial sequence
  molecule of the complex
tacaagtaataattttttttttttttttttttttctgtctcttatacacatctctgggcacgcgtataagcagagatgtgtataagagacag

SEQ ID NO: 513 - DNA, 63 bp, Artificial sequence; identical to SEQ ID NO: 10
  molecule of the complex

SEQ ID NO: 514 - DNA, 63 bp, Artificial sequence
  molecule of the complex
acttggttaattaattttttttttttttttttttttctgtctcttatacacatctctactatc

SEQ ID NO: 515 - DNA, 57 bp, Artificial sequence
  molecule of the complex
ttaattaattttttttttttttttttttttagatgtgtataagagacagctactatc

SEQ ID NO: 516 - DNA, 57 bp, Artificial sequence
  molecule of the complex
ttaattaattttttttttttttttttttttctgtctcttatacacatctctactatc

SEQ ID NO: 517 - DNA, 69 bp, Artificial sequence
  molecule of the complex
acttggcatatgttaattaattttttttttttttttttttttagatgtgtataagagacagctactatc

SEQ ID NO: 518 - DNA, 69 bp, Artificial sequence
  molecule of the complex
acttggcatatgttaattaattttttttttttttttttttttctgtctcttatacacatctctactatc

SEQ ID NO: 519 - DNA, 62 bp, Artificial sequence
  molecule of the complex
tattcgtcctgtctcttatacacatctttttttttttttttttttattaattattacttgta

SEQ ID NO: 520 - DNA, 62 bp, Artificial sequence
  molecule of the complex
tattcgtcagatgtgtataagagacagttttttttttttttttttattaattattacttgta

SEQ ID NO: 521 - DNA, 56 bp, Artificial sequence
  molecule of the complex
tattcgtcctgtctcttatacacatctttttttttttttttttttattaattatta

SEQ ID NO: 522 - DNA, 56 bp, Artificial sequence
  molecule of the complex
tattcgtcagatgtgtataagagacagttttttttttttttttttattaattatta

SEQ ID NO: 523 - DNA, 68 bp, Artificial sequence
  molecule of the complex
tattcgtcctgtctcttatacacatctttttttttttttttttttattaattattatccggacttgta

SEQ ID NO: 524 - DNA, 68 bp, Artificial sequence
  molecule of the complex
tattcgtcagatgtgtataagagacagttttttttttttttttttattaattattatccggacttgta

SEQ ID NO: 525 - DNA, 83 bp, Artificial sequence
  molecule of the complex
gtgcccagctgtctcttatacacatcttttctcgatcattttttttttttttttggcgatcgctttttttttttttttttttt

SEQ ID NO: 526 - DNA, 83 bp, Artificial sequence
  molecule of the complex
ttttttttttttttttttttgcgatcgcctttttttttttttttttttttttttttagatgtgtataagagacagctgcttat

SEQ ID NO: 527 - DNA, 83 bp, Artificial sequence
  molecule of the complex
gtgcccagagatgtgtataagagacagtttctcgatcattttttttttttttttggcgatcgctttttttttttttttttttt

SEQ ID NO: 528 - DNA, 83 bp, Artificial sequence
  molecule of the complex
ttttttttttttttttttttgcgatcgcctttttttttttttttttttttttttttctgtctcttatacacatctctgcttat

SEQ ID NO: 529 - DNA, 86 bp, Artificial sequence
  molecule of the complex
gtgcccagctgtctcttatacacatcttttctcgatcattttttttttttttttgcggcgatcggctttttttttttttttttttt

SEQ ID NO: 530 - DNA, 86 bp, Artificial sequence
  molecule of the complex
ttttttttttttttttttttgccgatcgccgctttttttttttttttttttttttttttagatgtgtataagagacagctgcttat

SEQ ID NO: 531 - DNA, 86 bp, Artificial sequence
  molecule of the complex
gtgcccagagatgtgtataagagacagtttctcgatcattttttttttttttttgcggcgatcggctttttttttttttttttttt

SEQ ID NO: 532 - DNA, 86 bp, Artificial sequence
  molecule of the complex
ttttttttttttttttttttgccgatcgccgctttttttttttttttttttttttttttctgtctcttatacacatctctgcttat

SEQ ID NO: 533 - DNA, 116 bp, Artificial sequence; identical to SEQ ID NO: 9
  molecule of the complex

SEQ ID NO: 534 - DNA, 72 bp, Artificial sequence; identical to SEQ ID NO: 444
  molecule of the complex

SEQ ID NO: 535 - DNA, 116 bp, Artificial sequence
  molecule of the complex
atcatcagatgtgtataagagacagtttttttttttttttttgatagtagagatgtgtataagagacagttttttttttttttttttttttggcgatcgctttttttttttttttt

SEQ ID NO: 536 - DNA, 72 bp, Artificial sequence
  molecule of the complex
ttttttttttttttttgcgatcgcctttttttttttttgatacatttctgtctcttatacacatctgatgat

SEQ ID NO: 537 - DNA, 113 bp, Artificial sequence
  molecule of the complex
atcatcctgtctcttatacacatcttttttttttttttttttgatagtagctgtctcttatacacatctttttttttttttttttttttttggtcgctttttttttttttttt

SEQ ID NO: 538 - DNA, 69 bp, Artificial sequence
  molecule of the complex
ttttttttttttttttgcgacctttttttttttttgatacatttagatgtgtataagagacaggatgat

SEQ ID NO: 539 - DNA, 113 bp, Artificial sequence
  molecule of the complex
atcatcagatgtgtataagagacagtttttttttttttttttgatagtagagatgtgtataagagacagttttttttttttttttttttttggtcgctttttttttttttttt

SEQ ID NO: 540 - DNA, 69 bp, Artificial sequence
  molecule of the complex
ttttttttttttttttgcgacctttttttttttttgatacatttctgtctcttatacacatctgatgat

SEQ ID NO: 541 - DNA, 119 bp, Artificial sequence
  molecule of the complex
atcatcctgtctcttatacacatcttttttttttttttttttgatagtagctgtctcttatacacatctttttttttttttttttttttttgcggcgatcggctttttttttttttttt

SEQ ID NO: 542 - DNA, 75 bp, Artificial sequence
  molecule of the complex
ttttttttttttttttgccgatcgccgctttttttttttttgatacatttagatgtgtataagagacaggatgat

SEQ ID NO: 543 - DNA, 119 bp, Artificial sequence
  molecule of the complex
atcatcagatgtgtataagagacagtttttttttttttttttgatagtagagatgtgtataagagacagttttttttttttttttttttttgcggcgatcggctttttttttttttttt

SEQ ID NO: 544 - DNA, 75 bp, Artificial sequence
  molecule of the complex
ttttttttttttttttgccgatcgccgctttttttttttttgatacatttctgtctcttatacacatctgatgat

SEQ ID NO: 545 - DNA, 74 bp, Artificial sequence
  molecule of the complex
gatagtagctgtctcttatacacatctttttttttttttttttttttttggcgatcgctttttttttttttttt

SEQ ID NO: 546 - DNA, 114 bp, Artificial sequence
  molecule of the complex
ttttttttttttttttgcgatcgcctttttttttttttgatacatttagatgtgtataagagacaggatgattttttttttttttttttatcatcctgtctcttatacacatct

SEQ ID NO: 547 - DNA, 74 bp, Artificial sequence
  molecule of the complex
gatagtagagatgtgtataagagacagttttttttttttttttttttttggcgatcgctttttttttttttttt

SEQ ID NO: 548 - DNA, 114 bp, Artificial sequence
  molecule of the complex
ttttttttttttttttgcgatcgcctttttttttttttgatacatttctgtctcttatacacatctgatgattttttttttttttttttatcatcagatgtgtataagagacag

SEQ ID NO: 549 - DNA, 71 bp, Artificial sequence
  molecule of the complex
gatagtagctgtctcttatacacatctttttttttttttttttttttttggtcgctttttttttttttttt

SEQ ID NO: 550 - DNA, 111 bp, Artificial sequence
  molecule of the complex
ttttttttttttttttgcgacctttttttttttttgatacatttagatgtgtataagagacaggatgattttttttttttttttttatcatcctgtctcttatacacatct

SEQ ID NO: 551 - DNA, 71 bp, Artificial sequence
  molecule of the complex
gatagtagagatgtgtataagagacagttttttttttttttttttttttggtcgctttttttttttttttt

SEQ ID NO: 552 - DNA, 111 bp, Artificial sequence
  molecule of the complex
ttttttttttttttttgcgacctttttttttttttgatacatttctgtctcttatacacatctgatgattttttttttttttttttatcatcagatgtgtataagagacag

SEQ ID NO: 553 - DNA, 77 bp, Artificial sequence
  molecule of the complex
gatagtagctgtctcttatacacatctttttttttttttttttttttttgcggcgatcggctttttttttttttttt

SEQ ID NO: 554 - DNA, 117 bp, Artificial sequence
  molecule of the complex
ttttttttttttttttgccgatcgccgctttttttttttttgatacatttagatgtgtataagagacaggatgattttttttttttttttttatcatcctgtctcttatacacatct

SEQ ID NO: 555 - DNA, 77 bp, Artificial sequence
  molecule of the complex
gatagtagagatgtgtataagagacagttttttttttttttttttttttgcggcgatcggctttttttttttttttt

SEQ ID NO: 556 - DNA, 117 bp, Artificial sequence
  molecule of the complex
ttttttttttttttttgccgatcgccgctttttttttttttgatacatttctgtctcttatacacatctgatgattttttttttttttttttatcatcagatgtgtataagagacag

SEQ ID NO: 557 - DNA, 73 bp, Artificial sequence
  molecule of the complex
cacgtgctgtctcttatacacatcttttctcgatcattattattttttggcgatcgctttttttttttttttt

SEQ ID NO: 558 - DNA, 118 bp, Artificial sequence
  molecule of the complex
ttttttttttttttttgcgatcgcctttttttttttttttttttttagatgtgtataagagacaggacgaatattttttttttttttttttttagatgtgtataagagacagcacgtg

SEQ ID NO: 559 - DNA, 73 bp, Artificial sequence
  molecule of the complex
cacgtgagatgtgtataagagacagtttctcgatcattattattttttggcgatcgctttttttttttttttt

SEQ ID NO: 560 - DNA, 118 bp, Artificial sequence
  molecule of the complex
ttttttttttttttttgcgatcgcctttttttttttttttttttttctgtctcttatacacatctgacgaatattttttttttttttttttttctgtctcttatacacatctcacgtg

SEQ ID NO: 561 - DNA, 70 bp, Artificial sequence
  molecule of the complex
cacgtgctgtctcttatacacatcttttctcgatcattattattttttggtcgctttttttttttttttt

SEQ ID NO: 562 - DNA, 115 bp, Artificial sequence
  molecule of the complex
ttttttttttttttttgcgacctttttttttttttttttttttagatgtgtataagagacaggacgaatattttttttttttttttttttagatgtgtataagagacagcacgtg

SEQ ID NO: 563 - DNA, 70 bp, Artificial sequence
  molecule of the complex
cacgtgagatgtgtataagagacagtttctcgatcattattattttttggtcgctttttttttttttttt

SEQ ID NO: 564 - DNA, 115 bp, Artificial sequence
  molecule of the complex
ttttttttttttttttgcgacctttttttttttttttttttttctgtctcttatacacatctgacgaatattttttttttttttttttttctgtctcttatacacatctcacgtg

SEQ ID NO: 565 - DNA, 76 bp, Artificial sequence
  molecule of the complex
cacgtgctgtctcttatacacatcttttctcgatcattattattttttgcggcgatcggctttttttttttttttt

SEQ ID NO: 566 - DNA, 121 bp, Artificial sequence
  molecule of the complex
ttttttttttttttttgccgatcgccgctttttttttttttttttttttagatgtgtataagagacaggacgaatattttttttttttttttttttagatgtgtataagagacagcacgtg

SEQ ID NO: 567 - DNA, 76 bp, Artificial sequence
  molecule of the complex
cacgtgagatgtgtataagagacagtttctcgatcattattattttttgcggcgatcggctttttttttttttttt

SEQ ID NO: 568 - DNA, 121 bp, Artificial sequence
  molecule of the complex
ttttttttttttttttgccgatcgccgctttttttttttttttttttttctgtctcttatacacatctgacgaatattttttttttttttttttttctgtctcttatacacatctcacgtg

SEQ ID NO: 569 - DNA, 118 bp, Artificial sequence
  molecule of the complex
agatgtgtataagagacagcacgtgttttttttttttttttttttcacgtgctgtctcttatacacatcttttctcgatcattattattttttggcgatcgctttttttttttttttt

SEQ ID NO: 570 - DNA, 73 bp, Artificial sequence
  molecule of the complex
ttttttttttttttttgcgatcgcctttttttttttttttttttttagatgtgtataagagacaggacgaata

SEQ ID NO: 571 - DNA, 118 bp, Artificial sequence
  molecule of the complex
ctgtctcttatacacatctcacgtgttttttttttttttttttttcacgtgagatgtgtataagagacagtttctcgatcattattattttttggcgatcgctttttttttttttttt

SEQ ID NO: 572 - DNA, 73 bp, Artificial sequence
  molecule of the complex
ttttttttttttttttgcgatcgcctttttttttttttttttttttctgtctcttatacacatctgacgaata

SEQ ID NO: 573 - DNA, 115 bp, Artificial sequence
  molecule of the complex
agatgtgtataagagacagcacgtgttttttttttttttttttttcacgtgctgtctcttatacacatcttttctcgatcattattattttttggtcgctttttttttttttttt

SEQ ID NO: 574 - DNA, 70 bp, Artificial sequence
  molecule of the complex
ttttttttttttttttgcgacctttttttttttttttttttttagatgtgtataagagacaggacgaata

SEQ ID NO: 575 - DNA, 115 bp, Artificial sequence
  molecule of the complex
ctgtctcttatacacatctcacgtgttttttttttttttttttttcacgtgagatgtgtataagagacagtttctcgatcattattattttttggtcgctttttttttttttttt

SEQ ID NO: 576 - DNA, 70 bp, Artificial sequence
  molecule of the complex
ttttttttttttttttgcgacctttttttttttttttttttttctgtctcttatacacatctgacgaata

SEQ ID NO: 577 - DNA, 121 bp, Artificial sequence
  molecule of the complex
agatgtgtataagagacagcacgtgttttttttttttttttttttcacgtgctgtctcttatacacatcttttctcgatcattattattttttgcggcgatcggctttttttttttttttt

SEQ ID NO: 578 - DNA, 76 bp, Artificial sequence
  molecule of the complex
ttttttttttttttttgccgatcgccgctttttttttttttttttttttagatgtgtataagagacaggacgaata

SEQ ID NO: 579 - DNA, 121 bp, Artificial sequence
  molecule of the complex
ctgtctcttatacacatctcacgtgttttttttttttttttttttcacgtgagatgtgtataagagacagtttctcgatcattattattttttgcggcgatcggctttttttttttttttt

SEQ ID NO: 580 - DNA, 76 bp, Artificial sequence
  molecule of the complex
ttttttttttttttttgccgatcgccgctttttttttttttttttttttctgtctcttatacacatctgacgaata

SEQ ID NO: 581 - DNA, 83 bp, Artificial sequence
  molecule of the complex
tgcagctgctgactcttatacacaagttttttttttttttttttttttttttttggcgatcgctttttttttttttttttttt

SEQ ID NO: 582 - DNA, 83 bp, Artificial sequence
  molecule of the complex
ttttttttttttttttttttgcgatcgccttttttttttttttttgatacatgtttacttgtgtataagagtcagctgtaagc

SEQ ID NO: 583 - DNA, 83 bp, Artificial sequence
  molecule of the complex
tgcagctgacttgtgtataagagtcagtttttttttttttttttttttttttttggcgatcgctttttttttttttttttttt

SEQ ID NO: 584 - DNA, 83 bp, Artificial sequence
  molecule of the complex
ttttttttttttttttttttgcgatcgccttttttttttttttttgatacatgtttctgactcttatacacaagtctgtaagc

SEQ ID NO: 585 - DNA, 80 bp, Artificial sequence
  molecule of the complex
tgcagctgctgactcttatacacaagttttttttttttttttttttttttttttggtcgctttttttttttttttttttt

SEQ ID NO: 586 - DNA, 80 bp, Artificial sequence
  molecule of the complex
ttttttttttttttttttttgcgaccttttttttttttttttgatacatgtttacttgtgtataagagtcagctgtaagc

SEQ ID NO: 587 - DNA, 80 bp, Artificial sequence
  molecule of the complex
tgcagctgacttgtgtataagagtcagtttttttttttttttttttttttttttggtcgctttttttttttttttttttt

SEQ ID NO: 588 - DNA, 80 bp, Artificial sequence
  molecule of the complex
ttttttttttttttttttttgcgaccttttttttttttttttgatacatgtttctgactcttatacacaagtctgtaagc

SEQ ID NO: 589 - DNA, 86 bp, Artificial sequence
  molecule of the complex
tgcagctgctgactcttatacacaagttttttttttttttttttttttttttttgcggcgatcggctttttttttttttttttttt

SEQ ID NO: 590 - DNA, 86 bp, Artificial sequence
  molecule of the complex
ttttttttttttttttttttgccgatcgccgcttttttttttttttttgatacatgtttacttgtgtataagagtcagctgtaagc

SEQ ID NO: 591 - DNA, 86 bp, Artificial sequence
  molecule of the complex
tgcagctgacttgtgtataagagtcagtttttttttttttttttttttttttttgcggcgatcggctttttttttttttttttttt

SEQ ID NO: 592 - DNA, 86 bp, Artificial sequence
  molecule of the complex
ttttttttttttttttttttgccgatcgccgcttttttttttttttttgatacatgtttctgactcttatacacaagtctgtaagc

SEQ ID NO: 593 - DNA, 80 bp, Artificial sequence
  molecule of the complex
gtgcccagctgactcttatacacaagttttctcgatcattttttttttttttttggtcgctttttttttttttttttttt

SEQ ID NO: 594 - DNA, 80 bp, Artificial sequence
  molecule of the complex
ttttttttttttttttttttgcgacctttttttttttttttttttttttttttacttgtgtataagagtcagctgcttat

SEQ ID NO: 595 - DNA, 80 bp, Artificial sequence
  molecule of the complex
gtgcccagacttgtgtataagagtcagtttctcgatcattttttttttttttttggtcgctttttttttttttttttttt

SEQ ID NO: 596 - DNA, 80 bp, Artificial sequence
  molecule of the complex
ttttttttttttttttttttgcgacctttttttttttttttttttttttttttctgactcttatacacaagtctgcttat

SEQ ID NO: 597 - DNA, 116 bp, Artificial sequence
  molecule of the complex
atcatcctgactcttatacacaagttttttttttttttttttgatagtagctgactcttatacacaagtttttttttttttttttttttttggcgatcgctttttttttttttttt

SEQ ID NO: 598 - DNA, 72 bp, Artificial sequence
  molecule of the complex
ttttttttttttttttgcgatcgcctttttttttttttgatacatttacttgtgtataagagtcaggatgat

SEQ ID NO: 599 - DNA, 116 bp, Artificial sequence
  molecule of the complex
atcatcacttgtgtataagagtcagtttttttttttttttttgatagtagacttgtgtataagagtcagttttttttttttttttttttttggcgatcgctttttttttttttttt

SEQ ID NO: 600 - DNA, 72 bp, Artificial sequence
  molecule of the complex
ttttttttttttttttgcgatcgcctttttttttttttgatacatttctgactcttatacacaagtgatgat

SEQ ID NO: 601 - DNA, 113 bp, Artificial sequence
  molecule of the complex
atcatcctgactcttatacacaagttttttttttttttttttgatagtagctgactcttatacacaagtttttttttttttttttttttttggtcgctttttttttttttttt

SEQ ID NO: 602 - DNA, 69 bp, Artificial sequence
  molecule of the complex
ttttttttttttttttgcgacctttttttttttttgatacatttacttgtgtataagagtcaggatgat

SEQ ID NO: 603 - DNA, 113 bp, Artificial sequence
  molecule of the complex
atcatcacttgtgtataagagtcagtttttttttttttttttgatagtagacttgtgtataagagtcagttttttttttttttttttttttggtcgctttttttttttttttt

SEQ ID NO: 604 - DNA, 69 bp, Artificial sequence
  molecule of the complex
ttttttttttttttttgcgacctttttttttttttgatacatttctgactcttatacacaagtgatgat

SEQ ID NO: 605 - DNA, 119 bp, Artificial sequence
  molecule of the complex
atcatcctgactcttatacacaagttttttttttttttttttgatagtagctgactcttatacacaagtttttttttttttttttttttttgcggcgatcggctttttttttttttttt

SEQ ID NO: 606 - DNA, 75 bp, Artificial sequence
  molecule of the complex
ttttttttttttttttgccgatcgccgctttttttttttttgatacatttacttgtgtataagagtcaggatgat

SEQ ID NO: 607 - DNA, 119 bp, Artificial sequence
  molecule of the complex
atcatcacttgtgtataagagtcagtttttttttttttttttgatagtagacttgtgtataagagtcagttttttttttttttttttttttgcggcgatcggctttttttttttttttt

SEQ ID NO: 608 - DNA, 75 bp, Artificial sequence
  molecule of the complex
ttttttttttttttttgccgatcgccgctttttttttttttgatacatttctgactcttatacacaagtgatgat

SEQ ID NO: 609 - DNA, 74 bp, Artificial sequence
  molecule of the complex
gatagtagctgactcttatacacaagtttttttttttttttttttttttggcgatcgctttttttttttttttt

SEQ ID NO: 610 - DNA, 114 bp, Artificial sequence
  molecule of the complex
ttttttttttttttttgcgatcgcctttttttttttttgatacatttacttgtgtataagagtcaggatgattttttttttttttttttatcatcctgactcttatacacaagt

SEQ ID NO: 611 - DNA, 74 bp, Artificial sequence
  molecule of the complex
gatagtagacttgtgtataagagtcagttttttttttttttttttttttggcgatcgctttttttttttttttt

SEQ ID NO: 612 - DNA, 114 bp, Artificial sequence
  molecule of the complex
ttttttttttttttttgcgatcgcctttttttttttttgatacatttctgactcttatacacaagtgatgattttttttttttttttttatcatcacttgtgtataagagtcag

SEQ ID NO: 613 - DNA, 71 bp, Artificial sequence
  molecule of the complex
gatagtagctgactcttatacacaagtttttttttttttttttttttttggtcgctttttttttttttttt

SEQ ID NO: 614 - DNA, 111 bp, Artificial sequence
  molecule of the complex
ttttttttttttttttgcgacctttttttttttttgatacatttacttgtgtataagagtcaggatgattttttttttttttttttatcatcctgactcttatacacaagt

SEQ ID NO: 615 - DNA, 71 bp, Artificial sequence
  molecule of the complex
gatagtagacttgtgtataagagtcagttttttttttttttttttttttggtcgctttttttttttttttt

SEQ ID NO: 616 - DNA, 111 bp, Artificial sequence
  molecule of the complex
ttttttttttttttttgcgacctttttttttttttgatacatttctgactcttatacacaagtgatgattttttttttttttttttatcatcacttgtgtataagagtcag

SEQ ID NO: 617 - DNA, 77 bp, Artificial sequence
  molecule of the complex
gatagtagctgactcttatacacaagtttttttttttttttttttttttgcggcgatcggctttttttttttttttt

SEQ ID NO: 618 - DNA, 117 bp, Artificial sequence
  molecule of the complex
ttttttttttttttttgccgatcgccgctttttttttttttgatacatttacttgtgtataagagtcaggatgattttttttttttttttttatcatcctgactcttatacacaagt

SEQ ID NO: 619 - DNA, 77 bp, Artificial sequence
  molecule of the complex
gatagtagacttgtgtataagagtcagttttttttttttttttttttttgcggcgatcggctttttttttttttttt

SEQ ID NO: 620 - DNA, 117 bp, Artificial sequence
  molecule of the complex
ttttttttttttttttgccgatcgccgctttttttttttttgatacatttctgactcttatacacaagtgatgattttttttttttttttttatcatcacttgtgtataagagtcag

SEQ ID NO: 621 - DNA, 73 bp, Artificial sequence
  molecule of the complex
cacgtgctgactcttatacacaagttttctcgatcattattattttttggcgatcgctttttttttttttttt

SEQ ID NO: 622 - DNA, 118 bp, Artificial sequence
  molecule of the complex
ttttttttttttttttgcgatcgcctttttttttttttttttttttacttgtgtataagagtcaggacgaatattttttttttttttttttttacttgtgtataagagtcagcacgtg

SEQ ID NO: 623 - DNA, 73 bp, Artificial sequence
  molecule of the complex
cacgtgacttgtgtataagagtcagtttctcgatcattattattttttggcgatcgctttttttttttttttt

SEQ ID NO: 624 - DNA, 118 bp, Artificial sequence
  molecule of the complex
ttttttttttttttttgcgatcgcctttttttttttttttttttttctgactcttatacacaagtgacgaatattttttttttttttttttttctgactcttatacacaagtcacgtg

SEQ ID NO: 625 - DNA, 70 bp, Artificial sequence
  molecule of the complex
cacgtgctgactcttatacacaagttttctcgatcattattattttttggtcgctttttttttttttttt

SEQ ID NO: 626 - DNA, 115 bp, Artificial sequence
  molecule of the complex
ttttttttttttttttgcgacctttttttttttttttttttttacttgtgtataagagtcaggacgaatattttttttttttttttttttacttgtgtataagagtcagcacgtg

SEQ ID NO: 627 - DNA, 70 bp, Artificial sequence
  molecule of the complex
cacgtgacttgtgtataagagtcagtttctcgatcattattattttttggtcgctttttttttttttttt

SEQ ID NO: 628 - DNA, 115 bp, Artificial sequence
  molecule of the complex
ttttttttttttttttgcgacctttttttttttttttttttttctgactcttatacacaagtgacgaatattttttttttttttttttttctgactcttatacacaagtcacgtg

SEQ ID NO: 629 - DNA, 76 bp, Artificial sequence
  molecule of the complex
cacgtgctgactcttatacacaagttttctcgatcattattattttttgcggcgatcggctttttttttttttttt

SEQ ID NO: 630 - DNA, 121 bp, Artificial sequence
  molecule of the complex
ttttttttttttttttgccgatcgccgctttttttttttttttttttttacttgtgtataagagtcaggacgaatattttttttttttttttttttacttgtgtataagagtcagcacgtg

SEQ ID NO: 631 - DNA, 76 bp, Artificial sequence
  molecule of the complex
cacgtgacttgtgtataagagtcagtttctcgatcattattattttttgcggcgatcggctttttttttttttttt

SEQ ID NO: 632 - DNA, 121 bp, Artificial sequence
  molecule of the complex
ttttttttttttttttgccgatcgccgctttttttttttttttttttttctgactcttatacacaagtgacgaatattttttttttttttttttttctgactcttatacacaagtcacgtg

SEQ ID NO: 633 - DNA, 118 bp, Artificial sequence
  molecule of the complex
acttgtgtataagagtcagcacgtgttttttttttttttttttttcacgtgctgactcttatacacaagttttctcgatcattattattttttggcgatcgctttttttttttttttt

SEQ ID NO: 634 - DNA, 73 bp, Artificial sequence
  molecule of the complex
ttttttttttttttttgcgatcgcctttttttttttttttttttttacttgtgtataagagtcaggacgaata

SEQ ID NO: 635 - DNA, 118 bp, Artificial sequence
  molecule of the complex
ctgactcttatacacaagtcacgtgttttttttttttttttttttcacgtgacttgtgtataagagtcagtttctcgatcattattattttttggcgatcgctttttttttttttttt

SEQ ID NO: 636 - DNA, 73 bp, Artificial sequence
  molecule of the complex
ttttttttttttttttgcgatcgcctttttttttttttttttttttctgactcttatacacaagtgacgaata

SEQ ID NO: 637 - DNA, 115 bp, Artificial sequence
  molecule of the complex
acttgtgtataagagtcagcacgtgttttttttttttttttttttcacgtgctgactcttatacacaagttttctcgatcattattattttttggtcgctttttttttttttttt

SEQ ID NO: 638 - DNA, 70 bp, Artificial sequence
  molecule of the complex
ttttttttttttttttgcgacctttttttttttttttttttttacttgtgtataagagtcaggacgaata

SEQ ID NO: 639 - DNA, 115 bp, Artificial sequence
  molecule of the complex
ctgactcttatacacaagtcacgtgttttttttttttttttttttcacgtgacttgtgtataagagtcagtttctcgatcattattattttttggtcgctttttttttttttttt

SEQ ID NO: 640 - DNA, 70 bp, Artificial sequence
  molecule of the complex
ttttttttttttttttgcgacctttttttttttttttttttttctgactcttatacacaagtgacgaata

SEQ ID NO: 641 - DNA, 121 bp, Artificial sequence
  molecule of the complex
acttgtgtataagagtcagcacgtgttttttttttttttttttttcacgtgctgactcttatacacaagttttctcgatcattattattttttgcggcgatcggctttttttttttttttt

SEQ ID NO: 642 - DNA, 76 bp, Artificial sequence
  molecule of the complex
ttttttttttttttttgccgatcgccgctttttttttttttttttttttacttgtgtataagagtcaggacgaata

SEQ ID NO: 643 - DNA, 121 bp, Artificial sequence
  molecule of the complex
ctgactcttatacacaagtcacgtgttttttttttttttttttttcacgtgacttgtgtataagagtcagtttctcgatcattattattttttgcggcgatcggctttttttttttttttt

SEQ ID NO: 644 - DNA, 76 bp, Artificial sequence
  molecule of the complex
ttttttttttttttttgccgatcgccgctttttttttttttttttttttctgactcttatacacaagtgacgaata

SEQ ID NO: 645 - DNA, 98 bp, Artificial sequence
  molecule of the complex
tacaagtccggataataattttttttttttttttttttttacttgtgtataagagtcagctgggcacgcgtataagcagctgactcttatacacaagt

SEQ ID NO: 646 - DNA, 98 bp, Artificial sequence
  molecule of the complex
tacaagtccggataataattttttttttttttttttttttctgactcttatacacaagtctgggcacgcgtataagcagacttgtgtataagagtcag

SEQ ID NO: 647 - DNA, 86 bp, Artificial sequence
  molecule of the complex
taataattttttttttttttttttttttacttgtgtataagagtcagctgggcacgcgtataagcagctgactcttatacacaagt

SEQ ID NO: 648 - DNA, 86 bp, Artificial sequence
  molecule of the complex
taataattttttttttttttttttttttctgactcttatacacaagtctgggcacgcgtataagcagacttgtgtataagagtcag

SEQ ID NO: 649 - DNA, 92 bp, Artificial sequence
  molecule of the complex
tacaagtaataattttttttttttttttttttttacttgtgtataagagtcagctgggcacgcgtataagcagctgactcttatacacaagt

SEQ ID NO: 650 - DNA, 92 bp, Artificial sequence
  molecule of the complex
tacaagtaataattttttttttttttttttttttctgactcttatacacaagtctgggcacgcgtataagcagacttgtgtataagagtcag

SEQ ID NO: 651 - DNA, 63 bp, Artificial sequence
  molecule of the complex
acttggttaattaattttttttttttttttttttttacttgtgtataagagtcagctactatc

SEQ ID NO: 652 - DNA, 63 bp, Artificial sequence
  molecule of the complex
acttggttaattaattttttttttttttttttttttctgactcttatacacaagtctactatc

SEQ ID NO: 653 - DNA, 57 bp, Artificial sequence
  molecule of the complex
ttaattaattttttttttttttttttttttacttgtgtataagagtcagctactatc

SEQ ID NO: 654 - DNA, 57 bp, Artificial sequence
  molecule of the complex
ttaattaattttttttttttttttttttttctgactcttatacacaagtctactatc

SEQ ID NO: 655 - DNA, 69 bp, Artificial sequence
  molecule of the complex
acttggcatatgttaattaattttttttttttttttttttttacttgtgtataagagtcagctactatc

SEQ ID NO: 656 - DNA, 69 bp, Artificial sequence
  molecule of the complex
acttggcatatgttaattaattttttttttttttttttttttctgactcttatacacaagtctactatc

SEQ ID NO: 657 - DNA, 62 bp, Artificial sequence
  molecule of the complex
tattcgtcctgactcttatacacaagtttttttttttttttttttattaattattacttgta

SEQ ID NO: 658 - DNA, 62 bp, Artificial sequence
  molecule of the complex
tattcgtcacttgtgtataagagtcagttttttttttttttttttattaattattacttgta

SEQ ID NO: 659 - DNA, 56 bp, Artificial sequence
  molecule of the complex
tattcgtcctgactcttatacacaagtttttttttttttttttttattaattatta

SEQ ID NO: 660 - DNA, 56 bp, Artificial sequence
  molecule of the complex
tattcgtcacttgtgtataagagtcagttttttttttttttttttattaattatta

SEQ ID NO: 661 - DNA, 68 bp, Artificial sequence
  molecule of the complex
tattcgtcctgactcttatacacaagtttttttttttttttttttattaattattatccggacttgta

SEQ ID NO: 662 - DNA, 68 bp, Artificial sequence
  molecule of the complex
tattcgtcacttgtgtataagagtcagttttttttttttttttttattaattattatccggacttgta

SEQ ID NO: 663 - DNA, 86 bp, Artificial sequence
  molecule of the complex
tgcagctgctgtctcttgatcagatcttttttttttttttttttttttttttttgcggcgatcggctttttttttttttttttttt

SEQ ID NO: 664 - DNA, 86 bp, Artificial sequence
  molecule of the complex
ttttttttttttttttttttgccgatcgccgcttttttttttttttttgatacatgtttagatctgatcaagagacagctgtaagc

SEQ ID NO: 665 - DNA, 86 bp, Artificial sequence
  molecule of the complex
tgcagctgagatctgatcaagagacagtttttttttttttttttttttttttttgcggcgatcggctttttttttttttttttttt

SEQ ID NO: 666 - DNA, 86 bp, Artificial sequence
  molecule of the complex
ttttttttttttttttttttgccgatcgccgcttttttttttttttttgatacatgtttctgtctcttgatcagatctctgtaagc

SEQ ID NO: 667 - DNA, 80 bp, Artificial sequence
  molecule of the complex
gtgcccagctgtctcttgatcagatcttttctcgatcattttttttttttttttggtcgctttttttttttttttttttt

SEQ ID NO: 668 - DNA, 80 bp, Artificial sequence
  molecule of the complex
ttttttttttttttttttttgcgacctttttttttttttttttttttttttttagatctgatcaagagacagctgcttat

SEQ ID NO: 669 - DNA, 80 bp, Artificial sequence
  molecule of the complex
gtgcccagagatctgatcaagagacagtttctcgatcattttttttttttttttggtcgctttttttttttttttttttt

SEQ ID NO: 670 - DNA, 80 bp, Artificial sequence
  molecule of the complex
ttttttttttttttttttttgcgacctttttttttttttttttttttttttttctgtctcttgatcagatctctgcttat

SEQ ID NO: 671 - DNA, 83 bp, Artificial sequence
  molecule of the complex
gtgcccagctgtctcttgatcagatcttttctcgatcattttttttttttttttggcgatcgctttttttttttttttttttt

SEQ ID NO: 672 - DNA, 83 bp, Artificial sequence
  molecule of the complex
ttttttttttttttttttttgcgatcgcctttttttttttttttttttttttttttagatctgatcaagagacagctgcttat

SEQ ID NO: 673 - DNA, 83 bp, Artificial sequence
  molecule of the complex
gtgcccagagatctgatcaagagacagtttctcgatcattttttttttttttttggcgatcgctttttttttttttttttttt

SEQ ID NO: 674 - DNA, 83 bp, Artificial sequence
  molecule of the complex
ttttttttttttttttttttgcgatcgcctttttttttttttttttttttttttttctgtctcttgatcagatctctgcttat

SEQ ID NO: 675 - DNA, 86 bp, Artificial sequence
  molecule of the complex
gtgcccagctgtctcttgatcagatcttttctcgatcattttttttttttttttgcggcgatcggctttttttttttttttttttt

SEQ ID NO: 676 - DNA, 86 bp, Artificial sequence
  molecule of the complex
ttttttttttttttttttttgccgatcgccgctttttttttttttttttttttttttttagatctgatcaagagacagctgcttat

SEQ ID NO: 677 - DNA, 86 bp, Artificial sequence
  molecule of the complex
gtgcccagagatctgatcaagagacagtttctcgatcattttttttttttttttgcggcgatcggctttttttttttttttttttt

SEQ ID NO: 678 - DNA, 86 bp, Artificial sequence
  molecule of the complex
ttttttttttttttttttttgccgatcgccgctttttttttttttttttttttttttttctgtctcttgatcagatctctgcttat

SEQ ID NO: 679 - DNA, 116 bp, Artificial sequence
  molecule of the complex
atcatcctgtctcttgatcagatcttttttttttttttttttgatagtagctgtctcttgatcagatctttttttttttttttttttttttggcgatcgctttttttttttttttt

SEQ ID NO: 680 - DNA, 72 bp, Artificial sequence
  molecule of the complex
ttttttttttttttttgcgatcgcctttttttttttttgatacatttagatctgatcaagagacaggatgat

SEQ ID NO: 681 - DNA, 116 bp, Artificial sequence
  molecule of the complex
atcatcagatctgatcaagagacagtttttttttttttttttgatagtagagatctgatcaagagacagttttttttttttttttttttttggcgatcgctttttttttttttttt

SEQ ID NO: 682 - DNA, 72 bp, Artificial sequence
  molecule of the complex
ttttttttttttttttgcgatcgcctttttttttttttgatacatttctgtctcttgatcagatctgatgat

SEQ ID NO: 683 - DNA, 113 bp, Artificial sequence
  molecule of the complex
atcatcctgtctcttgatcagatcttttttttttttttttttgatagtagctgtctcttgatcagatctttttttttttttttttttttttggtcgctttttttttttttttt

SEQ ID NO: 684 - DNA, 69 bp, Artificial sequence
  molecule of the complex
ttttttttttttttttgcgacctttttttttttttgatacatttagatctgatcaagagacaggatgat

SEQ ID NO: 685 - DNA, 113 bp, Artificial sequence
  molecule of the complex
atcatcagatctgatcaagagacagtttttttttttttttttgatagtagagatctgatcaagagacagttttttttttttttttttttttggtcgctttttttttttttttt

SEQ ID NO: 686 - DNA, 69 bp, Artificial sequence
  molecule of the complex
ttttttttttttttttgcgacctttttttttttttgatacatttctgtctcttgatcagatctgatgat

SEQ ID NO: 687 - DNA, 119 bp, Artificial sequence
  molecule of the complex
atcatcctgtctcttgatcagatcttttttttttttttttttgatagtagctgtctcttgatcagatctttttttttttttttttttttttgcggcgatcggctttttttttttttttt

SEQ ID NO: 688 - DNA, 75 bp, Artificial sequence
  molecule of the complex
ttttttttttttttttgccgatcgccgctttttttttttttgatacatttagatctgatcaagagacaggatgat

SEQ ID NO: 689 - DNA, 119 bp, Artificial sequence
  molecule of the complex
atcatcagatctgatcaagagacagtttttttttttttttttgatagtagagatctgatcaagagacagttttttttttttttttttttttgcggcgatcggctttttttttttttttt

SEQ ID NO: 690 - DNA, 75 bp, Artificial sequence
  molecule of the complex
ttttttttttttttttgccgatcgccgctttttttttttttgatacatttctgtctcttgatcagatctgatgat

SEQ ID NO: 691 - DNA, 74 bp, Artificial sequence
  molecule of the complex
gatagtagctgtctcttgatcagatctttttttttttttttttttttttggcgatcgctttttttttttttttt

SEQ ID NO: 692 - DNA, 114 bp, Artificial sequence
  molecule of the complex
ttttttttttttttttgcgatcgcctttttttttttttgatacatttagatctgatcaagagacaggatgattttttttttttttttttatcatcctgtctcttgatcagatct

SEQ ID NO: 693 - DNA, 74 bp, Artificial sequence
  molecule of the complex
gatagtagagatctgatcaagagacagttttttttttttttttttttttggcgatcgctttttttttttttttt

SEQ ID NO: 694 - DNA, 114 bp, Artificial sequence
  molecule of the complex
ttttttttttttttttgcgatcgcctttttttttttttgatacatttctgtctcttgatcagatctgatgattttttttttttttttttatcatcagatctgatcaagagacag

SEQ ID NO: 695 - DNA, 71 bp, Artificial sequence
  molecule of the complex
gatagtagctgtctcttgatcagatctttttttttttttttttttttttggtcgctttttttttttttttt

SEQ ID NO: 696 - DNA, 111 bp, Artificial sequence
  molecule of the complex
ttttttttttttttttgcgacctttttttttttttgatacatttagatctgatcaagagacaggatgattttttttttttttttttatcatcctgtctcttgatcagatct

SEQ ID NO: 697 - DNA, 71 bp, Artificial sequence
  molecule of the complex
gatagtagagatctgatcaagagacagttttttttttttttttttttttggtcgctttttttttttttttt

SEQ ID NO: 698 - DNA, 111 bp, Artificial sequence
  molecule of the complex
ttttttttttttttttgcgacctttttttttttttgatacatttctgtctcttgatcagatctgatgattttttttttttttttttatcatcagatctgatcaagagacag

SEQ ID NO: 699 - DNA, 77 bp, Artificial sequence
  molecule of the complex
gatagtagctgtctcttgatcagatctttttttttttttttttttttttgcggcgatcggctttttttttttttttt

SEQ ID NO: 700 - DNA, 117 bp, Artificial sequence
  molecule of the complex
ttttttttttttttttgccgatcgccgctttttttttttttgatacatttagatctgatcaagagacaggatgattttttttttttttttttatcatcctgtctcttgatcagatct

SEQ ID NO: 701 - DNA, 77 bp, Artificial sequence
  molecule of the complex
gatagtagagatctgatcaagagacagttttttttttttttttttttttgcggcgatcggctttttttttttttttt

SEQ ID NO: 702 - DNA, 117 bp, Artificial sequence
  molecule of the complex
ttttttttttttttttgccgatcgccgctttttttttttttgatacatttctgtctcttgatcagatctgatgattttttttttttttttttatcatcagatctgatcaagagacag

SEQ ID NO: 703 - DNA, 73 bp, Artificial sequence
  molecule of the complex
cacgtgctgtctcttgatcagatcttttctcgatcattattattttttggcgatcgctttttttttttttttt

SEQ ID NO: 704 - DNA, 118 bp, Artificial sequence
  molecule of the complex
ttttttttttttttttgcgatcgcctttttttttttttttttttttagatctgatcaagagacaggacgaatattttttttttttttttttttagatctgatcaagagacagcacgtg

SEQ ID NO: 705 - DNA, 73 bp, Artificial sequence
  molecule of the complex
cacgtgagatctgatcaagagacagtttctcgatcattattattttttggcgatcgctttttttttttttttt

SEQ ID NO: 706 - DNA, 118 bp, Artificial sequence
  molecule of the complex
ttttttttttttttttgcgatcgcctttttttttttttttttttttctgtctcttgatcagatctgacgaatattttttttttttttttttttctgtctcttgatcagatctcacgtg

SEQ ID NO: 707 - DNA, 70 bp, Artificial sequence
  molecule of the complex
cacgtgctgtctcttgatcagatcttttctcgatcattattattttttggtcgctttttttttttttttt

SEQ ID NO: 708 - DNA, 115 bp, Artificial sequence
  molecule of the complex
ttttttttttttttttgcgacctttttttttttttttttttttagatctgatcaagagacaggacgaatattttttttttttttttttttagatctgatcaagagacagcacgtg

SEQ ID NO: 709 - DNA, 70 bp, Artificial sequence
  molecule of the complex
cacgtgagatctgatcaagagacagtttctcgatcattattattttttggtcgctttttttttttttttt

SEQ ID NO: 710 - DNA, 115 bp, Artificial sequence
  molecule of the complex
ttttttttttttttttgcgacctttttttttttttttttttttctgtctcttgatcagatctgacgaatattttttttttttttttttttctgtctcttgatcagatctcacgtg

SEQ ID NO: 711 - DNA, 76 bp, Artificial sequence
  molecule of the complex
cacgtgctgtctcttgatcagatcttttctcgatcattattattttttgcggcgatcggctttttttttttttttt

SEQ ID NO: 712 - DNA, 121 bp, Artificial sequence
  molecule of the complex
ttttttttttttttttgccgatcgccgctttttttttttttttttttttagatctgatcaagagacaggacgaatattttttttttttttttttttagatctgatcaagagacagcacgtg

SEQ ID NO: 713 - DNA, 76 bp, Artificial sequence
  molecule of the complex
cacgtgagatctgatcaagagacagtttctcgatcattattattttttgcggcgatcggctttttttttttttttt

SEQ ID NO: 714 - DNA, 121 bp, Artificial sequence
  molecule of the complex
ttttttttttttttttgccgatcgccgctttttttttttttttttttttctgtctcttgatcagatctgacgaatattttttttttttttttttttctgtctcttgatcagatctcacgtg

SEQ ID NO: 715 - DNA, 118 bp, Artificial sequence
  molecule of the complex
agatctgatcaagagacagcacgtgttttttttttttttttttttcacgtgctgtctcttgatcagatcttttctcgatcattattattttttggcgatcgctttttttttttttttt

SEQ ID NO: 716 - DNA, 73 bp, Artificial sequence
  molecule of the complex
ttttttttttttttttgcgatcgcctttttttttttttttttttttagatctgatcaagagacaggacgaata

SEQ ID NO: 717 - DNA, 118 bp, Artificial sequence
  molecule of the complex
ctgtctcttgatcagatctcacgtgttttttttttttttttttttcacgtgagatctgatcaagagacagtttctcgatcattattattttttggcgatcgctttttttttttttttt

SEQ ID NO: 718 - DNA, 73 bp, Artificial sequence
  molecule of the complex
ttttttttttttttttgcgatcgcctttttttttttttttttttttctgtctcttgatcagatctgacgaata

SEQ ID NO: 719 - DNA, 115 bp, Artificial sequence
  molecule of the complex
agatctgatcaagagacagcacgtgttttttttttttttttttttcacgtgctgtctcttgatcagatcttttctcgatcattattattttttggtcgctttttttttttttttt

SEQ ID NO: 720 - DNA, 70 bp, Artificial sequence
  molecule of the complex
ttttttttttttttttgcgacctttttttttttttttttttttagatctgatcaagagacaggacgaata

SEQ ID NO: 721 - DNA, 115 bp, Artificial sequence
  molecule of the complex
ctgtctcttgatcagatctcacgtgttttttttttttttttttttcacgtgagatctgatcaagagacagtttctcgatcattattattttttggtcgctttttttttttttttt

SEQ ID NO: 722 - DNA, 70 bp, Artificial sequence
  molecule of the complex
ttttttttttttttttgcgacctttttttttttttttttttttctgtctcttgatcagatctgacgaata

SEQ ID NO: 723 - DNA, 121 bp, Artificial sequence
  molecule of the complex
agatctgatcaagagacagcacgtgttttttttttttttttttttcacgtgctgtctcttgatcagatcttttctcgatcattattattttttgcggcgatcggctttttttttttttttt

SEQ ID NO: 724 - DNA, 76 bp, Artificial sequence
  molecule of the complex
ttttttttttttttttgccgatcgccgctttttttttttttttttttttagatctgatcaagagacaggacgaata

SEQ ID NO: 725 - DNA, 121 bp, Artificial sequence
  molecule of the complex
ctgtctcttgatcagatctcacgtgttttttttttttttttttttcacgtgagatctgatcaagagacagtttctcgatcattattattttttgcggcgatcggctttttttttttttttt

SEQ ID NO: 726 - DNA, 76 bp, Artificial sequence
  molecule of the complex
ttttttttttttttttgccgatcgccgctttttttttttttttttttttctgtctcttgatcagatctgacgaata

SEQ ID NO: 727 - DNA, 98 bp, Artificial sequence
  molecule of the complex
tacaagtccggataataattttttttttttttttttttttagatctgatcaagagacagctgggcacgcgtataagcagctgtctcttgatcagatct

SEQ ID NO: 728 - DNA, 98 bp, Artificial sequence
  molecule of the complex
tacaagtccggataataattttttttttttttttttttttctgtctcttgatcagatctctgggcacgcgtataagcagagatctgatcaagagacag

SEQ ID NO: 729 - DNA, 86 bp, Artificial sequence
  molecule of the complex
taataattttttttttttttttttttttagatctgatcaagagacagctgggcacgcgtataagcagctgtctcttgatcagatct

SEQ ID NO: 730 - DNA, 86 bp, Artificial sequence
  molecule of the complex
taataattttttttttttttttttttttctgtctcttgatcagatctctgggcacgcgtataagcagagatctgatcaagagacag

SEQ ID NO: 731 - DNA, 92 bp, Artificial sequence
  molecule of the complex
tacaagtaataattttttttttttttttttttttagatctgatcaagagacagctgggcacgcgtataagcagctgtctcttgatcagatct

SEQ ID NO: 732 - DNA, 92 bp, Artificial sequence
  molecule of the complex
tacaagtaataattttttttttttttttttttttctgtctcttgatcagatctctgggcacgcgtataagcagagatctgatcaagagacag

SEQ ID NO: 733 - DNA, 63 bp, Artificial sequence
  molecule of the complex
acttggttaattaattttttttttttttttttttttagatctgatcaagagacagctactatc

SEQ ID NO: 734 - DNA, 63 bp, Artificial sequence
  molecule of the complex
acttggttaattaattttttttttttttttttttttctgtctcttgatcagatctctactatc

SEQ ID NO: 735 - DNA, 57 bp, Artificial sequence
  molecule of the complex
ttaattaattttttttttttttttttttttagatctgatcaagagacagctactatc

SEQ ID NO: 736 - DNA, 57 bp, Artificial sequence
  molecule of the complex
ttaattaattttttttttttttttttttttctgtctcttgatcagatctctactatc

SEQ ID NO: 737 - DNA, 69 bp, Artificial sequence
  molecule of the complex
acttggcatatgttaattaattttttttttttttttttttttagatctgatcaagagacagctactatc

SEQ ID NO: 738 - DNA, 69 bp, Artificial sequence
  molecule of the complex
acttggcatatgttaattaattttttttttttttttttttttctgtctcttgatcagatctctactatc

SEQ ID NO: 739 - DNA, 62 bp, Artificial sequence
  molecule of the complex
tattcgtcctgtctcttgatcagatctttttttttttttttttttattaattattacttgta

SEQ ID NO: 740 - DNA, 62 bp, Artificial sequence
  molecule of the complex
tattcgtcagatctgatcaagagacagttttttttttttttttttattaattattacttgta

SEQ ID NO: 741 - DNA, 56 bp, Artificial sequence
  molecule of the complex
tattcgtcctgtctcttgatcagatctttttttttttttttttttattaattatta

SEQ ID NO: 742 - DNA, 56 bp, Artificial sequence
  molecule of the complex
tattcgtcagatctgatcaagagacagttttttttttttttttttattaattatta

SEQ ID NO: 743 - DNA, 68 bp, Artificial sequence
  molecule of the complex
tattcgtcctgtctcttgatcagatctttttttttttttttttttattaattattatccggacttgta

SEQ ID NO: 744 - DNA, 68 bp, Artificial sequence
  molecule of the complex
tattcgtcagatctgatcaagagacagttttttttttttttttttattaattattatccggacttgta

SEQ ID NO: 745 - DNA, 56 bp, Artificial sequence; identical to SEQ ID NO: 741
  molecule of the complex

SEQ ID NO: 746 - DNA, 56 bp, Artificial sequence; identical to SEQ ID NO: 742
  molecule of the complex

SEQ ID NO: 747 - DNA, 68 bp, Artificial sequence; identical to SEQ ID NO: 743
  molecule of the complex

SEQ ID NO: 748 - DNA, 68 bp, Artificial sequence; identical to SEQ ID NO: 744
  molecule of the complex

SEQ ID NO: 749 - DNA, 83 bp, Artificial sequence
  molecule of the complex
tgcagctgctgtctcttgatcagatcttttttttttttttttttttttttttttggcgatcgctttttttttttttttttttt

SEQ ID NO: 750 - DNA, 83 bp, Artificial sequence
  molecule of the complex
ttttttttttttttttttttgcgatcgccttttttttttttttttgatacatgtttagatctgatcaagagacagctgtaagc

SEQ ID NO: 751 - DNA, 83 bp, Artificial sequence
  molecule of the complex
tgcagctgagatctgatcaagagacagtttttttttttttttttttttttttttggcgatcgctttttttttttttttttttt

SEQ ID NO: 752 - DNA, 83 bp, Artificial sequence
  molecule of the complex
ttttttttttttttttttttgcgatcgccttttttttttttttttgatacatgtttctgtctcttgatcagatctctgtaagc

SEQ ID NO: 753 - DNA, 80 bp, Artificial sequence
  molecule of the complex
tgcagctgctgtctcttgatcagatcttttttttttttttttttttttttttttggtcgctttttttttttttttttttt

SEQ ID NO: 754 - DNA, 80 bp, Artificial sequence
  molecule of the complex
ttttttttttttttttttttgcgaccttttttttttttttttgatacatgtttagatctgatcaagagacagctgtaagc

SEQ ID NO: 755 - DNA, 80 bp, Artificial sequence
  molecule of the complex
tgcagctgagatctgatcaagagacagtttttttttttttttttttttttttttggtcgctttttttttttttttttttt

SEQ ID NO: 756 - DNA, 80 bp, Artificial sequence
  molecule of the complex
ttttttttttttttttttttgcgaccttttttttttttttttgatacatgtttctgtctcttgatcagatctctgtaagc

SEQ ID NO: 757 - DNA, 94 bp, Artificial sequence
  molecule of the complex
acttgtgtataagagtcagcagctgcagacaaagcttacagctgactcttatacacaagtttttttttttttttttttttttcatatgccaagt

SEQ ID NO: 758 - DNA, 94 bp, Artificial sequence
  molecule of the complex
ctgactcttatacacaagtcagctgcagacaaagcttacagacttgtgtataagagtcagttttttttttttttttttttttcatatgccaagt

SEQ ID NO: 759 - DNA, 82 bp, Artificial sequence
  molecule of the complex
acttgtgtataagagtcagcagctgcagacaaagcttacagctgactcttatacacaagttttttttttttttttttttttt

SEQ ID NO: 760 - DNA, 82 bp, Artificial sequence
  molecule of the complex
ctgactcttatacacaagtcagctgcagacaaagcttacagacttgtgtataagagtcagtttttttttttttttttttttt

SEQ ID NO: 761 - DNA, 68 bp, Artificial sequence; identical to SEQ ID NO: 661
  molecule of the complex

SEQ ID NO: 762 - DNA, 68 bp, Artificial sequence; identical to SEQ ID NO: 662
  molecule of the complex

SEQ ID NO: 763 - DNA, 83 bp, Artificial sequence; identical to SEQ ID NO: 749
  molecule of the complex

SEQ ID NO: 764 - DNA, 83 bp, Artificial sequence; identical to SEQ ID NO: 750
  molecule of the complex

SEQ ID NO: 765 - DNA, 83 bp, Artificial sequence; identical to SEQ ID NO: 751
  molecule of the complex

SEQ ID NO: 766 - DNA, 83 bp, Artificial sequence; identical to SEQ ID NO: 752
  molecule of the complex

SEQ ID NO: 767 - DNA, 80 bp, Artificial sequence; identical to SEQ ID NO: 753
  molecule of the complex

SEQ ID NO: 768 - DNA, 80 bp, Artificial sequence; identical to SEQ ID NO: 754
  molecule of the complex

SEQ ID NO: 769 - DNA, 80 bp, Artificial sequence; identical to SEQ ID NO: 755
  molecule of the complex

SEQ ID NO: 770 - DNA, 80 bp, Artificial sequence; identical to SEQ ID NO: 756
  molecule of the complex

SEQ ID NO: 771 - DNA, 94 bp, Artificial sequence
  molecule of the complex
agatctgatcaagagacagcagctgcagacaaagcttacagctgtctcttgatcagatctttttttttttttttttttttttcatatgccaagt

SEQ ID NO: 772 - DNA, 94 bp, Artificial sequence
  molecule of the complex
ctgtctcttgatcagatctcagctgcagacaaagcttacagagatctgatcaagagacagttttttttttttttttttttttcatatgccaagt

SEQ ID NO: 773 - DNA, 82 bp, Artificial sequence
  molecule of the complex
agatctgatcaagagacagcagctgcagacaaagcttacagctgtctcttgatcagatcttttttttttttttttttttttt

SEQ ID NO: 774 - DNA, 82 bp, Artificial sequence
  molecule of the complex
ctgtctcttgatcagatctcagctgcagacaaagcttacagagatctgatcaagagacagtttttttttttttttttttttt